SEQUENCE LISTING (262 sequences)

SEQ ID NO: 1 - protein, 118 aa, Artificial Sequence
  source: /note=“Description of Artificial Sequence Synthetic polypeptide”
QVQLQQPGSELVRPGASVKLSCKASGYTFTSYWMHWVKQRPGQGLEWIGNIYPRSGTTNYDGYFKSKATLTVDTSSSTAYMQLSSLTSEDSAVYFCTRSGYDYPFVYWGQGTLVTVSA

SEQ ID NO: 2 - protein, 109 aa, Artificial Sequence
  source: /note=“Description of Artificial Sequence Synthetic polypeptide”
QAVVTQESALTTSPGETVTLTCRTSTGAVSIRNYANWVQEKPDHLFTGLIGGTNNRAPGVPARFSGSLIGDKAALTITGAQPEDEAIYFCALLYSNYWVFGGGTKLTVL

SEQ ID NO: 3 - protein, 8 aa, Artificial Sequence
  source: /note=“Description of Artificial Sequence Synthetic peptide”
GYTFTSYW

SEQ ID NO: 4 - protein, 8 aa, Artificial Sequence
  source: /note=“Description of Artificial Sequence Synthetic peptide”
IYPRSGTT

SEQ ID NO: 5 - protein, 11 aa, Artificial Sequence
  source: /note=“Description of Artificial Sequence Synthetic peptide”
TRSGYDYPFVY

SEQ ID NO: 6 - protein, 9 aa, Artificial Sequence
  source: /note=“Description of Artificial Sequence Synthetic peptide”
TGAVSIRNY

SEQ ID NO: 7 - protein, 3 aa, Artificial Sequence
  source: /note=“Description of Artificial Sequence Synthetic peptide”
GTN

SEQ ID NO: 8 - protein, 9 aa, Artificial Sequence
  source: /note=“Description of Artificial Sequence Synthetic peptide”
ALLYSNYWV

SEQ ID NO: 9 - protein, 5 aa, Artificial Sequence
  source: /note=“Description of Artificial Sequence Synthetic peptide”
SYWMH

SEQ ID NO: 10 - protein, 17 aa, Artificial Sequence
  source: /note=“Description of Artificial Sequence Synthetic peptide”
NIYPRSGTTNYDGYFKS

SEQ ID NO: 11 - protein, 9 aa, Artificial Sequence
  source: /note=“Description of Artificial Sequence Synthetic peptide”
SGYDYPFVY

SEQ ID NO: 12 - protein, 14 aa, Artificial Sequence
  source: /note=“Description of Artificial Sequence Synthetic peptide”
RTSTGAVSIRNYAN

SEQ ID NO: 13 - protein, 7 aa, Artificial Sequence
  source: /note=“Description of Artificial Sequence Synthetic peptide”
GTNNRAP

SEQ ID NO: 14 - protein, 9 aa, Artificial Sequence
  source: /note=“Description of Artificial Sequence Synthetic peptide”
ALLYSNYWV

SEQ ID NO: 15 - protein, 7 aa, Artificial Sequence
  source: /note=“Description of Artificial Sequence Synthetic peptide”
GYTFTSY

SEQ ID NO: 16 - protein, 6 aa, Artificial Sequence
  source: /note=“Description of Artificial Sequence Synthetic peptide”
YPRSGT

SEQ ID NO: 17 - protein, 9 aa, Artificial Sequence
  source: /note=“Description of Artificial Sequence Synthetic peptide”
SGYDYPFVY

SEQ ID NO: 18 - protein, 14 aa, Artificial Sequence
  source: /note=“Description of Artificial Sequence Synthetic peptide”
RTSTGAVSIRNYAN

SEQ ID NO: 19 - protein, 7 aa, Artificial Sequence
  source: /note=“Description of Artificial Sequence Synthetic peptide”
GTNNRAP

SEQ ID NO: 20 - protein, 9 aa, Artificial Sequence
  source: /note=“Description of Artificial Sequence Synthetic peptide”
ALLYSNYWV

SEQ ID NO: 21 - DNA, 354 bp, Artificial Sequence
  source: /note=“Description of Artificial Sequence Synthetic polynucleotide”
caggtccaactgcagcaacctgggtctgaactggtgaggcctggagcttcagtgaagctgtcctgcaaggcttctggctacacattcaccagttactggatgcactgggtgaagcagaggcctgggcaaggccttgagtggattggaaatatttatcctcgtagtggtactactaactacgatgggtacttcaagagtaaagccacactgactgtagacacatcctccagcacggcctacatgcagctcagtagcctgacatctgaggactctgcggtctatttctgtacaagatcgggatatgattacccttttgtttactggggccagggtactctggtcactgtctctgca

SEQ ID NO: 22 - DNA, 327 bp, Artificial Sequence
  source: /note=“Description of Artificial Sequence Synthetic polynucleotide”
caggctgttgtgactcaggaatctgcactcaccacatcacctggtgaaacagtcacactcacttgtcgcacaagtactggggctgtttcaattagaaactatgccaactgggtccaagaaaaaccagatcatttattcactggtctaataggtggtaccaacaaccgagctccaggtgttcctgccagattctcaggctccctgattggagacaaggctgccctcaccatcacaggggcacagcctgaggatgaggcgatatatttctgtgctctattatacagcaattattgggtgttcggtggaggaaccaaactgactgtccta

SEQ ID NO: 23 - protein, 119 aa, Artificial Sequence
  source: /note=“Description of Artificial Sequence Synthetic polypeptide”
EVQLVESGGGLVKPGGSLKLSCAASGFTFSSYAMSWVRQSPERRLEWVAEISSGGSYTYYPDTVTGRFTISRDNAKNTLYLEMSSLRSEDTAMYYCARTVGEDWYFDVWGAGTTVTVSS

SEQ ID NO: 24 - protein, 107 aa, Artificial Sequence
  source: /note=“Description of Artificial Sequence Synthetic polypeptide”
DIQMTQTTSSLSASLGDRVTISCRASQDISHYLNWYQQKPDGAVKLLIYSTSRLHSGVPSRFSGSGSGTDYSLTISNLEQEDIATYFCQQGYTLPFTFGSGTKLEIK

SEQ ID NO: 25 - protein, 8 aa, Artificial Sequence
  source: /note=“Description of Artificial Sequence Synthetic peptide”
GFTFSSYA

SEQ ID NO: 26 - protein, 8 aa, Artificial Sequence
  source: /note=“Description of Artificial Sequence Synthetic peptide”
ISSGGSYT

SEQ ID NO: 27 - protein, 12 aa, Artificial Sequence
  source: /note=“Description of Artificial Sequence Synthetic peptide”
ARTVGEDWYFDV

SEQ ID NO: 28 - protein, 6 aa, Artificial Sequence
  source: /note=“Description of Artificial Sequence Synthetic peptide”
QDISHY

SEQ ID NO: 29 - protein, 3 aa, Artificial Sequence
  source: /note=“Description of Artificial Sequence Synthetic peptide”
STS

SEQ ID NO: 30 - protein, 9 aa, Artificial Sequence
  source: /note=“Description of Artificial Sequence Synthetic peptide”
QQGYTLPFT

SEQ ID NO: 31 - protein, 5 aa, Artificial Sequence
  source: /note=“Description of Artificial Sequence Synthetic peptide”
SYAMS

SEQ ID NO: 32 - protein, 17 aa, Artificial Sequence
  source: /note=“Description of Artificial Sequence Synthetic peptide”
EISSGGSYTYYPDTVTG

SEQ ID NO: 33 - protein, 10 aa, Artificial Sequence
  source: /note=“Description of Artificial Sequence Synthetic peptide”
TVGEDWYFDV

SEQ ID NO: 34 - protein, 11 aa, Artificial Sequence
  source: /note=“Description of Artificial Sequence Synthetic peptide”
RASQDISHYLN

SEQ ID NO: 35 - protein, 7 aa, Artificial Sequence
  source: /note=“Description of Artificial Sequence Synthetic peptide”
STSRLHS

SEQ ID NO: 36 - protein, 9 aa, Artificial Sequence
  source: /note=“Description of Artificial Sequence Synthetic peptide”
QQGYTLPFT

SEQ ID NO: 37 - protein, 7 aa, Artificial Sequence
  source: /note=“Description of Artificial Sequence Synthetic peptide”
GFTFSSY

SEQ ID NO: 38 - protein, 6 aa, Artificial Sequence
  source: /note=“Description of Artificial Sequence Synthetic peptide”
SSGGSY

SEQ ID NO: 39 - protein, 10 aa, Artificial Sequence
  source: /note=“Description of Artificial Sequence Synthetic peptide”
TVGEDWYFDV

SEQ ID NO: 40 - protein, 11 aa, Artificial Sequence
  source: /note=“Description of Artificial Sequence Synthetic peptide”
RASQDISHYLN

SEQ ID NO: 41 - protein, 7 aa, Artificial Sequence
  source: /note=“Description of Artificial Sequence Synthetic peptide”
STSRLHS

SEQ ID NO: 42 - protein, 9 aa, Artificial Sequence
  source: /note=“Description of Artificial Sequence Synthetic peptide”
QQGYTLPFT

SEQ ID NO: 43 - DNA, 357 bp, Artificial Sequence
  source: /note=“Description of Artificial Sequence Synthetic polynucleotide”
gaagtgcagctggtggagtctgggggaggcttagtgaagcctggagggtccctgaaactctcctgtgcagcctctggattcactttcagtagctatgccatgtcttgggttcgccagtctccagagaggaggctggagtgggtcgcagaaattagtagtggtggtagttatacctactatccagacactgtgacgggccgattcaccatctccagagacaatgccaagaacaccctgtacctggaaatgagcagtctgaggtctgaggacacggccatgtattactgtgcaaggacagtaggtgaggactggtacttcgatgtctggggcgcagggaccacggtcaccgtctcctca

SEQ ID NO: 44 - DNA, 321 bp, Artificial Sequence
  source: /note=“Description of Artificial Sequence Synthetic polynucleotide”
gatatccagatgacacagactacatcctccctgtctgcctctctgggagacagagtcaccatcagttgcagggcaagtcaggacattagtcattatttaaactggtatcagcagaaaccagatggagctgttaaactcctgatctactccacatcaagattacactcaggagtcccatcaaggttcagtggcagtgggtctggaacagattattctctcaccattagcaacctggagcaagaagatattgccacttacttttgccaacagggttatacgcttccattcacgttcggctcggggacaaaattggaaataaaa

SEQ ID NO: 45 - protein, 117 aa, Artificial Sequence
  source: /note=“Description of Artificial Sequence Synthetic polypeptide”
DVQLVESGGGLVQPGGSRKLSCAASGFTFSSFGMHWVRQAPEKGLEWVAYISSGSSTIYYADTVKGRFTISRDNPKNTLFLQMTSLRSEDTAMYYCARGSYRAMDYWGQGTSVTVSS

SEQ ID NO: 46 - protein, 106 aa, Artificial Sequence
  source: /note=“Description of Artificial Sequence Synthetic polypeptide”
QIVLTQSPALMSASPGEKVTMTCSASSSVNYMFWYQQKPRSSPKPWIYLTSNLASGVPARFSGSGSGTSYSLTISSMEAEDAATYYCQLWSSNPFTFGSGTKLEIK

SEQ ID NO: 47 - protein, 8 aa, Artificial Sequence
  source: /note=“Description of Artificial Sequence Synthetic peptide”
GFTFSSFG

SEQ ID NO: 48 - protein, 8 aa, Artificial Sequence
  source: /note=“Description of Artificial Sequence Synthetic peptide”
ISSGSSTI

SEQ ID NO: 49 - protein, 10 aa, Artificial Sequence
  source: /note=“Description of Artificial Sequence Synthetic peptide”
ARGSYRAMDY

SEQ ID NO: 50 - protein, 5 aa, Artificial Sequence
  source: /note=“Description of Artificial Sequence Synthetic peptide”
SSVNY

SEQ ID NO: 51 - protein, 3 aa, Artificial Sequence
  source: /note=“Description of Artificial Sequence Synthetic peptide”
LTS

SEQ ID NO: 52 - protein, 9 aa, Artificial Sequence
  source: /note=“Description of Artificial Sequence Synthetic peptide”
QLWSSNPFT

SEQ ID NO: 53 - protein, 5 aa, Artificial Sequence
  source: /note=“Description of Artificial Sequence Synthetic peptide”
SFGMH

SEQ ID NO: 54 - protein, 17 aa, Artificial Sequence
  source: /note=“Description of Artificial Sequence Synthetic peptide”
YISSGSSTIYYADTVKG

SEQ ID NO: 55 - protein, 8 aa, Artificial Sequence
  source: /note=“Description of Artificial Sequence Synthetic peptide”
GSYRAMDY

SEQ ID NO: 56 - protein, 10 aa, Artificial Sequence
  source: /note=“Description of Artificial Sequence Synthetic peptide”
SASSSVNYMF

SEQ ID NO: 57 - protein, 7 aa, Artificial Sequence
  source: /note=“Description of Artificial Sequence Synthetic peptide”
LTSNLAS

SEQ ID NO: 58 - protein, 9 aa, Artificial Sequence
  source: /note=“Description of Artificial Sequence Synthetic peptide”
QLWSSNPFT

SEQ ID NO: 59 - protein, 7 aa, Artificial Sequence
  source: /note=“Description of Artificial Sequence Synthetic peptide”
GFTFSSF

SEQ ID NO: 60 - protein, 6 aa, Artificial Sequence
  source: /note=“Description of Artificial Sequence Synthetic peptide”
SSGSST

SEQ ID NO: 61 - protein, 8 aa, Artificial Sequence
  source: /note=“Description of Artificial Sequence Synthetic peptide”
GSYRAMDY

SEQ ID NO: 62 - protein, 10 aa, Artificial Sequence
  source: /note=“Description of Artificial Sequence Synthetic peptide”
SASSSVNYMF

SEQ ID NO: 63 - protein, 7 aa, Artificial Sequence
  source: /note=“Description of Artificial Sequence Synthetic peptide”
LTSNLAS

SEQ ID NO: 64 - protein, 9 aa, Artificial Sequence
  source: /note=“Description of Artificial Sequence Synthetic peptide”
QLWSSNPFT

SEQ ID NO: 65 - DNA, 351 bp, Artificial Sequence
  source: /note=“Description of Artificial Sequence Synthetic polynucleotide”
gatgtgcagctggtggagtctgggggaggcttagtgcagcctggagggtcccggaaactctcctgtgcagcctctggattcactttcagtagctttggaatgcactgggttcgtcaggctccagagaaggggctggagtgggtcgcatacattagtagtggcagtagtaccatctactatgcagacacagtgaagggccgattcaccatctccagagacaatcccaagaacaccctgttcctgcaaatgaccagtctaaggtctgaggacacggccatgtattactgtgcaagagggagctacagggctatggactactggggtcaaggaacctcagtcaccgtctcctca

SEQ ID NO: 66 - DNA, 318 bp, Artificial Sequence
  source: /note=“Description of Artificial Sequence Synthetic polynucleotide”
caaattgttctcacccagtctccagcactcatgtctgcatctccaggggagaaggtcaccatgacctgcagtgccagctcaagtgtaaattacatgttctggtaccagcagaagccaagatcctcccccaaaccctggatttatctcacatccaacctggcttctggagtccctgctcgcttcagtggcagtgggtctgggacctcttactctctcacaatcagcagcatggaggctgaagatgctgccacttattactgccagctgtggagtagtaacccattcacgttcggctcggggacaaagttggaaataaaa

SEQ ID NO: 67 - protein, 125 aa, Artificial Sequence
  source: /note=“Description of Artificial Sequence Synthetic polypeptide”
DVQLVESGGGLVQPGGSRKLSCAASGFTFSSFGIHWVRQAPEKGLEWVAYISSGSSTIYYADTVKGRFTISRDNPKNTLFLQMTSLRSEDTAMYYCARGSKVVAKSRGYWYFDVWGAGTTVTVSS

SEQ ID NO: 68 - protein, 112 aa, Artificial Sequence
  source: /note=“Description of Artificial Sequence Synthetic polypeptide”
DVVVTQTPLFLPVSFGDQVSISCRSSQSLANNYGITYLSWYLHRPGQSPQLLIYGISNRFSGVPDRFSGSGSGTDFTLKISTIKPEDLGMYYCLQGTHQPWTFGGGTKLEIK

SEQ ID NO: 69 - protein, 8 aa, Artificial Sequence
  source: /note=“Description of Artificial Sequence Synthetic peptide”
GFTFSSFG

SEQ ID NO: 70 - protein, 8 aa, Artificial Sequence
  source: /note=“Description of Artificial Sequence Synthetic peptide”
ISSGSSTI

SEQ ID NO: 71 - protein, 18 aa, Artificial Sequence
  source: /note=“Description of Artificial Sequence Synthetic peptide”
ARGSKVVAKSRGYWYFDV

SEQ ID NO: 72 - protein, 11 aa, Artificial Sequence
  source: /note=“Description of Artificial Sequence Synthetic peptide”
QSLANNYGITY

SEQ ID NO: 73 - protein, 3 aa, Artificial Sequence
  source: /note=“Description of Artificial Sequence Synthetic peptide”
GIS

SEQ ID NO: 74 - protein, 9 aa, Artificial Sequence
  source: /note=“Description of Artificial Sequence Synthetic peptide”
LQGTHQPWT

SEQ ID NO: 75 - protein, 5 aa, Artificial Sequence
  source: /note=“Description of Artificial Sequence Synthetic peptide”
SFGIH

SEQ ID NO: 76 - protein, 17 aa, Artificial Sequence
  source: /note=“Description of Artificial Sequence Synthetic peptide”
YISSGSSTIYYADTVKG

SEQ ID NO: 77 - protein, 16 aa, Artificial Sequence
  source: /note=“Description of Artificial Sequence Synthetic peptide”
GSKVVAKSRGYWYFDV

SEQ ID NO: 78 - protein, 16 aa, Artificial Sequence
  source: /note=“Description of Artificial Sequence Synthetic peptide”
RSSQSLANNYGITYLS

SEQ ID NO: 79 - protein, 7 aa, Artificial Sequence
  source: /note=“Description of Artificial Sequence Synthetic peptide”
GISNRFS

SEQ ID NO: 80 - protein, 9 aa, Artificial Sequence
  source: /note=“Description of Artificial Sequence Synthetic peptide”
LQGTHQPWT

SEQ ID NO: 81 - protein, 7 aa, Artificial Sequence
  source: /note=“Description of Artificial Sequence Synthetic peptide”
GFTFSSF

SEQ ID NO: 82 - protein, 6 aa, Artificial Sequence
  source: /note=“Description of Artificial Sequence Synthetic peptide”
SSGSST

SEQ ID NO: 83 - protein, 16 aa, Artificial Sequence
  source: /note=“Description of Artificial Sequence Synthetic peptide”
GSKVVAKSRGYWYFDV

SEQ ID NO: 84 - protein, 16 aa, Artificial Sequence
  source: /note=“Description of Artificial Sequence Synthetic peptide”
RSSQSLANNYGITYLS

SEQ ID NO: 85 - protein, 7 aa, Artificial Sequence
  source: /note=“Description of Artificial Sequence Synthetic peptide”
GISNRFS

SEQ ID NO: 86 - protein, 9 aa, Artificial Sequence
  source: /note=“Description of Artificial Sequence Synthetic peptide”
LQGTHQPWT

SEQ ID NO: 87 - DNA, 375 bp, Artificial Sequence
  source: /note=“Description of Artificial Sequence Synthetic polynucleotide”
gatgtgcagctggtggagtctgggggaggcttagtgcagcctggagggtcccggaaactctcctgtgcagcctctggattcactttcagtagttttggaattcactgggttcgtcaggctccagagaaggggctggagtgggtcgcatacataagtagtggcagtagtaccatctactatgcagacacagtgaagggccgattcaccatctccagagacaatcccaagaacaccctgttcctgcaaatgaccagtctaaggtctgaggacacggccatgtattactgtgcaagagggtctaaggtagtagctaagtcaaggggctactggtacttcgatgtctggggcgcagggaccacggtcaccgtctcctca

SEQ ID NO: 88 - DNA, 335 bp, Artificial Sequence
  source: /note=“Description of Artificial Sequence Synthetic polynucleotide”
gatgttgtggtgactcaaactccactcttcctgcctgtcagctttggagatcaagtttctatctcttgcaggtctagtcagagtcttgaaacaattatgggatcacctatttgtcttggtacctgcacaggcctggccagtctccccagctcctcatctatgggatttccaacagattttctggggtgccagacaggttcagtggcagtggttcagggacagatttcacactcaagatcagcacaataaagcctgaggacttgggaatgtattactgcttacaaggtacacatcagccgtggacgttcggtggaggcaccaagctggaaatcaaa

SEQ ID NO: 89 - protein, 8 aa, Artificial Sequence
  source: /note=“Description of Artificial Sequence Synthetic peptide”
  VARIANT (2)..(2): /replace=“Phe”
  VARIANT (5)..(5): /replace=“Ser”
  VARIANT (7)..(7): /replace=“Phe”
  VARIANT (8)..(8): /replace=“Ala” or “Gly”
  SITE (1)..(8): /note=“Variant residues given in the sequence have no preference with respect to those in the annotations for variant positions”
GYTFTSYW

SEQ ID NO: 90 - protein, 7 aa, Artificial Sequence
  source: /note=“Description of Artificial Sequence Synthetic peptide”
  VARIANT (1)..(1): /replace=“Ser”
  VARIANT (2)..(2): /replace=“Ser”
  VARIANT (3)..(3): /replace=“Gly”
  VARIANT (4)..(4): /replace=“Gly”
  VARIANT (5)..(5): /replace=“Ser”
  VARIANT (6)..(6): /replace=“Tyr”
  VARIANT (7)..(7): /replace=“Ile”
  SITE (1)..(7): /note=“Variant residues given in the sequence have no preference with respect to those in the annotations for variant positions”
YPRSGTT

SEQ ID NO: 91 - protein, 3 aa, Artificial Sequence
  source: /note=“Description of Artificial Sequence Synthetic peptide”
  VARIANT (1)..(1): /replace=“Ser” or “Leu”
  VARIANT (2)..(2): /replace=“Ile”
  VARIANT (3)..(3): /replace=“Ser”
  SITE (1)..(3): /note=“Variant residues given in the sequence have no preference with respect to those in the annotations for variant positions”
GTN

SEQ ID NO: 92 - protein, 9 aa, Artificial Sequence
  source: /note=“Description of Artificial Sequence Synthetic peptide”
  VARIANT (1)..(1): /replace=“Gln” or “Leu”
  VARIANT (2)..(2): /replace=“Gln”
  VARIANT (3)..(3): /replace=“Gly” or “Trp”
  VARIANT (4)..(4): /replace=“Ser” or “Thr”
  VARIANT (5)..(5): /replace=“Thr” or “His”
  VARIANT (6)..(6): /replace=“Leu” or “Gln”
  VARIANT (7)..(7): /replace=“Pro”
  VARIANT (8)..(8): /replace=“Phe”
  VARIANT (9)..(9): /replace=“Thr”
  SITE (1)..(9): /note=“Variant residues given in the sequence have no preference with respect to those in the annotations for variant positions”
ALLYSNYWY

SEQ ID NO: 93 - protein, 5 aa, Artificial Sequence
  source: /note=“Description of Artificial Sequence Synthetic peptide”
  VARIANT (2)..(2): /replace=“Phe”
  VARIANT (3)..(3): /replace=“Ala” or “Gly”
  VARIANT (4)..(4): /replace=“Ile”
  VARIANT (5)..(5): /replace=“Ser”
  SITE (1)..(5): /note=“Variant residues given in the sequence have no preference with respect to those in the annotations for variant positions”
SYWMH

SEQ ID NO: 94 - protein, 17 aa, Artificial Sequence
  source: /note=“Description of Artificial Sequence Synthetic peptide”
  VARIANT (1)..(1): /replace=“Glu” or “Tyr”
  VARIANT (3)..(3): /replace=“Ser”
  VARIANT (4)..(4): /replace=“Ser”
  VARIANT (5)..(5): /replace=“Gly”
  VARIANT (6)..(6): /replace=“Gly”
  VARIANT (7)..(7): /replace=“Ser”
  VARIANT (8)..(8): /replace=“Tyr”
  VARIANT (9)..(9): /replace=“Ile”
  VARIANT (10)..(10): /replace=“Tyr”
  VARIANT (12)..(12): /replace=“Pro” or “Ala”
  VARIANT (13)..(13): /replace=“Asp”
  VARIANT (14)..(14): /replace=“Thr”
  VARIANT (15)..(15): /replace=“Val”
  VARIANT (16)..(16): /replace=“Thr”
  VARIANT (17)..(17): /replace=“Gly”
  SITE (1)..(17): /note=“Variant residues given in the sequence have no preference with respect to those in the annotations for variant positions”
NIYPRSGTTNYDGYFKS

SEQ ID NO: 95 - protein, 7 aa, Artificial Sequence
  source: /note=“Description of Artificial Sequence Synthetic peptide”
  VARIANT (1)..(1): /replace=“Ser” or “Leu”
  VARIANT (2)..(2): /replace=“Ile”
  VARIANT (3)..(3): /replace=“Ser”
  VARIANT (4)..(4): /replace=“Arg”
  VARIANT (5)..(5): /replace=“Leu”
  VARIANT (6)..(6): /replace=“His” or “Phe”
  VARIANT (7)..(7): /replace=“Ser”
  SITE (1)..(7): /note=“Variant residues given in the sequence have no preference with respect to those in the annotations for variant positions”
GTNNRAP

SEQ ID NO: 96 - protein, 9 aa, Artificial Sequence
  source: /note=“Description of Artificial Sequence Synthetic peptide”
  VARIANT (1)..(1): /replace=“Gln” or “Leu”
  VARIANT (2)..(2): /replace=“Gln”
  VARIANT (3)..(3): /replace=“Gly” or “Trp”
  VARIANT (4)..(4): /replace=“Ser” or “Thr”
  VARIANT (5)..(5): /replace=“Thr” or “His”
  VARIANT (6)..(6): /replace=“Leu” or “Gln”
  VARIANT (7)..(7): /replace=“Pro”
  VARIANT (8)..(8): /replace=“Phe”
  VARIANT (9)..(9): /replace=“Thr”
  SITE (1)..(9): /note=“Variant residues given in the sequence have no preference with respect to those in the annotations for variant positions”
ALLYSNYWY

SEQ ID NO: 97 - protein, 7 aa, Artificial Sequence
  source: /note=“Description of Artificial Sequence Synthetic peptide”
  VARIANT (2)..(2): /replace=“Phe”
  VARIANT (5)..(5): /replace=“Ser”
  VARIANT (7)..(7): /replace=“Phe”
  SITE (1)..(7): /note=“Variant residues given in the sequence have no preference with respect to those in the annotations for variant positions”
GYTFTSY

SEQ ID NO: 98 - protein, 6 aa, Artificial Sequence
  source: /note=“Description of Artificial Sequence Synthetic peptide”
  VARIANT (1)..(1): /replace=“Ser”
  VARIANT (2)..(2): /replace=“Ser”
  VARIANT (3)..(3): /replace=“Gly”
  VARIANT (4)..(4): /replace=“Gly”
  VARIANT (5)..(5): /replace=“Ser”
  VARIANT (6)..(6): /replace=“Tyr”
  SITE (1)..(6): /note=“Variant residues given in the sequence have no preference with respect to those in the annotations for variant positions”
YPRSGT

SEQ ID NO: 99 - protein, 7 aa, Artificial Sequence
  source: /note=“Description of Artificial Sequence Synthetic peptide”
  VARIANT (1)..(1): /replace=“Ser” or “Leu”
  VARIANT (2)..(2): /replace=“Ile”
  VARIANT (3)..(3): /replace=“Ser”
  VARIANT (4)..(4): /replace=“Arg”
  VARIANT (5)..(5): /replace=“Leu”
  VARIANT (6)..(6): /replace=“His” or “Phe”
  VARIANT (7)..(7): /replace=“Ser”
  SITE (1)..(7): /note=“Variant residues given in the sequence have no preference with respect to those in the annotations for variant positions”
GTNNRAP

SEQ ID NO: 100 - protein, 9 aa, Artificial Sequence
  source: /note=“Description of Artificial Sequence Synthetic peptide”
  VARIANT (1)..(1): /replace=“Gln” or “Leu”
  VARIANT (2)..(2): /replace=“Gln”
  VARIANT (3)..(3): /replace=“Gly” or “Trp”
  VARIANT (4)..(4): /replace=“Ser” or “Thr”
  VARIANT (5)..(5): /replace=“Thr” or “His”
  VARIANT (6)..(6): /replace=“Leu” or “Gln”
  VARIANT (7)..(7): /replace=“Pro”
  VARIANT (8)..(8): /replace=“Phe”
  VARIANT (9)..(9): /replace=“Thr”
  SITE (1)..(9): /note=“Variant residues given in the sequence have no preference with respect to those in the annotations for variant positions”
ALLYSNYWY

SEQ ID NO: 101 - protein, 10 aa, Artificial Sequence
  source: /note=“Description of Artificial Sequence Synthetic peptide”
GYTFTSYWMH

SEQ ID NO: 102 - protein, 17 aa, Artificial Sequence
  source: /note=“Description of Artificial Sequence Synthetic peptide”
NIYPRSGTTNYDGYFKS

SEQ ID NO: 103 - protein, 11 aa, Artificial Sequence
  source: /note=“Description of Artificial Sequence Synthetic peptide”
TRSGYDYPFVY

SEQ ID NO: 104 - protein, 14 aa, Artificial Sequence
  source: /note=“Description of Artificial Sequence Synthetic peptide”
RTSTGAVSIRNYAN

SEQ ID NO: 105 - protein, 7 aa, Artificial Sequence
  source: /note=“Description of Artificial Sequence Synthetic peptide”
GTNNRAP

SEQ ID NO: 106 - protein, 9 aa, Artificial Sequence
  source: /note=“Description of Artificial Sequence Synthetic peptide”
ALLYSNYWV

SEQ ID NO: 107 - protein, 10 aa, Artificial Sequence
  source: /note=“Description of Artificial Sequence Synthetic peptide”
GFTFSSYAMS

SEQ ID NO: 108 - protein, 17 aa, Artificial Sequence
  source: /note=“Description of Artificial Sequence Synthetic peptide”
EISSGGSYTYYPDTVTG

SEQ ID NO: 109 - protein, 12 aa, Artificial Sequence
  source: /note=“Description of Artificial Sequence Synthetic peptide”
ARTVGEDWYFDV

SEQ ID NO: 110 - protein, 11 aa, Artificial Sequence
  source: /note=“Description of Artificial Sequence Synthetic peptide”
RASQDISHYLN

SEQ ID NO: 111 - protein, 7 aa, Artificial Sequence
  source: /note=“Description of Artificial Sequence Synthetic peptide”
STSRLHS

SEQ ID NO: 112 - protein, 9 aa, Artificial Sequence
  source: /note=“Description of Artificial Sequence Synthetic peptide”
QQGYTLPFT

SEQ ID NO: 113 - protein, 10 aa, Artificial Sequence
  source: /note=“Description of Artificial Sequence Synthetic peptide”
GFTFSSFGMH

SEQ ID NO: 114 - protein, 17 aa, Artificial Sequence
  source: /note=“Description of Artificial Sequence Synthetic peptide”
YISSGSSTIYYADTVKG

SEQ ID NO: 115 - protein, 10 aa, Artificial Sequence
  source: /note=“Description of Artificial Sequence Synthetic peptide”
ARGSYRAMDY

SEQ ID NO: 116 - protein, 10 aa, Artificial Sequence
  source: /note=“Description of Artificial Sequence Synthetic peptide”
SASSSVNYMF

SEQ ID NO: 117 - protein, 7 aa, Artificial Sequence
  source: /note=“Description of Artificial Sequence Synthetic peptide”
LTSNLAS

SEQ ID NO: 118 - protein, 9 aa, Artificial Sequence
  source: /note=“Description of Artificial Sequence Synthetic peptide”
QLWSSNPFT

SEQ ID NO: 119 - protein, 10 aa, Artificial Sequence
  source: /note=“Description of Artificial Sequence Synthetic peptide”
GFTFSSFGIH

SEQ ID NO: 120 - protein, 17 aa, Artificial Sequence
  source: /note=“Description of Artificial Sequence Synthetic peptide”
YISSGSSTIYYADTVKG

SEQ ID NO: 121 - protein, 18 aa, Artificial Sequence
  source: /note=“Description of Artificial Sequence Synthetic peptide”
ARGSKVVAKSRGYWYFDV

SEQ ID NO: 122 - protein, 16 aa, Artificial Sequence
  source: /note=“Description of Artificial Sequence Synthetic peptide”
RSSQSLANNYGITYLS

SEQ ID NO: 123 - protein, 7 aa, Artificial Sequence
  source: /note=“Description of Artificial Sequence Synthetic peptide”
GISNRFS

SEQ ID NO: 124 - protein, 9 aa, Artificial Sequence
  source: /note=“Description of Artificial Sequence Synthetic peptide”
LQGTHQPWT

SEQ ID NO: 125 - protein, 10 aa, Artificial Sequence
  source: /note=“Description of Artificial Sequence Synthetic peptide”
  VARIANT (2)..(2): /replace=“Phe”
  VARIANT (5)..(5): /replace=“Ser”
  VARIANT (7)..(7): /replace=“Phe”
  VARIANT (8)..(8): /replace=“Ala” or “Gly”
  VARIANT (9)..(9): /replace=“Ile”
  VARIANT (10)..(10): /replace=“Ser”
  SITE (1)..(10): /note=“Variant residues given in the sequence have no preference with respect to those in the annotations for variant positions”
GYTFTSYWMH

SEQ ID NO: 126 - protein, 17 aa, Artificial Sequence
  source: /note=“Description of Artificial Sequence Synthetic peptide”
  VARIANT (1)..(1): /replace=“Glu” or “Tyr”
  VARIANT (3)..(3): /replace=“Ser”
  VARIANT (4)..(4): /replace=“Ser”
  VARIANT (5)..(5): /replace=“Gly”
  VARIANT (6)..(6): /replace=“Gly”
  VARIANT (7)..(7): /replace=“Ser”
  VARIANT (8)..(8): /replace=“Tyr”
  VARIANT (9)..(9): /replace=“Ile”
  VARIANT (10)..(10): /replace=“Tyr”
  VARIANT (12)..(12): /replace=“Pro” or “Ala”
  VARIANT (13)..(13): /replace=“Asp”
  VARIANT (14)..(14): /replace=“Thr”
  VARIANT (15)..(15): /replace=“Val”
  VARIANT (16)..(16): /replace=“Lys”
  VARIANT (17)..(17): /replace=“Gly”
  SITE (1)..(17): /note=“Variant residues given in the sequence have no preference with respect to those in the annotations for variant positions”
NIYPRSGTTNYDGYFTS

SEQ ID NO: 127 - protein, 7 aa, Artificial Sequence
  source: /note=“Description of Artificial Sequence Synthetic peptide”
  VARIANT (1)..(1): /replace=“Ser” or “Leu”
  VARIANT (2)..(2): /replace=“Ile”
  VARIANT (3)..(3): /replace=“Ser”
  VARIANT (4)..(4): /replace=“Arg”
  VARIANT (5)..(5): /replace=“Leu”
  VARIANT (6)..(6): /replace=“His” or “Phe”
  VARIANT (7)..(7): /replace=“Ser”
  SITE (1)..(7): /note=“Variant residues given in the sequence have no preference with respect to those in the annotations for variant positions”
GTNNRAP

SEQ ID NO: 128 - protein, 9 aa, Artificial Sequence
  source: /note=“Description of Artificial Sequence Synthetic peptide”
  VARIANT (1)..(1): /replace=“Gln” or “Leu”
  VARIANT (2)..(2): /replace=“Gln”
  VARIANT (3)..(3): /replace=“Gly” or “Trp”
  VARIANT (4)..(4): /replace=“Ser” or “Thr”
  VARIANT (5)..(5): /replace=“Thr” or “His”
  VARIANT (6)..(6): /replace=“Leu” or “Gln”
  VARIANT (7)..(7): /replace=“Pro”
  VARIANT (8)..(8): /replace=“Phe”
  VARIANT (9)..(9): /replace=“Thr”
  SITE (1)..(9): /note=“Variant residues given in the sequence have no preference with respect to those in the annotations for variant positions”
ALLYSNYWY

SEQ ID NO: 129 - protein, 2 aa, Artificial Sequence
  source: /note=“Description of Artificial Sequence Synthetic peptide”
SY

SEQ ID NO: 130 - protein, 6 aa, Artificial Sequence
  source: /note=“Description of Artificial Sequence Synthetic peptide”
YPRSGT

SEQ ID NO: 131 - protein, 9 aa, Artificial Sequence
  source: /note=“Description of Artificial Sequence Synthetic peptide”
SGYDYPFVY

SEQ ID NO: 132 - protein, 9 aa, Artificial Sequence
  source: /note=“Description of Artificial Sequence Synthetic peptide”
TGAVSIRNY

SEQ ID NO: 133 - protein, 3 aa, Artificial Sequence
  source: /note=“Description of Artificial Sequence Synthetic peptide”
GTN

SEQ ID NO: 134 - protein, 9 aa, Artificial Sequence
  source: /note=“Description of Artificial Sequence Synthetic peptide”
ALLYSNYWV

SEQ ID NO: 135 - protein, 2 aa, Artificial Sequence
  source: /note=“Description of Artificial Sequence Synthetic peptide”
SY

SEQ ID NO: 136 - protein, 6 aa, Artificial Sequence
  source: /note=“Description of Artificial Sequence Synthetic peptide”
SSGGSY

SEQ ID NO: 137 - protein, 10 aa, Artificial Sequence
  source: /note=“Description of Artificial Sequence Synthetic peptide”
TVGEDWYFDV

SEQ ID NO: 138 - protein, 6 aa, Artificial Sequence
  source: /note=“Description of Artificial Sequence Synthetic peptide”
QDISHY

SEQ ID NO: 139 - protein, 3 aa, Artificial Sequence
  source: /note=“Description of Artificial Sequence Synthetic peptide”
STS

SEQ ID NO: 140 - protein, 9 aa, Artificial Sequence
  source: /note=“Description of Artificial Sequence Synthetic peptide”
QQGYTLPFT

SEQ ID NO: 141 - protein, 2 aa, Artificial Sequence
  source: /note=“Description of Artificial Sequence Synthetic peptide”
SF

SEQ ID NO: 142 - protein, 6 aa, Artificial Sequence
  source: /note=“Description of Artificial Sequence Synthetic peptide”
SSGSST

SEQ ID NO: 143 - protein, 8 aa, Artificial Sequence
  source: /note=“Description of Artificial Sequence Synthetic peptide”
GSYRAMDY

SEQ ID NO: 144 - protein, 5 aa, Artificial Sequence
  source: /note=“Description of Artificial Sequence Synthetic peptide”
SSVNY

SEQ ID NO: 145 - protein, 3 aa, Artificial Sequence
  source: /note=“Description of Artificial Sequence Synthetic peptide”
LTS

SEQ ID NO: 146 - protein, 9 aa, Artificial Sequence
  source: /note=“Description of Artificial Sequence Synthetic peptide”
QLWSSNPFT

SEQ ID NO: 147 - protein, 2 aa, Artificial Sequence
  source: /note=“Description of Artificial Sequence Synthetic peptide”
SF

SEQ ID NO: 148 - protein, 6 aa, Artificial Sequence
  source: /note=“Description of Artificial Sequence Synthetic peptide”
SSGSST

SEQ ID NO: 149 - protein, 16 aa, Artificial Sequence
  source: /note=“Description of Artificial Sequence Synthetic peptide”
GSKVVAKSRGYWYFDV

SEQ ID NO: 150 - protein, 11 aa, Artificial Sequence
  source: /note=“Description of Artificial Sequence Synthetic peptide”
QSLANNYGITY

SEQ ID NO: 151 - protein, 3 aa, Artificial Sequence
  source: /note=“Description of Artificial Sequence Synthetic peptide”
GIS

SEQ ID NO: 152 - protein, 9 aa, Artificial Sequence
  source: /note=“Description of Artificial Sequence Synthetic peptide”
LQGTHQPWT

SEQ ID NO: 153 - protein, 2 aa, Artificial Sequence
  source: /note=“Description of Artificial Sequence Synthetic peptide”
  VARIANT (2)..(2): /replace=“Phe”
  SITE (1)..(2): /note=“Variant residues given in the sequence have no preference with respect to those in the annotations for variant positions”
SY

SEQ ID NO: 154 - protein, 6 aa, Artificial Sequence
  source: /note=“Description of Artificial Sequence Synthetic peptide”
  VARIANT (1)..(1): /replace=“Ser”
  VARIANT (2)..(2): /replace=“Ser”
  VARIANT (3)..(3): /replace=“Gly”
  VARIANT (4)..(4): /replace=“Gly”
  VARIANT (5)..(5): /replace=“Ser”
  VARIANT (6)..(6): /replace=“Tyr”
  SITE (1)..(6): /note=“Variant residues given in the sequence have no preference with respect to those in the annotations for variant positions”
YPRSGT

SEQ ID NO: 155 - protein, 3 aa, Artificial Sequence
  source: /note=“Description of Artificial Sequence Synthetic peptide”
  VARIANT (1)..(1): /replace=“Ser” or “Leu”
  VARIANT (2)..(2): /replace=“Ile”
  VARIANT (3)..(3): /replace=“Ser”
  SITE (1)..(3): /note=“Variant residues given in the sequence have no preference with respect to those in the annotations for variant positions”
GTN

SEQ ID NO: 156 - protein, 9 aa, Artificial Sequence
  source: /note=“Description of Artificial Sequence Synthetic peptide”
  VARIANT (1)..(1): /replace=“Gln” or “Leu”
  VARIANT (2)..(2): /replace=“Gln”
  VARIANT (3)..(3): /replace=“Gly” or “Trp”
  VARIANT (4)..(4): /replace=“Ser” or “Thr”
  VARIANT (5)..(5): /replace=“Thr” or “His”
  VARIANT (6)..(6): /replace=“Leu” or “Gln”
  VARIANT (7)..(7): /replace=“Pro”
  VARIANT (8)..(8): /replace=“Phe”
  VARIANT (9)..(9): /replace=“Thr”
  SITE (1)..(9): /note=“Variant residues given in the sequence have no preference with respect to those in the annotations for variant positions”
ALLYSNYWY

SEQ ID NO: 157 - protein, 493 aa, Artificial Sequence
  source: /note=“Description of Artificial Sequence Synthetic polypeptide”
MALPVTALLLPLALLLHAARPQAVVTQESALTTSPGETVTLTCRTSTGAVSIRNYANWVQEKPDHLFTGLIGGTNNRAPGVPARFSGSLIGDKAALTITGAQPEDEAIYFCALLYSNYWVFGGGTKLTVLGGGGSGGGGSGGGGSQVQLQQPGSELVRPGASVKLSCKASGYTFTSYWMHWVKQRPGQGLEWIGNIYPRSGTTNYDGYFKSKATLTVDTSSSTAYMQLSSLTSEDSAVYFCTRSGYDYPFVYWGQGTLVTVSATTTPAPRPPTPAPTIASPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRKRGSG

SEQ ID NO: 158 - protein, 461 aa, Artificial Sequence
  source: /note=“Description of Artificial Sequence Synthetic polypeptide”
MALPVTALLLPLALLLHAARPQAVVTQESALTTSPGETVTLTCRTSTGAVSIRNYANWVQEKPDHLFTGLIGGTNNRAPGVPARFSGSLIGDKAALTITGAQPEDEAIYFCALLYSNYWVFGGGTKLTVLGGGGSGGGGSGGGGSQVQLQQPGSELVRPGASVKLSCKASGYTFTSYWMHWVKQRPGQGLEWIGNIYPRSGTTNYDGYFKSKATLTVDTSSSTAYMQLSSLTSEDSAVYFCTRSGYDYPFVYWGQGTLVTVSAESKYGPPCPSCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRKRGSG

SEQ ID NO: 159 - protein, 678 aa, Artificial Sequence
  source: /note=“Description of Artificial Sequence Synthetic polypeptide”
MALPVTALLLPLALLLHAARPQAVVTQESALTTSPGETVTLTCRTSTGAVSIRNYANWVQEKPDHLFTGLIGGTNNRAPGVPARFSGSLIGDKAALTITGAQPEDEAIYFCALLYSNYWVFGGGTKLTVLGGGGSGGGGSGGGGSQVQLQQPGSELVRPGASVKLSCKASGYTFTSYWMHWVKQRPGQGLEWIGNIYPRSGTTNYDGYFKSKATLTVDTSSSTAYMQLSSLTSEDSAVYFCTRSGYDYPFVYWGQGTLVTVSAESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRKRGSG

SEQ ID NO: 160 - protein, 493 aa, Artificial Sequence
  source: /note=“Description of Artificial Sequence Synthetic polypeptide”
MALPVTALLLPLALLLHAARPQVQLQQPGSELVRPGASVKLSCKASGYTFTSYWMHWVKQRPGQGLEWIGNIYPRSGTTNYDGYFKSKATLTVDTSSSTAYMQLSSLTSEDSAVYFCTRSGYDYPFVYWGQGTLVTVSAGGGGSGGGGSGGGGSQAVVTQESALTTSPGETVTLTCRTSTGAVSIRNYANWVQEKPDHLFTGLIGGTNNRAPGVPARFSGSLIGDKAALTITGAQPEDEAIYFCALLYSNYWVFGGGTKLTVLTTTPAPRPPTPAPTIASPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRKRGSG

SEQ ID NO: 161 - protein, 461 aa, Artificial Sequence
  source: /note=“Description of Artificial Sequence Synthetic polypeptide”
MALPVTALLLPLALLLHAARPQVQLQQPGSELVRPGASVKLSCKASGYTFTSYWMHWVKQRPGQGLEWIGNIYPRSGTTNYDGYFKSKATLTVDTSSSTAYMQLSSLTSEDSAVYFCTRSGYDYPFVYWGQGTLVTVSAGGGGSGGGGSGGGGSQAVVTQESALTTSPGETVTLTCRTSTGAVSIRNYANWVQEKPDHLFTGLIGGTNNRAPGVPARFSGSLIGDKAALTITGAQPEDEAIYFCALLYSNYWVFGGGTKLTVLESKYGPPCPSCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRKRGSG

SEQ ID NO: 162 - protein, 678 aa, Artificial Sequence
  source: /note=“Description of Artificial Sequence Synthetic polypeptide”
MALPVTALLLPLALLLHAARPQVQLQQPGSELVRPGASVKLSCKASGYTFTSYWMHWVKQRPGQGLEWIGNIYPRSGTTNYDGYFKSKATLTVDTSSSTAYMQLSSLTSEDSAVYFCTRSGYDYPFVYWGQGTLVTVSAGGGGSGGGGSGGGGSQAVVTQESALTTSPGETVTLTCRTSTGAVSIRNYANWVQEKPDHLFTGLIGGTNNRAPGVPARFSGSLIGDKAALTITGAQPEDEAIYFCALLYSNYWVFGGGTKLTVLESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRKRGSG

SEQ ID NO: 163 - protein, 730 aa, Artificial Sequence
  source: /note=“Description of Artificial Sequence Synthetic polypeptide”
MALPVTALLLPLALLLHAARPQAVVTQESALTTSPGETVTLTCRTSTGAVSIRNYANWVQEKPDHLFTGLIGGTNNRAPGVPARFSGSLIGDKAALTITGAQPEDEAIYFCALLYSNYWVFGGGTKLTVLGGGGSQVQLQQPGSELVRPGASVKLSCKASGYTFTSYWMHWVKQRPGQGLEWIGNIYPRSGTTNYDGYFKSKATLTVDTSSSTAYMQLSSLTSEDSAVYFCTRSGYDYPFVYWGQGTLVTVSAGGGGSGGGGSGGGGSQAVVTQESALTTSPGETVTLTCRTSTGAVSIRNYANWVQEKPDHLFTGLIGGTNNRAPGVPARFSGSLIGDKAALTITGAQPEDEAIYFCALLYSNYWVFGGGTKLTVLGGGGSQVQLQQPGSELVRPGASVKLSCKASGYTFTSYWMHWVKQRPGQGLEWIGNIYPRSGTTNYDGYFKSKATLTVDTSSSTAYMQLSSLTSEDSAVYFCTRSGYDYPFVYWGQGTLVTVSATTTPAPRPPTPAPTIASPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRKRGSG

SEQ ID NO: 164 - protein, 730 aa, Artificial Sequence
  source: /note=“Description of Artificial Sequence Synthetic polypeptide”
MALPVTALLLPLALLLHAARPQVQLQQPGSELVRPGASVKLSCKASGYTFTSYWMHWVKQRPGQGLEWIGNIYPRSGTTNYDGYFKSKATLTVDTSSSTAYMQLSSLTSEDSAVYFCTRSGYDYPFVYWGQGTLVTVSAGGGGSQAVVTQESALTTSPGETVTLTCRTSTGAVSIRNYANWVQEKPDHLFTGLIGGTNNRAPGVPARFSGSLIGDKAALTITGAQPEDEAIYFCALLYSNYWVFGGGTKLTVLGGGGSGGGGSGGGGSQVQLQQPGSELVRPGASVKLSCKASGYTFTSYWMHWVKQRPGQGLEWIGNIYPRSGTTNYDGYFKSKATLTVDTSSSTAYMQLSSLTSEDSAVYFCTRSGYDYPFVYWGQGTLVTVSAGGGGSQAVVTQESALTTSPGETVTLTCRTSTGAVSIRNYANWVQEKPDHLFTGLIGGTNNRAPGVPARFSGSLIGDKAALTITGAQPEDEAIYFCALLYSNYWVFGGGTKLTVLTTTPAPRPPTPAPTIASPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRKRGSG

SEQ ID NO: 165 - protein, 19 aa, Artificial Sequence
  source: /note=“Description of Artificial Sequence Synthetic peptide”
  MOD_RES (5)..(5): Glycosylated Thr
  MOD_RES (12)..(12): Glycosylated Ser
GYRQXPKEDSHXTTGTAAA

SEQ ID NO: 166 - protein, 127 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQ

SEQ ID NO: 167 - DNA, 2955 bp, Artificial Sequence
  source: /note=“Description of Artificial Sequence Synthetic polynucleotide”
atggccctgcccgtgaccgccctgctgctgcccctggccctgctgctgcacgccgccaggccccaggccgtggtgacccaggagagcgccctgaccaccagccccggcgagaccgtgaccctgacctgcaggaccagcaccggcgccgtgagcatcaggaactacgccaactgggtgcaggagaagcccgaccacctgttcaccggcctgatcggcggcaccaacaacagggcccccggcgtgcccgccaggttcagcggcagcctgatcggcgacaaggccgccctgaccatcaccggcgcccagcccgaggacgaggccatctacttctgcgccctgctgtacagcaactactgggtgttcggcggcggcaccaagctgaccgtgctgggcggcggcggcagccaggtgcagctgcagcagcccggcagcgagctggtgaggcccggcgccagcgtgaagctgagctgcaaggccagcggctacaccttcaccagctactggatgcactgggtgaagcagaggcccggccagggcctggagtggatcggcaacatctaccccaggagcggcaccaccaactacgacggctacttcaagagcaaggccaccctgaccgtggacaccagcagcagcaccgcctacatgcagctgagcagcctgaccagcgaggacagcgccgtgtacttctgcaccaggagcggctacgactaccccttcgtgtactggggccagggcaccctggtgaccgtgagcgccggcggcggcggcagcggcggcggcggcagcggcggcggcggcagccaggccgtggtgacccaggagagcgccctgaccaccagccccggcgagaccgtgaccctgacctgcaggaccagcaccggcgccgtgagcatcaggaactacgccaactgggtgcaggagaagcccgaccacctgttcaccggcctgatcggcggcaccaacaacagggcccccggcgtgcccgccaggttcagcggcagcctgatcggcgacaaggccgccctgaccatcaccggcgcccagcccgaggacgaggccatctacttctgcgccctgctgtacagcaactactgggtgttcggcggcggcaccaagctgaccgtgctgggcggcggcggcagccaggtgcagctgcagcagcccggcagcgagctggtgaggcccggcgccagcgtgaagctgagctgcaaggccagcggctacaccttcaccagctactggatgcactgggtgaagcagaggcccggccagggcctggagtggatcggcaacatctaccccaggagcggcaccaccaactacgacggctacttcaagagcaaggccaccctgaccgtggacaccagcagcagcaccgcctacatgcagctgagcagcctgaccagcgaggacagcgccgtgtacttctgcaccaggagcggctacgactaccccttcgtgtactggggccagggcaccctggtgaccgtgagcgccaccaccacccccgcccccaggccccccacccccgcccccaccatcgccagccccctgagcctgaggcccgaggcctgcaggcccgccgccggcggcgccgtgcacaccaggggcctggacttcgcctgcgacttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggagaaagagaggaagcggagagggcagaggaagtctgctaacatgcggtgacgtcgaggagaatcctggacctatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag

SEQ ID NO: 168 - DNA, 2148 bp, Artificial Sequence
  source: /note=“Description of Artificial Sequence Synthetic polynucleotide”
atggccctgcccgtgaccgccctgctgctgcccctggccctgctgctgcacgccgccaggccccaggccgtggtgacccaggagagcgccctgaccaccagccccggcgagaccgtgaccctgacctgcaggaccagcaccggcgccgtgagcatcaggaactacgccaactgggtgcaggagaagcccgaccacctgttcaccggcctgatcggcggcaccaacaacagggcccccggcgtgcccgccaggttcagcggcagcctgatcggcgacaaggccgccctgaccatcaccggcgcccagcccgaggacgaggccatctacttctgcgccctgctgtacagcaactactgggtgttcggcggcggcaccaagctgaccgtgctgggcggcggcggcagcggcggcggcggcagcggcggcggcggcagccaggtgcagctgcagcagcccggcagcgagctggtgaggcccggcgccagcgtgaagctgagctgcaaggccagcggctacaccttcaccagctactggatgcactgggtgaagcagaggcccggccagggcctggagtggatcggcaacatctaccccaggagcggcaccaccaactacgacggctacttcaagagcaaggccaccctgaccgtggacaccagcagcagcaccgcctacatgcagctgagcagcctgaccagcgaggacagcgccgtgtacttctgcaccaggagcggctacgactaccccttcgtgtactggggccagggcaccctggtgaccgtgagcgccgagagcaagtacggccccccctgccccagctgccccttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggagaaagagaggaagcggagagggcagaggaagtctgctaacatgcggtgacgtcgaggagaatcctggacctatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag

SEQ ID NO: 169 - DNA, 2799 bp, Artificial Sequence
  source: /note=“Description of Artificial Sequence Synthetic polynucleotide”
atggccctgcccgtgaccgccctgctgctgcccctggccctgctgctgcacgccgccaggccccaggccgtggtgacccaggagagcgccctgaccaccagccccggcgagaccgtgaccctgacctgcaggaccagcaccggcgccgtgagcatcaggaactacgccaactgggtgcaggagaagcccgaccacctgttcaccggcctgatcggcggcaccaacaacagggcccccggcgtgcccgccaggttcagcggcagcctgatcggcgacaaggccgccctgaccatcaccggcgcccagcccgaggacgaggccatctacttctgcgccctgctgtacagcaactactgggtgttcggcggcggcaccaagctgaccgtgctgggcggcggcggcagcggcggcggcggcagcggcggcggcggcagccaggtgcagctgcagcagcccggcagcgagctggtgaggcccggcgccagcgtgaagctgagctgcaaggccagcggctacaccttcaccagctactggatgcactgggtgaagcagaggcccggccagggcctggagtggatcggcaacatctaccccaggagcggcaccaccaactacgacggctacttcaagagcaaggccaccctgaccgtggacaccagcagcagcaccgcctacatgcagctgagcagcctgaccagcgaggacagcgccgtgtacttctgcaccaggagcggctacgactaccccttcgtgtactggggccagggcaccctggtgaccgtgagcgccgagagcaagtacggccccccctgccccccctgccccgccccccccgtggccggccccagcgtgttcctgttcccccccaagcccaaggacaccctgatgatcagcaggacccccgaggtgacctgcgtggtggtggacgtgagccaggaggaccccgaggtgcagttcaactggtacgtggacggcgtggaggtgcacaacgccaagaccaagcccagggaggagcagttccagagcacctacagggtggtgagcgtgctgaccgtgctgcaccaggactggctgaacggcaaggagtacaagtgcaaggtgagcaacaagggcctgcccagcagcatcgagaagaccatcagcaaggccaagggccagcccagggagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaggctgaccgtggacaagagcaggtggcaggagggcaacgtgttcagctgcagcgtgatgcacgaggccctgcacaaccactacacccagaagagcctgagcctgagcctgggcaagatgttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggagaaagagaggaagcggagagggcagaggaagtctgctaacatgcggtgacgtcgaggagaatcctggacctatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag

SEQ ID NO: 170 - DNA, 2244 bp, Artificial Sequence
  source: /note=“Description of Artificial Sequence Synthetic polynucleotide”
atggccctgcccgtgaccgccctgctgctgcccctggccctgctgctgcacgccgccaggccccaggtgcagctgcagcagcccggcagcgagctggtgaggcccggcgccagcgtgaagctgagctgcaaggccagcggctacaccttcaccagctactggatgcactgggtgaagcagaggcccggccagggcctggagtggatcggcaacatctaccccaggagcggcaccaccaactacgacggctacttcaagagcaaggccaccctgaccgtggacaccagcagcagcaccgcctacatgcagctgagcagcctgaccagcgaggacagcgccgtgtacttctgcaccaggagcggctacgactaccccttcgtgtactggggccagggcaccctggtgaccgtgagcgccggcggcggcggcagcggcggcggcggcagcggcggcggcggcagccaggccgtggtgacccaggagagcgccctgaccaccagccccggcgagaccgtgaccctgacctgcaggaccagcaccggcgccgtgagcatcaggaactacgccaactgggtgcaggagaagcccgaccacctgttcaccggcctgatcggcggcaccaacaacagggcccccggcgtgcccgccaggttcagcggcagcctgatcggcgacaaggccgccctgaccatcaccggcgcccagcccgaggacgaggccatctacttctgcgccctgctgtacagcaactactgggtgttcggcggcggcaccaagctgaccgtgctgaccaccacccccgcccccaggccccccacccccgcccccaccatcgccagccccctgagcctgaggcccgaggcctgcaggcccgccgccggcggcgccgtgcacaccaggggcctggacttcgcctgcgacttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggagaaagagaggaagcggagagggcagaggaagtctgctaacatgcggtgacgtcgaggagaatcctggacctatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag

SEQ ID NO: 171 - DNA, 2148 bp, Artificial Sequence
  source: /note=“Description of Artificial Sequence Synthetic polynucleotide”
atggccctgcccgtgaccgccctgctgctgcccctggccctgctgctgcacgccgccaggccccaggtgcagctgcagcagcccggcagcgagctggtgaggcccggcgccagcgtgaagctgagctgcaaggccagcggctacaccttcaccagctactggatgcactgggtgaagcagaggcccggccagggcctggagtggatcggcaacatctaccccaggagcggcaccaccaactacgacggctacttcaagagcaaggccaccctgaccgtggacaccagcagcagcaccgcctacatgcagctgagcagcctgaccagcgaggacagcgccgtgtacttctgcaccaggagcggctacgactaccccttcgtgtactggggccagggcaccctggtgaccgtgagcgccggcggcggcggcagcggcggcggcggcagcggcggcggcggcagccaggccgtggtgacccaggagagcgccctgaccaccagccccggcgagaccgtgaccctgacctgcaggaccagcaccggcgccgtgagcatcaggaactacgccaactgggtgcaggagaagcccgaccacctgttcaccggcctgatcggcggcaccaacaacagggcccccggcgtgcccgccaggttcagcggcagcctgatcggcgacaaggccgccctgaccatcaccggcgcccagcccgaggacgaggccatctacttctgcgccctgctgtacagcaactactgggtgttcggcggcggcaccaagctgaccgtgctggagagcaagtacggccccccctgccccagctgccccttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggagaaagagaggaagcggagagggcagaggaagtctgctaacatgcggtgacgtcgaggagaatcctggacctatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag

SEQ ID NO: 172 - DNA, 2799 bp, Artificial Sequence
  source: /note=“Description of Artificial Sequence Synthetic polynucleotide”
atggccctgcccgtgaccgccctgctgctgcccctggccctgctgctgcacgccgccaggccccaggtgcagctgcagcagcccggcagcgagctggtgaggcccggcgccagcgtgaagctgagctgcaaggccagcggctacaccttcaccagctactggatgcactgggtgaagcagaggcccggccagggcctggagtggatcggcaacatctaccccaggagcggcaccaccaactacgacggctacttcaagagcaaggccaccctgaccgtggacaccagcagcagcaccgcctacatgcagctgagcagcctgaccagcgaggacagcgccgtgtacttctgcaccaggagcggctacgactaccccttcgtgtactggggccagggcaccctggtgaccgtgagcgccggcggcggcggcagcggcggcggcggcagcggcggcggcggcagccaggccgtggtgacccaggagagcgccctgaccaccagccccggcgagaccgtgaccctgacctgcaggaccagcaccggcgccgtgagcatcaggaactacgccaactgggtgcaggagaagcccgaccacctgttcaccggcctgatcggcggcaccaacaacagggcccccggcgtgcccgccaggttcagcggcagcctgatcggcgacaaggccgccctgaccatcaccggcgcccagcccgaggacgaggccatctacttctgcgccctgctgtacagcaactactgggtgttcggcggcggcaccaagctgaccgtgctggagagcaagtacggccccccctgccccccctgccccgccccccccgtggccggccccagcgtgttcctgttcccccccaagcccaaggacaccctgatgatcagcaggacccccgaggtgacctgcgtggtggtggacgtgagccaggaggaccccgaggtgcagttcaactggtacgtggacggcgtggaggtgcacaacgccaagaccaagcccagggaggagcagttccagagcacctacagggtggtgagcgtgctgaccgtgctgcaccaggactggctgaacggcaaggagtacaagtgcaaggtgagcaacaagggcctgcccagcagcatcgagaagaccatcagcaaggccaagggccagcccagggagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaggctgaccgtggacaagagcaggtggcaggagggcaacgtgttcagctgcagcgtgatgcacgaggccctgcacaaccactacacccagaagagcctgagcctgagcctgggcaagatgttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggagaaagagaggaagcggagagggcagaggaagtctgctaacatgcggtgacgtcgaggagaatcctggacctatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag

SEQ ID NO: 173 - DNA, 2955 bp, Artificial Sequence; identical to SEQ ID NO: 167
  source: /note=“Description of Artificial Sequence Synthetic polynucleotide”

SEQ ID NO: 174 - DNA, 2955 bp, Artificial Sequence
  source: /note=“Description of Artificial Sequence Synthetic polynucleotide”
atggccctgcccgtgaccgccctgctgctgcccctggccctgctgctgcacgccgccaggccccaggtgcagctgcagcagcccggcagcgagctggtgaggcccggcgccagcgtgaagctgagctgcaaggccagcggctacaccttcaccagctactggatgcactgggtgaagcagaggcccggccagggcctggagtggatcggcaacatctaccccaggagcggcaccaccaactacgacggctacttcaagagcaaggccaccctgaccgtggacaccagcagcagcaccgcctacatgcagctgagcagcctgaccagcgaggacagcgccgtgtacttctgcaccaggagcggctacgactaccccttcgtgtactggggccagggcaccctggtgaccgtgagcgccggcggcggcggcagccaggccgtggtgacccaggagagcgccctgaccaccagccccggcgagaccgtgaccctgacctgcaggaccagcaccggcgccgtgagcatcaggaactacgccaactgggtgcaggagaagcccgaccacctgttcaccggcctgatcggcggcaccaacaacagggcccccggcgtgcccgccaggttcagcggcagcctgatcggcgacaaggccgccctgaccatcaccggcgcccagcccgaggacgaggccatctacttctgcgccctgctgtacagcaactactgggtgttcggcggcggcaccaagctgaccgtgctgggcggcggcggcagcggcggcggcggcagcggcggcggcggcagccaggtgcagctgcagcagcccggcagcgagctggtgaggcccggcgccagcgtgaagctgagctgcaaggccagcggctacaccttcaccagctactggatgcactgggtgaagcagaggcccggccagggcctggagtggatcggcaacatctaccccaggagcggcaccaccaactacgacggctacttcaagagcaaggccaccctgaccgtggacaccagcagcagcaccgcctacatgcagctgagcagcctgaccagcgaggacagcgccgtgtacttctgcaccaggagcggctacgactaccccttcgtgtactggggccagggcaccctggtgaccgtgagcgccggcggcggcggcagccaggccgtggtgacccaggagagcgccctgaccaccagccccggcgagaccgtgaccctgacctgcaggaccagcaccggcgccgtgagcatcaggaactacgccaactgggtgcaggagaagcccgaccacctgttcaccggcctgatcggcggcaccaacaacagggcccccggcgtgcccgccaggttcagcggcagcctgatcggcgacaaggccgccctgaccatcaccggcgcccagcccgaggacgaggccatctacttctgcgccctgctgtacagcaactactgggtgttcggcggcggcaccaagctgaccgtgctgaccaccacccccgcccccaggccccccacccccgcccccaccatcgccagccccctgagcctgaggcccgaggcctgcaggcccgccgccggcggcgccgtgcacaccaggggcctggacttcgcctgcgacttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccaggagaaagagaggaagcggagagggcagaggaagtctgctaacatgcggtgacgtcgaggagaatcctggacctatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag

SEQ ID NO: 175 - protein, 21 aa, Homo sapiens
MALPVTALLLPLALLLHAARP

SEQ ID NO: 176 - protein, 44 aa, Homo sapiens
TTTPAPRPPTPAPTIASPLSLRPEACRPAAGGAVHTRGLDFACD

SEQ ID NO: 177 - protein, 12 aa, Homo sapiens
ESKYGPPCPSCP

SEQ ID NO: 178 - protein, 229 aa, Homo sapiens
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKM

SEQ ID NO: 179 - protein, 41 aa, Artificial Sequence
  source: /note=“Description of Artificial Sequence Synthetic polypeptide”
RSKRSRLLHSDYMNMTPRRPGPTRKHYQPYAPPRDFAAYRS

SEQ ID NO: 180 - protein, 112 aa, Artificial Sequence
  source: /note=“Description of Artificial Sequence Synthetic polypeptide”
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 181 - protein, 4 aa, Artificial Sequence
  source: /note=“Description of Artificial Sequence Synthetic peptide”
GFLG

SEQ ID NO: 182 - protein, 4 aa, Artificial Sequence
  source: /note=“Description of Artificial Sequence Synthetic peptide”
ALAL

SEQ ID NO: 183 - protein, 5 aa, Artificial Sequence
  source: /note=“Description of Artificial Sequence Synthetic peptide”
GGGGS

SEQ ID NO: 184 - protein, 15 aa, Artificial Sequence
  source: /note=“Description of Artificial Sequence Synthetic peptide”
GGGGSGGGGSGGGGS

SEQ ID NO: 185 - protein, 24 aa, Artificial Sequence
  source: /note=“Description of Artificial Sequence Synthetic peptide”
YLHLGALGRDLWGPSPVTGYHPLL

SEQ ID NO: 186 - protein, 27 aa, Artificial Sequence
  source: /note=“Description of Artificial Sequence Synthetic peptide”
FWVLVVVGGVLACYSLLVTVAFIIFWV

SEQ ID NO: 187 - protein, 44 aa, Artificial Sequence
  source: /note=“Description of Artificial Sequence Synthetic polypeptide”
TTTPAPRPPTPAPTIASQPLSLRPEACRPAAGGAVHTRGLDFAC

SEQ ID NO: 188 - protein, 11 aa, Artificial Sequence
  source: /note=“Description of Artificial Sequence Synthetic peptide”
APTSSSGGGGS

SEQ ID NO: 189 - protein, 4 aa, Artificial Sequence
  source: /note=“Description of Artificial Sequence Synthetic peptide”
GGGS

SEQ ID NO: 190 - protein, 182 aa, Artificial Sequence
  source: /note=“Description of Artificial Sequence Synthetic polypeptide”
EPHSLRYNLTVLSWDGSVQSGFLTEVHLDGQPFLRCDRQKCRAKPQGQWAEDVLGNKTWDRETRDLTGWGTTLLMTLAHIKDQKEGLHSLQEIRVCEIHEDNSTRSSQHFYYDGELFLSQNLETLEWTMPQSSRAQTLAMNVRNFLKEDAMETDIGYRLMRADCLSELRRYLKSGVVLRRTV

SEQ ID NO: 191 - protein, 182 aa, Artificial Sequence
  source: /note=“Description of Artificial Sequence Synthetic polypeptide”
EPHSLRYNLTVLSWDGSVQSGFLTEVHLDGQPFLRCDRQKCRAKPQGQWAEDVLGNKTWDRETRDLTGWGTFLRMTLAHIKDQKEGLHSLQEIRVCEIHEDNSTRSSQHFYYDGELFLSQNLETLEWTMPQSSRAQTLAMNVRNFLKEDAMETDRSGLLMRADCLSELRRYLKSGVVLRRTV

SEQ ID NO: 192 - protein, 186 aa, Artificial Sequence
  source: /note=“Description of Artificial Sequence Synthetic polypeptide”
AAEPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATTLYTWSMGDCIGWLEDFLMGMDSTLEPSAGAP

SEQ ID NO: 193 - protein, 186 aa, Artificial Sequence
  source: /note=“Description of Artificial Sequence Synthetic polypeptide”
AAEPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATLMRIWSMGDCIGWLEDFLMGMDSTLEPSAGAP

SEQ ID NO: 194 - protein, 186 aa, Artificial Sequence
  source: /note=“Description of Artificial Sequence Synthetic polypeptide”
AAEPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATKLYLWSMGDCIGWLEDFLMGMDSTLEPSAGAP

SEQ ID NO: 195 - protein, 184 aa, Artificial Sequence
  source: /note=“Description of Artificial Sequence Synthetic polypeptide”
AAEPHSLWYNFTIIHLPRHGQQWCEVQSQVDQKNFLSYDCGSDKVLSMGHLEEQLYATDAWGKQLEMLREVGQRLRLELADTELEDFTPSGPLTLQVRMSCESEADGYIRGSWQFSFDGRKFLLFDSNNRKWTVVHAGARRMKEKWEKDSGLTTDLIRRSMGDCKSWLRDFLMHRKKRLEPTAP

SEQ ID NO: 196 - protein, 184 aa, Artificial Sequence
  source: /note=“Description of Artificial Sequence Synthetic polypeptide”
AAEPHSLWYNFTIIHLPRHGQQWCEVQSQVDQKNFLSYDCGSDKVLSMGHLEEQLYATDAWGKQLEMLREVGQRLRLELADTELEDFTPSGPLTLQVRMSCESEADGYIRGSWQFSFDGRKFLLFDSNNRKWTVVHAGARRMKEKWEKDSGLTTYFYLRSMGDCKSWLRDFLMHRKKRLEPTAP

SEQ ID NO: 197 - protein, 180 aa, Artificial Sequence
  source: /note=“Description of Artificial Sequence Synthetic polypeptide”
EPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATILWQTSMGDCIGWLEDFLMGMDSTLEPS

SEQ ID NO: 198 - protein, 180 aa, Artificial Sequence
  source: /note=“Description of Artificial Sequence Synthetic polypeptide”
EPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATLLWGWSMGDCIGWLEDFLMGMDSTLEPS

SEQ ID NO: 199 - protein, 180 aa, Artificial Sequence
  source: /note=“Description of Artificial Sequence Synthetic polypeptide”
EPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATMFWSWSMGDCIGWLEDFLMGMDSTLEPS

SEQ ID NO: 200 - protein, 180 aa, Artificial Sequence
  source: /note=“Description of Artificial Sequence Synthetic polypeptide”
EPHSLSYDITVIPKFRPGPRWCAVQGQVDEKTFLHYDCGNKTVTPVSPLGKKLNVTTAWKAQNPVLREVVDILTEQLWDIQLENYTPKEPLTLQARMSCEQKAEGHSSGSWQFSFDGQIFLLFDSEKRMWTTVHPGARKMKEKWENDKVVATLMWQWSMGDCIGWLEDFLMGMDSTLEPS

SEQ ID NO: 201 - protein, 137 aa, Artificial Sequence
  source: /note=“Description of Artificial Sequence Synthetic polypeptide”
NSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 202 - protein, 139 aa, Artificial Sequence
  source: /note=“Description of Artificial Sequence Synthetic polypeptide”
FLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 203 - protein, 43 aa, Homo sapiens
TTTPAPRPPTPAPTIASPLSLRPEACRPAAGGAVHTRGLDFAC

SEQ ID NO: 204 - RNA, 70 nt, Artificial Sequence
  source: /note=“Description of Artificial Sequence Synthetic oligonucleotide”
  misc_feature (1)..(70): /note=“The sequence can include 50-70 nucleotides”
aaaaaaaaaaaaaaaaaaaaaaaaaaaaaaaaaaaaaaaaaaaaaaaaaaaaaaaaaaaaaaaaaaaaaa

SEQ ID NO: 205 - protein, 60 aa, Artificial Sequence
  source: /note=“Description of Artificial Sequence Synthetic polypeptide”
VTSAPDTRPAPGSTAPPAHGVTSAPDTRPAPGSTAPPAHGVTSAPDTRPAPGSTAPPAHG

SEQ ID NO: 206 - protein, 115 aa, Artificial Sequence
  source: /note=“Description of Artificial Sequence Synthetic polypeptide”
QSLEESGGRLVTPGTPLTLTCTASGFTINTYHMGWFRQAPGKGLQYIGIVSHDVGTYYATWAKGRFTISKTSSTTVDLRMPSPTTEDTATYICARGPGYWTFNLWGQGTLVTVSS

SEQ ID NO: 207 - protein, 110 aa, Artificial Sequence
  source: /note=“Description of Artificial Sequence Synthetic polypeptide”
AQVLTQTPASVSAAVGGTVTINCQASQSVYNNNQLSWYQQKPGQPLKQLIYKASTLASGVPSRFKGSGSGSQFTLTISDLECDDAATYFCAGGYKGDIHPFGGGTEVVVK

SEQ ID NO: 208 - protein, 8 aa, Artificial Sequence
  source: /note=“Description of Artificial Sequence Synthetic peptide”
GFTINTYH

SEQ ID NO: 209 - protein, 7 aa, Artificial Sequence
  source: /note=“Description of Artificial Sequence Synthetic peptide”
VSHDVGT

SEQ ID NO: 210 - protein, 11 aa, Artificial Sequence
  source: /note=“Description of Artificial Sequence Synthetic peptide”
ARGPGYWTFNL

SEQ ID NO: 211 - protein, 8 aa, Artificial Sequence
  source: /note=“Description of Artificial Sequence Synthetic peptide”
QSVYNNNQ

SEQ ID NO: 212 - protein, 3 aa, Artificial Sequence
  source: /note=“Description of Artificial Sequence Synthetic peptide”
KAS

SEQ ID NO: 213 - protein, 10 aa, Artificial Sequence
  source: /note=“Description of Artificial Sequence Synthetic peptide”
AGGYKGDIHP

SEQ ID NO: 214 - protein, 5 aa, Artificial Sequence
  source: /note=“Description of Artificial Sequence Synthetic peptide”
TYHMG

SEQ ID NO: 215 - protein, 16 aa, Artificial Sequence
  source: /note=“Description of Artificial Sequence Synthetic peptide”
IVSHDVGTYYATWAKG

SEQ ID NO: 216 - protein, 9 aa, Artificial Sequence
  source: /note=“Description of Artificial Sequence Synthetic peptide”
GPGYWTFNL

SEQ ID NO: 217 - protein, 13 aa, Artificial Sequence
  source: /note=“Description of Artificial Sequence Synthetic peptide”
QASQSVYNNNQLS

SEQ ID NO: 218 - protein, 7 aa, Artificial Sequence
  source: /note=“Description of Artificial Sequence Synthetic peptide”
KASTLAS

SEQ ID NO: 219 - protein, 10 aa, Artificial Sequence
  source: /note=“Description of Artificial Sequence Synthetic peptide”
AGGYKGDIHP

SEQ ID NO: 220 - protein, 7 aa, Artificial Sequence
  source: /note=“Description of Artificial Sequence Synthetic peptide”
GFTINTY

SEQ ID NO: 221 - protein, 5 aa, Artificial Sequence
  source: /note=“Description of Artificial Sequence Synthetic peptide”
SHDVG

SEQ ID NO: 222 - protein, 9 aa, Artificial Sequence
  source: /note=“Description of Artificial Sequence Synthetic peptide”
GPGYWTFNL

SEQ ID NO: 223 - protein, 13 aa, Artificial Sequence
  source: /note=“Description of Artificial Sequence Synthetic peptide”
QASQSVYNNNQLS

SEQ ID NO: 224 - protein, 7 aa, Artificial Sequence
  source: /note=“Description of Artificial Sequence Synthetic peptide”
KASTLAS

SEQ ID NO: 225 - protein, 10 aa, Artificial Sequence
  source: /note=“Description of Artificial Sequence Synthetic peptide”
AGGYKGDIHP

SEQ ID NO: 226 - DNA, 345 bp, Artificial Sequence
  source: /note=“Description of Artificial Sequence Synthetic polynucleotide”
cagagcctggaggagagcggcggcaggctggtgacccccggcacccccctgaccctgacctgcaccgccagcggcttcaccatcaacacctaccacatgggctggttcaggcaggcccccggcaagggcctgcagtacatcggcatcgtgagccacgacgtgggcacctactacgccacctgggccaagggcaggttcaccatcagcaagaccagcagcaccaccgtggacctgaggatgcccagccccaccaccgaggacaccgccacctacatctgcgccaggggccccggctactggaccttcaacctgtggggccagggcaccctggtgaccgtgagcagc

SEQ ID NO: 227 - DNA, 330 bp, Artificial Sequence
  source: /note=“Description of Artificial Sequence Synthetic polynucleotide”
gcccaggtgctgacccagacccccgccagcgtgagcgccgccgtgggcggcaccgtgaccatcaactgccaggccagccagagcgtgtacaacaacaaccagctgagctggtaccagcagaagcccggccagcccctgaagcagctgatctacaaggccagcaccctggccagcggcgtgcccagcaggttcaagggcagcggcagcggcagccagttcaccctgaccatcagcgacctggagtgcgacgacgccgccacctacttctgcgccggcggctacaagggcgacatccaccccttcggcggcggcaccgaggtggtggtgaag

SEQ ID NO: 228 - protein, 8 aa, Artificial Sequence
  source: /note=“Description of Artificial Sequence Synthetic peptide”
  VARIANT (2)..(2): /replace=“Phe”
  VARIANT (4)..(4): /replace=“Ile”
  VARIANT (5)..(5): /replace=“Ser” or “Asn”
  VARIANT (6)..(6): /replace=“Thr”
  VARIANT (7)..(7): /replace=“Tyr”
  VARIANT (8)..(8): /replace=“Ala” or “Gly” or “His”
  SITE (1)..(8): /note=“Variant residues given in the sequence have no preference with respect to those in the annotations for variant positions”
GYTFTSFW

SEQ ID NO: 229 - protein, 7 aa, Artificial Sequence
  source: /note=“Description of Artificial Sequence Synthetic peptide”
  VARIANT (1)..(1): /replace=“Ser”
  VARIANT (2)..(2): /replace=“Ser” or “His”
  VARIANT (3)..(3): /replace=“Gly” or “Asp”
  VARIANT (4)..(4): /replace=“Gly”
  VARIANT (5)..(5): /replace=“Ser”
  VARIANT (6)..(6): /replace=“Tyr”
  VARIANT (7)..(7): /replace=“Ile” or “ ”
  SITE (1)..(7): /note=“Variant residues given in the sequence have no preference with respect to those in the annotations for variant positions”
YPRSGTT

SEQ ID NO: 230 - protein, 3 aa, Artificial Sequence
  source: /note=“Description of Artificial Sequence Synthetic peptide”
  VARIANT (1)..(1): /replace=“Ser” or “Leu” or “Lys”
  VARIANT (2)..(2): /replace=“Ile” or “Ala”
  VARIANT (3)..(3): /replace=“Ser”
  SITE (1)..(3): /note=“Variant residues given in the sequence have no preference with respect to those in the annotations for variant positions”
GTN

SEQ ID NO: 231 - protein, 10 aa, Artificial Sequence
  source: /note=“Description of Artificial Sequence Synthetic peptide”
  VARIANT (1)..(1): /replace=“Gln” or “Leu”
  VARIANT (2)..(2): /replace=“Gln” or “Gly”
  VARIANT (3)..(3): /replace=“Gly” or “Trp”
  VARIANT (4)..(4): /replace=“Ser” or “Thr”
  VARIANT (5)..(5): /replace=“Thr” or “His” or “Lys”
  VARIANT (6)..(6): /replace=“Leu” or “Gln” or “Gly”
  VARIANT (7)..(7): /replace=“Pro” or “Asp”
  VARIANT (8)..(8): /replace=“Phe” or “Ile”
  VARIANT (9)..(9): /replace=“Thr” or “His”
  VARIANT (10)..(10): /replace=“ ”
  SITE (1)..(10): /note=“Variant residues given in the sequence have no preference with respect to those in the annotations for variant positions”
ALLYSNYWYP

SEQ ID NO: 232 - protein, 5 aa, Artificial Sequence
  source: /note=“Description of Artificial Sequence Synthetic peptide”
  VARIANT (1)..(1): /replace=“Thr”
  VARIANT (2)..(2): /replace=“Phe”
  VARIANT (3)..(3): /replace=“Ala” or “Gly” or “His”
  VARIANT (4)..(4): /replace=“Ile”
  VARIANT (5)..(5): /replace=“Ser” or “Gly”
  SITE (1)..(5): /note=“Variant residues given in the sequence have no preference with respect to those in the annotations for variant positions”
SYWMH

SEQ ID NO: 233 - protein, 17 aa, Artificial Sequence
  source: /note=“Description of Artificial Sequence Synthetic peptide”
  VARIANT (1)..(1): /replace=“Glu” or “Tyr” or “Ile”
  VARIANT (2)..(2): /replace=“Val”
  VARIANT (3)..(3): /replace=“Ser”
  VARIANT (4)..(4): /replace=“Ser” or “His”
  VARIANT (5)..(5): /replace=“Gly” or “Asp”
  VARIANT (6)..(6): /replace=“Gly”
  VARIANT (7)..(7): /replace=“Ser”
  VARIANT (8)..(8): /replace=“Tyr”
  VARIANT (9)..(9): /replace=“Ile” or “ ”
  VARIANT (10)..(10): /replace=“Tyr”
  VARIANT (12)..(12): /replace=“Pro” or “Ala”
  VARIANT (13)..(13): /replace=“Asp” or “Thr”
  VARIANT (14)..(14): /replace=“Thr” or “Trp”
  VARIANT (15)..(15): /replace=“Val” or “Ala”
  VARIANT (16)..(16): /replace=“Thr”
  VARIANT (17)..(17): /replace=“Gly”
  SITE (1)..(17): /note=“Variant residues given in the sequence have no preference with respect to those in the annotations for variant positions”
NIYPRSGTTNYDGYFKS

SEQ ID NO: 234 - protein, 7 aa, Artificial Sequence
  source: /note=“Description of Artificial Sequence Synthetic peptide”
  VARIANT (1)..(1): /replace=“Ser” or “Leu” or “Lys”
  VARIANT (2)..(2): /replace=“Ile” or “Ala”
  VARIANT (3)..(3): /replace=“Ser”
  VARIANT (4)..(4): /replace=“Arg” or “Thr”
  VARIANT (5)..(5): /replace=“Leu”
  VARIANT (6)..(6): /replace=“His” or “Phe”
  VARIANT (7)..(7): /replace=“Ser”
  SITE (1)..(7): /note=“Variant residues given in the sequence have no preference with respect to those in the annotations for variant positions”
GTNNRAP

SEQ ID NO: 235 - protein, 10 aa, Artificial Sequence
  source: /note=“Description of Artificial Sequence Synthetic peptide”
  VARIANT (1)..(1): /replace=“Gln” or “Leu”
  VARIANT (2)..(2): /replace=“Gln” or “Gly”
  VARIANT (3)..(3): /replace=“Gly” or “Trp”
  VARIANT (4)..(4): /replace=“Ser” or “Thr”
  VARIANT (5)..(5): /replace=“Thr” or “His” or “Lys”
  VARIANT (6)..(6): /replace=“Leu” or “Gln” or “Gly”
  VARIANT (7)..(7): /replace=“Pro” or “Asp”
  VARIANT (8)..(8): /replace=“Phe” or “Ile”
  VARIANT (9)..(9): /replace=“Thr” or “His”
  VARIANT (10)..(10): /replace=“ ”
  SITE (1)..(10): /note=“Variant residues given in the sequence have no preference with respect to those in the annotations for variant positions”
ALLYSNYWYP

SEQ ID NO: 236 - protein, 7 aa, Artificial Sequence
  source: /note=“Description of Artificial Sequence Synthetic peptide”
  VARIANT (2)..(2): /replace=“Phe”
  VARIANT (4)..(4): /replace=“Ile”
  VARIANT (5)..(5): /replace=“Ser” or “Asn”
  VARIANT (6)..(6): /replace=“Thr”
  VARIANT (7)..(7): /replace=“Phe”
  SITE (1)..(7): /note=“Variant residues given in the sequence have no preference with respect to those in the annotations for variant positions”
GYTFTSY

SEQ ID NO: 237 - protein, 6 aa, Artificial Sequence
  source: /note=“Description of Artificial Sequence Synthetic peptide”
  VARIANT (1)..(1): /replace=“Ser”
  VARIANT (2)..(2): /replace=“Ser” or “His”
  VARIANT (3)..(3): /replace=“Gly” or “Asp”
  VARIANT (4)..(4): /replace=“Gly”
  VARIANT (5)..(5): /replace=“Ser”
  VARIANT (6)..(6): /replace=“Tyr”
  SITE (1)..(6): /note=“Variant residues given in the sequence have no preference with respect to those in the annotations for variant positions”
YPRSGT

SEQ ID NO: 238 - protein, 7 aa, Artificial Sequence
  source: /note=“Description of Artificial Sequence Synthetic peptide”
  VARIANT (1)..(1): /replace=“Ser” or “Leu” or “Lys”
  VARIANT (2)..(2): /replace=“Ile” or “Ala”
  VARIANT (3)..(3): /replace=“Ser”
  VARIANT (4)..(4): /replace=“Arg” or “Thr”
  VARIANT (5)..(5): /replace=“Leu”
  VARIANT (6)..(6): /replace=“His” or “Phe”
  VARIANT (7)..(7): /replace=“Ser”
  SITE (1)..(7): /note=“Variant residues given in the sequence have no preference with respect to those in the annotations for variant positions”
GTNNRAP

SEQ ID NO: 239 - protein, 10 aa, Artificial Sequence
  source: /note=“Description of Artificial Sequence Synthetic peptide”
  VARIANT (1)..(1): /replace=“Gln” or “Leu”
  VARIANT (2)..(2): /replace=“Gln” or “Gly”
  VARIANT (3)..(3): /replace=“Gly” or “Trp”
  VARIANT (4)..(4): /replace=“Ser” or “Thr”
  VARIANT (5)..(5): /replace=“Thr” or “His” or “Lys”
  VARIANT (6)..(6): /replace=“Leu” or “Gln” or “Gly”
  VARIANT (7)..(7): /replace=“Pro” or “Asp”
  VARIANT (8)..(8): /replace=“Phe” or “Ile”
  VARIANT (9)..(9): /replace=“Thr” or “His”
  VARIANT (10)..(10): /replace=“ ”
  SITE (1)..(10): /note=“Variant residues given in the sequence have no preference with respect to those in the annotations for variant positions”
ALLYSNYWYP

SEQ ID NO: 240 - protein, 10 aa, Artificial Sequence
  source: /note=“Description of Artificial Sequence Synthetic peptide”
GFTINTYHMG

SEQ ID NO: 241 - protein, 16 aa, Artificial Sequence
  source: /note=“Description of Artificial Sequence Synthetic peptide”
IVSHDVGTYYATWAKG

SEQ ID NO: 242 - protein, 11 aa, Artificial Sequence
  source: /note=“Description of Artificial Sequence Synthetic peptide”
ARGPGYWTFNL

SEQ ID NO: 243 - protein, 13 aa, Artificial Sequence
  source: /note=“Description of Artificial Sequence Synthetic peptide”
QASQSVYNNNQLS

SEQ ID NO: 244 - protein, 7 aa, Artificial Sequence
  source: /note=“Description of Artificial Sequence Synthetic peptide”
KASTLAS

SEQ ID NO: 245 - protein, 10 aa, Artificial Sequence
  source: /note=“Description of Artificial Sequence Synthetic peptide”
AGGYKGDIHP

SEQ ID NO: 246 - protein, 10 aa, Artificial Sequence
  source: /note=“Description of Artificial Sequence Synthetic peptide”
  VARIANT (2)..(2): /replace=“Phe”
  VARIANT (4)..(4): /replace=“Ile”
  VARIANT (5)..(5): /replace=“Ser” or “Asn”
  VARIANT (6)..(6): /replace=“Thr”
  VARIANT (7)..(7): /replace=“Phe”
  VARIANT (8)..(8): /replace=“Ala” or “Gly” or “His”
  VARIANT (9)..(9): /replace=“Ile”
  VARIANT (10)..(10): /replace=“Ser” or “Gly”
  SITE (1)..(10): /note=“Variant residues given in the sequence have no preference with respect to those in the annotations for variant positions”
GYTFTSYWMH

SEQ ID NO: 247 - protein, 17 aa, Artificial Sequence
  source: /note=“Description of Artificial Sequence Synthetic peptide”
  VARIANT (1)..(1): /replace=“Glu” or “Tyr” or “Ile”
  VARIANT (2)..(2): /replace=“Val”
  VARIANT (3)..(3): /replace=“Ser”
  VARIANT (4)..(4): /replace=“Ser” or “His”
  VARIANT (5)..(5): /replace=“Gly” or “Asp”
  VARIANT (6)..(6): /replace=“Gly”
  VARIANT (7)..(7): /replace=“Ser”
  VARIANT (8)..(8): /replace=“Tyr”
  VARIANT (9)..(9): /replace=“Ile” or “ ”
  VARIANT (10)..(10): /replace=“Tyr”
  VARIANT (12)..(12): /replace=“Pro” or “Ala”
  VARIANT (13)..(13): /replace=“Asp” or “Thr”
  VARIANT (14)..(14): /replace=“Thr” or “Trp”
  VARIANT (15)..(15): /replace=“Val” or “Ala”
  VARIANT (16)..(16): /replace=“Thr”
  VARIANT (17)..(17): /replace=“Gly”
  SITE (1)..(17): /note=“Variant residues given in the sequence have no preference with respect to those in the annotations for variant positions”
NIYPRSGTTNYDGYFKS

SEQ ID NO: 248 - protein, 7 aa, Artificial Sequence
  source: /note=“Description of Artificial Sequence Synthetic peptide”
  VARIANT (1)..(1): /replace=“Ser” or “Leu” or “Lys”
  VARIANT (2)..(2): /replace=“Ile” or “Ala”
  VARIANT (3)..(3): /replace=“Ser”
  VARIANT (4)..(4): /replace=“Arg” or “Thr”
  VARIANT (5)..(5): /replace=“Leu”
  VARIANT (6)..(6): /replace=“His” or “Phe”
  VARIANT (7)..(7): /replace=“Ser”
  SITE (1)..(7): /note=“Variant residues given in the sequence have no preference with respect to those in the annotations for variant positions”
GTNNRAP

SEQ ID NO: 249 - protein, 10 aa, Artificial Sequence
  source: /note=“Description of Artificial Sequence Synthetic peptide”
  VARIANT (1)..(1): /replace=“Gln” or “Leu”
  VARIANT (2)..(2): /replace=“Gln” or “Gly”
  VARIANT (3)..(3): /replace=“Gly” or “Trp”
  VARIANT (4)..(4): /replace=“Ser” or “Thr”
  VARIANT (5)..(5): /replace=“Thr” or “His” or “Lys”
  VARIANT (6)..(6): /replace=“Leu” or “Gln” or “Gly”
  VARIANT (7)..(7): /replace=“Pro” or “Asp”
  VARIANT (8)..(8): /replace=“Phe” or “Ile”
  VARIANT (9)..(9): /replace=“Thr” or “His”
  VARIANT (10)..(10): /replace=“ ”
  SITE (1)..(10): /note=“Variant residues given in the sequence have no preference with respect to those in the annotations for variant positions”
ALLYSNYWYP

SEQ ID NO: 250 - protein, 2 aa, Artificial Sequence
  source: /note=“Description of Artificial Sequence Synthetic peptide”
TY

SEQ ID NO: 251 - protein, 5 aa, Artificial Sequence
  source: /note=“Description of Artificial Sequence Synthetic peptide”
SHDVG

SEQ ID NO: 252 - protein, 9 aa, Artificial Sequence
  source: /note=“Description of Artificial Sequence Synthetic peptide”
GPGYWTFNL

SEQ ID NO: 253 - protein, 8 aa, Artificial Sequence
  source: /note=“Description of Artificial Sequence Synthetic peptide”
QSVYNNNQ

SEQ ID NO: 254 - protein, 3 aa, Artificial Sequence
  source: /note=“Description of Artificial Sequence Synthetic peptide”
KAS

SEQ ID NO: 255 - protein, 10 aa, Artificial Sequence
  source: /note=“Description of Artificial Sequence Synthetic peptide”
AGGYKGDIHP

SEQ ID NO: 256 - protein, 2 aa, Artificial Sequence
  source: /note=“Description of Artificial Sequence Synthetic peptide”
  VARIANT (1)..(1): /replace=“Thr”
  VARIANT (2)..(2): /replace=“Phe”
  SITE (1)..(2): /note=“Variant residues given in the sequence have no preference with respect to those in the annotations for variant positions”
SY

SEQ ID NO: 257 - protein, 6 aa, Artificial Sequence
  source: /note=“Description of Artificial Sequence Synthetic peptide”
  VARIANT (1)..(1): /replace=“Ser”
  VARIANT (2)..(2): /replace=“Ser” or “His”
  VARIANT (3)..(3): /replace=“Gly” or “Asp”
  VARIANT (4)..(4): /replace=“Gly”
  VARIANT (5)..(5): /replace=“Ser”
  VARIANT (6)..(6): /replace=“Tyr”
  SITE (1)..(6): /note=“Variant residues given in the sequence have no preference with respect to those in the annotations for variant positions”
YPRSGT

SEQ ID NO: 258 - protein, 3 aa, Artificial Sequence
  source: /note=“Description of Artificial Sequence Synthetic peptide”
  VARIANT (1)..(1): /replace=“Ser” or “Leu” or “Lys”
  VARIANT (2)..(2): /replace=“Ile” or “Ala”
  VARIANT (3)..(3): /replace=“Ser”
  SITE (1)..(3): /note=“Variant residues given in the sequence have no preference with respect to those in the annotations for variant positions”
GTN

SEQ ID NO: 259 - protein, 9 aa, Artificial Sequence
  source: /note=“Description of Artificial Sequence Synthetic peptide”
  VARIANT (1)..(1): /replace=“Gln” or “Leu”
  VARIANT (2)..(2): /replace=“Gln” or “Gly”
  VARIANT (3)..(3): /replace=“Gly” or “Trp”
  VARIANT (4)..(4): /replace=“Ser” or “Thr”
  VARIANT (5)..(5): /replace=“Thr” or “His” or “Lys”
  VARIANT (6)..(6): /replace=“Leu” or “Gln” or “Gly”
  VARIANT (7)..(7): /replace=“Pro” or “Asp”
  VARIANT (8)..(8): /replace=“Phe” or “Ile”
  VARIANT (9)..(9): /replace=“Thr” or “His”
  SITE (1)..(9): /note=“Variant residues given in the sequence have no preference with respect to those in the annotations for variant positions”
ALLYSNYWY

SEQ ID NO: 260 - protein, 29 aa, Artificial Sequence
  source: /note=“Description of Artificial Sequence Synthetic peptide”
  MOD_RES (9)..(9): Glycosylated Thr
  MOD_RES (10)..(10): Glycosylated Ser
  MOD_RES (14)..(14): Glycosylated Thr
  MOD_RES (20)..(20): Glycosylated Ser
  MOD_RES (21)..(21): Glycosylated Thr
TAPPAHGVXXAPDXRPAPGXXAPPAHGVT

SEQ ID NO: 261 - DNA, 1488 bp, Artificial Sequence
  source: /note=“Description of Artificial Sequence Synthetic polynucleotide”
atggctctgcctgtcaccgcgctgctccttcccttagccctgctgctgcacgctgctcgtccagcgcaggtcctgactcagacacccgcgtccgtgtccgcagcggtcgggggcactgtgaccatcaactgccaggcaagccagtccgtgtacaataacaaccagctgagttggtaccaacagaagcctgggcagcccctaaagcagctgatttacaaggcttccacccttgcttccggcgtcccctctaggtttaagggctctgggagcggttcccagttcaccctgaccatcagcgatctggaatgcgatgacgccgccacctacttctgtgccggcggctacaagggggacatccaccccttcggtggcggtactgaggtggtagtcaagggagacccggtgggcggtggcggctccggaggcggtggttctggtggaggaggcagccagtgtcagtctctggaggagagcgggggccgcctggtgacccccggtactccgctcactctgacctgcactgcatctggcttcaccattaacacgtaccacatgggatggttccgccaggcccctggcaagggcctccagtatattggcatcgtgagtcatgacgtgggcacctactacgctacctgggcaaaaggacgcttcacgatctccaagacctcctcgaccacagtggacctgcgcatgcccagcccaacgaccgaggacaccgccacttacatctgcgcccgtgggccgggctattggaccttcaacctgtggggccagggcaccctggtgacagtttcctcgggacagccaaagaccacaacccccgccccgcgccctcctaccccggcccctacgatagcgtcacccctgtctctccgccccgaggcttgtaggcctgccgctggtggggccgtccacacccgtggcttggatttcgcttgcgacttctgggtcttggtggtggtggggggagtgctggcgtgttactcgttgctggtcaccgtggcgttcatcatcttctgggtacgcagcaagcgcagccgtctcctgcactccgactacatgaatatgacacctagacggcccggccctacccggaagcactaccagccctatgctcccccgagggactttgctgcgtaccgctcccgcgtgaaattttcacgctccgcggacgctccggcctatcagcagggtcagaaccagctttacaacgagctcaacctgggccgacgtgaagagtacgatgtgctggacaagcgccggggacgcgatccagagatgggcggcaagcccagacgcaagaaccctcaggagggcttgtacaacgagctgcagaaggacaaaatggccgaggcctactccgagatcggtatgaagggggagcggcgccgcggcaaaggccacgacggcctctatcaggggctttcgaccgccactaaggacacctacgacgcacttcatatgcaagccctgcccccgcgctag

SEQ ID NO: 262 - protein, 495 aa, Artificial Sequence
  source: /note=“Description of Artificial Sequence Synthetic polypeptide”
MALPVTALLLPLALLLHAARPAQVLTQTPASVSAAVGGTVTINCQASQSVYNNNQLSWYQQKPGQPLKQLIYKASTLASGVPSRFKGSGSGSQFTLTISDLECDDAATYFCAGGYKGDIHPFGGGTEVVVKGDPVGGGGSGGGGSGGGGSQCQSLEESGGRLVTPGTPLTLTCTASGFTINTYHMGWFRQAPGKGLQYIGIVSHDVGTYYATWAKGRFTISKTSSTTVDLRMPSPTTEDTATYICARGPGYWTFNLWGQGTLVTVSSGQPKTTTPAPRPPTPAPTIASPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2023/0126689 A1
(43) Pub. Date: Apr. 27, 2023

(54) ANTI-GLYCO-CD44 ANTIBODIES AND THEIR USES

(71) Applicant: GO Therapeutics, Inc., Cambridge, MA (US)

(72) Inventors: Julia SCHNABEL, Cambridge, MA (US); Edwin TAN, Hacienda Heights, CA (US); Hans H. WANDALL, Charlottelund (DK); Aaron GROEN, Melrose, MA (US)

(73) Assignee: GO Therapeutics, Inc., Cambridge, MA (US)

(21) Appl. No.: 17/909,045

(22) PCT Filed: Mar. 5, 2021
(86) PCT No.: PCT/US2021/021211

§ 371 Date: Sep. 2, 2022

Related U.S. Application Data
(60) Provisional application No. 62/986,083, filed on Mar. 6, 2020.

(51) Int. Cl. C07K 16/28; A61P 35/00; A61K 39/395; A61K 39/39; C07K 14/725; C12N 15/62; G01N 33/574

(52) U.S. Cl. CPC C07K 16/2884; A61P 35/00; A61K 39/3955; A61K 39/39; C07K 14/7051; C12N 15/625; G01N 33/57484; A61K 2039/55505

(57) ABSTRACT

The present disclosure relates to anti-glyco-CD44 antibodies and antigen binding fragments thereof that specifically bind to a cancer-specific glycosylation variant of CD44 and related fusion proteins and antibody-drug conjugates, as well as nucleic acids encoding such biomolecules. The present disclosure further relates to use of the antibodies, antigen-binding fragments, fusion proteins, antibody-drug conjugates and nucleic acids for cancer therapy.

### 1. CROSS-REFERENCE TO RELATED APPLICATIONS

This application claims the priority benefit of U.S. provisional application No. 62/986,083, filed Mar. 6, 2020, the contents of which are incorporated herein in their entireties by reference thereto.

### 2. SEQUENCE LISTING

The instant application contains a Sequence Listing which has been submitted electronically in ASCII format and is hereby incorporated by reference in its entirety. Said ASCII copy, created on Mar. 5, 2021, is named GOT-003-WO_SL.txt and is 202,651 bytes in size.

### 3. BACKGROUND

Therapies redirecting T cell responses using chimeric antigen receptors (CARs) have emerged as a potent tool in cancer immunotherapies and have proved highly effective in haematological cancers, targeting antigens shared with nonessential tissues such as CD19 in B cell malignancies (Brentjens et al., 2013, Sci Transl Med. 5(177):177ra38-177ra38; Grupp et al., 2013, N Engl J Med. 368(16):1509-1518; Kalos et al., 2011, Sci Transl Med. 3(95):95ra73-95ra73; Kochenderfer et al., 2010, Blood. 116(20):4099-4102; Porter et al., 2011, N Engl J Med. 365(8):725-733). However, adopting CAR therapies to solid tumours has been challenging because the majority of CAR targets are normal self-antigens overexpressed in solid cancers. As such, adverse effects due to cross-reactions with essential healthy tissues are often reported in studies targeting solid tumours with CART cells (Bin Hou et al., 2019, Dis Markers, Article ID 3425291). To overcome the challenges of adopting CAR therapies to solid tumours, new cancer-specific antigens allowing selective targeting are required.

Many cancers express aberrantly glycosylated proteins that are distinct from healthy tissues. Such aberrantly glycosylated proteins contain glycopeptide epitopes that may be suitable for immunotherapy of solid tumors, but few such glycopeptide epitopes have been identified. CD44 is a heavily glycosylated transmembrane protein that is involved in cell-cell interactions, cell adhesion and migration, and has additionally been suggested as a marker for cancer stem cells. There are ten CD44 variants in humans, including the standard variant. The ten variants are differentially expressed in a variety of tumors (see Chen, et al., 2018, J Hematol Oncol. 11(1):64). There are 117 potential O-linked glycosylation sites within the CD44 variant region, including 54 serines and 63 threonines.

Antibodies targeting CD44, such as Bivatuzumab, which recognizes the cancer-associated isoform CD44v6, are known in the art. Yet, Bivatuzumab induces severe skin toxicities due to a low expression of CD44v6 in healthy skin. See, Börjesson et al., 2003. Clin Cancer Res. 9(10 Pt 2):3961S-72S; Brentjens et al., 2013. Sci Transl Med. 5(177):177ra38-177ra38; Goodison et al., 1999, Mol Pathol. 52(4):189-196; Grupp, et al. 2013, N Engl J Med. 368(16):1509-1518; Hou et al., 2019, Dis Markers. 2019:1-11; Julien et al., 2012, Biomolecules. 2(4):435-466; Kalos et al., 2011, Sci Transl Med. 3(95):95ra73-95ra73; King et al., 2017, Blood Adv. 1(7):429-442; Kochenderfer et al., 2010, Blood. 116(20):4099-4102; Porter et al., 2011, N Engl J Med. 365(8):725-733; Posey et al., 2016, Immunity. 44(6):1444-1454; Prochazka et al., 2014, Cell Signal. 26(10):2234-2239; Radhakrishnan et al., 2014, Proc Natl Acad Sci. 111(39):E4066-E4075; Sneath et al., 1998, Mol Pathol. 51(4):191-200; Sorensen et al., 2006, Glycobiology. 16(2):96-107; Stanley, 2011, Cold Spring Harb Perspect Biol. 3(4); Steentoft et al., 2013, EMBO J. 32(10):1478-1488; Steentoft et al., 2011, Nat Methods. 8(11):977-982; Stroomer et al., 2000, Clin Cancer Res. 6(8):3046-3055; Thapa et al., 2016, Stem Cells Int. 2016:1-15; Tijink et al., 2006, Clin Cancer Res. 12(20):6064-6072; Wandall et al., 2010, Cancer Res. 70(4):1306-1313.

Thus, there is a need for identification of glyco-CD44 epitopes that are overexpressed in cancer cells and new therapeutic modalities, such as antibodies and CARs, which utilize such glyco-CD44 epitopes.

### 4. SUMMARY

The disclosure captures the tumor specificity of glycopeptide variants by providing therapeutic and diagnostic agents based on antibodies and antigen binding fragments that are selective for cancer-specific epitopes of glyco-CD44.

The present disclosure provides anti-glyco-CD44 antibodies and antigen binding fragments thereof that bind to a cancer-specific glycosylation variant of CD44. The present disclosure further provides fusion proteins and antibody-drug conjugates comprising anti-glyco-CD44 antibodies and antigen binding fragments, and nucleic acids encoding the anti-glyco-CD44 antibodies, antigen binding fragments and fusion proteins.

The present disclosure further provides methods of using the anti-glyco-CD44 antibodies, antigen-binding fragments, fusion proteins, antibody-drug conjugates and nucleic acids for cancer therapy.

In certain aspects, the disclosure provides bispecific and other multispecific anti-glyco-CD44 antibodies and antigen binding fragments that bind to a cancer-specific glycosylation variant of CD44 and to a second epitope. The second epitope can either be on CD44 itself, on another protein co-expressed on cancer cells with CD44, or on another protein presented on a different cell, such as an activated T cell. Further, also disclosed are nucleic acids encoding such antibodies, including nucleic acids comprising codon-optimized coding regions and nucleic acids comprising coding regions that are not codon-optimized for expression in a particular host cell.

The anti-glyco-CD44 antibodies and binding fragments can be in the form of fusion proteins containing a fusion partner. The fusion partner can be useful to provide a second function, such as a signaling function of the signaling domain of a T cell signaling protein, a peptide modulator of T cell activation or an enzymatic component of a labeling system. Exemplary T cell signaling proteins include 4-1BB, CO3C, and fusion peptides, e.g., CD28-CD3-zeta and 4-1BB-CD3-zeta. 4-1BB, or CD137, is a co-stimulatory receptor of T cells; CD3-zeta is a signal-transduction component of the T-cell antigen receptor. The moiety providing a second function can be a modulator of T cell activation, such as IL-15, IL-15Ra, or an IL-15/IL-15Ra fusion, can be an MHC-class I-chain-related (MIC) protein domain useful for making a MicAbody, or it can encode a label or an enzymatic component of a labeling system useful in monitoring the extent and/or location of binding in vivo or in vitro. Constructs encoding these prophylactically and therapeutically active biomolecules placed in the context of T cells, such as autologous T cells, provide a powerful platform for recruiting adoptively transferred T cells to prevent or treat a variety of cancers in some embodiments of the disclosure.

In certain aspects, an anti-glyco-CD44 antibody or antigen-binding fragment of the disclosure comprises heavy and/or light chain variable sequences (or encoded by the nucleotide sequences) set forth in Tables 1A through 1E. For clarity, when the term “anti-glyco-CD44 antibody” is used in this document, it is intended to include monospecific and multi-specific (including bispecific) anti-glyco-CD44 antibodies, antigen-binding fragments of the monospecific and multi-specific antibodies, and fusion proteins and conjugates containing the antibodies and their antigen-binding fragments, unless the context dictates otherwise. Likewise, when the term “anti-glyco-CD44 antibody or antigen-binding fragment” is used, it is also intended to include monospecific and multi-specific (including bispecific) anti-glyco-CD44 antibodies and their antigen-binding fragments, together with fusion proteins and conjugates containing such antibodies and antigen-binding fragments, unless the context dictates otherwise.

In other aspects, an anti-glyco-CD44 antibody or antigen-binding fragment of the disclosure comprises heavy and/or light chain CDR sequences (or encoded by the nucleotide sequences) set forth in Tables 1-3. The CDR sequences set forth in Tables 1A-1E include CDR sequences defined according to the IMGT (Lefranc et al., 2003, Dev Comparat Immunol 27:55-77), Kabat (Kabat et al., 1991, Sequences of Proteins of Immunological Interest, 5th Ed. Public Health Service, National Institutes of Health, Bethesda, Md.), and Chothia (Al-Lazikani et al., 1997, J. Mol. Biol 273:927-948) schemes for defining CDR boundaries. The CDR sequences set forth in Tables 1F, 1G, and 1H are consensus sequences derived from the CDR sequences set forth in Tables 1A through 1D according to the IMGT, Kabat, and Chothia definitions, respectively. The CDR sequences set forth in Tables 1I, 1J, and 1K are consensus sequences derived from the CDR sequences set forth in Tables 1A through 1E according to the IMGT, Kabat, and Chothia definitions, respectively. The CDR sequences set forth in Tables 2A through 2E are the combined regions of overlap for the CDR sequences set forth in Tables 1A through 1E, respectively, with the IMGT, Kabat and Chothia sequences shown in underlined bold text. The CDR sequences set forth in Table 2F are the combined regions of overlap for the consensus CDR sequences set forth in Tables 1F-1H. The CDR sequences set forth in Table 2G are the combined regions of overlap for the consensus CDR sequences set forth in Tables 11-1K. The CDR sequences set forth in Tables 3A-3E are the common regions of overlap for the CDR sequences shown in Tables 1A-1E, respectively. The CDR sequences set forth in Table 3F are the common regions of overlap for the CDR sequences set forth in Tables 1F-1H. The CDR sequences set forth in Table 3G are the common regions of overlap for the CDR sequences set forth in Tables 1I-1K. The framework sequences for such anti-glyco-CD44 antibody and antigen-binding fragment can be the native murine framework sequences of the VH and VL sequences set forth in Tables 1A-1D, can be the native rabbit framework sequences of the VH and VL sequences set forth in Table 1E, or can be non-native (e.g., humanized or human) framework sequences.

TABLE 1A
4C8 Sequences

| | | SEQ |
|---|---|---|
| Description | Sequence | ID NO: |
| VH amino acid | QVQLQQPGSELVRPGASVKLSCKA | 1 |
| sequence | SGYTFTSYWMHWVKQRPGQGLEWI | |
| (predicted | GNIYPRSGTTNYDGYFKSKATLTV | |
| mature) | DTSSSTAYMQLSSLTSEDSAVYFC | |
| | TRSGYDYPFVYWGQGTLVTVSA | |
| VL amino acid | QAVVTQESALTTSPGETVTLTCRT | 2 |
| sequence | STGAVSIRNYANWVQEKPDHLFTG | |
| (predicted | LIGGTNNRAPGVPARFSGSLIGDK | |
| mature) | AALTITGAQPEDEAIYFCALLYSN | |
| | YWVFGGGTKLTVL | |
| CDR-H1 amino | GYTFTSYW | 3 |
| acid sequence | | |
| (IMGT | | |
| definition) | | |
| CDR-H2 amino | IYPRSGTT | 4 |
| acid sequence | | |
| (IMGT | | |
| definition) | | |
| CDR-H3 amino | TRSGYDYPFVY | 5 |
| acid sequence | | |
| (IMGT | | |
| definition) | | |
| CDR-L1 amino | TGAVSIRNY | 6 |
| acid sequence | | |
| (IMGT | | |
| definition) | | |
| CDR-L2 amino | GTN | 7 |
| acid sequence | | |
| (IMGT | | |
| definition) | | |
| CDR-L3 amino | ALLYSNYWV | 8 |
| acid sequence | | |
| (IMGT | | |
| definition) | | |
| CDR-H1 amino | SYWMH | 9 |
| acid sequence | | |
| (Kabat | | |
| definition) | | |
| CDR-H2 amino | NIYPRSGTTNYDGYFKS | 10 |
| acid sequence | | |
| (Kabat | | |
| definition) | | |
| CDR-H3 amino | SGYDYPFVY | 11 |
| acid sequence | | |
| (Kabat | | |
| definition) | | |
| CDR-L1 amino | RTSTGAVSIRNYAN | 12 |
| acid sequence | | |
| (Kabat | | |
| definition) | | |
| CDR-L2 amino | GTNNRAP | 13 |
| acid sequence | | |
| (Kabat | | |
| definition) | | |
| CDR-L3 amino | ALLYSNYWV | 14 |
| acid sequence | | |
| (Kabat | | |
| definition) | | |
| CDR-H1 amino | GYTFTSY | 15 |
| acid sequence | | |
| (Chothia | | |
| definition) | | |
| CDR-H2 amino | YPRSGT | 16 |
| acid sequence | | |
| (Chothia | | |
| definition) | | |
| CDR-H3 amino | SGYDYPFVY | 17 |
| acid sequence | | |
| (Chothia | | |
| definition) | | |
| CDR-L1 amino | RTSTGAVSIRNYAN | 18 |
| acid sequence | | |
| (Chothia | | |
| definition) | | |
| CDR-L2 amino | GTNNRAP | 19 |
| acid sequence | | |
| (Chothia | | |
| definition) | | |
| CDR-L3 amino | ALLYSNYWV | 20 |
| acid sequence | | |
| (Chothia | | |
| definition) | | |
| VH nucleotide | CAGGTCCAACTGCAGCAACCTGGG | 21 |
| sequence | TCTGAACTGGTGAGGCCTGGAGCT | |
| (excl. signal | TCAGTGAAGCTGTCCTGCAAGGCT | |
| sequence) | TCTGGCTACACATTCACCAGTTAC | |
| | TGGATGCACTGGGTGAAGCAGAGG | |
| | CCTGGGCAAGGCCTTGAGTGGATT | |
| | GGAAATATTTATCCTCGTAGTGGT | |
| | ACTACTAACTACGATGGGTACTTC | |
| | AAGAGTAAAGCCACACTGACTGTA | |
| | GACACATCCTCCAGCACGGCCTAC | |
| | ATGCAGCTCAGTAGCCTGACATCT | |
| | GAGGACTCTGCGGTCTATTTCTGT | |
| | ACAAGATCGGGATATGATTACCCT | |
| | TTTGTTTACTGGGGCCAGGGTACT | |
| | CTGGTCACTGTCTCTGCA | |
| VL nucleotide | CAGGCTGTTGTGACTCAGGAATCT | 22 |
| sequence | GCACTCACCACATCACCTGGTGAA | |
| (excl. signal | ACAGTCACACTCACTTGTCGCACA | |
| sequence) | AGTACTGGGGCTGTTTCAATTAGA | |
| | AACTATGCCAACTGGGTCCAAGAA | |
| | AAACCAGATCATTTATTCACTGGT | |
| | CTAATAGGTGGTACCAACAACCGA | |
| | GCTCCAGGTGTTCCTGCCAGATTC | |
| | TCAGGCTCCCTGATTGGAGACAAG | |
| | GCTGCCCTCACCATCACAGGGGCA | |
| | CAGCCTGAGGATGAGGCGATATAT | |
| | TTCTGTGCTCTATTATACAGCAAT | |
| | TATTGGGTGTTCGGTGGAGGAACC | |
| | AAACTGACTGTCCTA | |

TABLE 1B
2B2 Sequences

| | | SEQ |
|---|---|---|
| Description | Sequence | ID NO: |
| VH amino acid | EVQLVESGGGLVKPGGSLKLSCAA | 23 |
| sequence | SGFTFSSYAMSWVRQSPERRLEWV | |
| (predicted | AEISSGGSYTYYPDTVTGRFTISR | |
| mature) | DNAKNTLYLEMSSLRSEDTAMYYC | |
| | ARTVGEDWYFDVWGAGTTVTVSS | |
| VL amino acid | DIQMTQTTSSLSASLGDRVTISCR | 24 |
| sequence | ASQDISHYLNWYQQKPDGAVKLLI | |
| (predicted | YSTSRLHSGVPSRFSGSGSGTDYS | |
| mature) | LTISNLEQEDIATYFCQQGYTLPF | |
| | TFGSGTKLEIK | |
| CDR-H1 amino | GFTFSSYA | 25 |
| acid sequence | | |
| (IMGT | | |
| definition) | | |
| CDR-H2 amino | ISSGGSYT | 26 |
| acid sequence | | |
| (IMGT | | |
| definition) | | |
| CDR-H3 amino | ARTVGEDWYFDV | 27 |
| acid sequence | | |
| (IMGT | | |
| definition) | | |
| CDR-L1 amino | QDISHY | 28 |
| acid sequence | | |
| (IMGT | | |
| definition) | | |
| CDR-L2 amino | STS | 29 |
| acid sequence | | |
| (IMGT | | |
| definition) | | |
| CDR-L3 amino | QQGYTLPFT | 30 |
| acid sequence | | |
| (IMGT | | |
| definition) | | |
| CDR-H1 amino | SYAMS | 31 |
| acid sequence | | |
| (Kabat | | |
| definition) | | |
| CDR-H2 amino | EISSGGSYTYYPDTVTG | 32 |
| acid sequence | | |
| (Kabat | | |
| definition) | | |
| CDR-H3 amino | TVGEDWYFDV | 33 |
| acid sequence | | |
| (Kabat | | |
| definition) | | |
| CDR-L1 amino | RASQDISHYLN | 34 |
| acid sequence | | |
| (Kabat | | |
| definition) | | |
| CDR-L2 amino | STSRLHS | 35 |
| acid sequence | | |
| (Kabat | | |
| definition) | | |
| CDR-L3 amino | QQGYTLPFT | 36 |
| acid sequence | | |
| (Kabat | | |
| definition) | | |
| CDR-H1 amino | GFTFSSY | 37 |
| acid sequence | | |
| (Chothia | | |
| definition) | | |
| CDR-H2 amino | SSGGSY | 38 |
| acid sequence | | |
| (Chothia | | |
| definition) | | |
| CDR-H3 amino | TVGEDWYFDV | 39 |
| acid sequence | | |
| (Chothia | | |
| definition) | | |
| CDR-L1 amino | RASQDISHYLN | 40 |
| acid sequence | | |
| (Chothia | | |
| definition) | | |
| CDR-L2 amino | STSRLHS | 41 |
| acid sequence | | |
| (Chothia | | |
| definition) | | |
| CDR-L3 amino | QQGYTLPFT | 42 |
| acid sequence | | |
| (Chothia | | |
| definition) | | |
| VH nucleotide | GAAGTGCAGCTGGTGGAGTCTGGG | 43 |
| sequence | GGAGGCTTAGTGAAGCCTGGAGGG | |
| (excl. signal | TCCCTGAAACTCTCCTGTGCAGCC | |
| sequence) | TCTGGATTCACTTTCAGTAGCTAT | |
| | GCCATGTCTTGGGTTCGCCAGTCT | |
| | CCAGAGAGGAGGCTGGAGTGGGTC | |
| | GCAGAAATTAGTAGTGGTGGTAGT | |
| | TATACCTACTATCCAGACACTGTG | |
| | ACGGGCCGATTCACCATCTCCAGA | |
| | GACAATGCCAAGAACACCCTGTAC | |
| | CTGGAAATGAGCAGTCTGAGGTCT | |
| | GAGGACACGGCCATGTATTACTGT | |
| | GCAAGGACAGTAGGTGAGGACTGG | |
| | TACTTCGATGTCTGGGGCGCAGGG | |
| | ACCACGGTCACCGTCTCCTCA | |
| VL nucleotide | GATATCCAGATGACACAGACTACA | 44 |
| sequence | TCCTCCCTGTCTGCCTCTCTGGGA | |
| (excl. signal | GACAGAGTCACCATCAGTTGCAGG | |
| sequence) | GCAAGTCAGGACATTAGTCATTAT | |
| | TTAAACTGGTATCAGCAGAAACCA | |
| | GATGGAGCTGTTAAACTCCTGATC | |
| | TACTCCACATCAAGATTACACTCA | |
| | GGAGTCCCATCAAGGTTCAGTGGC | |
| | AGTGGGTCTGGAACAGATTATTCT | |
| | CTCACCATTAGCAACCTGGAGCAA | |
| | GAAGATATTGCCACTTACTTTTGC | |
| | CAACAGGGTTATACGCTTCCATTC | |
| | ACGTTCGGCTCGGGGACAAAATTG | |
| | GAAATAAAA | |

TABLE 1C
18G9 Sequences

| | | SEQ |
|---|---|---|
| Description | Sequence | ID NO: |
| VH amino acid | DVQLVESGGGLVQPGGSRKLSCAA | 45 |
| sequence | SGFTFSSFGMHWVRQAPEKGLEWV | |
| (predicted | AYISSGSSTIYYADTVKGRFTISR | |
| mature) | DNPKNTLFLQMTSLRSEDTAMYYC | |
| | ARGSYRAMDYWGQGTSVTVSS | |
| VL amino acid | QIVLTQSPALMSASPGEKVTMTCS | 46 |
| sequence | ASSSVNYMFWYQQKPRSSPKPWIY | |
| (predicted | LTSNLASGVPARFSGSGSGTSYSL | |
| mature) | TISSMEAEDAATYYCQLWSSNPFT | |
| | FGSGTKLEIK | |
| CDR-H1 amino | GFTFSSFG | 47 |
| acid sequence | | |
| (IMGT | | |
| definition) | | |
| CDR-H2 amino | ISSGSSTI | 48 |
| acid sequence | | |
| (IMGT | | |
| definition) | | |
| CDR-H3 amino | ARGSYRAMDY | 49 |
| acid sequence | | |
| (IMGT | | |
| definition) | | |
| CDR-L1 amino | SSVNY | 50 |
| acid sequence | | |
| (IMGT | | |
| definition) | | |
| CDR-L2 amino | LTS | 51 |
| acid sequence | | |
| (IMGT | | |
| definition) | | |
| CDR-L3 amino | QLWSSNPFT | 52 |
| acid sequence | | |
| (IMGT | | |
| definition) | | |
| CDR-H1 amino | SFGMH | 53 |
| acid sequence | | |
| (Kabat | | |
| definition) | | |
| CDR-H2 amino | YISSGSSTIYYADTVKG | 54 |
| acid sequence | | |
| (Kabat | | |
| definition) | | |
| CDR-H3 amino | GSYRAMDY | 55 |
| acid sequence | | |
| (Kabat | | |
| definition) | | |
| CDR-L1 amino | SASSSVNYMF | 56 |
| acid sequence | | |
| (Kabat | | |
| definition) | | |
| CDR-L2 amino | LTSNLAS | 57 |
| acid sequence | | |
| (Kabat | | |
| definition) | | |
| CDR-L3 amino | QLWSSNPFT | 58 |
| acid sequence | | |
| (Kabat | | |
| definition) | | |
| CDR-H1 amino | GFTFSSF | 59 |
| acid sequence | | |
| (Chothia | | |
| definition) | | |
| CDR-H2 amino | SSGSST | 60 |
| acid sequence | | |
| (Chothia | | |
| definition) | | |
| CDR-H3 amino | GSYRAMDY | 61 |
| acid sequence | | |
| (Chothia | | |
| definition) | | |
| CDR-L1 amino | SASSSVNYMF | 62 |
| acid sequence | | |
| (Chothia | | |
| definition) | | |
| CDR-L2 amino | LTSNLAS | 63 |
| acid sequence | | |
| (Chothia | | |
| definition) | | |
| CDR-L3 amino | QLWSSNPFT | 64 |
| acid sequence | | |
| (Chothia | | |
| definition) | | |
| VH nucleotide | GATGTGCAGCTGGTGGAGTCTGGG | 65 |
| sequence | GGAGGCTTAGTGCAGCCTGGAGGG | |
| (excl. signal | TCCCGGAAACTCTCCTGTGCAGCC | |
| sequence) | TCTGGATTCACTTTCAGTAGCTTT | |
| | GGAATGCACTGGGTTCGTCAGGCT | |
| | CCAGAGAAGGGGCTGGAGTGGGTC | |
| | GCATACATTAGTAGTGGCAGTAGT | |
| | ACCATCTACTATGCAGACACAGTG | |
| | AAGGGCCGATTCACCATCTCCAGA | |
| | GACAATCCCAAGAACACCCTGTTC | |
| | CTGCAAATGACCAGTCTAAGGTCT | |
| | GAGGACACGGCCATGTATTACTGT | |
| | GCAAGAGGGAGCTACAGGGCTATG | |
| | GACTACTGGGGTCAAGGAACCTCA | |
| | GTCACCGTCTCCTCA | |
| VL nucleotide | CAAATTGTTCTCACCCAGTCTCCA | 66 |
| sequence | GCACTCATGTCTGCATCTCCAGGG | |
| (excl. signal | GAGAAGGTCACCATGACCTGCAGT | |
| sequence) | GCCAGCTCAAGTGTAAATTACATG | |
| | TTCTGGTACCAGCAGAAGCCAAGA | |
| | TCCTCCCCCAAACCCTGGATTTAT | |
| | CTCACATCCAACCTGGCTTCTGGA | |
| | GTCCCTGCTCGCTTCAGTGGCAGT | |
| | GGGTCTGGGACCTCTTACTCTCTC | |
| | ACAATCAGCAGCATGGAGGCTGAA | |
| | GATGCTGCCACTTATTACTGCCAG | |
| | CTGTGGAGTAGTAACCCATTCACG | |
| | TTCGGCTCGGGGACAAAGTTGGAA | |
| | ATAAAA | |

TABLE 1D
1D12 Sequences

| | | SEQ |
|---|---|---|
| Description | Sequence | ID NO: |
| VH amino acid | DVQLVESGGGLVQPGGSRKLSCAA | 67 |
| sequence | SGFTFSSFGIHWVRQAPEKGLEWV | |
| (predicted | AYISSGSSTIYYADTVKGRFTISR | |
| mature) | DNPKNTLFLQMTSLRSEDTAMYYC | |
| | ARGSKVVAKSRGYWYFDVWGAGTT | |
| | VTVSS | |
| VL amino acid | DVVVTQTPLFLPVSFGDQVSISCR | 68 |
| sequence | SSQSLANNYGITYLSWYLHRPGQS | |
| (predicted | PQLLIYGISNRFSGVPDRFSGSGS | |
| mature) | GTDFTLKISTIKPEDLGMYYCLQG | |
| | THQPWTFGGGTKLEIK | |
| CDR-H1 amino | GFTFSSFG | 69 |
| acid sequence | | |
| (IMGT | | |
| definition) | | |
| CDR-H2 amino | ISSGSSTI | 70 |
| acid sequence | | |
| (IMGT | | |
| definition) | | |
| CDR-H3 amino | ARGSKVVAKSRGYWYFDV | 71 |
| acid sequence | | |
| (IMGT | | |
| definition) | | |
| CDR-L1 amino | QSLANNYGITY | 72 |
| acid sequence | | |
| (IMGT | | |
| definition) | | |
| CDR-L2 amino | GIS | 73 |
| acid sequence | | |
| (IMGT | | |
| definition) | | |
| CDR-L3 amino | LQGTHQPWT | 74 |
| acid sequence | | |
| (IMGT | | |
| definition) | | |
| CDR-H1 amino | SFGIH | 75 |
| acid sequence | | |
| (Kabat | | |
| definition) | | |
| CDR-H2 amino | YISSGSSTIYYADTVKG | 76 |
| acid sequence | | |
| (Kabat | | |
| definition) | | |
| CDR-H3 amino | GSKVVAKSRGYWYFDV | 77 |
| acid sequence | | |
| (Kabat | | |
| definition) | | |
| CDR-L1 amino | RSSQSLANNYGITYLS | 78 |
| acid sequence | | |
| (Kabat | | |
| definition) | | |
| CDR-L2 amino | GISNRFS | 79 |
| acid sequence | | |
| (Kabat | | |
| definition) | | |
| CDR-L3 amino | LQGTHQPWT | 80 |
| acid sequence | | |
| (Kabat | | |
| definition) | | |
| CDR-H1 amino | GFTFSSF | 81 |
| acid sequence | | |
| (Chothia | | |
| definition) | | |
| CDR-H2 amino | SSGSST | 82 |
| acid sequence | | |
| (Chothia | | |
| definition) | | |
| CDR-H3 amino | GSKVVAKSRGYWYFDV | 83 |
| acid sequence | | |
| (Chothia | | |
| definition) | | |
| CDR-L1 amino | RSSQSLANNYGITYLS | 84 |
| acid sequence | | |
| (Chothia | | |
| definition) | | |
| CDR-L2 amino | GISNRFS | 85 |
| acid sequence | | |
| (Chothia | | |
| definition) | | |
| CDR-L3 amino | LQGTHQPWT | 86 |
| acid sequence | | |
| (Chothia | | |
| definition) | | |
| VH nucleotide | GATGTGCAGCTGGTGGAGTCTGGG | 87 |
| sequence | GGAGGCTTAGTGCAGCCTGGAGGG | |
| (excl. signal | TCCCGGAAACTCTCCTGTGCAGCC | |
| sequence) | TCTGGATTCACTTTCAGTAGTTTT | |
| | GGAATTCACTGGGTTCGTCAGGCT | |
| | CCAGAGAAGGGGCTGGAGTGGGTC | |
| | GCATACATAAGTAGTGGCAGTAGT | |
| | ACCATCTACTATGCAGACACAGTG | |
| | AAGGGCCGATTCACCATCTCCAGA | |
| | GACAATCCCAAGAACACCCTGTTC | |
| | CTGCAAATGACCAGTCTAAGGTCT | |
| | GAGGACACGGCCATGTATTACTGT | |
| | GCAAGAGGGTCTAAGGTAGTAGCT | |
| | AAGTCAAGGGGCTACTGGTACTTC | |
| | GATGTCTGGGGCGCAGGGACCACG | |
| | GTCACCGTCTCCTCA | |
| VL nucleotide | GATGTTGTGGTGACTCAAACTCCA | 88 |
| sequence | CTCTTCCTGCCTGTCAGCTTTGGA | |
| (excl. signal | GATCAAGTTTCTATCTCTTGCAGG | |
| sequence) | TCTAGTCAGAGTCTTGAAACAATT | |
| | ATGGGATCACCTATTTGTCTTGGT | |
| | ACCTGCACAGGCCTGGCCAGTCTC | |
| | CCCAGCTCCTCATCTATGGGATTT | |
| | CCAACAGATTTTCTGGGGTGCCAG | |
| | ACAGGTTCAGTGGCAGTGGTTCAG | |
| | GGACAGATTTCACACTCAAGATCA | |
| | GCACAATAAAGCCTGAGGACTTGG | |
| | GAATGTATTACTGCTTACAAGGTA | |
| | CACATCAGCCGTGGACGTTCGGTG | |
| | GAGGCACCAAGCTGGAAATCAAA | |

TABLE 1E
10H4 Sequences

| | | SEQ |
|---|---|---|
| Description | Sequence | ID NO: |
| VH amino acid | QSLEESGGRLVTPGTPLTLTCTAS | 206 |
| sequence | GFTINTYHMGWFRQAPGKGLQYIG | |
| (predicted | IVSHDVGTYYATWAKGRFTISKTS | |
| mature) | STTVDLRMPSPTTEDTATYICARG | |
| | PGYWTFNLWGQGTLVTVSS | |
| VL amino acid | AQVLTQTPASVSAAVGGTVTINCQ | 207 |
| sequence | ASQSVYNNNQLSWYQQKPGQPLKQ | |
| (predicted | LIYKASTLASGVPSRFKGSGSGSQ | |
| mature) | FTLTISDLECDDAATYFCAGGYKG | |
| | DIHPFGGGTEVVVK | |
| CDR-H1 amino | GFTINTYH | 208 |
| acid sequence | | |
| (IMGT | | |
| definition) | | |
| CDR-H2 amino | VSHDVGT | 209 |
| acid sequence | | |
| (IMGT | | |
| definition) | | |
| CDR-H3 amino | ARGPGYWTFNL | 210 |
| acid sequence | | |
| (IMGT | | |
| definition) | | |
| CDR-L1 amino | QSVYNNNQ | 211 |
| acid sequence | | |
| (IMGT | | |
| definition) | | |
| CDR-L2 amino | KAS | 212 |
| acid sequence | | |
| (IMGT | | |
| definition) | | |
| CDR-L3 amino | AGGYKGDIHP | 213 |
| acid sequence | | |
| (IMGT | | |
| definition) | | |
| CDR-H1 amino | TYHMG | 214 |
| acid sequence | | |
| (Kabat | | |
| definition) | | |
| CDR-H2 amino | IVSHDVGTYYATWAKG | 215 |
| acid sequence | | |
| (Kabat | | |
| definition) | | |
| CDR-H3 amino | GPGYWTFNL | 216 |
| acid sequence | | |
| (Kabat | | |
| definition) | | |
| CDR-L1 amino | QASQSVYNNNQLS | 217 |
| acid sequence | | |
| (Kabat | | |
| definition) | | |
| CDR-L2 amino | KASTLAS | 218 |
| acid sequence | | |
| (Kabat | | |
| definition) | | |
| CDR-L3 amino | AGGYKGDIHP | 219 |
| acid sequence | | |
| (Kabat | | |
| definition) | | |
| CDR-H1 amino | GFTINTY | 220 |
| acid sequence | | |
| (Chothia | | |
| definition) | | |
| CDR-H2 amino | SHDVG | 221 |
| acid sequence | | |
| (Chothia | | |
| definition) | | |
| CDR-H3 amino | GPGYWTFNL | 222 |
| acid sequence | | |
| (Chothia | | |
| definition) | | |
| CDR-L1 amino | QASQSVYNNNQLS | 223 |
| acid sequence | | |
| (Chothia | | |
| definition) | | |
| CDR-L2 amino | KASTLAS | 224 |
| acid sequence | | |
| (Chothia | | |
| definition) | | |
| CDR-L3 amino | AGGYKGDIHP | 225 |
| acid sequence | | |
| (Chothia | | |
| definition) | | |
| VH nucleotide | CAGAGCCTGGAGGAGAGCGGCGGC | 226 |
| sequence | AGGCTGGTGACCCCCGGCACCCCC | |
| (excl. signal | CTGACCCTGACCTGCACCGCCAGC | |
| sequence) | GGCTTCACCATCAACACCTACCAC | |
| | ATGGGCTGGTTCAGGCAGGCCCCC | |
| | GGCAAGGGCCTGCAGTACATCGGC | |
| | ATCGTGAGCCACGACGTGGGCACC | |
| | TACTACGCCACCTGGGCCAAGGGC | |
| | AGGTTCACCATCAGCAAGACCAGC | |
| | AGCACCACCGTGGACCTGAGGATG | |
| | CCCAGCCCCACCACCGAGGACACC | |
| | GCCACCTACATCTGCGCCAGGGGC | |
| | CCCGGCTACTGGACCTTCAACCTG | |
| | TGGGGCCAGGGCACCCTGGTGACC | |
| | GTGAGCAGC | |
| VL nucleotide | GCCCAGGTGCTGACCCAGACCCCC | 227 |
| sequence | GCCAGCGTGAGCGCCGCCGTGGGC | |
| (excl. signal | GGCACCGTGACCATCAACTGCCAG | |
| sequence) | GCCAGCCAGAGCGTGTACAACAAC | |
| | AACCAGCTGAGCTGGTACCAGCAG | |
| | AAGCCCGGCCAGCCCCTGAAGCAG | |
| | CTGATCTACAAGGCCAGCACCCTG | |
| | GCCAGCGGCGTGCCCAGCAGGTTC | |
| | AAGGGCAGCGGCAGCGGCAGCCAG | |
| | TTCACCCTGACCATCAGCGACCTG | |
| | GAGTGCGACGACGCCGCCACCTAC | |
| | TTCTGCGCCGGCGGCTACAAGGGC | |
| | GACATCCACCCCTTCGGCGGCGGC | |
| | ACCGAGGTGGTGGTGAAG | |

TABLE 1F
Murine CDR Consensus sequences-IMGT definition

| | | SEQ |
|---|---|---|
| Description | Sequence | ID NO: |
| CDR-H1 amino | GX₁TFX₂SX₃X₄ | 89 |
| acid sequence | | |
| (IMGT | | |
| definition) | | |
| CDR-H2 amino | X₈X₉X₁₀X₁₁X₁₂X₁₃X₁₄ | 90 |
| acid sequence | | |
| (IMGT | | |
| definition) | | |
| CDR-L2 amino | X₂₂X₂₃X₂₄ | 91 |
| acid sequence | | |
| (IMGT | | |
| definition) | | |
| CDR-L3 amino | X₂₉X₃₀X₃₁X₃₂X₃₃X₃₄X₃₅X₃₆X₃₇ | 92 |
| acid sequence | | |
| (IMGT | | |
| definition) | | |

X₁ = Y or F;
X₂ = T or S;
X₃ = Y or F;
X₄ = W, A, or G;
X₈ = Y or S;
X₉ = P or S;
X₁₀ = R or G;
X₁₁ = S or G;
X₁₂ = G or S;
X₁₃ = T or Y;
X₁₄ = T or I;
X₂₂ = G, S, or L;
X₂₃ = T or I;
X₂₄ = N or S;
X₂₉ = A, Q, or L;
X₃₀ = L or Q;
X₃₁ = L, G, or W;
X₃₂ = Y, S, or T;
X₃₃ = S, T, or H;
X₃₄ = N, L, or Q;
X₃₅ = Y or P;
X₃₆ = W or F;
X₃₇ = Y or T

TABLE 1G
Murine CDR Consensus sequences-Kabat definition

| | | SEQ |
|---|---|---|
| Description | Sequence | ID NO: |
| CDR-H1 amino | SX₃X₄X₅X₆ | 93 |
| acid sequence | | |
| (Kabat | | |
| definition) | | |
| CDR-H2 amino | X₇IX₈X₉X₁₀X₁₁X₁₂X₁₃X₁₄X₁₅Y | 94 |
| acid sequence | X₁₆X₁₇X₁₈X₁₉X₂₀X₂₁ | |
| (Kabat | | |
| definition) | | |
| CDR-L2 amino | X₂₂X₂₃X₂₄X₂₅X₂₆X₂₇X₂₈ | 95 |
| acid sequence | | |
| (Kabat | | |
| definition) | | |
| CDR-L3 amino | X₂₉X₃₀X₃₁X₃₂X₃₃X₃₄X₃₅X₃₆X₃₇ | 96 |
| acid sequence | | |
| (Kabat | | |
| definition) | | |

X₃ = Y or F;
X₄ = W, A, or G;
X₅ = M or I;
X₆ = H or S;
X₇ = N, E, or Y;
X₈ = Y or S;
X₉ = P or S;
X₁₀ = R or G;
X₁₁ = S or G;
X₁₂ = G or S;
X₁₃ = T or Y;
X₁₄ = T or I;
X₁₅ = N or Y;
X₁₆ = D, P, or A;
X₁₇ = G or D;
X₁₈ = Y or T;
X₁₉ = F or V;
X₂₀ = K or T;
X₂₁ = S or G;
X₂₂ = G, S, or L;
X₂₃ = T or I;
X₂₄ = N or S;
X₂₅ = N or R;
X₂₆ = R or L;
X₂₇ = A, H, or F;
X₂₈ = P or S;
X₂₉ = A, Q, or L;
X₃₀ = L or Q;
X₃₁ = L, G, or W;
X₃₂ = Y, S, or T;
X₃₃ = S, T, or H;
X₃₄ = N, L, or Q;
X₃₅ = Y or P;
X₃₆ = W or F;
X₃₇ = Y or T

TABLE 1H
Murine CDR Consensus sequences-Chothia definition

| | | SEQ |
|---|---|---|
| Description | Sequence | ID NO: |
| CDR-H1 amino | GX₁TFX₂SX₃ | 97 |
| acid sequence | | |
| (Chothia | | |
| definition) | | |
| CDR-H2 amino | X₈X₉X₁₀X₁₁X₁₂X₁₃ | 98 |
| acid sequence | | |
| (Chothia | | |
| definition) | | |
| CDR-L2 amino | X₂₂X₂₃X₂₄X₂₅X₂₆X₂₇X₂₈ | 99 |
| acid sequence | | |
| (Chothia | | |
| definition) | | |
| CDR-L3 amino | X₂₉X₃₀X₃₁X₃₂X₃₃X₃₄X₃₅X₃₆X₃₇ | 100 |
| acid sequence | | |
| (Chothia | | |
| definition) | | |

X₁ = Y or F;
X₂ = T or S;
X₃ = Y or F;
X₈ = Y or S;
X₉ = P or S;
X₁₀ = R or G;
X₁₁ = S or G;
X₁₂ = G or S;
X₁₃ = T or Y;
X₂₂ = G, S, or L;
X₂₃ = T or I;
X₂₄ = N or S;
X₂₅ = N or R;
X₂₆ = R or L;
X₂₇ = A, H, or F;
X₂₈ = P or S;
X₂₉ = A, Q, or L;
X₃₀ = L or Q;
X₃₁ = L, G, or W;
X₃₂ = Y, S, or T;
X₃₃ = S, T, or H;
X₃₄ = N, L, or Q;
X₃₅ = Y or P;
X₃₆ = W or F;
X₃₇ = Y or T

TABLE 1I
Overall CDR Consensus sequences-IMGT definition

| | | SEQ |
|---|---|---|
| Description | Sequence | ID NO: |
| CDR-H1 amino | GX₄₁TX₄₂X₄₃X₄₄X₄₅X₄₆ | 228 |
| acid sequence | | |
| (IMGT | | |
| definition) | | |
| CDR-H2 amino | X₅₁X₅₂X₅₃X₅₄X₅₅X₅₆X₅₇ | 229 |
| acid sequence | | |
| (IMGT | | |
| definition) | | |
| CDR-L2 amino | X₆₅X₆₆X₆₇ | 230 |
| acid sequence | | |
| (IMGT | | |
| definition) | | |
| CDR-L3 amino | X₇₂X₇₃X₇₄X₇₅X₇₆X₇₇X₇₈X₇₉X₈₀X₈₁ | 231 |
| acid sequence | | |
| (IMGT | | |
| definition) | | |

X₄₁ = Y or F;
X₄₂ = F or I;
X₄₃ = T, S, or N;
X₄₄ = S or T;
X₄₅ = Y or F;
X₄₆ = W, A, G, or H;
X₅₁ = Y or S;
X₅₂ = P, S, or H;
X₅₃ = R, G, or D;
X₅₄ = S or G;
X₅₅ = G or S;
X₅₆ = T or Y;
X₅₇ = T, I, or gap;
X₆₅ = G, S, L, or K;
X₆₆ = T, I, or A;
X₆₇ = N or S;
X₇₂ = A, Q, or L;
X₇₃ = L, Q, or G;
X₇₄ = L, G, or W;
X₇₅ = Y, S, or T;
X₇₆ = S, T, H, or K;
X₇₇ = N, L, Q, or G;
X₇₈ = Y, P, or D;
X₇₉ = W, F, or I;
X₈₀ = Y, T, or H;
X₈₁ = P or absent

TABLE 1J
Overall CDR Consensus sequences-Kabat definition

| | | SEQ |
|---|---|---|
| Description | Sequence | ID NO: |
| CDR-H1 amino | X₄₄X₄₅X₄₆X₄₇X₄₈ | 232 |
| acid sequence | | |
| (Kabat | | |
| definition) | | |
| CDR-H2 amino | X₄₉X₅₀X₅₁X₅₂X₅₃X₅₄X₅₅X₅₆X₅₇ | 233 |
| acid sequence | X₅₈YX₅₉X₆₀X₆₁X₆₂X₆₃X₆₄ | |
| (Kabat | | |
| definition) | | |
| CDR-L2 amino | X₆₅X₆₆X₆₇X₆₈X₆₉X₇₀X₇₁ | 234 |
| acid sequence | | |
| (Kabat | | |
| definition) | | |
| CDR-L3 amino | X₇₂X₇₃X₇₄X₇₅X₇₆X₇₇X₇₈X₇₉X₈₀X₈₁ | 235 |
| acid sequence | | |
| (Kabat | | |
| definition) | | |

X₄₄ = S or T;
X₄₅ = Y or F;
X₄₆ = W, A, G, or H;
X₄₇ = M or I;
X₄₈ = H, S, or G;
X₄₉ = N, E, Y, or I;
X₅₀ = I or V;
X₅₁ = Y or S;
X₅₂ = P, S, or H;
X₅₃ = R, G, or D;
X₅₄ = S or G;
X₅₅ = G or S;
X₅₆ = T or Y;
X₅₇ = T, I, or gap;
X₅₈ = N or Y;
X₅₉ = D, P, or A;
X₆₀ = G, D, or T;
X₆₁ = Y, T or W;
X₆₂ = F, V, or A;
X₆₃ = K or T;
X₆₄ = S or G;
X₆₅ = G, S, L, or K;
X₆₆ = T, I, or A;
X₆₇ = N or S;
X₆₈ = N, R, or T;
X₆₉ = R or L;
X₇₀ = A, H, or F;
X₇₁ = P or S;
X₇₂ = A, Q, or L;
X₇₃ = L, Q, or G;
X₇₄ = L, G, or W;
X₇₅ = Y, S, or T;
X₇₆ = S, T, H, or K;
X₇₇ = N, L, Q, or G;
X₇₈ = Y, P, or D;
X₇₉ = W, F, or I;
X₈₀ = Y, T, or H;
X₈₁ = P or absent

TABLE 1K
Overall CDR Consensus sequences-Chothia definition

| | | SEQ |
|---|---|---|
| Description | Sequence | ID NO: |
| CDR-H1 amino | GX₄₁TX₄₂X₄₃X₄₄X₄₅ | 236 |
| acid sequence | | |
| (Chothia | | |
| definition) | | |
| CDR-H2 amino | X₅₁X₅₂X₅₃X₅₄X₅₅X₅₆ | 237 |
| acid sequence | | |
| (Chothia | | |
| definition) | | |
| CDR-L2 amino | X₆₅X₆₆X₆₇X₆₈X₆₉X₇₀X₇₁ | 238 |
| acid sequence | | |
| (Chothia | | |
| definition) | | |
| CDR-L3 amino | X₇₂X₇₃X₇₄X₇₅X₇₆X₇₇X₇₈X₇₉X₈₀X₈₁ | 239 |
| acid sequence | | |
| (Chothia | | |
| definition) | | |

X₄₁ = Y or F;
X₄₂ = F or I;
X₄₃ = T, S, or N;
X₄₄ = S or T;
X₄₅ = Y or F;
X₅₁ = Y or S;
X₅₂ = P, S, or H;
X₅₃ = R, G, or D;
X₅₄ = S or G;
X₅₅ = G or S;
X₅₆ = T or Y;
X₆₅ = G, S, L, or K;
X₆₆ = T, I, or A;
X₆₇ = N or S;
X₆₈ = N, R, or T;
X₆₉ = R or L;
X₇₀ = A, H, or F;
X₇₁ = P or S;
X₇₂ = A, Q, or L;
X₇₃ = L, Q, or G;
X₇₄ = L, G, or W;
X₇₅ = Y, S, or T;
X₇₆ = S, T, H, or K;
X₇₇ = N, L, Q, or G;
X₇₈ = Y, P, or D;
X₇₉ = W, F, or I;
X₈₀ = Y, T, or H;
X₈₁ = P or absent

TABLE 2A
4C8 IMGT, Kabat, and
Chothia CDR combined overlap sequences

| | | SEQ |
|---|---|---|
| Description | Sequence | ID NO: |
| CDR-H1 amino | GYTFTSYWMH (IMGT) | 101 |
| acid sequence | GYTFTSYWMH (Kabat) | |
| (combined | GYTFTSYWMH (Chothia) | |
| overlap) | | |
| CDR-H2 amino | NIYPRSGTTNYDGYFKS (IMGT) | 102 |
| acid sequence | NIYPRSGTTNYDGYFKS (Kabat) | |
| (combined | NIYPRSGTTNYDGYFKS (Chothia) | |
| overlap) | | |
| CDR-H3 amino | TRSGYDYPFVY (IMGT) | 103 |
| acid sequence | TRSGYDYPFVY (Kabat) | |
| (combined | TRSGYDYPFVY (Chothia) | |
| overlap) | | |
| CDR-L1 amino | RTSTGAVSIRNYAN (IMGT) | 104 |
| acid sequence | RTSTGAVSIRNYAN (Kabat) | |
| (combined | RTSTGAVSIRNYAN (Chothia) | |
| overlap) | | |
| CDR-L2 amino | GTNNRAP (IMGT) | 105 |
| acid sequence | GTNNRAP (Kabat) | |
| (combined | GTNNRAP (Chothia) | |
| overlap) | | |
| CDR-L3 amino | ALLYSNYWV (IMGT) | 106 |
| acid sequence | ALLYSNYWV (Kabat) | |
| (combined | ALLYSNYWV (Chothia) | |
| overlap) | | |

TABLE 2B
2B2 IMGT, Kabat, and
Chothia CDR combined overlap sequences

| | | SEQ |
|---|---|---|
| Description | Sequence | ID NO: |
| CDR-H1 amino | GFTFSSYAMS (IMGT) | 107 |
| acid sequence | GFTFSSYAMS (Kabat) | |
| (combined | GFTFSSYAMS (Chothia) | |
| overlap) | | |
| CDR-H2 amino | EISSGGSYTYYPDTVTG (IMGT) | 108 |
| acid sequence | EISSGGSYTYYPDTVTG (Kabat) | |
| (combined | EISSGGSYTYYPDTVTG (Chothia) | |
| overlap) | | |
| CDR-H3 amino | ARTVGEDWYFDV (IMGT) | 109 |
| acid sequence | ARTVGEDWYFDV (Kabat) | |
| (combined | ARTVGEDWYFDV (Chothia) | |
| overlap) | | |
| CDR-L1 amino | RASQDISHYLN (IMGT) | 110 |
| acid sequence | RASQDISHYLN (Kabat) | |
| (combined | RASQDISHYLN (Chothia) | |
| overlap) | | |
| CDR-L2 amino | STSRLHS (IMGT) | 111 |
| acid sequence | STSRLHS (Kabat) | |
| (combined | STSRLHS (Chothia) | |
| overlap) | | |
| CDR-L3 amino | QQGYTLPFT (IMGT) | 112 |
| acid sequence | QQGYTLPFT (Kabat) | |
| (combined | QQGYTLPFT (Chothia) | |
| overlap) | | |

TABLE 2C
18G9 IMGT, Kabat, and
Chothia CDR combined overlap sequences

| | | SEQ |
|---|---|---|
| Description | Sequence | ID NO: |
| CDR-H1 amino | GFTFSSFGMH (IMGT) | 113 |
| acid sequence | GFTFSSFGMH (Kabat) | |
| (combined | GFTFSSFGMH (Chothia) | |
| overlap) | | |
| CDR-H2 amino | YISSGSSTIYYADTVKG (IMGT) | 114 |
| acid sequence | YISSGSSTIYYADTVKG (Kabat) | |
| (combined | YISSGSSTIYYADTVKG (Chothia) | |
| overlap) | | |
| CDR-H3 amino | ARGSYRAMDY (IMGT) | 115 |
| acid sequence | ARGSYRAMDY (Kabat) | |
| (combined | ARGSYRAMDY (Chothia) | |
| overlap) | | |
| CDR-L1 amino | SASSSVNYMF (IMGT) | 116 |
| acid sequence | SASSSVNYMF (Kabat) | |
| (combined | SASSSVNYMF (Chothia) | |
| overlap) | | |
| CDR-L2 amino | LTSNLAS (IMGT) | 117 |
| acid sequence | LTSNLAS (Kabat) | |
| (combined | LTSNLAS (Chothia) | |
| overlap) | | |
| CDR-L3 amino | QLWSSNPFT (IMGT) | 118 |
| acid sequence | QLWSSNPFT (Kabat) | |
| (combined | QLWSSNPFT (Chothia) | |
| overlap) | | |

TABLE 2D
1D12 IMGT, Kabat, and
Chothia CDR combined overlap sequences

| | | SEQ |
|---|---|---|
| Description | Sequence | ID NO: |
| CDR-H1 amino | GFTFSSFGIH (IMGT) | 119 |
| acid sequence | GFTFSSFGIH (Kabat) | |
| (combined | GFTFSSFGIH (Chothia) | |
| overlap) | | |
| CDR-H2 amino | YISSGSSTIYYADTVKG (IMGT) | 120 |
| acid sequence | YISSGSSTIYYADTVKG (Kabat) | |
| (combined | YISSGSSTIYYADTVKG (Chothia) | |
| overlap) | | |
| CDR-H3 amino | ARGSKVVAKSRGYWYFDV (IMGT) | 121 |
| acid sequence | ARGSKVVAKSRGYWYFDV (Kabat) | |
| (combined | ARGSKVVAKSRGYWYFDV (Chothia) | |
| overlap) | | |
| CDR-L1 amino | RSSQSLANNYGITYLS (IMGT) | 122 |
| acid sequence | RSSQSLANNYGITYLS (Kabat) | |
| (combined | RSSQSLANNYGITYLS (Chothia) | |
| overlap) | | |
| CDR-L2 amino | GISNRFS (IMGT) | 123 |
| acid sequence | GISNRFS (Kabat) | |
| (combined | GISNRFS (Chothia) | |
| overlap) | | |
| CDR-L3 amino | LQGTHQPWT (IMGT) | 124 |
| acid sequence | LQGTHQPWT (Kabat) | |
| (combined | LQGTHQPWT (Chothia) | |
| overlap) | | |

TABLE 2E
10H4 IMGT, Kabat, and
Chothia CDR combined overlap sequences

| | | SEQ |
|---|---|---|
| Description | Sequence | ID NO: |
| CDR-H1 amino acid | GFTINTYHMG (IMGT) | 240 |
| sequence | GFTINTYHMG (Kabat) | |
| (combined overlap) | GFTINTYHMG (Chothia) | |
| CDR-H2 amino acid | IVSHDVGTYYATWAKG (IMGT) | 241 |
| sequence | IVSHDVGTYYATWAKG (Kabat) | |
| (combined overlap) | IVSHDVGTYYATWAKG (Chothia) | |
| CDR-H3 amino acid | ARGPGYWTFNL (IMGT) | 242 |
| sequence | ARGPGYWTFNL (Kabat) | |
| (combined overlap) | ARGPGYWTFNL (Chothia) | |
| CDR-L1 amino acid | QASQSVYNNNQLS (IMGT) | 243 |
| sequence | QASQSVYNNNQLS (Kabat) | |
| (combined overlap) | QASQSVYNNNQLS (Chothia) | |
| CDR-L2 amino acid | KASTLAS (IMGT) | 244 |
| sequence | KASTLAS (Kabat) | |
| (combined overlap) | KASTLAS (Chothia) | |
| CDR-L3 amino acid | AGGYKGDIHP (IMGT) | 245 |
| sequence | AGGYKGDIHP (Kabat) | |
| (combined overlap) | AGGYKGDIHP (Chothia) | |

TABLE 2F
Murine Consensus CDR combined overlap sequences

| Description | Sequence | SEQ ID NO: |
|---|---|---|
| CDR-H1 amino acid | GX₁TFX₂SX₃X₄X₅X₆ | 125 |
| sequence | | |
| (combined overlap) | | |
| CDR-H2 amino acid | X₇IX₈X₉X₁₀X₁₁X₁₂ | 126 |
| sequence | X₁₃X₁₄X₁₅YX₁₆X₁₇ | |
| (combined overlap) | X₁₈X₁₉X₂₀X₂₁ | |
| CDR-L2 amino acid | X₂₂X₂₃X₂₄X₂₅X₂₆ | 127 |
| sequence | X₂₇X₂₈ | |
| (combined overlap) | | |
| CDR-L3 amino acid | X₂₉X₃₀X₃₁X₃₂X₃₃ | 128 |
| sequence | X₃₄X₃₅X₃₆X₃₇ | |
| (combined overlap) | | |

X₁ = Y or F;
X₂ = T or S;
X₃ = Y or F;
X₄ = W, A, or G;
X₅ = M or I;
X₆ = H or S;
X₇ = N, E, or Y;
X₈ = Y or S;
X₉ = P or S;
X₁₀ = R or G;
X₁₁ = S or G;
X₁₂ = G or S;
X₁₃ = T or Y;
X₁₄ = T or I;
X₁₅ = N or Y;
X₁₆ = D, P, or A;
X₁₇ = G or D;
X₁₈ = Y or T;
X₁₉ = F or V;
X₂₀ = K or T;
X₂₁ = S or G;
X₂₂ = G, S, or L;
X₂₃ = T or I;
X₂₄ = N or S;
X₂₅ = N or R;
X₂₆ = R or L;
X₂₇ = A, H, or F;
X₂₈ = P or S;
X₂₉ = A, Q, or L;
X₃₀ = L or Q;
X₃₁ = L, G, or W;
X₃₂ = Y, S, or T;
X₃₃ = S, T, or H;
X₃₄ = N, L, or Q;
X₃₅ = Y or P;
X₃₈ = W or F;
X₃₇ = Y or T

TABLE 2G
Overall Consensus CDR combined overlap sequences

| Description | Sequence | SEQ ID NO: |
|---|---|---|
| CDR-H1 amino acid | GX₄₁TX₄₂X₄₃X₄₄X₄₅X₄₆X₄₇X₄₈ | 246 |
| sequence | | |
| (combined overlap) | | |
| CDR-H2 amino acid | X₄₉X₅₀X₅₁X₅₂X₅₃X₅₄X₅₅X₅₆X₅₇ | 247 |
| sequence | X₅₈YX₅₉X₆₀X₆₁X₆₂X₆₃X₆₄ | |
| (combined overlap) | | |
| CDR-L2 amino acid | X₆₅X₆₆X₆₇X₆₈X₆₉X₇₀X₇₁ | 248 |
| sequence | | |
| (combined overlap) | | |
| CDR-L3 amino acid | X₇₂X₇₃X₇₄X₇₅X₇₆X₇₇X₇₈X₇₉X₈₀ | 249 |
| sequence | X₈₁ | |
| (combined overlap) | | |

X₄₁ = Y or F;
X₄₂ = F or I;
X₄₃ = T, S, or N;
X₄₄ = S or T;
X₄₅ = Y or F;
X₄₆ = W, A, G, or H;
X₄₇ = M or I;
X₄₈ = H, S, or G;
X₄₉ = N, E, Y, or I;
X₅₀ = I or V;
X₅₁ = Y or S;
X₅₂ = P, S, or H;
X₅₃ = R, G, or D;
X₅₄ = S or G;
X₅₅ = G or S;
X₅₆ = T or Y;
X₅₇ = T, I, or gap;
X₅₈ = N or Y;
X₅₉ = D, P, or A;
X₆₀ = G, D, or T;
X₆₁ = Y, T or W;
X₆₂ = F, V, or A;
X₆₃ = K or T;
X₆₄ = S or G;
X₆₅ = G, S, L, or K;
X₆₆ = T, I, or A;
X₆₇ = N or S;
X₆₈ = N, R, or T;
X₆₉ = R or L;
X₇₀ = A, H, or F;
X₇₁ = P or S;
X₇₂ = A, Q, or L;
X₇₃ = L, Q, or G;
X₇₄ = L, G, or W;
X₇₅ = Y, S, or T;
X₇₆ = S, T, H, or K;
X₇₇ = N, L, Q, or G;
X₇₈ = Y, P, or D;
X₇₉ = W, F, or I;
X₈₀ = Y, T, or H;
X₈₁ = P or absent

TABLE 3A
4C8 IMGT, Kabat, and
Chothia CDR common sequences

| Description | Sequence | SEQ ID NO: |
|---|---|---|
| CDR-H1 amino acid | SY | 129 |
| sequence | | |
| (common sequence) | | |
| CDR-H2 amino acid | YPRSGT | 130 |
| sequence | | |
| (common sequence) | | |
| CDR-H3 amino acid | SGYDYPFVY | 131 |
| sequence | | |
| (common sequence) | | |
| CDR-L1 amino acid | TGAVSIRNY | 132 |
| sequence | | |
| (common sequence) | | |
| CDR-L2 amino acid | GTN | 133 |
| sequence | | |
| (common sequence) | | |
| CDR-L3 amino acid | ALLYSNYWV | 134 |
| sequence | | |
| (common sequence) | | |

TABLE 3B
2B2 IMGT, Kabat, and
Chothia CDR common sequences

| Description | Sequence | SEQ ID NO: |
|---|---|---|
| CDR-H1 amino acid | SY | 135 |
| sequence | | |
| (common sequence) | | |
| CDR-H2 amino acid | SSGGSY | 136 |
| sequence | | |
| (common sequence) | | |
| CDR-H3 amino acid | TVGEDWYFDV | 137 |
| sequence | | |
| (common sequence) | | |
| CDR-L1 amino acid | QDISHY | 138 |
| sequence | | |
| (common sequence) | | |
| CDR-L2 amino acid | STS | 139 |
| sequence | | |
| (common sequence) | | |
| CDR-L3 amino acid | QQGYTLPFT | 140 |
| sequence | | |
| (common sequence) | | |

TABLE 3C
18G9 IMGT, Kabat, and Chothia CDR common sequences

| Description | Sequence | SEQ ID NO: |
|---|---|---|
| CDR-H1 amino acid sequence | SF | 141 |
| (common sequence) | | |
| CDR-H2 amino acid sequence | SSGSST | 142 |
| (common sequence) | | |
| CDR-H3 amino acid sequence | GSYRAMDY | 143 |
| (common sequence) | | |
| CDR-L1 amino acid sequence | SSVNY | 144 |
| (common sequence) | | |
| CDR-L2 amino acid sequence | LTS | 145 |
| (common sequence) | | |
| CDR-L3 amino acid sequence | QLWSSNPFT | 146 |
| (common sequence) | | |

TABLE 3D
1D12 IMGT, Kabat, and Chothia CDR common sequences

| Description | Sequence | SEQ ID NO: |
|---|---|---|
| CDR-H1 amino acid | SF | 147 |
| sequence | | |
| (common sequence) | | |
| CDR-H2 amino acid | SSGSST | 148 |
| sequence | | |
| (common sequence) | | |
| CDR-H3 amino acid | GSKVVAKSRGYWYFDV | 149 |
| sequence | | |
| (common sequence) | | |
| CDR-L1 amino acid | QSLANNYGITY | 150 |
| sequence | | |
| (common sequence) | | |
| CDR-L2 amino acid | GIS | 151 |
| sequence | | |
| (common sequence) | | |
| CDR-L3 amino acid | LQGTHQPWT | 152 |
| sequence | | |
| (common sequence) | | |

TABLE 3E
10H4 IMGT, Kabat, and Chothia CDR common sequences

| Description | Sequence | SEQ ID NO: |
|---|---|---|
| CDR-H1 amino acid sequence | TY | 250 |
| (common sequence) | | |
| CDR-H2 amino acid sequence | SHDVG | 251 |
| (common sequence) | | |
| CDR-H3 amino acid sequence | GPGYWTFNL | 252 |
| (common sequence) | | |
| CDR-L1 amino acid sequence | QSVYNNNQ | 253 |
| (common sequence) | | |
| CDR-L2 amino acid sequence | KAS | 254 |
| (common sequence) | | |
| CDR-L3 amino acid sequence | AGGYKGDIHP | 255 |
| (common sequence) | | |

TABLE 3F
Murine Consensus CDR common sequences

| Description | Sequence | SEQ ID NO: |
|---|---|---|
| CDR-H1 amino acid | SX₃ | 153 |
| sequence | | |
| (common sequence) | | |
| CDR-H2 amino acid | X₈X₉X₁₀X₁₁X₁₂X₁₃ | 154 |
| sequence | | |
| (common sequence) | | |
| CDR-L2 amino acid | X₂₂X₂₃X₂₄ | 155 |
| sequence | | |
| (common sequence) | | |
| CDR-L3 amino acid | X₂₉X₃₀X₃₁X₃₂X₃₃X₃₄X₃₅X₃₆X₃₇ | 156 |
| sequence | | |
| (common sequence) | | |

X₃ = Y or F;
X₈ = Y or S;
X₉ = P or S;
X₁₀ = R or G;
X₁₁ = S or G;
X₁₂ = G or S;
X₁₃ = T or Y;
X₂₂ ⁼ G, S, or L;
X₂₃ ⁼ T or I;
X₂₄ ⁼ N or S;
X₂₉ = A, Q, or L;
X₃₀ = L or Q;
X₃₁ = L, G, or W;
X₃₂ = Y, S, or T;
X₃₃ = S, T, or H;
X₃₄ = N, L, or Q;
X₃₅ = Y or P;
X₃₆ = W or F;
X₃₇ = Y or T

TABLE 3G
Overall Consensus CDR common sequences

| Description | Sequence | SEQ ID NO: |
|---|---|---|
| CDR-H1 amino acid | X₄₄X₄₅ | 256 |
| sequence | | |
| (common sequence) | | |
| CDR-H2 amino acid | X₅₁X₅₂X₅₃X₅₄X₅₅X₅₆ | 257 |
| sequence | | |
| (common sequence) | | |
| CDR-L2 amino acid | X₆₅X₆₆X₆₇ | 258 |
| sequence | | |
| (common sequence) | | |
| CDR-L3 amino acid | X₇₂X₇₃X₇₄X₇₅X₇₆X₇₇ | 259 |
| sequence | X₇₈X₇₉X₈₀ | |
| (common sequence) | | |

X₄₄ = S or T;
X₄₅ = Y or F;
X₅₁ = Y or S;
X₅₂ = P, S, or H;
X₅₃ = R, G, or D;
X₅₄ = S or G;
X₅₅ = G or S;
X₅₆ = T or Y;
X₆₅ = G, S, L, or K;
X₆₆ = T, I, or A;
X₆₇ = N or S;
X₇₂ = A, Q, or L;
X₇₃ = L, Q, or G;
X₇₄ = L, G, or W;
X₇₅ = Y, S, or T;
X₇₆ = S, T, H, or K;
X₇₇ = N, L, Q, or G;
X₇₈ = Y, P, or D;
X₇₉ = W, F, or I;
X₈₀ = Y, T, or H

In certain aspects, an anti-glyco-CD44 antibody or antigen-binding fragment of the disclosure comprises a combination of CDRs selected from CDR sequences set forth in Tables 1-3. In some embodiments, CDR-H1 comprises the amino acid sequence of SEQ ID NO: 3, 9, 15, 25, 31, 37, 47, 53, 59, 69, 75, 81, 89, 93, 97, 101, 107, 113, 119, 125, 129, 135, 141, 147, 153, 208, 214, 220, 228, 232, 236, 240, 246, 250, or 256. In some embodiments, CDR-H2 comprises the amino acid sequence of SEQ ID NO: 4, 10, 16, 26, 32, 38, 48, 54, 60, 70, 76, 82, 90, 94, 98, 102, 108, 114, 120, 126, 130, 136, 142, 148, 154, 209. 215, 221, 229, 233, 237, 241, 247, 251, or 257. In some embodiments, CDR-H3 comprises the amino acid sequence of SEQ ID NO: 5, 11, 17, 27, 33, 39, 49, 55, 61, 71, 77, 83, 103, 109, 115, 121, 131, 137, 143, 149, 210, 216, 222, 242, 252. In some embodiments, CDR-L1 comprises the amino acid sequence of SEQ ID NO: 6, 12, 18, 28, 34, 40, 50, 56, 62, 72, 78, 84, 104, 110, 116, 122, 132, 138, 144, 150, 211, 217, 223, 243, 253. In some embodiments, CDR-L2 comprises the amino acid sequence of SEQ ID NO: 7, 13, 19, 29, 35, 41, 51, 57, 63, 73, 79, 85, 91, 95, 99, 105, 111, 117, 123, 127, 133, 139, 145, 151, 155, 212, 218, 224, 230, 234, 238, 244, 248, 254, 258. In some embodiments, CDR-L3 comprises the amino acid sequence of SEQ ID NO: 8, 14, 20, 30, 36, 42, 52, 58, 64, 74, 80, 86, 92, 96, 100, 106, 112, 118, 124, 128, 134, 140, 146, 152, 156, 213, 219, 225, 231, 235, 239, 245, 249, 255. 259.

In certain aspects, an anti-glyco-CD44 antibody or antigen-binding fragment of the disclosure comprises CDRs comprising the amino acid sequences of any of the CDR combinations set forth in numbered embodiments 13 to 275. Thus, in certain embodiments, an anti-glyco-CD44 antibody or antigen-binding fragment of the disclosure comprises a CDR-H1 comprising the amino acid sequence of SEQ ID NO:89, SEQ ID NO:93, SEQ ID NO:97, SEQ ID NO:125, SEQ ID NO:153, SEQ ID NO:228, SEQ ID NO:232, SEQ ID NO:236, SEQ ID NO:246, or SEQ ID NO:256; a CDR-H2 comprising the amino acid sequence of SEQ ID NO:90, SEQ ID NO:94, SEQ ID NO:98, SEQ ID NO:229, SEQ ID NO:233, SEQ ID NO:237; a CDR-H3 comprising the amino acid sequence of SEQ ID NO:103, SEQ ID NO: 109, SEQ ID NO: 115, SEQ ID NO:121, SEQ ID NO:131, SEQ ID NO:137, SEQ ID NO:143, SEQ ID NO: 149, SEQ ID NO:242, or SEQ ID NO:252; a CDR-L1 comprising the amino acid sequence of SEQ ID NO:104, SEQ ID NO:110, SEQ ID NO:116, SEQ ID NO:122, SEQ ID NO:132, SEQ ID NO:138, SEQ ID NO:144, SEQ ID NO:150, SEQ ID NO:234, or SEQ ID NO:253; a CDR-L2 comprising the amino acid sequence of SEQ ID NO:91, SEQ ID NO:95, SEQ ID NO:230, or SEQ ID NO:234; and a CDR-L3 comprising the amino acid sequence of SEQ ID NO:92 or SEQ ID NO:231.

In other aspects, an anti-glyco-CD44 antibody or antigen-binding fragment of the disclosure comprises heavy chain CDRs of SEQ ID NOS:3-5 and light chain CDRs of SEQ ID NOS:6-8. In other aspects, an anti-glyco-CD44 antibody or antigen-binding fragment of the disclosure comprises heavy chain CDRs of SEQ ID NOS:9-11 and light chain CDRs of SEQ ID NOS:12-14. In other aspects, an anti-glyco-CD44 antibody or antigen-binding fragment of the disclosure comprises heavy chain CDRs of SEQ ID NOS:15-17 and light chain CDRs of SEQ ID NOS:18-20.

In other aspects, an anti-glyco-CD44 antibody or antigen-binding fragment of the disclosure comprises heavy chain CDRs of SEQ ID NOS:25-27 and light chain CDRs of SEQ ID NOS:28-30. In other aspects, an anti-glyco-CD44 antibody or antigen-binding fragment of the disclosure comprises heavy chain CDRs of SEQ ID NOS:31-33 and light chain CDRs of SEQ ID NOS:34-36. In other aspects, an anti-glyco-CD44 antibody or antigen-binding fragment of the disclosure comprises heavy chain CDRs of SEQ ID NOS:37-39 and light chain CDRs of SEQ ID NOS:40-42.

In other aspects, an anti-glyco-CD44 antibody or antigen-binding fragment of the disclosure comprises heavy chain CDRs of SEQ ID NOS:47-49 and light chain CDRs of SEQ ID NOS:50-52. In other aspects, an anti-glyco-CD44 antibody or antigen-binding fragment of the disclosure comprises heavy chain CDRs of SEQ ID NOS:53-55 and light chain CDRs of SEQ ID NOS:56-58. In other aspects, an anti-glyco-CD44 antibody or antigen-binding fragment of the disclosure comprises heavy chain CDRs of SEQ ID NOS:59-61 and light chain CDRs of SEQ ID NOS:62-64.

In other aspects, an anti-glyco-CD44 antibody or antigen-binding fragment of the disclosure comprises heavy chain CDRs of SEQ ID NOS:69-71 and light chain CDRs of SEQ ID NOS:72-74. In other aspects, an anti-glyco-CD44 antibody or antigen-binding fragment of the disclosure comprises heavy chain CDRs of SEQ ID NOS:75-77 and light chain CDRs of SEQ ID NOS:78-80. In other aspects, an anti-glyco-CD44 antibody or antigen-binding fragment of the disclosure comprises heavy chain CDRs of SEQ ID NOS:81-83 and light chain CDRs of SEQ ID NOS:84-86.

In other aspects, an anti-glyco-CD44 antibody or antigen-binding fragment of the disclosure comprises heavy chain CDRs of SEQ ID NOS:208-210 and light chain CDRs of SEQ ID NOS:211-213. In other aspects, an anti-glyco-CD44 antibody or antigen-binding fragment of the disclosure comprises heavy chain CDRs of SEQ ID NOS:214-216 and light chain CDRs of SEQ ID NOS:217-219. In other aspects, an anti-glyco-CD44 antibody or antigen-binding fragment of the disclosure comprises heavy chain CDRs of SEQ ID NOS:220-222 and light chain CDRs of SEQ ID NOS:223-225.

In other aspects, an anti-glyco-CD44 antibody or antigen-binding fragment of the disclosure comprises heavy chain CDRs of SEQ ID NOS:101-103 and light chain CDRs of SEQ ID NOS:104-106. In other aspects, an anti-glyco-CD44 antibody or antigen-binding fragment of the disclosure comprises heavy chain CDRs of SEQ ID NOS:107-109 and light chain CDRs of SEQ ID NOS:110-112. In other aspects, an anti-glyco-CD44 antibody or antigen-binding fragment of the disclosure comprises heavy chain CDRs of SEQ ID NOS:113-115 and light chain CDRs of SEQ ID NOS:116-118. In other aspects, an anti-glyco-CD44 antibody or antigen-binding fragment of the disclosure comprises heavy chain CDRs of SEQ ID NOS:119-121 and light chain CDRs of SEQ ID NOS:122-124. In other aspects, an anti-glyco-CD44 antibody or antigen-binding fragment of the disclosure comprises heavy chain CDRs of SEQ ID NOS:240-242 and light chain CDRs of SEQ ID NOS:243-245.

In other aspects, an anti-glyco-CD44 antibody or antigen-binding fragment of the disclosure comprises heavy chain CDRs of SEQ ID NOS:129-131 and light chain CDRs of SEQ ID NOS:132-134. In other aspects, an anti-glyco-CD44 antibody or antigen-binding fragment of the disclosure comprises heavy chain CDRs of SEQ ID NOS:135-137 and light chain CDRs of SEQ ID NOS:138-140. In other aspects, an anti-glyco-CD44 antibody or antigen-binding fragment of the disclosure comprises heavy chain CDRs of SEQ ID NOS:141-143 and light chain CDRs of SEQ ID NOS:144-146. In other aspects, an anti-glyco-CD44 antibody or antigen-binding fragment of the disclosure comprises heavy chain CDRs of SEQ ID NOS:147-149 and light chain CDRs of SEQ ID NOS:150-152. In other aspects, an anti-glyco-CD44 antibody or antigen-binding fragment of the disclosure comprises heavy chain CDRs of SEQ ID NOS:250-252 and light chain CDRs of SEQ ID NOS:253-255.

The antibodies and antigen-binding fragments of the disclosure can be murine, rabbit, chimeric, humanized or human.

In further aspects, an anti-glyco-CD44 antibody or antigen binding fragment of the disclosure competes with an antibody or antigen binding fragment comprising heavy and light chain variable regions of SEQ ID NOS:1 and 2, respectively. In yet other aspects, the disclosure provides an anti-CD44 antibody or antigen binding fragment having heavy and light chain variable regions having at least 95%, 98%, 99%, or 99.5% sequence identity of SEQ ID NOS:1 and 2, respectively.

In yet other aspects, an anti-glyco-CD44 antibody or antigen binding fragment of the disclosure competes with an antibody or antigen binding fragment comprising heavy and light chain variable regions of SEQ ID NOS:23 and 24, respectively. In yet other aspects, the disclosure provides an anti-CD44 antibody or antigen binding fragment having heavy and light chain variable regions having at least 95%, 98%, 99%, or 99.5% sequence identity of SEQ ID NOS:23 and 24, respectively.

In yet other aspects, an anti-glyco-CD44 antibody or antigen binding fragment of the disclosure competes with an antibody or antigen binding fragment comprising heavy and light chain variable regions of SEQ ID NOS:45 and 46, respectively. In yet other aspects, the disclosure provides an anti-CD44 antibody or antigen binding fragment having heavy and light chain variable regions having at least 95%, 98%, 99%, or 99.5% sequence identity of SEQ ID NOS:45 and 46, respectively.

In yet other aspects, an anti-glyco-CD44 antibody or antigen binding fragment of the disclosure competes with an antibody or antigen binding fragment comprising heavy and light chain variable regions of SEQ ID NOS:67 and 68, respectively. In yet other aspects, the disclosure provides an anti-CD44 antibody or antigen binding fragment having heavy and light chain variable regions having at least 95%, 98%, 99%, or 99.5% sequence identity of SEQ ID NOS:67 and 68, respectively.

In yet other aspects, an anti-glyco-CD44 antibody or antigen binding fragment of the disclosure competes with an antibody or antigen binding fragment comprising heavy and light chain variable regions of SEQ ID NOS:206 and 207, respectively. In yet other aspects, the disclosure provides an anti-CD44 antibody or antigen binding fragment having heavy and light chain variable regions having at least 95%, 98%, 99%, or 99.5% sequence identity of SEQ ID NOS:206 and 207, respectively.

In yet other aspects, an anti-glyco-CD44 antibody or antigen-binding fragment of the disclosure is a single-chain variable fragment (scFv). An exemplary scFv comprises the heavy chain variable fragment N-terminal to the light chain variable fragment. In some embodiments, the scFv heavy chain variable fragment and light chain variable fragment are covalently bound to a linker sequence of 4-15 amino acids. The scFv can be in the form of a bi-specific T-cell engager or within a chimeric antigen receptor (CAR).

The anti-glyco-CD44 antibodies and antigen-binding fragments can be in the form of a multimer of a single-chain variable fragment, a bispecific single-chain variable fragment and a multimer of a bispecific single-chain variable fragment. In some embodiments, the multimer of a single chain variable fragment is selected a divalent single-chain variable fragment, a tribody or a tetrabody. In some of these embodiments, the multimer of a bispecific single-chain variable fragment is a bispecific T-cell engager.

Other aspects of the disclosure are drawn to nucleic acids encoding the anti-glyco-CD44 antibodies and antibody-binding fragments of the disclosure. In some embodiments, the portion of the nucleic acid nucleic acid encoding an anti-glyco-CD44 antibody or antigen-binding fragment is codon-optimized for expression in a human cell. In certain aspects, the disclosure provides an anti-glyco-CD44 antibody or antigen binding fragment having heavy and light chain variable regions encoded by a heavy chain nucleotide sequence having at least 95%, 98%, 99%, or 99.5% sequence identity to SEQ ID NO:1, SEQ ID NO:23, SEQ ID NO:45, SEQ ID NO:67, or SEQ ID NO:206 and a light chain nucleotide sequence having at least 95%, 98%, 99%, or 99.5% sequence identity to SEQ ID NO:2, SEQ ID NO:24, SEQ ID NO:46, SEQ ID NO:68, or SEQ ID NO:207. Vectors (e.g., a viral vector such as a lentiviral vector) and host cells comprising the nucleic acids are also within the scope of the disclosure. The heavy and light chains coding sequences can be present on a single vector or on separate vectors.

Yet another aspect of the disclosure is a pharmaceutical composition comprising an anti-glyco-CD44 antibody, antigen-binding fragment, nucleic acid (or pair of nucleic acids), vector (or pair of vectors) or host cell according to the disclosure, and a physiologically suitable buffer, adjuvant, or diluent.

Still another aspect of the disclosure is a method of making a chimeric antigen receptor comprising incubating a cell comprising a nucleic acid or a vector according to the disclosure, under conditions suitable for expression of the coding region and collecting the chimeric antigen receptor.

Another aspect of the disclosure is a method of detecting cancer comprising contacting a cell or tissue sample with an anti-glyco-CD44 antibody or antigen-binding fragment of the disclosure and detecting whether the antibody is bound to the cell or tissue sample.

Yet another aspect of the disclosure is an anti-glyco-CD44 antibody or antigen-binding fragment according to the disclosure of the disclosure for use in detecting cancer.

Yet another aspect of the disclosure is a method of treating cancer comprising administering a prophylactically or therapeutically effective amount of an anti-glyco-CD44 antibody, antigen-binding fragment, nucleic acid, vector, host cell or pharmaceutical composition according to the disclosure to a subject in need thereof.

Yet another aspect of the disclosure is an anti-glyco-CD44 antibody, antigen-binding fragment, nucleic acid, vector, host cell or pharmaceutical composition according to the disclosure for use in the treatment of cancer.

Yet another aspect of the disclosure is use of an anti-glyco-CD44 antibody, antigen-binding fragment, nucleic acid, vector, host cell or pharmaceutical composition according to the disclosure for the manufacture of a medicament for the treatment of cancer.

CD44v6 peptides are also provided herein. The peptides can be 12-30 amino acids in length and comprise amino acids 4-13 of SEQ ID NO:165. The CD44v6 peptides are described in Section 6.8 and numbered embodiments 534 to 539. The peptides can be included in a composition, as described in Section 6.8.1 and numbered embodiments 540 to 541. The CD44v6 peptides can be used in methods for producing antibodies in an animal and/or eliciting an immune response in an animal. Methods for using the CD44v6 peptides are described in Section 6.8.2 and numbered embodiments 542 to 545.

### 5. BRIEF DESCRIPTION OF THE FIGURES

FIGS. 1A-F: shows that antibody 4C8 specifically binds Tn-glycosylated CD44. FIG. 1A: ELISA performed with 1 μg/mL 4C8 mAb against various concentrations of non-glycosylated and Tn-glycosylated CD44 and MUC1. FIG. 1B: The affinity of 4C8 mAb for CD44v6 glycopeptide determined using Biacore and Octet technologies. FIG. 1C: HaCaT WT and COSMC KO cell staining using α-Golgi, various dilutions of 4C8 mAb supernatant, α-CD44v6, and a mouse IgG isotype control. FIG. 1D: HaCaT WT and COSMC KO cell immunofluorescence staining using 4C8 mAb, α-CD44v6, and α-Tn. FIG. 1E: HaCaT WT and COSMC KO cells grown into an organotypic skin model on a collagen-gel containing human fibroblasts, fixed, embedded in paraffin, and stained for immunofluorescence using 4C8 mAb and α-CD44v6. FIG. 1F: Biopsies from healthy human skin stained for immunofluorescence using 4C8 mAb and α-CD44v6.

FIGS. 2A-2B: shows that antibody 4C8 selectively stains several primary cancer tissues. FIG. 2A: Tissue microarrays for several carcinomas and adjacent healthy tissues stained for immunohistochemistry using 4C8 mAb, α-CD44, and a mouse IgG isotype control. FIG. 2B: Tables showing the distribution of strong, weak and negative stained tissue sections observed in the immunohistochemistry portrayed in FIG. 2A, divided into grade 1, grade 2, and grade 3 carcinomas for each cancer type.

FIG. 3A-3C: shows that 4C8 CAR T cells selectively kill Tn-positive cancer cells. FIG. 3A: Results of cytotoxity assay performed with 4C8 CAR T cells (Construct 1) co-cultured with HaCaT WT and COSMC KO cells. FIG. 3B: Concentration of IFN-γ in co-culture supernatants analysed by ELISA. FIG. 3C: Expression of T cell activation markers asssedded using flow cytometry.

FIG. 4: shows results of a cytotoxicity assay performed with 4C8 CAR T cells co-cultured with HaCaT WT and COSMC KO cells at a 3 to 1 ratio. NV (no vector) is T cells alone with no 4C8 CAR expressed. The orientation of light chain (L) at the N-terminus (Construct 1) was found to be more effective than the heavy chain at the N-terminus (Construct 4).

FIG. 5A-5H: schematic representations of representative 4C8 CAR constructs 1-8. FIG. 5A: Construct 1 (LH-4C8-CD8a-CART); FIG. 5B: Construct 2 (LH-408-IgG4-CART); FIG. 5C: Construct 3 (LH-4C8-IgG4-Long-CART); FIG. 5D: Construct 4 (HL-4C8-CD8a-CART); FIG. 5E: Construct 5 (HL-4C8-IgG4-CART); FIG. 5F: Construct 6 (HL-4C8-IgG4-Long-CART); FIG. 5G: Construct 7 (LHx2-4C8-CD8-CART); FIG. 5H: Construct 8 (HLx2-4C8-CD8-CART). FIGS. 5A-5H disclose “(GGGGS)3” as SEQ ID NO: 184 and “(GGGGS)1” as SEQ ID NO: 183.

FIG. 6: schematic representation of a representative 10H4 CAR construct.

### 6. DETAILED DESCRIPTION

6.1 Antibodies

Each of the potential 117 O-linked glycosylation sites within the CD44 variant region have the potential to be targets for therapeutic antibodies. It is unknown which glycosylation sites can be effectively targeted. The CD44v6 domain alone includes 13 potential O-linked glycosylation sites, including 4 serines and 9 threonines. Each of these sites could potentially be used as antibody targets. The disclosure provides novel antibodies that are directed to a specific glycoform of CD44v6 present on tumor cells. These are exemplified by the antibodies 4C8, 2B2, 18G9, 1D12, and 10H4. 4C8, 2B2, 18G9, and 1D12 were identified in a screen for murine antibodies that bind to a glycosylated peptide present in a particular glycoform of CD44v6, GYRQTPKEDSHSTTGTAAA (SEQ ID NO:165), glycosylated with GalNAc on the serine and threonine residues shown in bold underlined text (the “CD44v6 glycopeptide”) so as to mimic the glycosylation pattern of CD44v6 present on tumor cells. 10H4 was identified in a screen for rabbit antibodies that bind to the same CD44v6 glycopeptide.

The anti-glyco-CD44 antibodies of the disclosure, exemplified by antibodies 4C8, 2B2, 18G9, 1D12, and 10H4, are useful as tools in cancer diagnosis and therapy.

Thus, in certain aspects, the disclosure provides antibodies and antigen binding fragments that bind to a glycoform of CD44 present on tumor cells (referred to herein as “glyco-CD44”), and preferably to the CD44v6 glycopeptide.

The anti-glyco-CD44 antibodies of the disclosure may be polyclonal, monoclonal, genetically engineered, and/or otherwise modified in nature, including but not limited to chimeric antibodies, humanized antibodies, human antibodies, primatized antibodies, single chain antibodies, bispecific antibodies, dual-variable domain antibodies, etc. In various embodiments, the antibodies comprise all or a portion of a constant region of an antibody. In some embodiments, the constant region is an isotype selected from: IgA (e.g., IgA₁ or IgA₂), IgD, IgE, IgG (e.g., IgG₁, IgG₂, IgG₃ or IgG₄), and IgM. In specific embodiments, the anti-glyco-CD44 antibodies of the disclosure comprise an IgG₁ constant region isotyope.

The term “monoclonal antibody” as used herein is not limited to antibodies produced through hybridoma technology. A monoclonal antibody is derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof. In many uses of the present disclosure, including in vivo use of the anti-glyco-CD44 antibodies in humans, chimeric, primatized, humanized, or human antibodies can suitably be used.

The term “chimeric” antibody as used herein refers to an antibody having variable sequences derived from a non-human immunoglobulin, such as a rat or a mouse antibody, and human immunoglobulin constant regions, typically chosen from a human immunoglobulin template. Methods for producing chimeric antibodies are known in the art. See, e.g., Morrison, 1985, Science 229(4719):1202-7; Oi et al., 1986, BioTechniques 4:214-221; Gillies et al., 1985, J. Immunol. Methods 125:191-202; U.S. Pat. Nos. 5,807,715; 4,816,567; and 4,816397, which are incorporated herein by reference in their entireties.

“Humanized” forms of non-human (e.g., murine) antibodies are chimeric immunoglobulins that contain minimal sequences derived from non-human immunoglobulin. In general, a humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody can also comprise at least a portion of an immunoglobulin constant region (Fc), typically that of a human immunoglobulin consensus sequence. Methods of antibody humanization are known in the art. See, e.g., Riechmann et al., 1988, Nature 332:323-7; U.S. Pat. Nos. 5,530,101; 5,585,089; 5,693,761; 5,693,762; and 6,180,370 to Queen et al.; EP239400; PCT publication WO 91/09967; U.S. Pat. No. 5,225,539; EP592106; EP519596; Padlan, 1991, Mol. Immunol., 28:489-498; Studnicka et al., 1994, Prot. Eng. 7:805-814; Roguska et al., 1994, Proc. Natl. Acad. Sci. 91:969-973; and U.S. Pat. No. 5,565,332, all of which are hereby incorporated by reference in their entireties.

“Human antibodies” include antibodies having the amino acid sequence of a human immunoglobulin and include antibodies isolated from human immunoglobulin libraries or from animals transgenic for one or more human immunoglobulin and that do not express endogenous immunoglobulins. Human antibodies can be made by a variety of methods known in the art including phage display methods using antibody libraries derived from human immunoglobulin sequences. See U.S. Pat. Nos. 4,444,887 and 4,716,111; and PCT publications WO 98/46645; WO 98/50433; WO 98/24893; WO 98/16654; WO 96/34096; WO 96/33735; and WO 91/10741, each of which is incorporated herein by reference in its entirety. Human antibodies can also be produced using transgenic mice which are incapable of expressing functional endogenous immunoglobulins but which can express human immunoglobulin genes. See, e.g., PCT publications WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entireties. Fully human antibodies that recognize a selected epitope can be generated using a technique referred to as “guided selection.” In this approach, a selected non-human monoclonal antibody, e.g., a mouse antibody, is used to guide the selection of a completely human antibody recognizing the same epitope (see, Jespers et al., 1988, Biotechnology 12:899-903).

“Primatized antibodies” comprise monkey variable regions and human constant regions. Methods for producing primatized antibodies are known in the art. See, e.g., U.S. Pat. Nos. 5,658,570; 5,681,722; and 5,693,780, which are incorporated herein by reference in their entireties.

Anti-glyco-CD44 antibodies of the disclosure include both full-length (intact) antibody molecules, as well as antigen-binding fragments that are capable of binding glyco-CD44. Examples of antigen-binding fragments include by way of example and not limitation, Fab, Fab′, F (ab′)₂, Fv fragments, single chain Fv fragments and single domain fragments.

A Fab fragment contains the constant domain of the light chain (CL) and the first constant domain (CH1) of the heavy chain. Fab′ fragments differ from Fab fragments by the addition of a few residues at the carboxyl terminus of the heavy chain CH1 domain including one or more cysteines from the antibody hinge region. F(ab′) fragments are produced by cleavage of the disulfide bond at the hinge cysteines of the F(ab′)₂ pepsin digestion product. Additional chemical couplings of antibody fragments are known to those of ordinary skill in the art. Fab and F(ab′)₁ fragments lack the Fc fragment of intact antibody, clear more rapidly from the circulation of animals, and may have less non-specific tissue binding than an intact antibody (see, e.g., Wahl et al., 1983, J. Nucl. Med. 24:316).

An “Fv” fragment is the minimum fragment of an antibody that contains a complete target recognition and binding site. This region consists of a dimer of one heavy and one light chain variable domain in a tight, non-covalent association (V_{H}-V_{L} dimer). It is in this configuration that the three CDRs of each variable domain interact to define a target binding site on the surface of the V_{H}-V_{L} dimer. Often, the six CDRs confer target binding specificity to the antibody. However, in some instances even a single variable domain (or half of an Fv comprising only three CDRs specific for a target) can have the ability to recognize and bind target, although at a lower affinity than the entire binding site.

“Single-chain Fv” or “scFv” antigen-binding fragments comprise the V_{H} and V_{L} domains of an antibody, where these domains are present in a single polypeptide chain. Generally, the Fv polypeptide further comprises a polypeptide linker between the V_{H} and V_{L} domains which enables the scFv to form the desired structure for target binding.

“Single domain antibodies” are composed of single V_{H} or V_{L} domains which exhibit sufficient affinity to glyco-CD44. In a specific embodiment, the single domain antibody is a camelized antibody (See, e.g., Riechmann, 1999, Journal of Immunological Methods 231:25-38).

The anti-glyco-CD44 antibodies of the disclosure may also be bispecific and other multiple specific antibodies. Bispecific antibodies are monoclonal, often human or humanized, antibodies that have binding specificities for two different epitopes on the same or different antigen. In the present disclosure, one of the binding specificities can be directed towards glyco-CD44, the other can be for any other antigen, e.g., for a cell-surface protein, receptor, receptor subunit, tissue-specific antigen, virally derived protein, virally encoded envelope protein, bacterially derived protein, or bacterial surface protein, etc. In certain embodiments, the bispecific and other multispecific anti-glyco-CD44 antibodies and antigen binding fragments specifically bind to a second CD44 epitope, an epitope on another protein co-expressed on cancer cells with CD44, or an epitope on another protein presented on a different cell, such as an activated T cell. Bispecific antibodies of the disclosure include IgG format bispecific antibodies and single chain-based bispecific antibodies.

IgG format bispecific antibodies of the disclosure can be any of the various types of IgG format bispecific antibodies known in the art, such as quadroma bispecific antibodies, “knobs-in-holes” bispecific antibodies, CrossMab bispecific antibodies, charge paired bispecific antibodies, common light chain bispecific antibodies, one-arm single-chain Fab-immunoglobulin gamma bispecific antibodies, disulfide stabilized Fv bispecific antibodies, DuetMabs, controlled Fab-arm exchange bispecific antibodies, strand-exchange engineered domain body bispecific antibodies, two-arm leucine zipper heterodimeric monoclonal bispecific antibodies, KA-body bispecific antibodies, dual variable domain bispecific antibodies, and cross-over dual variable domain bispecific antibodies. See, e.g., Köhler and Milstein, 1975, Nature 256:495-497; Milstein and Cuello, 1983, Nature 305:537-40; Ridgway et al., 1996, Protein Eng. 9:617-621; Schaefer et al., 2011, Proc Natl Acad Sci USA 108:11187-92; Gunasekaran et al., 2010, J Biol Chem 285:19637-46; Fischer et al., 2015 Nature Commun 6:6113; Schanzer et al., 2014, J Biol Chem 289:18693-706; Metz et al., 2012 Protein Eng Des Sel 25:571-80; Mazor et al., 2015 MAbs 7:377-89; Labrijn et al., 2013 Proc Natl Acad Sci USA 110:5145-50; Davis et al., 2010 Protein Eng Des Sel 23:195-202; Wranik et al., 2012, J Biol Chem 287:43331-9; Gu et al., 2015, PLoS One 10(5):e0124135; Steinmetz et al., 2016, MAbs 8(5):867-78; Klein et al., 2016, mAbs, 8(6):1010-1020; Liu et al., 2017, Front. Immunol. 8:38; and Yang et al., 2017, Int. J. Mol. Sci. 18:48, which are incorporated herein by reference in their entireties.

In some embodiments, the bispecific antibodies of the disclosure are CrossMabs. The CrossMab technology is described in detail in WO 2009/080251, WO 2009/080252, WO 2009/080253, WO 2009/080254, WO 2013/026833, WO 2016/020309, and Schaefer et al., 2011, Proc Natl Acad Sci USA 108:11187-92, which are incorporated herein by reference in their entireties. Briefly, the CrossMab technology is based on a domain crossover between heavy and light chains within one Fab-arm of a bispecific IgG, which promotes correct chain association. A CrossMab bispecific antibody of the disclosure can be a “CrossMab^{FAB}” antibody, in which the heavy and light chains of the Fab portion of one arm of a bispecific IgG antibody are exchanged. In other embodiments, a CrossMab bispecific antibody of the disclosure can be a “CrossMab^{VH-VL}” antibody, in which the only the variable domains of the heavy and light chains of the Fab portion of one arm of a bispecific IgG antibody are exchanged. In yet other embodiments, a CrossMab bispecific antibody of the disclosure can be a “CrossMab^{CH1-CL}” antibody, in which only the constant domains of the heavy and light chains of the Fab portion of one arm of a bispecific IgG antibody are exchanged. CrossMab^{CH1-CL} antibodies, in contrast to CrossMab^{FAB} and CrossMab^{VH-VL}, do not have predicted side products and, therefore, in some embodiments CrossMab^{CH1-CL} bispecific antibodies are preferred. See, Klein et al., 2016, mAbs, 8(6):1010-1020.

In some embodiments, the bispecific antibodies of the disclosure are controlled Fab-arm exchange bispecific antibodies. Methods for making Fab-arm exchange bispecific antibodies are described in PCT Publication No. WO2011/131746 and Labrijn et al., 2014 Nat Protoc. 9(10):2450-63, incorporated herein by reference in their entireties. Briefly, controlled Fab-arm exchange bispecific antibodies can be made by separately expressing two parental IgG1s containing single matching point mutations in the CH3 domain, mixing the parental IgG1s under redox conditions in vitro to enable recombination of half-molecules, and removing the reductant to allow reoxidation of interchain disulfide bonds, thereby forming the bispecific antibodies.

In some embodiments, the bispecific antibodies of the disclosure are “bottle opener,” “mAb-Fv,” “mAb-scFv,” “central-scFv,” “central-Fv,” “one-armed central-scFv” or “dual scFv” format bispecific antibodies. Bispecific antibodies of these formats are described in PCT Publication No. WO 2016/182751, the contents of which are incorporated herein by reference in their entireties. Each of these formats relies on the self-assembling nature of Fc domains of antibody heavy chains, whereby two Fc subunit containing “monomers” assemble into a Fc domain containing “dimer.”

In the bottle opener format, the first monomer comprises a scFv covalently linked to the N-terminus of a Fc subunit, optionally via a linker, and the second monomer comprises a heavy chain (comprising a VH, CH1, and second Fc subunit). A bottle opener format bispecific antibody further comprises a light chain capable of pairing with the second monomer to form a Fab.

The mAb-Fv bispecific antibody format relies upon an “extra” VH domain attached to the C-terminus of one heavy chain monomer and an “extra” VL domain attached to the other heavy chain monomer, forming a third antigen binding domain. In some embodiments, a mAb-Fv bispecific antibody comprises a first monomer comprising a first VH domain, CH1 domain and a first Fc subunit, with a VL domain covalently attached to the C-terminus. The second monomer comprises a VH domain, a CH1 domain a second Fc subunit, and a VH covalently attached to the C-terminus of the second monomer. The two C-terminally attached variable domains make up a Fv. The mAb-Fv further comprises two light chains, which when associated with the first and second monomers form Fabs.

The mAb-scFv bispecific format relies on the use of a C-terminal attachment of a scFv to one of the monomers of a mAb, thus forming a third antigen binding domain. Thus, the first monomer comprises a first heavy chain (comprising a VH, CH1 and a first Fc subunit), with a C-terminally covalently attached scFv. mAb-scFv bispecific antibodies further comprise a second monomer (comprising a VH, CH1, and first Fc subunit) and two light chains, which when associated with the first and second monomers form Fabs.

The central-scFv bispecific format relies on the use of an inserted scFv domain in a mAb, thus forming a third antigen binding domain. The scFv domain is inserted between the Fc subunit and the CH1 domain of one of the monomers, thus providing a third antigen binding domain. Thus, the first monomer can comprise a VH domain, a CH1 domain (and optional hinge) and a first Fc subunit, with a scFv covalently attached between the C-terminus of the CH1 domain and the N-terminus of the first Fc subunit using optional domain linkers. The other monomer can be a standard Fab side monomer. Central-scFv bispecific antibodies further comprise two light chains, which when associated with the first and second monomers form Fabs.

The central-Fv bispecific format relies on the use of an inserted Fv domain thus forming a third antigen binding domain. Each monomer can contain a component of the Fv (e.g. one monomer comprises a variable heavy domain and the other a variable light domain). Thus, one monomer can comprise a VH domain, a CH1 domain, a first Fc subunit and a VL domain covalently attached between the C-terminus of the CH1 domain and the N-terminus of the first Fc subunit, optionally using domain linkers. The other monomer can comprise a VH domain, a CH1 domain, a second Fc subunit and an additional VH domain covalently attached between the C-terminus of the CH1 domain and the N-terminus of the second Fc domain, optionally using domain linkers. Central-Fv bispecific antibodies further comprise two light chains, which when associated with the first and second monomers form Fabs.

The one-armed central-scFv bispecific format comprises one monomer comprising just a Fc subunit, while the other monomer comprises an inserted scFv domain thus forming a second antigen binding domain. Thus, one monomer can comprise a VH domain, a CH1 domain and a first Fc subunit, with a scFv covalently attached between the C-terminus of the CH1 domain and the N-terminus of the first Fc subunit, optionally using domain linkers. The second monomer can comprise an Fc domain. This embodiment further utilizes a light chain comprising a variable light domain and a constant light domain, that associates with the first monomer to form a Fab.

The dual scFv bispecific format comprises a first monomer comprising a scFv covalently attached to the N-terminus of a first Fc subunit, optionally via a linker, and second monomer comprising a scFv covalently attached to the N-terminus of a second Fc subunit, optionally via a linker.

Bispecific antibodies of the disclosure can comprise an Fc domain composed of a first and a second subunit. In one embodiment, the Fc domain is an IgG Fc domain. In a particular embodiment, the Fc domain is an IgG₁ Fc domain. In another embodiment the Fc domain is an IgG₄ Fc domain. In a more specific embodiment, the Fc domain is an IgG₄ Fc domain comprising an amino acid substitution at position S228 (Kabat EU index numbering), particularly the amino acid substitution S228P. Unless otherwise specified herein, numbering of amino acid residues in an Fc domain or constant region is according to the EU numbering system, also called the EU index, as described in Kabat et al., 1991, Sequences of Proteins of Immunological Interest, 5th Ed. Public Health Service, National Institutes of Health, Bethesda, Md. This amino acid substitution reduces in vivo Fab arm exchange of IgG₄ antibodies (see Stubenrauch et al., 2010, Drug Metabolism and Disposition 38:84-91). In a further particular embodiment, the Fc domain is a human Fc domain. In an even more particular embodiment, the Fc domain is a human IgG₁ Fc domain. An exemplary sequence of a human IgG₁ Fc region is given in SEQ ID NO:166.

In particular embodiments, the Fc domain comprises a modification promoting the association of the first and the second subunit of the Fc domain. The site of most extensive protein-protein interaction between the two subunits of a human IgG Fc domain is in the CH3 domain. Thus, in one embodiment said modification is in the CH3 domain of the Fc domain.

In a specific embodiment said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain. The knob-into-hole technology is described e.g., in U.S. Pat. Nos. 5,731,168; 7,695,936; Ridgway et al., 1996, Prot Eng 9:617-621, and Carter, J, 2001, Immunol Meth 248:7-15. Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

Accordingly, in some embodiments, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable. Preferably said amino acid residue having a larger side chain volume is selected from the group consisting of arginine (R), phenylalanine (F), tyrosine (Y), and tryptophan (W). Preferably said amino acid residue having a smaller side chain volume is selected from the group consisting of alanine (A), serine (S), threonine (T), and valine (V). The protuberance and cavity can be made by altering the nucleic acid encoding the polypeptides, e.g. by site-specific mutagenesis, or by peptide synthesis.

In a specific such embodiment, in the first subunit of the Fc domain the threonine residue at position 366 is replaced with a tryptophan residue (T366W), and in the second subunit of the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numbering according to Kabat EU index). In a further embodiment, in the first subunit of the Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second subunit of the Fc domain additionally the tyrosine residue at position 349 is replaced by a cysteine residue (Y349C) (numbering according to Kabat EU index). In a particular embodiment, the first subunit of the Fc domain comprises the amino acid substitutions S354C and T366W, and the second subunit of the Fc domain comprises the amino acid substitutions Y349C, T366S, L368A and Y407V (numbering according to Kabat EU index).

In some embodiments, electrostatic steering (e.g., as described in Gunasekaran et al., 2010, J Biol Chem 285(25):19637-46) can be used to promote the association of the first and the second subunit of the Fc domain.

In some embodiments, the Fc domain comprises one or more amino acid substitutions that reduces binding to an Fc receptor and/or effector function.

In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of complement dependent cytotoxicity (CDC), antibody-dependent cell-mediated cytotoxicity (ADCC), antibody-dependent cellular phagocytosis (ADCP), and cytokine secretion. In a particular embodiment, the effector function is ADCC.

Typically, the same one or more amino acid substitution is present in each of the two subunits of the Fc domain. In one embodiment, the one or more amino acid substitution reduces the binding affinity of the Fc domain to an Fc receptor. In one embodiment, the one or more amino acid substitution reduces the binding affinity of the Fc domain to an Fc receptor by at least 2-fold, at least 5-fold, or at least 10-fold.

In one embodiment, the Fc domain comprises an amino acid substitution at a position selected from the group of E233, L234, L235, N297, P331 and P329 (numberings according to Kabat EU index). In a more specific embodiment, the Fc domain comprises an amino acid substitution at a position selected from the group of L234, L235 and P329 (numberings according to Kabat EU index). In some embodiments, the Fc domain comprises the amino acid substitutions L234A and L235A (numberings according to Kabat EU index). In one such embodiment, the Fc domain is an IgG₁ Fc domain, particularly a human IgG₁ Fc domain. In one embodiment, the Fc domain comprises an amino acid substitution at position P329. In a more specific embodiment, the amino acid substitution is P329A or P329G, particularly P329G (numberings according to Kabat EU index). In one embodiment, the Fc domain comprises an amino acid substitution at position P329 and a further amino acid substitution at a position selected from E233, L234, L235, N297 and P331 (numberings according to Kabat EU index). In a more specific embodiment, the further amino acid substitution is E233P, L234A, L235A, L235E, N297A, N297D or P331S. In particular embodiments, the Fc domain comprises amino acid substitutions at positions P329, L234 and L235 (numberings according to Kabat EU index). In more particular embodiments, the Fc domain comprises the amino acid mutations L234A, L235A and P329G (“P329G LALA”, “PGLALA” or “LALAPG”). Specifically, in particular embodiments, each subunit of the Fc domain comprises the amino acid substitutions L234A, L235A and P329G (Kabat EU index numbering), i.e. in each of the first and the second subunit of the Fc domain the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index). In one such embodiment, the Fc domain is an IgG₁ Fc domain, particularly a human IgG, Fc domain.

Single chain-based bispecific antibodies of the disclosure can be any of the various types of single chain-based bispecific antibodies known in the art, such as bispecific T-cell engagers (BiTEs), diabodies, tandem diabodies (tandabs), dual-affinity retargeting molecules (DARTs), and bispecific killer cell engagers. See, e.g., Löffler et al., 2000, Blood 95:2098-103; Holliger et al., 1993, Proc Natl Acad Sci USA, 90:6444-8; Kipriyanov et al., 1999, Mol Biol 293:41-56; Johnson et al., 2010, Mol Biol 399:436-49; Wiernik et al., 2013, Clin Cancer Res 19:3844-55; Liu et al., 2017, Front. Immunol. 8:38; and Yang et al., 2017, Int. J. Mol. Sci. 18:48, which are incorporated herein by reference in their entireties.

In some embodiments, the bispecific antibodies of the disclosure are bispecific T-cell engagers (BiTEs). BiTEs are single polypeptide chain molecules that having two antigen-binding domains, one of which binds to a T-cell antigen and the second of which binds to an antigen present on the surface of a target (See, PCT Publication WO 05/061547; Baeuerle et al., 2008, Drugs of the Future 33: 137-147; Bargou, et al., 2008, Science 321:974-977, incorporated herein by reference in their entireties). Thus, the BiTEs of the disclosure have an antigen binding domain that binds to a T-cell antigen, and a second antigen binding domain that is directed towards glyco-CD44.

In some embodiments, the bispecific antibodies of the disclosure are dual-affinity retargeting molecules (DARTs). DARTs comprise at least two polypeptide chains that associate (especially through a covalent interaction) to form at least two epitope binding sites, which may recognize the same or different epitopes. Each of the polypeptide chains of a DART comprise an immunoglobulin light chain variable region and an immunoglobulin heavy chain variable region, but these regions do not interact to form an epitope binding site. Rather, the immunoglobulin heavy chain variable region of one (e.g., the first) of the DART polypeptide chains interacts with the immunoglobulin light chain variable region of a different (e.g., the second) DART™ polypeptide chain to form an epitope binding site. Similarly, the immunoglobulin light chain variable region of one (e.g., the first) of the DART polypeptide chains interacts with the immunoglobulin heavy chain variable region of a different (e.g., the second) DART polypeptide chain to form an epitope binding site. DARTs may be monospecific, bispecific, trispecific, etc., thus being able to simultaneously bind one, two, three or more different epitopes (which may be of the same or of different antigens). DARTs may additionally be monovalent, bivalent, trivalent, tetravalent, pentavalent, hexavalent, etc., thus being able to simultaneously bind one, two, three, four, five, six or more molecules. These two attributes of DARTs (i.e., degree of specificity and valency may be combined, for example to produce bispecific antibodies (i.e., capable of binding two epitopes) that are tetravalent (i.e., capable of binding four sets of epitopes), etc. DART molecules are disclosed in PCT Publications WO 2006/113665, WO 2008/157379, and WO 2010/080538, which are incorporated herein by reference in their entireties.

In some embodiments of the bispecific antibodies of the disclosure, one of the binding specificities is directed towards glyco-CD44, and the other is directed to an antigen expressed on immune effector cells. The term “immune effector cell” or “effector cell” as used herein refers to a cell within the natural repertoire of cells in the mammalian immune system which can be activated to affect the viability of a target cell. Immune effector cells include cells of the lymphoid lineage such as natural killer (NK) cells, T cells including cytotoxic T cells, or B cells, but also cells of the myeloid lineage can be regarded as immune effector cells, such as monocytes or macrophages, dendritic cells and neutrophilic granulocytes. Hence, said effector cell is preferably an NK cell, a T cell, a B cell, a monocyte, a macrophage, a dendritic cell or a neutrophilic granulocyte. Recruitment of effector cells to aberrant cells means that immune effector cells are brought in close vicinity to the aberrant target cells such that the effector cells can directly kill, or indirectly initiate the killing of the aberrant cells that they are recruited to. In order to avoid non specific interactions it is preferred that the bispecific antibodies of the disclosure specifically recognize antigens on immune effector cells that are at least overexpressed by these immune effector cells compared to other cells in the body. Target antigens present on immune effector cells may include CD3, CD8, CD16, CD25, CD28, CD64, CD89, NKG2D and NKp46. Preferably, the antigen on immune effector cells is CD3 expressed on T cells.

As used herein, “CD3” refers to any native CD3 from any vertebrate source, including mammals such as primates (e.g. humans), non-human primates (e.g. cynomolgus monkeys) and rodents (e.g. mice and rats), unless otherwise indicated. The term encompasses “full-length,” unprocessed CD3 as well as any form of CD3 that results from processing in the cell. The term also encompasses naturally occurring variants of CD3, e.g., splice variants or allelic variants. The most preferred antigen on an immune effector cell is the CD3 epsilon chain. This antigen has been shown to be very effective in recruiting T cells to aberrant cells. Hence, a bispecific antibody of the disclosure preferably specifically recognizes CD3 epsilon. The amino acid sequence of human CD3 epsilon is shown in UniProt (www.uniprot.org) accession no. P07766 (version 144), or NCBI (www.ncbi.nlm.nih.gov/) RefSeq NP_000724.1. The amino acid sequence of cynomolgus [Macaca fascicularis] CD3 epsilon is shown in NCBI GenBank no. BAB71849.1. For human therapeutic use, bispecific antibodies in which the CD3-binding domain specifically binds to human CD3 (e.g., the human CD3 epsilon chain) are used. For preclinical testing in non-human animals and cell lines, bispecific antibodies in which the CD3-binding domain specifically binds to the CD3 in the species utilized for the preclinical testing (e.g., cynomolgus CD3 for primate testing) can be used.

As used herein, a binding domain that “specifically binds to” or “specifically recognizes” a target antigen from a particular species does not preclude the binding to or recognition of the antigen from other species, and thus encompasses antibodies in which one or more of the binding domains have inter-species cross-reactivity. For example, a CD3-binding domain that “specifically binds to” or “specifically recognizes” human CD3 may also bind to or recognize cyomolgus CD3, and vice versa.

In some embodiments, a bispecific antibody of the disclosure can compete with monoclonal antibody H2C (described in PCT publication no. WO2008/119567) for binding an epitope of CD3. In other embodiments, a bispecific antibody of the disclosure can compete with monoclonal antibody V9 (described in Rodrigues et al., 1992, Int J Cancer Suppl 7:45-50 and U.S. Pat. No. 6,054,297) for binding an epitope of CD3. In yet other embodiments, a bispecific antibody of the disclosure can compete with monoclonal antibody FN18 (described in Nooij et al., 1986, Eur J Immunol 19:981-984) for binding an epitope of CD3. In yet other embodiments, a bispecific antibody of the disclosure can compete with monoclonal antibody SP34 (described in Pessano et al., 1985, EMBO J 4:337-340) for binding an epitope of CD3.

The anti-glyco-CD44 antibodies of the disclosure include derivatized antibodies. For example, but not by way of limitation, derivatized antibodies are typically modified by glycosylation, acetylation, pegylation, phosphorylation, amidation, derivatization by known protecting/blocking groups, proteolytic cleavage, linkage to a cellular ligand or other protein. Any of numerous chemical modifications can be carried out by known techniques, including, but not limited to, specific chemical cleavage, acetylation, formylation, metabolic synthesis of tunicamycin, etc. Additionally, the derivative can contain one or more non-natural amino acids, e.g., using ambrx technology (See, e.g., Wolfson, 2006, Chem. Biol. 13(10):1011-2).

The anti-glyco-CD44 antibodies or binding fragments may be antibodies or fragments whose sequences have been modified to alter at least one constant region-mediated biological effector function. For example, in some embodiments, an anti-glyco-CD44 antibody may be modified to reduce at least one constant region-mediated biological effector function relative to the unmodified antibody, e.g., reduced binding to the Fc receptor (FcγR). FcγR binding can be reduced by mutating the immunoglobulin constant region segment of the antibody at particular regions necessary for FcγR interactions (See, e.g., Canfield and Morrison, 1991, J. Exp. Med. 173:1483-1491; and Lund et al., 1991, J. Immunol. 147:2657-2662). Reduction in FcγR binding ability of the antibody can also reduce other effector functions which rely on FcγR interactions, such as opsonization, phagocytosis and antigen-dependent cellular cytotoxicity (“ADCC”).

The anti-glyco-CD44 antibody or binding fragments described herein include antibodies and/or binding fragments that have been modified to acquire or improve at least one constant region-mediated biological effector function relative to an unmodified antibody, e.g., to enhance FcγR interactions (See, e.g., US 2006/0134709). For example, an anti-glyco-CD44 antibody of the disclosure can have a constant region that binds FcγRIIA, FcγRIIB and/or FcγRIIIA with greater affinity than the corresponding wild type constant region.

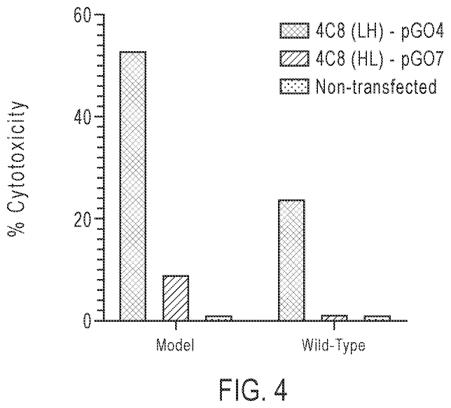

Thus, antibodies of the disclosure may have alterations in biological activity that result in increased or decreased opsonization, phagocytosis, or ADCC. Such alterations are known in the art. For example, modifications in antibodies that reduce ADCC activity are described in U.S. Pat. No. 5,834,597. An exemplary ADCC lowering variant corresponds to “mutant 3” (shown in FIG. 4 of U.S. Pat. No. 5,834,597) in which residue 236 is deleted and residues 234, 235 and 237 (using EU numbering) are substituted with alanines. Another exemplary ADCC lowering variant comprises amino acid mutations L234A, L235A and P329G (“P329G LALA”). The “P329G LALA” combination of amino acid substitutions almost completely abolishes Fcγ receptor (as well as complement) binding of a human IgG₁ Fc domain, as described in PCT publication no. WO 2012/130831, incorporated herein by reference in its entirety. WO 2012/130831 also describes methods of preparing such mutant Fc domains and methods for determining its properties such as Fc receptor binding or effector functions.

In some embodiments, the anti-glyco-CD44 antibodies of the disclosure have low levels of, or lack, fucose. Antibodies lacking fucose have been correlated with enhanced ADCC activity, especially at low doses of antibody. See Shields et al., 2002, J. Biol. Chem. 277:26733-26740; Shinkawa et al., 2003, J. Biol. Chem. 278:3466-73. Methods of preparing fucose-less antibodies include growth in rat myeloma YB2/0 cells (ATCC CRL 1662). YB2/0 cells express low levels of FUT8 mRNA, which encodes α-1, 6-fucosyltransferase, an enzyme necessary for fucosylation of polypeptides.

In some embodiments, the anti-glyco-CD44 antibodies or binding fragments include bisected oligosaccharides, e.g., in which a biantennary oligosaccharide attached to an Fc domain is bisected by GlcNAc. Such variants may have reduced fucosylation and/or improved ADCC function as described above. Examples of such antibody variants are described, e.g., in Umana et al., 1999, Nat Biotechnol 17:176-180; Ferrara et al., 2006, Biotechn Bioeng 93: 851-861; WO 99/54342; WO 2004/065540; and WO 2003/011878.

In yet another aspect, the anti-glyco-CD44 antibodies or binding fragments include modifications that increase or decrease their binding affinities to the fetal Fc receptor, FcRn, for example, by mutating the immunoglobulin constant region segment at particular regions involved in FcRn interactions (see, e.g., WO 2005/123780). In particular embodiments, an anti-glyco-CD44 antibody of the IgG class is mutated such that at least one of amino acid residues 250, 314, and 428 of the heavy chain constant region is substituted alone, or in any combinations thereof, such as at positions 250 and 428, or at positions 250 and 314, or at positions 314 and 428, or at positions 250, 314, and 428, with positions 250 and 428 a specific combination. For position 250, the substituting amino acid residue can be any amino acid residue other than threonine, including, but not limited to, alanine, cysteine, aspartic acid, glutamic acid, phenylalanine, glycine, histidine, isoleucine, lysine, leucine, methionine, asparagine, proline, glutamine, arginine, serine, valine, tryptophan, or tyrosine. For position 314, the substituting amino acid residue can be any amino acid residue other than leucine, including, but not limited to, alanine, cysteine, aspartic acid, glutamic acid, phenylalanine, glycine, histidine, isoleucine, lysine, methionine, asparagine, proline, glutamine, arginine, serine, threonine, valine, tryptophan, or tyrosine. For position 428, the substituting amino acid residues can be any amino acid residue other than methionine, including, but not limited to, alanine, cysteine, aspartic acid, glutamic acid, phenylalanine, glycine, histidine, isoleucine, lysine, leucine, asparagine, proline, glutamine, arginine, serine, threonine, valine, tryptophan, or tyrosine. Specific combinations of suitable amino acid substitutions are identified in Table 1 of U.S. Pat. No. 7,217,797, which is incorporated herein by reference. Such mutations increase binding to FcRn, which protects the antibody from degradation and increases its half-life.

In yet other aspects, an anti-glyco-CD44 antibody of antigen-binding fragment of the disclosure has one or more amino acids inserted into one or more of its hypervariable regions, for example as described in Jung and Pluckthun, 1997, Protein Engineering 10:9, 959-966; Yazaki et al., 2004, Protein Eng. Des Sel. 17(5):481-9. Epub 2004 Aug. 17; and U.S. Pat. App. No. 2007/0280931.

In yet other aspects, particularly useful for diagnostic applications, an anti-glyco-CD44 antibody of antigen-binding fragment of the disclosure is attached to a detectable moiety. Detectable moieties include a radioactive moiety, a colorimetric molecule, a fluorescent moiety, a chemiluminescent moiety, an antigen, an enzyme, a detectable bead (such as a magnetic or electrodense (e.g., gold) bead), or a molecule that binds to another molecule (e.g., biotin or streptavidin)).

Radioisotopes or radionuclides may include ³H, ¹⁴C, ¹⁵N, ³⁵S, ⁹⁰Y, ⁹⁹Tc, ¹¹¹In, ¹²⁵I, ¹³¹I.

Fluorescent labels may include rhodamine, lanthanide phosphors, fluorescein and its derivatives, fluorochrome, GFP (GFP for “Green Fluorescent Protein”), dansyl, umbelliferone, phycoerythrin, phycocyanin, allophycocyanin, o-phthaldehyde, and fluorescamine.

Enzymatic labels may include horseradish peroxidase, β galactosidase, luciferase, alkaline phosphatase, glucose-6-phosphate dehydrogenase (“G6PDH”), alpha-D-galactosidase, glucose oxydase, glucose amylase, carbonic anhydrase, acetylcholinesterase, lysozyme, malate dehydrogenase and peroxidase.

Chemiluminescent labels or chemiluminescers, such as isoluminol, luminol and the dioxetanes.

Other detectable moieties include molecules such as biotin, digoxygenin or 5-bromodeoxyuridine.

In certain aspects, an anti-glyco-CD44 antibody or antigen binding fragment of the disclosure competes with 4C8 or an antibody or antigen binding fragment comprising heavy and light chain variable regions of 4C8 (SEQ ID NOS:1-2, respectively).

In other aspects, an anti-glyco-CD44 antibody or antigen binding fragment of the disclosure competes with 2B2 or an antibody or antigen binding fragment comprising heavy and light chain variable regions of 2B2 (SEQ ID NOS:23-24, respectively).

In other aspects, an anti-glyco-CD44 antibody or antigen binding fragment of the disclosure competes with 18G9 or an antibody or antigen binding fragment comprising heavy and light chain variable regions of 18G9 (SEQ ID NOS:45-46, respectively).

In other aspects, an anti-glyco-CD44 antibody or antigen binding fragment of the disclosure competes with 1D12 or an antibody or antigen binding fragment comprising heavy and light chain variable regions of 1D12 (SEQ ID NOS:67-68, respectively).

In other aspects, an anti-glyco-CD44 antibody or antigen binding fragment of the disclosure competes with 10H4 or an antibody or antigen binding fragment comprising heavy and light chain variable regions of 10H4 (SEQ ID NOS:206-207, respectively).

Competition can be assayed on cells that express the glyco-CD44 epitope bound by 4C8, 2B2, 18G9, 1D12, or 10H4 or on a glycosylated CD44 peptide containing the epitope bound by 4C8, 2B2, 18G9, 1D12, or 10H4, e.g., the CD44v6 glycopeptide. Cells that do not express the epitope or unglycosylated peptides can be used as controls.

Cells on which a competition assay can be carried out include but are not limited to COSMC knock-out HaCaT cells and recombinant cells (e.g., COSMC knock-out HEK293 cells) that are engineered to express the glyco-CD44 epitope. In one non-limiting example, HEK293 cells, which are inherently Tn-negative but can be induced to express the Tn-antigen by knock-out of the COSMC chaperone, are engineered to express CD44, yielding cells expressing the Tn glycoform of CD44 to which 4C8, 2B2, 18G9, 1D12, and 10H4 bind. Cells expressing the unglycosylated form of CD44 can be used as a negative control. Cells expressing the Tn-antigen can also be generated, for example, by treating CD44 expressing cells with a glycosylation inhibitor, knock out of core-1 synthase or ZIP9, or by cleavage of existing glycans.

Assays for competition include, but are not limited to, a radioactive material labeled immunoassay (RIA), an enzyme-linked immunosorbent assay (ELISA), a sandwich ELISA, fluorescence activated cell sorting (FACS) assays, surface plasmon resonance (e.g., Biacore) assays, and bio-layer interferometry (BLI) assays. In some embodiments, antibody competition assays can be carried out using BLI (e.g., using an Octet-HTX system (Molecular Devices)). Antibody competition or epitope binning of monoclonal antibodies can be assessed in tandem against their specific antigen using BLI. In a BLI assay, the antigen can be immobilized onto a biosensor and presented to two competing antibodies in consecutive steps. The binding to non-overlapping epitopes occurs if saturation with the first antibody does not block the binding of the second antibody. In some embodiments, antibody competition assays can be carried out using surface plasmon resonance (e.g., using a Biacore system (Cytiva)). In a surface plasmon resonance assay, one or more antibodies can be immobilized onto a biosensor and presented with an analyte (e.g., the glyco-CD44v6 peptide of SEQ ID NO:165 or a negative control analyte such as a glyco-MUC1 peptide of SEQ ID NO:205 or SEQ ID NO:260 or an unglycosylated CD44v6 peptide of SEQ ID NO:165). In some embodiments, the antibodies are contacted with a saturating concentration of the analyte, for example a concentration of at least about 0.5 μM. In some embodiments the saturating concentration is about 1 μM, about 1.5 μM, or about 2 μM. When comparing the binding affinities of two antibodies, the affinities of both antibodies are preferably measured using the same concentration of both antibodies, e.g., measured using a 1 μM concentration of each antibody.

In conducting an antibody competition assay between a reference antibody and a test antibody (irrespective of species or isotype), one may first label the reference with a detectable label, such as a fluorophore, biotin or an enzymatic (or even radioactive) label to enable subsequent identification. In this case, cells expressing glyco-CD44 are incubated with unlabeled test antibody, labeled reference antibody is added, and the intensity of the bound label is measured. If the test antibody competes with the labeled reference antibody by binding to an overlapping epitope, the intensity will be decreased relative to a control reaction carried out without test antibody.

In a specific embodiment of this assay, the concentration of labeled reference antibody that yields 80% of maximal binding (“conc_{80%}”) under the assay conditions (e.g., a specified density of cells) is first determined, and a competition assay carried out with 10×conc_{80%} of unlabeled test antibody and conc_{80%} of labeled reference antibody.

The inhibition can be expressed as an inhibition constant, or Kᵢ, which is calculated according to the following formula:

Kᵢ=IC₅₀/(1+[reference Ab concentration]/K_{D}),

where IC₅₀ is the concentration of test antibody that yields a 50% reduction in binding of the reference antibody and K_{D} is the dissociation constant of the reference antibody, a measure of its affinity for glyco-CD44. Antibodies that compete with anti-glyco-CD44 antibodies disclosed herein can have a Kᵢ from 10 pM to 10 nM under assay conditions described herein.

In various embodiments, a test antibody is considered to compete with a reference antibody if it decreases binding of the reference antibody by at least about 20% or more, for example, by at least about 20%, 30%, 40%, 50%, 60%, 70%, 80%, 90%, 95% or even more, or by a percentage ranging between any of the foregoing values, at a reference antibody concentration that is 80% of maximal binding under the specific assay conditions used, and a test antibody concentration that is 10-fold higher than the reference antibody concentration.

In one example of a competition assay, the CD44v6 glycopeptide is adhered onto a solid surface, e.g., a microwell plate, by contacting the plate with a solution of the peptide (e.g., at a concentration of 1 μg/mL in PBS over night at 4° C.). The plate is washed (e.g., 0.1% Tween 20 in PBS) and blocked (e.g., in Superblock, Thermo Scientific, Rockford, Ill.). A mixture of sub-saturating amount of biotinylated 4C8, 2B2, 18G9, 1D12, or 10H4 (e.g., at a concentration of 80 ng/mL) and unlabeled antibody (the “reference” antibody) or competing anti-glyco-CD44 antibody (the “test” antibody) antibody in serial dilution (e.g., at a concentration of 2.8 μg/mL, 8.3 μg/mL, or 25 μg/mL) in ELISA buffer (e.g., 1% BSA and 0.1% Tween 20 in PBS) is added to wells and plates are incubated for 1 hour with gentle shaking. The plate is washed, 1 μg/mL HRP-conjugated Streptavidin diluted in ELISA buffer is added to each well and the plates incubated for 1 hour. Plates are washed and bound antibodies detected by addition of substrate (e.g., TMB, Biofx Laboratories Inc., Owings Mills, Md.). The reaction is terminated by addition of stop buffer (e.g., Bio FX Stop Reagents, Biofx Laboratories Inc., Owings Mills, Md.) and the absorbance is measured at 650 nm using microplate reader (e.g., VERSAmax, Molecular Devices, Sunnyvale, Calif.).

Variations on this competition assay can also be used to test competition between 4C8, 2B2, 18G9, 1D12, 10H4 and another anti-glyco-CD44 antibody. For example, in certain aspects, the anti-glyco-CD44 antibody is used as a reference antibody and 4C8, 2B2, 18G9, 1D12, or 10H4 is used as a test antibody. Additionally, instead of a glycosylated CD44 peptide of SEQ ID NO:165, membrane-bound glyco-CD44 expressed on cell surface (for example on the surface of one of the cell types mentioned above) in culture can be used. Generally, about 10⁴ to 10⁶ transfectants, e.g., about 10⁵ transfectants, are used. Other formats for competition assays are known in the art and can be employed.

In various embodiments, an anti-glyco-CD44 antibody of the disclosure reduces the binding of labeled 4C8, 2B2, 18G9, 1D12, or 10H4 by at least 40%, by at least 50%, by at least 60%, by at least 70%, by at least 80%, by at least 90%, or by a percentage ranging between any of the foregoing values (e.g., an anti-glyco-CD44 antibody of the disclosure reduces the binding of labeled 4C8, 2B2, 18G9, 1D12, or 10H4 by 50% to 70%) when the anti-glyco-CD44 antibody is used at a concentration of 0.08 μg/mL, 0.4 μg/mL, 2 μg/mL, 10 μg/mL, 50 μg/mL, 100 μg/mL or at a concentration ranging between any of the foregoing values (e.g., at a concentration ranging from 2 μg/mL to 10 μg/mL).

In other embodiments, 4C8, 2B2, 18G9, 1D12, or 10H4 reduces the binding of a labeled anti-glyco-CD44 antibody of the disclosure by at least 40%, by at least 50%, by at least 60%, by at least 70%, by at least 80%, by at least 90%, or by a percentage ranging between any of the foregoing values (e.g., 4C8, 2B2, 18G9, 1D12, or 10H4 reduces the binding of a labeled an anti-glyco-CD44 antibody of the disclosure by 50% to 70%) when 4C8, 2B2, 18G9, 1D12, or 10H4 is used at a concentration of 0.4 μg/mL, 2 μg/mL, 10 μg/mL, 50 μg/mL, 250 μg/mL or at a concentration ranging between any of the foregoing values (e.g., at a concentration ranging from 2 μg/mL to 10 μg/mL).

In the foregoing assays, the 4C8, 2B2, 18G9, 1D12, or 10H4 antibody can be replaced by any antibody or antigen-binding fragment comprising the CDRs or the heavy and light chain variable regions of 4C8, 2B2, 18G9, 1D12, or 10H4, such as a humanized or chimeric counterpart of 4C8, 2B2, 18G9, 1D12, or 10H4.

In certain aspects, an anti-glyco-CD44 antibody or antigen-binding fragment of the disclosure has an epitope which is the same or similar to the epitope of 4C8, 2B2, 18G9, 1D12, or 10H4. The epitope of an anti-glyco-CD44 antibody or antigen-binding fragment of the disclosure can be characterized by performing alanine scanning. A library of glycopeptides, each varying from the CD44v6 glycopeptide by an alanine point mutation at one position of SEQ ID NO:165 (or, where the CD44 peptide has an alanine, by a glycine point mutation). By measuring an antibody or antigen binding fragment's binding to each of the peptides by ELISA, the antibody or antigen binding fragment's epitope can be mapped.

In certain aspects, an anti-glyco-CD44 antibody or antigen-binding fragment of the disclosure comprises heavy and/or light chain variable sequences (or encoded by the nucleotide sequences) set forth in Tables 1A-1E. In other aspects, an anti-glyco-CD44 antibody or antigen-binding fragment of the disclosure comprises heavy and/or light chain CDR sequences (or encoded by the nucleotide sequences) set forth in Tables 1-3. The framework sequences for such anti-glyco-CD44 antibody and antigen-binding fragment can be the native murine framework sequences of the VH and VL sequences set forth in Tables 1A-1D, the native rabbit framework sequence of the VH and VL sequence set forth in Table 1E, or can be non-native (e.g., humanized or human) framework sequences.

In yet other aspects, the disclosure provides an anti-CD44 antibody or antigen binding fragment having heavy and light chain variable regions having at least 95%, 98%, 99%, or 99.5% sequence identity of SEQ ID NOS:1-2, respectively.

In yet other aspects, the disclosure provides an anti-CD44 antibody or antigen binding fragment having heavy and light chain variable regions having at least 95%, 98%, 99%, or 99.5% sequence identity of SEQ ID NOS:23-24, respectively.

In yet other aspects, the disclosure provides an anti-CD44 antibody or antigen binding fragment having heavy and light chain variable regions having at least 95%, 98%, 99%, or 99.5% sequence identity of SEQ ID NOS:45-46, respectively.

In yet other aspects, the disclosure provides an anti-CD44 antibody or antigen binding fragment having heavy and light chain variable regions having at least 95%, 98%, 99%, or 99.5% sequence identity of SEQ ID NOS:67-68, respectively.

In yet other aspects, the disclosure provides an anti-CD44 antibody or antigen binding fragment having heavy and light chain variable regions having at least 95%, 98%, 99%, or 99.5% sequence identity of SEQ ID NOS:206-207, respectively.

In yet other aspects, an anti-glyco-CD44 antibody or antigen-binding fragment of the disclosure is a single-chain variable fragment (scFv). An exemplary scFv comprises the heavy chain variable fragment N-terminal to the light chain variable fragment. Another exemplary scFv comprises the light chain variable fragment N-terminal to the heavy chain variable fragment. In some embodiments, the scFv heavy chain variable fragment and light chain variable fragment are covalently bound to a linker sequence of 4-15 amino acids. The scFv can be in the form of a bi-specific T-cell engager or within a chimeric antigen receptor (CAR).

6.2 Antibody-Drug Conjugates

Another aspect of the disclosure concerns antibody drug conjugates (ADCs) including the anti-glyco-CD44 antibodies and antigen-binding fragments of the disclosure. The ADCs generally comprise an anti-glyco-CD44 antibody and/or binding fragment as described herein having one or more cytotoxic and/or cytostatic agents linked thereto by way of one or more linkers. In specific embodiments, the ADCs are compounds according to structural formula (I):

[D-L-XY]ₙ-Ab

or salts thereof, where each “D” represents, independently of the others, a cytotoxic and/or cytostatic agent (“drug”); each “L” represents, independently of the others, a linker; “Ab” represents an anti-glyco-CD44 antigen binding domain, such as an anti-glyco-CD44 antibody or binding fragment described herein; each “XY” represents a linkage formed between a functional group R^{x} on the linker and a “complementary” functional group R^{y} on the antibody, and n represents the number of drugs linked to, or drug-to-antibody ratio (DAR), of the ADC.

Specific embodiments of the various antibodies (Ab) that can comprise the ADCs include the various embodiments of anti-glyco-CD44 antibodies and/or binding fragments described above.

In some specific embodiments of the ADCs and/or salts of structural formula (I), each D is the same and/or each L is the same.

Specific embodiments of cytotoxic and/or cytostatic agents (D) and linkers (L) that can comprise the anti-glyco-CD44 ADCs of the disclosure, as well as the number of cytotoxic and/or cytostatic agents linked to the ADCs, are described in more detail below.

6.2.1. Cytotoxic and/or Cytostatic Agents

The cytotoxic and/or cytostatic agents may be any agents known to inhibit the growth and/or replication of and/or kill cells, and in particular cancer and/or tumor cells. Numerous agents having cytotoxic and/or cytostatic properties are known in the literature. Non-limiting examples of classes of cytotoxic and/or cytostatic agents include, by way of example and not limitation, radionuclides, alkylating agents, topoisomerase I inhibitors, topoisomerase II inhibitors, DNA intercalating agents (e.g., groove binding agents such as minor groove binders), RNA/DNA antimetabolites, cell cycle modulators, kinase inhibitors, protein synthesis inhibitors, histone deacetylase inhibitors, mitochondria inhibitors, and antimitotic agents.

Specific non-limiting examples of agents within certain of these various classes are provided below.

Alkylating Agents: asaley ((L-Leucine, N—[N-acetyl-4-[bis-(2-chloroethyl)amino]-DL-phenylalanyl]-, ethylester; NSC 167780; CAS Registry No. 3577897)); AZQ ((1,4-cyclohexadiene-1,4-dicarbamic acid, 2,5-bis(1-aziridinyl)-3,6-dioxo-, diethyl ester; NSC 182986; CAS Registry No. 57998682)); BCNU ((N,N′-Bis(2-chloroethyl)-N-nitrosourea; NSC 409962; CAS Registry No. 154938)); busulfan (1,4-butanediol dimethanesulfonate; NSC 750; CAS Registry No. 55981); (carboxyphthalato)platinum (NSC 27164; CAS Registry No. 65296813); CBDCA ((cis-(1,1-cyclobutanedicarboxylato)diammineplatinum(II)); NSC 241240; CAS Registry No. 41575944)); CCNU ((N-(2-chloroethyl)-N′-cyclohexyl-N-nitrosourea; NSC 79037; CAS Registry No. 13010474)); CHIP (iproplatin; NSC 256927); chlorambucil (NSC 3088; CAS Registry No. 305033); chlorozotocin ((2-[[[(2-chloroethyl) nitrosoamino]carbonyl]amino]-2-deoxy-D-glucopyranose; NSC 178248; CAS Registry No. 54749905)); cis-platinum (cisplatin; NSC 119875; CAS Registry No. 15663271); clomesone (NSC 338947; CAS Registry No. 88343720); cyanomorpholinodoxorubicin (NCS 357704; CAS Registry No. 88254073); cyclodisone (NSC 348948; CAS Registry No. 99591738); dianhydrogalactitol (5,6-diepoxydulcitol; NSC 132313; CAS Registry No. 23261203); fluorodopan ((5-[(2-chloroethyl)-(2-fluoroethyl)amino]-6-methyl-uracil; NSC 73754; CAS Registry No. 834913); hepsulfam (NSC 329680; CAS Registry No. 96892578); hycanthone (NSC 142982; CAS Registry No. 23255938); melphalan (NSC 8806; CAS Registry No. 3223072); methyl CCNU ((1-(2-chloroethyl)-3-(trans-4-methylcyclohexane)-1-nitrosourea; NSC 95441; 13909096); mitomycin C (NSC 26980; CAS Registry No. 50077); mitozolamide (NSC 353451; CAS Registry No. 85622953); nitrogen mustard ((bis(2-chloroethyl)methylamine hydrochloride; NSC 762; CAS Registry No. 55867); PCNU ((1-(2-chloroethyl)-3-(2,6-dioxo-3-piperidyl)-1-nitrosourea; NSC 95466; CAS Registry No. 13909029)); piperazine alkylator ((1-(2-chloroethyl)-4-(3-chloropropyl)-piperazine dihydrochloride; NSC 344007)); piperazinedione (NSC 135758; CAS Registry No. 41109802); pipobroman ((N,N-bis(3-bromopropionyl) piperazine; NSC 25154; CAS Registry No. 54911)); porfiromycin (N-methylmitomycin C; NSC 56410; CAS Registry No. 801525); spirohydantoin mustard (NSC 172112; CAS Registry No. 56605164); teroxirone (triglycidylisocyanurate; NSC 296934; CAS Registry No. 2451629); tetraplatin (NSC 363812; CAS Registry No. 62816982); thio-tepa (N,N′,N″-tri-1,2-ethanediylthio phosphoramide; NSC 6396; CAS Registry No. 52244); triethylenemelamine (NSC 9706; CAS Registry No. 51183); uracil nitrogen mustard (desmethyldopan; NSC 34462; CAS Registry No. 66751); Yoshi-864 ((bis(3-mesyloxy propyl)amine hydrochloride; NSC 102627; CAS Registry No. 3458228).

Topoisomerase I Inhibitors: camptothecin (NSC 94600; CAS Registry No. 7689-03-4); various camptothecin derivatives and analogs (for example, NSC 100880, NSC 603071, NSC 107124, NSC 643833, NSC 629971, NSC 295500, NSC 249910, NSC 606985, NSC 74028, NSC 176323, NSC 295501, NSC 606172, NSC 606173, NSC 610458, NSC 618939, NSC 610457, NSC 610459, NSC 606499, NSC 610456, NSC 364830, and NSC 606497); morpholinisoxorubicin (NSC 354646; CAS Registry No. 89196043); SN-38 (NSC 673596; CAS Registry No. 86639-52-3).

Topoisomerase II Inhibitors: doxorubicin (NSC 123127; CAS Registry No. 25316409); amonafide (benzisoquinolinedione; NSC 308847; CAS Registry No. 69408817); m-AMSA ((4′-(9-acridinylamino)-3′-methoxymethanesulfonanilide; NSC 249992; CAS Registry No. 51264143)); anthrapyrazole derivative ((NSC 355644); etoposide (VP-16; NSC 141540; CAS Registry No. 33419420); pyrazoloacridine ((pyrazolo[3,4,5-kl]acridine-2(6H)-propanamine, 9-methoxy-N, N-dimethyl-5-nitro-, monomethanesulfonate; NSC 366140; CAS Registry No. 99009219); bisantrene hydrochloride (NSC 337766; CAS Registry No. 71439684); daunorubicin (NSC 821151; CAS Registry No. 23541506); deoxydoxorubicin (NSC 267469; CAS Registry No. 63950061); mitoxantrone (NSC 301739; CAS Registry No. 70476823); menogaril (NSC 269148; CAS Registry No. 71628961); N,N-dibenzyl daunomycin (NSC 268242; CAS Registry No. 70878512); oxanthrazole (NSC 349174; CAS Registry No. 105118125); rubidazone (NSC 164011; CAS Registry No. 36508711); teniposide (VM-26; NSC 122819; CAS Registry No. 29767202).

DNA Intercalating Agents: anthramycin (CAS Registry No. 4803274); chicamycin A (CAS Registry No. 89675376); tomaymycin (CAS Registry No. 35050556); DC-81 (CAS Registry No. 81307246); sibiromycin (CAS Registry No. 12684332); pyrrolobenzodiazepine derivative (CAS Registry No. 945490095); SGD-1882 ((S)-2-(4-aminophenyl)-7-methoxy-8-(3-4(S)-7-methoxy-2-(4-methoxyphenyl)-5-oxo-5,11a-dihydro-1H-benzo[e]pyrrolo[1,2-a][1,4]diazepin-8-yl)oxy)propox-y)-1H-benzo[e]pyrrolo[1,2-a][1,4]diazepin-5(11aH)-one); SG2000 (SJG-136; (11aS,11a'S)-8,8′-(propane-1,3-diylbis(oxy))bis(7-methoxy-2-methylene-2,3-dihydro-1H-benzo[e]pyrrolo[1,2-a][1,4]diazepin-5(11aH)-one); NSC 694501; CAS Registry No. 232931576).

RNA/DNA Antimetabolites: L-alanosine (NSC 153353; CAS Registry No. 59163416); 5-azacytidine (NSC 102816; CAS Registry No. 320672); 5-fluorouracil (NSC 19893; CAS Registry No. 51218); acivicin (NSC 163501; CAS Registry No. 42228922); aminopterin derivative N42-chloro-5-[[(2,4-diamino-5-methyl-6-quinazolinyl)methyl]amino]benzoyl-]L-aspartic acid (NSC 132483); aminopterin derivative N44-[[(2,4-diamino-5-ethyl-6-quinazolinyl)methyl]amino]benzoyl]L-asparti-c acid (NSC 184692); aminopterin derivative N-[2-chloro-4-[[(2,4-diamino-6-pteridinyl)methyl]amino]benzoyl]L-aspartic acid monohydrate (NSC 134033); an antifo ((N^{α}-(4-amino-4-deoxypteroyl)-N⁷-hemiphthaloyl-L-ornithin-e; NSC 623017)); Baker's soluble antifol (NSC 139105; CAS Registry No. 41191042); dichlorallyl lawsone ((2-(3,3-dichloroallyl)-3-hydroxy-1,4-naphthoquinone; NSC 126771; CAS Registry No. 36417160); brequinar (NSC 368390; CAS Registry No. 96201886); ftorafur ((pro-drug; 5-fluoro-1-(tetrahydro-2-furyl)-uracil; NSC 148958; CAS Registry No. 37076689); 5,6-dihydro azacytidine (NSC 264880; CAS Registry No. 62402317); methotrexate (NSC 740; CAS Registry No. 59052); methotrexate derivative (N-[[4-[[(2,4-diamino pteridinyl)methyl]methylamino]-1-naphthalenyl]car-bonyl]L-glutamic acid; NSC 174121); PALA ((N-(phosphonoacetyl)-L-aspartate; NSC 224131; CAS Registry No. 603425565); pyrazofurin (NSC 143095; CAS Registry No. 30868305); trimetrexate (NSC 352122; CAS Registry No. 82952645).

DNA Antimetabolites: 3-HP (NSC 95678; CAS Registry No. 3814797); 2′-deoxy fluorouridine (NSC 27640; CAS Registry No. 50919); 5-HP (NSC 107392; CAS Registry No. 19494894); α-TGDR (α-2′-deoxy-6-thioguanosine; NSC 71851 CAS Registry No. 2133815); aphidicolin glycinate (NSC 303812; CAS Registry No. 92802822); ara C (cytosine arabinoside; NSC 63878; CAS Registry No. 69749); 5-aza-2′-deoxycytidine (NSC 127716; CAS Registry No. 2353335); β-TGDR (β-2′-deoxy-6-thioguanosine; NSC 71261; CAS Registry No. 789617); cyclocytidine (NSC 145668; CAS Registry No. 10212256); guanazole (NSC 1895; CAS Registry No. 1455772); hydroxyurea (NSC 32065; CAS Registry No. 127071); inosine glycodialdehyde (NSC 118994; CAS Registry No. 23590990); macbecin II (NSC 330500; CAS Registry No. 73341738); pyrazoloimidazole (NSC 51143; CAS Registry No. 6714290); thioguanine (NSC 752; CAS Registry No. 154427); thiopurine (NSC 755; CAS Registry No. 50442).

Cell Cycle Modulators: silibinin (CAS Registry No. 22888-70-6); epigallocatechin gallate (EGCG; CAS Registry No. 989515); procyanidin derivatives (e.g., procyanidin A1 [CAS Registry No. 103883030], procyanidin B1 [CAS Registry No. 20315257], procyanidin B4 [CAS Registry No. 29106512], arecatannin B1 [CAS Registry No. 79763283]); isoflavones (e.g., genistein [4%5,7-trihydroxyisoflavone; CAS Registry No. 446720], daidzein [4′,7-dihydroxyisoflavone, CAS Registry No. 486668]; indole-3-carbinol (CAS Registry No. 700061); quercetin (NSC 9219; CAS Registry No. 117395); estramustine (NSC 89201; CAS Registry No. 2998574); nocodazole (CAS Registry No. 31430189); podophyllotoxin (CAS Registry No. 518285); vinorelbine tartrate (NSC 608210; CAS Registry No. 125317397); cryptophycin (NSC 667642; CAS Registry No. 124689652).

Kinase Inhibitors: afatinib (CAS Registry No. 850140726); axitinib (CAS Registry No. 319460850); ARRY-438162 (binimetinib) (CAS Registry No. 606143899); bosutinib (CAS Registry No. 380843754); cabozantinib (CAS Registry No. 1140909483); ceritinib (CAS Registry No. 1032900256); crizotinib (CAS Registry No. 877399525); dabrafenib (CAS Registry No. 1195765457); dasatinib (NSC 732517; CAS Registry No. 302962498); erlotinib (NSC 718781; CAS Registry No. 183319699); everolimus (NSC 733504; CAS Registry No. 159351696); fostamatinib (NSC 745942; CAS Registry No. 901119355); gefitinib (NSC 715055; CAS Registry No. 184475352); ibrutinib (CAS Registry No. 936563961); imatinib (NSC 716051; CAS Registry No. 220127571); lapatinib (CAS Registry No. 388082788); lenvatinib (CAS Registry No. 857890392); mubritinib (CAS 366017096); nilotinib (CAS Registry No. 923288953); nintedanib (CAS Registry No. 656247175); palbociclib (CAS Registry No. 571190302); pazopanib (NSC 737754; CAS Registry No. 635702646); pegaptanib (CAS Registry No. 222716861); ponatinib (CAS Registry No. 1114544318); rapamycin (NSC 226080; CAS Registry No. 53123889); regorafenib (CAS Registry No. 755037037); AP 23573 (ridaforolimus) (CAS Registry No. 572924540); INCB018424 (ruxolitinib) (CAS Registry No. 1092939177); ARRY-142886 (selumetinib) (NSC 741078; CAS Registry No. 606143-52-6); sirolimus (NSC 226080; CAS Registry No. 53123889); sorafenib (NSC 724772; CAS Registry No. 475207591); sunitinib (NSC 736511; CAS Registry No. 341031547); tofacitinib (CAS Registry No. 477600752); temsirolimus (NSC 683864; CAS Registry No. 163635043); trametinib (CAS Registry No. 871700173); vandetanib (CAS Registry No. 443913733); vemurafenib (CAS Registry No. 918504651); SU6656 (CAS Registry No. 330161870); CEP-701 (lesaurtinib) (CAS Registry No. 111358884); XL019 (CAS Registry No. 945755566); PD-325901 (CAS Registry No. 391210109); PD-98059 (CAS Registry No. 167869218); ATP-competitive TORC1/TORC2 inhibitors including PI-103 (CAS Registry No. 371935749), PP242 (CAS Registry No. 1092351671), PP30 (CAS Registry No. 1092788094), Torin 1 (CAS Registry No. 1222998368), LY294002 (CAS Registry No. 154447366), XL-147 (CAS Registry No. 934526893), CAL-120 (CAS Registry No. 870281348), ETP-45658 (CAS Registry No. 1198357797), PX 866 (CAS Registry No. 502632668), GDC-0941 (CAS Registry No. 957054307), BGT226 (CAS Registry No. 1245537681), BEZ235 (CAS Registry No. 915019657), XL-765 (CAS Registry No. 934493762).

Protein Synthesis Inhibitors: acriflavine (CAS Registry No. 65589700); amikacin (NSC 177001; CAS Registry No. 39831555); arbekacin (CAS Registry No. 51025855); astromicin (CAS Registry No. 55779061); azithromycin (NSC 643732; CAS Registry No. 83905015); bekanamycin (CAS Registry No. 4696768); chlortetracycline (NSC 13252; CAS Registry No. 64722); clarithromycin (NSC 643733; CAS Registry No. 81103119); clindamycin (CAS Registry No. 18323449); clomocycline (CAS Registry No. 1181540); cycloheximide (CAS Registry No. 66819); dactinomycin (NSC 3053; CAS Registry No. 50760); dalfopristin (CAS Registry No. 112362502); demeclocycline (CAS Registry No. 127333); dibekacin (CAS Registry No. 34493986); dihydrostreptomycin (CAS Registry No. 128461); dirithromycin (CAS Registry No. 62013041); doxycycline (CAS Registry No. 17086281); emetine (NSC 33669; CAS Registry No. 483181); erythromycin (NSC 55929; CAS Registry No. 114078); flurithromycin (CAS Registry No. 83664208); framycetin (neomycin B; CAS Registry No. 119040); gentamycin (NSC 82261; CAS Registry No. 1403663); glycylcyclines, such as tigecycline (CAS Registry No. 220620097); hygromycin B (CAS Registry No. 31282049); isepamicin (CAS Registry No. 67814760); josamycin (NSC 122223; CAS Registry No. 16846245); kanamycin (CAS Registry No. 8063078); ketolides such as telithromycin (CAS Registry No. 191114484), cethromycin (CAS Registry No. 205110481), and solithromycin (CAS Registry No. 760981837); lincomycin (CAS Registry No. 154212); lymecycline (CAS Registry No. 992212); meclocycline (NSC 78502; CAS Registry No. 2013583); metacycline (rondomycin; NSC 356463; CAS Registry No. 914001); midecamycin (CAS Registry No. 35457808); minocycline (NSC 141993; CAS Registry No. 10118908); miocamycin (CAS Registry No. 55881077); neomycin (CAS Registry No. 119040); netilmicin (CAS Registry No. 56391561); oleandomycin (CAS Registry No. 3922905); oxazolidinones, such as eperezolid (CAS Registry No. 165800044), linezolid (CAS Registry No. 165800033), posizolid (CAS Registry No. 252260029), radezolid (CAS Registry No. 869884786), ranbezolid (CAS Registry No. 392659380), sutezolid (CAS Registry No. 168828588), tedizolid (CAS Registry No. 856867555); oxytetracycline (NSC 9169; CAS Registry No. 2058460); paromomycin (CAS Registry No. 7542372); penimepicycline (CAS Registry No. 4599604); peptidyl transferase inhibitors, e.g., chloramphenicol (NSC 3069; CAS Registry No. 56757) and derivatives such as azidamfenicol (CAS Registry No. 13838089), florfenicol (CAS Registry No. 73231342), and thiamphenicol (CAS Registry No. 15318453), and pleuromutilins such as retapamulin (CAS Registry No. 224452668), tiamulin (CAS Registry No. 55297955), valnemulin (CAS Registry No. 101312929); pirlimycin (CAS Registry No. 79548735); puromycin (NSC 3055; CAS Registry No. 53792); quinupristin (CAS Registry No. 120138503); ribostamycin (CAS Registry No. 53797356); rokitamycin (CAS Registry No. 74014510); rolitetracycline (CAS Registry No. 751973); roxithromycin (CAS Registry No. 80214831); sisomicin (CAS Registry No. 32385118); spectinomycin (CAS Registry No. 1695778); spiramycin (CAS Registry No. 8025818); streptogramins such as pristinamycin (CAS Registry No. 270076603), quinupristin/dalfopristin (CAS Registry No. 126602899), and virginiamycin (CAS Registry No. 11006761); streptomycin (CAS Registry No. 57921); tetracycline (NSC 108579; CAS Registry No. 60548); tobramycin (CAS Registry No. 32986564); troleandomycin (CAS Registry No. 2751099); tylosin (CAS Registry No. 1401690); verdamicin (CAS Registry No. 49863481).

Histone Deacetylase Inhibitors: abexinostat (CAS Registry No. 783355602); belinostat (NSC 726630; CAS Registry No. 414864009); chidamide (CAS Registry No. 743420022); entinostat (CAS Registry No. 209783802); givinostat (CAS Registry No. 732302997); mocetinostat (CAS Registry No. 726169739); panobinostat (CAS Registry No. 404950807); quisinostat (CAS Registry No. 875320299); resminostat (CAS Registry No. 864814880); romidepsin (CAS Registry No. 128517077); sulforaphane (CAS Registry No. 4478937); thioureidobutyronitrile (Kevetrin™; CAS Registry No. 6659890); valproic acid (NSC 93819; CAS Registry No. 99661); vorinostat (NSC 701852; CAS Registry No. 149647789); ACY-1215 (rocilinostat; CAS Registry No. 1316214524); CUDC-101 (CAS Registry No. 1012054599); CHR-2845 (tefinostat; CAS Registry No. 914382608); CHR-3996 (CAS Registry No. 1235859138); 4SC-202 (CAS Registry No. 910462430); CG200745 (CAS Registry No. 936221339); SB939 (pracinostat; CAS Registry No. 929016966).

Mitochondria Inhibitors: pancratistatin (NSC 349156; CAS Registry No. 96281311); rhodamine-123 (CAS Registry No. 63669709); edelfosine (NSC 324368; CAS Registry No. 70641519); d-alpha-tocopherol succinate (NSC 173849; CAS Registry No. 4345033); compound 11β (CAS Registry No. 865070377); aspirin (NSC 406186; CAS Registry No. 50782); ellipticine (CAS Registry No. 519233); berberine (CAS Registry No. 633658); cerulenin (CAS Registry No. 17397896); GX015-070 (Obatoclax®; 1H-Indole, 2-(2-((3,5-dimethyl-1H-pyrrol-2-yl)methylene)-3-methoxy-2H-pyrrol-5-yl)-; NSC 729280; CAS Registry No. 803712676); celastrol (tripterine; CAS Registry No. 34157830); metformin (NSC 91485; CAS Registry No. 1115704); Brilliant green (NSC 5011; CAS Registry No. 633034); ME-344 (CAS Registry No. 1374524556).

Antimitotic Agents: allocolchicine (NSC 406042); auristatins, such as MMAE (monomethyl auristatin E; CAS Registry No. 474645-27-7) and MMAF (monomethyl auristatin F; CAS Registry No. 745017-94-1; halichondrin B (NSC 609395); colchicine (NSC 757; CAS Registry No. 64868); cholchicine derivative (N-benzoyl-deacetyl benzamide; NSC 33410; CAS Registry No. 63989753); dolastatin 10 (NSC 376128; CAS Registry No 110417-88-4); maytansine (NSC 153858; CAS Registry No. 35846-53-8); rhozoxin (NSC 332598; CAS Registry No. 90996546); taxol (NSC 125973; CAS Registry No. 33069624); taxol derivative ((2′-N-[3-(dimethylamino)propyl]glutaramate taxol; NSC 608832); thiocolchicine (3-demethylthiocolchicine; NSC 361792); trityl cysteine (NSC 49842; CAS Registry No. 2799077); vinblastine sulfate (NSC 49842; CAS Registry No. 143679); vincristine sulfate (NSC 67574; CAS Registry No. 2068782).

Any of these agents that include or that may be modified to include a site of attachment to an antibody may be included in the ADCs disclosed herein.

In a specific embodiment, the cytotoxic and/or cytostatic agent is an antimitotic agent.

In another specific embodiment, the cytotoxic and/or cytostatic agent is an auristatin, for example, monomethyl auristatin E (“MMAE”) or monomethyl auristatin F (“MMAF”).

6.2.2. Linkers

In the anti-glyco-CD44 ADCs of the disclosure, the cytotoxic and/or cytostatic agents are linked to the antibody by way of linkers. The linker linking a cytotoxic and/or cytostatic agent to the antibody of an ADC may be short, long, hydrophobic, hydrophilic, flexible or rigid, or may be composed of segments that each independently have one or more of the above-mentioned properties such that the linker may include segments having different properties. The linkers may be polyvalent such that they covalently link more than one agent to a single site on the antibody, or monovalent such that covalently they link a single agent to a single site on the antibody.

As will be appreciated by skilled artisans, the linkers link cytotoxic and/or cytostatic agents to the antibody by forming a covalent linkage to the cytotoxic and/or cytostatic agent at one location and a covalent linkage to antibody at another. The covalent linkages are formed by reaction between functional groups on the linker and functional groups on the agents and antibody. As used herein, the expression “linker” is intended to include (i) unconjugated forms of the linker that include a functional group capable of covalently linking the linker to a cytotoxic and/or cytostatic agent and a functional group capable of covalently linking the linker to an antibody; (ii) partially conjugated forms of the linker that includes a functional group capable of covalently linking the linker to an antibody and that is covalently linked to a cytotoxic and/or cytostatic agent, or vice versa; and (iii) fully conjugated forms of the linker that is covalently linked to both a cytotoxic and/or cytostatic agent and an antibody. In some specific embodiments of linkers and anti-glyco-CD44 ADCs of the disclosure, as well as synthons used to conjugate linker-agents to antibodies, moieties comprising the functional groups on the linker and covalent linkages formed between the linker and antibody are specifically illustrated as Rₓ and XY, respectively.

The linkers are preferably, but need not be, chemically stable to conditions outside the cell, and may be designed to cleave, immolate and/or otherwise specifically degrade inside the cell. Alternatively, linkers that are not designed to specifically cleave or degrade inside the cell may be used. Choice of stable versus unstable linker may depend upon the toxicity of the cytotoxic and/or cytostatic agent. For agents that are toxic to normal cells, stable linkers are preferred. Agents that are selective or targeted and have lower toxicity to normal cells may utilize, chemical stability of the linker to the extracellular milieu is less important. A wide variety of linkers useful for linking drugs to antibodies in the context of ADCs are known in the art. Any of these linkers, as well as other linkers, may be used to link the cytotoxic and/or cytostatic agents to the antibody of the anti-glyco-CD44 ADCs of the disclosure.

Exemplary polyvalent linkers that may be used to link many cytotoxic and/or cytostatic agents to a single antibody molecule are described, for example, in WO 2009/073445; WO 2010/068795; WO 2010/138719; WO 2011/120053; WO 2011/171020; WO 2013/096901; WO 2014/008375; WO 2014/093379; WO 2014/093394; WO 2014/093640, the content of which are incorporated herein by reference in their entireties. For example, the Fleximer linker technology developed by Mersana et al. has the potential to enable high-DAR ADCs with good physicochemical properties. As shown below, the Mersana technology is based on incorporating drug molecules into a solubilizing poly-acetal backbone via a sequence of ester bonds. The methodology renders highly-loaded ADCs (DAR up to 20) while maintaining good physicochemical properties.

Additional examples of dendritic type linkers can be found in US 2006/116422; US 2005/271615; de Groot et al. (2003) Angew. Chem. Int. Ed. 42:4490-4494; Amir et al. (2003) Angew. Chem. Int. Ed. 42:4494-4499; Shamis et al. (2004) J. Am. Chem. Soc. 126:1726-1731; Sun et al. (2002) Bioorganic & Medicinal Chemistry Letters 12:2213-2215; Sun et al. (2003) Bioorganic & Medicinal Chemistry 11:1761-1768; King et al. (2002) Tetrahedron Letters 43:1987-1990, each of which is incorporated herein by reference.

Exemplary monovalent linkers that may be used are described, for example, in Nolting, 2013, Antibody-Drug Conjugates, Methods in Molecular Biology 1045:71-100; Kitson et al., 2013, CROs/CMOs—Chemica Oggi—Chemistry Today 31(4):30-38; Ducry et al., 2010, Bioconjugate Chem. 21:5-13; Zhao et al., 2011, J. Med. Chem. 54:3606-3623; U.S. Pat. Nos. 7,223,837; 8,568,728; 8,535,678; and WO2004010957, each of which is incorporated herein by reference.

By way of example and not limitation, some cleavable and noncleavable linkers that may be included in the anti-glyco-CD44 ADCs of the disclosure are described below.

6.2.3. Cleavable Linkers

In certain embodiments, the linker selected is cleavable in vivo. Cleavable linkers may include chemically or enzymatically unstable or degradable linkages. Cleavable linkers generally rely on processes inside the cell to liberate the drug, such as reduction in the cytoplasm, exposure to acidic conditions in the lysosome, or cleavage by specific proteases or other enzymes within the cell. Cleavable linkers generally incorporate one or more chemical bonds that are either chemically or enzymatically cleavable while the remainder of the linker is noncleavable. In certain embodiments, a linker comprises a chemically labile group such as hydrazone and/or disulfide groups. Linkers comprising chemically labile groups exploit differential properties between the plasma and some cytoplasmic compartments. The intracellular conditions to facilitate drug release for hydrazone containing linkers are the acidic environment of endosomes and lysosomes, while the disulfide containing linkers are reduced in the cytosol, which contains high thiol concentrations, e.g., glutathione. In certain embodiments, the plasma stability of a linker comprising a chemically labile group may be increased by introducing steric hindrance using substituents near the chemically labile group.

Acid-labile groups, such as hydrazone, remain intact during systemic circulation in the blood's neutral pH environment (pH 7.3-7.5) and undergo hydrolysis and release the drug once the ADC is internalized into mildly acidic endosomal (pH 5.0-6.5) and lysosomal (pH 4.5-5.0) compartments of the cell. This pH dependent release mechanism has been associated with nonspecific release of the drug. To increase the stability of the hydrazone group of the linker, the linker may be varied by chemical modification, e.g., substitution, allowing tuning to achieve more efficient release in the lysosome with a minimized loss in circulation.

Hydrazone-containing linkers may contain additional cleavage sites, such as additional acid-labile cleavage sites and/or enzymatically labile cleavage sites. ADCs including exemplary hydrazone-containing linkers include the following structures:

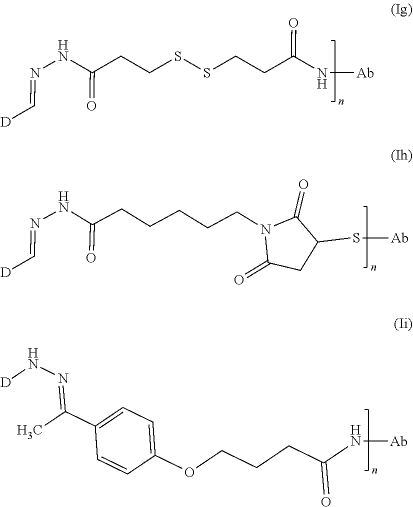

wherein D and Ab represent the cytotoxic and/or cytostatic agent (drug) and Ab, respectively, and n represents the number of drug-linkers linked to the antibody. In certain linkers such as linker (Ig), the linker comprises two cleavable groups—a disulfide and a hydrazone moiety. For such linkers, effective release of the unmodified free drug requires acidic pH or disulfide reduction and acidic pH. Linkers such as (Ih) and (Ii) have been shown to be effective with a single hydrazone cleavage site.

Additional linkers which remain intact during systemic circulation and undergo hydrolysis and release the drug when the ADC is internalized into acidic cellular compartments include carbonates. Such linkers can be useful in cases where the cytotoxic and/or cytostatic agent can be covalently attached through an oxygen.

Other acid-labile groups that may be included in linkers include cis-aconityl-containing linkers. cis-Aconityl chemistry uses a carboxylic acid juxtaposed to an amide bond to accelerate amide hydrolysis under acidic conditions.

Cleavable linkers may also include a disulfide group. Disulfides are thermodynamically stable at physiological pH and are designed to release the drug upon internalization inside cells, wherein the cytosol provides a significantly more reducing environment compared to the extracellular environment. Scission of disulfide bonds generally requires the presence of a cytoplasmic thiol cofactor, such as (reduced) glutathione (GSH), such that disulfide-containing linkers are reasonably stable in circulation, selectively releasing the drug in the cytosol. The intracellular enzyme protein disulfide isomerase, or similar enzymes capable of cleaving disulfide bonds, may also contribute to the preferential cleavage of disulfide bonds inside cells. GSH is reported to be present in cells in the concentration range of 0.5-10 mM compared with a significantly lower concentration of GSH or cysteine, the most abundant low-molecular weight thiol, in circulation at approximately 5 Tumor cells, where irregular blood flow leads to a hypoxic state, result in enhanced activity of reductive enzymes and therefore even higher glutathione concentrations. In certain embodiments, the in vivo stability of a disulfide-containing linker may be enhanced by chemical modification of the linker, e.g., use of steric hindrance adjacent to the disulfide bond.

ADCs including exemplary disulfide-containing linkers include the following structures:

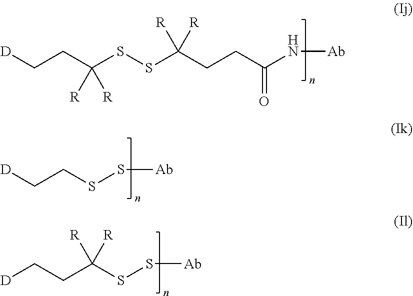

wherein D and Ab represent the drug and antibody, respectively, n represents the number of drug-linkers linked to the antibody and R is independently selected at each occurrence from hydrogen or alkyl, for example. In certain embodiments, increasing steric hindrance adjacent to the disulfide bond increases the stability of the linker. Structures such as (ID and (II) show increased in vivo stability when one or more R groups is selected from a lower alkyl such as methyl.

Another type of cleavable linker that may be used is a linker that is specifically cleaved by an enzyme. Such linkers are typically peptide-based or include peptidic regions that act as substrates for enzymes. Peptide based linkers tend to be more stable in plasma and extracellular milieu than chemically labile linkers. Peptide bonds generally have good serum stability, as lysosomal proteolytic enzymes have very low activity in blood due to endogenous inhibitors and the unfavorably high pH value of blood compared to lysosomes. Release of a drug from an antibody occurs specifically due to the action of lysosomal proteases, e.g., cathepsin and plasmin. These proteases may be present at elevated levels in certain tumor cells.

In exemplary embodiments, the cleavable peptide is selected from tetrapeptides such as Gly-Phe-Leu-Gly (SEQ ID NO:181), Ala-Leu-Ala-Leu (SEQ ID NO:182) or dipeptides such as Val-Cit, Val-Ala, Met-(D)Lys, Asn-(D)Lys, Val-(D)Asp, Phe-Lys, Ile-Val, Asp-Val, His-Val, NorVal-(D)Asp, Ala-(D)Asp 5, Met-Lys, Asn-Lys, Ile-Pro, Me3Lys-Pro, PhenylGly-(D)Lys, Met-(D)Lys, Asn-(D)Lys, Pro-(D)Lys, Met-(D)Lys, Asn-(D)Lys, AM Met-(D)Lys, Asn-(D)Lys, AW Met-(D)Lys, and Asn-(D)Lys. In certain embodiments, dipeptides are preferred over longer polypeptides due to hydrophobicity of the longer peptides.

A variety of dipeptide-based cleavable linkers useful for linking drugs such as doxorubicin, mitomycin, camptothecin, pyrrolobenzodiazepine, tallysomycin and auristatin/auristatin family members to antibodies have been described (see, Dubowchik et al., 1998, J. Org. Chem. 67:1866-1872; Dubowchik et al., 1998, Bioorg. Med. Chem. Lett. 8(21):3341-3346; Walker et al., 2002, Bioorg. Med. Chem. Lett. 12:217-219; Walker et al., 2004, Bioorg. Med. Chem. Lett. 14:4323-4327; Sutherland et al., 2013, Blood 122: 1455-1463; and Francisco et al., 2003, Blood 102:1458-1465, of each of which is incorporated herein by reference). All of these dipeptide linkers, or modified versions of these dipeptide linkers, may be used in the anti-glyco-CD44 ADCs of the disclosure. Other dipeptide linkers that may be used include those found in ADCs such as Seattle Genetics' Brentuximab Vendotin SGN-35 (Adcetris™), Seattle Genetics SGN-75 (anti-CD-70, Val-Cit-monomethyl auristatin F(MMAF), Seattle Genetics SGN-CD33A (anti-CD-33, Val-Ala-(SGD-1882)), Celldex Therapeutics glembatumumab (CDX-011) (anti-NMB, Val-Cit-monomethyl auristatin E (MMAE), and Cytogen PSMA-ADC (PSMA-ADC-1301) (anti-PSMA, Val-Cit-MMAE).

Enzymatically cleavable linkers may include a self-immolative spacer to spatially separate the drug from the site of enzymatic cleavage. The direct attachment of a drug to a peptide linker can result in proteolytic release of an amino acid adduct of the drug, thereby impairing its activity. The use of a self-immolative spacer allows for the elimination of the fully active, chemically unmodified drug upon amide bond hydrolysis.

One self-immolative spacer is the bifunctional para-aminobenzyl alcohol group, which is linked to the peptide through the amino group, forming an amide bond, while amine containing drugs may be attached through carbamate functionalities to the benzylic hydroxyl group of the linker (PABC). The resulting prodrugs are activated upon protease-mediated cleavage, leading to a 1,6-elimination reaction releasing the unmodified drug, carbon dioxide, and remnants of the linker group. The following scheme depicts the fragmentation of p-amidobenzyl ether and release of the drug:

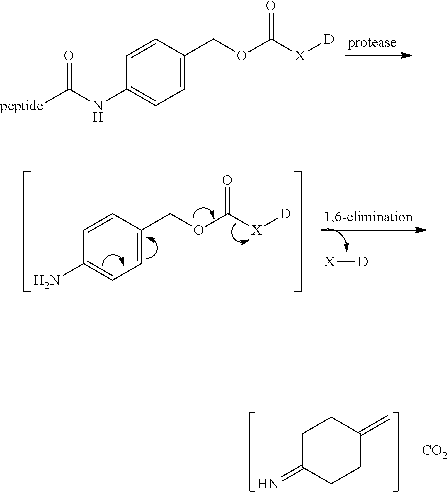

wherein X-D represents the unmodified drug.

Heterocyclic variants of this self-immolative group have also been described. See for example, U.S. Pat. No. 7,989,434, incorporated herein by reference.

In some embodiments, the enzymatically cleavable linker is a β-glucuronic acid-based linker. Facile release of the drug may be realized through cleavage of the β-glucuronide glycosidic bond by the lysosomal enzyme β-glucuronidase. This enzyme is present abundantly within lysosomes and is overexpressed in some tumor types, while the enzyme activity outside cells is low. β-Glucuronic acid-based linkers may be used to circumvent the tendency of an ADC to undergo aggregation due to the hydrophilic nature of β-glucuronides. In some embodiments, β-glucuronic acid-based linkers are preferred as linkers for ADCs linked to hydrophobic drugs. The following scheme depicts the release of the drug from and ADC containing a β-glucuronic acid-based linker:

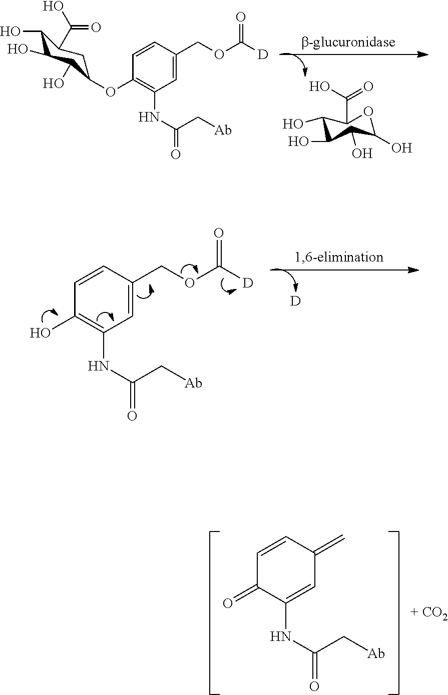

A variety of cleavable β-glucuronic acid-based linkers useful for linking drugs such as auristatins, camptothecin and doxorubicin analogues, CBI minor-groove binders, and psymberin to antibodies have been described (see, see Nolting, Chapter 5 “Linker Technology in Antibody-Drug Conjugates,” In: Antibody-Drug Conjugates: Methods in Molecular Biology, vol. 1045, pp. 71-100, Laurent Ducry (Ed.), Springer Science & Business Medica, LLC, 2013; Jeffrey et al., 2006, Bioconjug. Chem. 17:831-840; Jeffrey et al., 2007, Bioorg. Med. Chem. Lett. 17:2278-2280; and Jiang et al., 2005, J. Am. Chem. Soc. 127:11254-11255, each of which is incorporated herein by reference). All of these β-glucuronic acid-based linkers may be used in the anti-glyco-CD44 ADCs of the disclosure.

Additionally, cytotoxic and/or cytostatic agents containing a phenol group can be covalently bonded to a linker through the phenolic oxygen. One such linker, described in WO 2007/089149, relies on a methodology in which a diamino-ethane “SpaceLink” is used in conjunction with traditional “PABO”-based self-immolative groups to deliver phenols. The cleavage of the linker is depicted schematically below, where D represents a cytotoxic and/or cytostatic agent having a phenolic hydroxyl group.

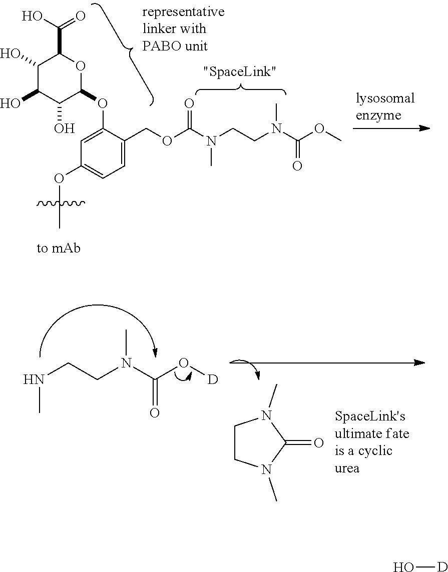

Cleavable linkers may include noncleavable portions or segments, and/or cleavable segments or portions may be included in an otherwise non-cleavable linker to render it cleavable. By way of example only, polyethylene glycol (PEG) and related polymers may include cleavable groups in the polymer backbone. For example, a polyethylene glycol or polymer linker may include one or more cleavable groups such as a disulfide, a hydrazone or a dipeptide.

Other degradable linkages that may be included in linkers include ester linkages formed by the reaction of PEG carboxylic acids or activated PEG carboxylic acids with alcohol groups on a biologically active agent, wherein such ester groups generally hydrolyze under physiological conditions to release the biologically active agent. Hydrolytically degradable linkages include, but are not limited to, carbonate linkages; imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol; and oligonucleotide linkages formed by a phosphoramidite group, including but not limited to, at the end of a polymer, and a 5′ hydroxyl group of an oligonucleotide.

In certain embodiments, the linker comprises an enzymatically cleavable peptide moiety, for example, a linker comprising structural formula (IVa) or (IVb):

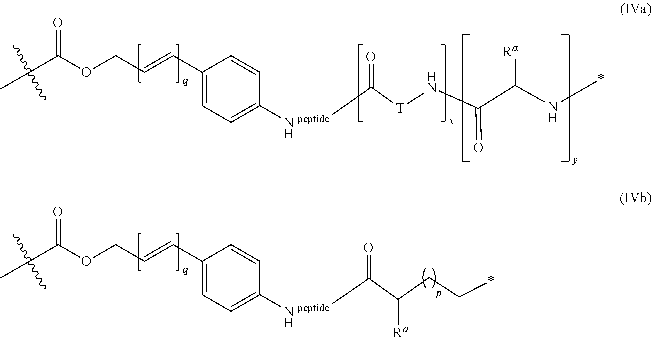

or a salt thereof, wherein: peptide represents a peptide (illustrated C→N and not showing the carboxy and amino “termini”) cleavable by a lysosomal enzyme; T represents a polymer comprising one or more ethylene glycol units or an alkylene chain, or combinations thereof; R^{a} is selected from hydrogen, alkyl, sulfonate and methyl sulfonate; p is an integer ranging from 0 to 5; q is 0 or 1; x is 0 or 1; y is 0 or 1; [symbol] represents the point of attachment of the linker to a cytotoxic and/or cytostatic agent; and * represents the point of attachment to the remainder of the linker.

In certain embodiments, the peptide is selected from a tripeptide or a dipeptide. In particular embodiments, the dipeptide is selected from: Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Val-Lys; Ala-Lys; Phe-Cit; Leu-Cit; Ile-Cit; Phe-Arg; and Trp-Cit. In certain embodiments, the dipeptide is selected from: Cit-Val; and Ala-Val.

Specific exemplary embodiments of linkers according to structural formula (IVa) that may be included in the anti-glyco-CD44 ADCs of the disclosure include the linkers illustrated below (as illustrated, the linkers include a group suitable for covalently linking the linker to an antibody):

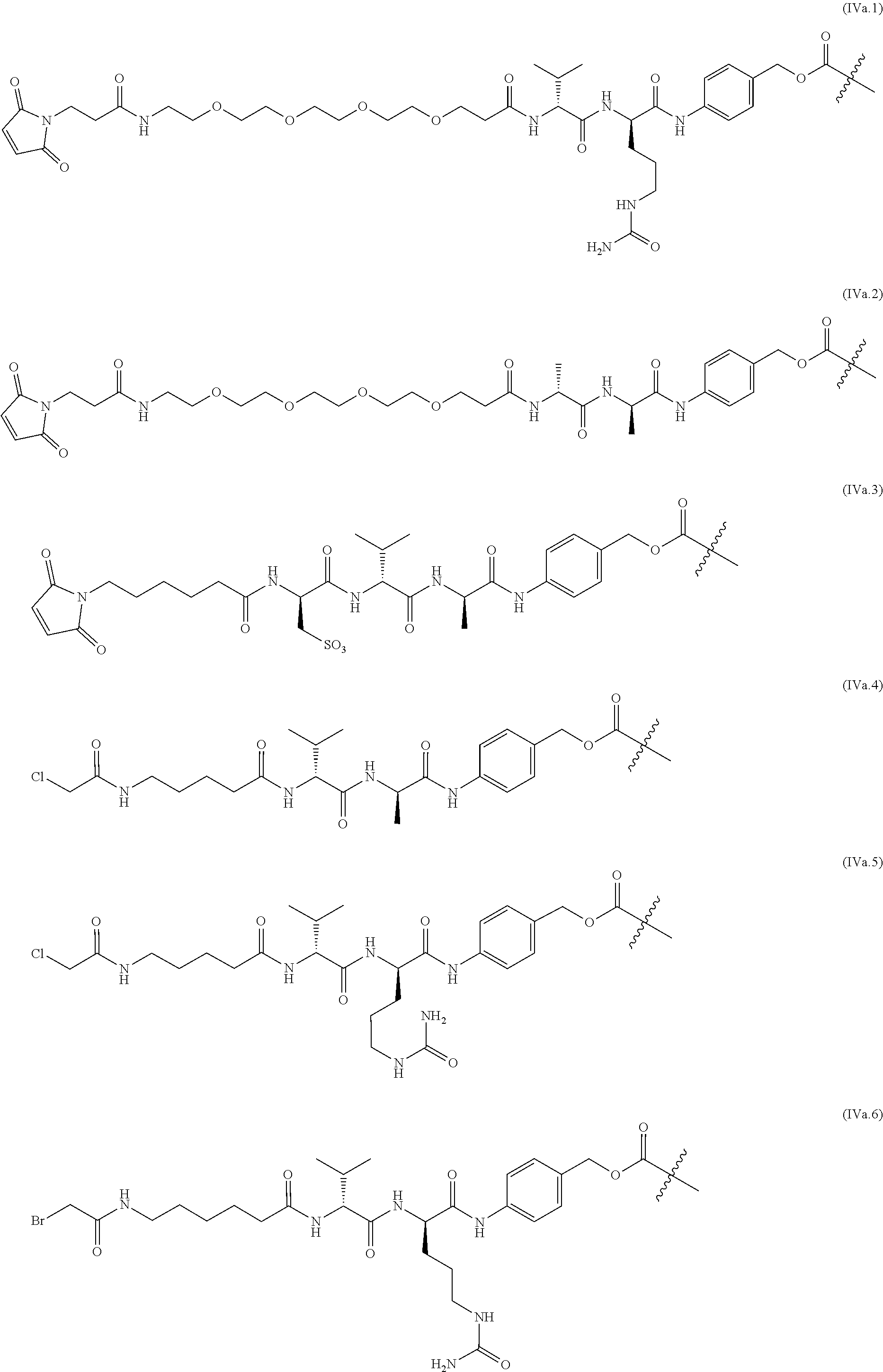

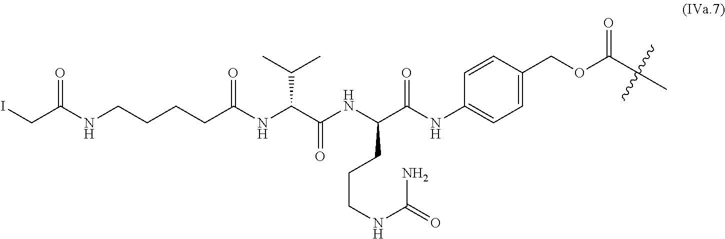 [SMILES: *C(=O)OCc1ccc(NC(=O)[C@@H](CCCNC(N)=O)NC(=O)[C@H](NC(=O)CCCCNC(=O)CI)C(C)C)cc1]

Specific exemplary embodiments of linkers according to structural formula (IVb) that may be included in the anti-glyco-CD44 ADCs of the disclosure include the linkers illustrated below (as illustrated, the linkers include a group suitable for covalently linking the linker to an antibody):

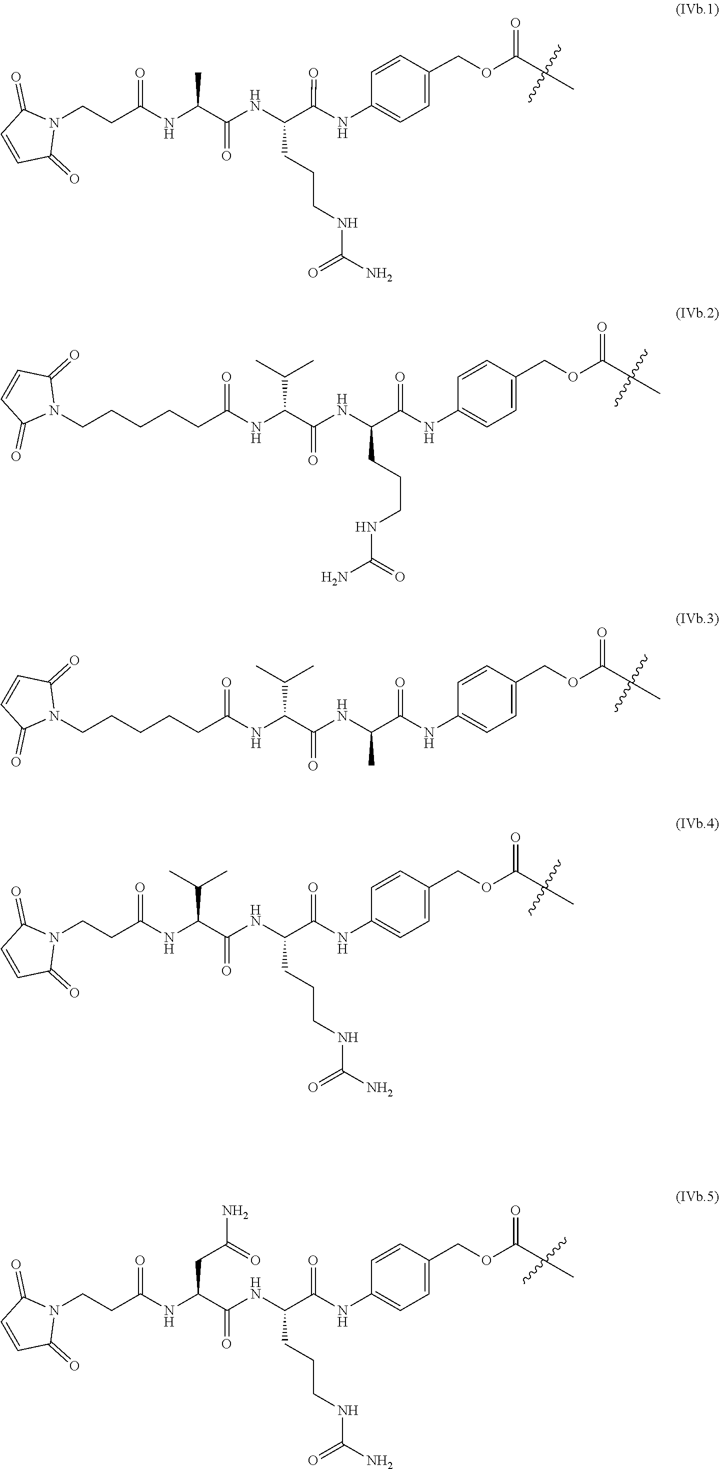 [SMILES: *C(=O)OCc1ccc(NC(=O)[C@H](CCCNC(N)=O)NC(=O)[C@H](C)NC(=O)CCN2C(=O)C=CC2=O)cc1 ; *C(=O)OCc1ccc(NC(=O)[C@@H](CCCNC(N)=O)NC(=O)[C@H](NC(=O)CCCCCN2C(=O)C=CC2=O)C(C)C)cc1 ; *C(=O)OCc1ccc(NC(=O)[C@@H](C)NC(=O)[C@H](NC(=O)CCCCCN2C(=O)C=CC2=O)C(C)C)cc1 ; *C(=O)OCc1ccc(NC(=O)[C@H](CCCNC(N)=O)NC(=O)[C@@H](NC(=O)CCN2C(=O)C=CC2=O)C(C)C)cc1 ; *C(=O)OCc1ccc(NC(=O)[C@H](CCCNC(N)=O)NC(=O)[C@H](CC(N)=O)NC(=O)CCN2C(=O)C=CC2=O)cc1]

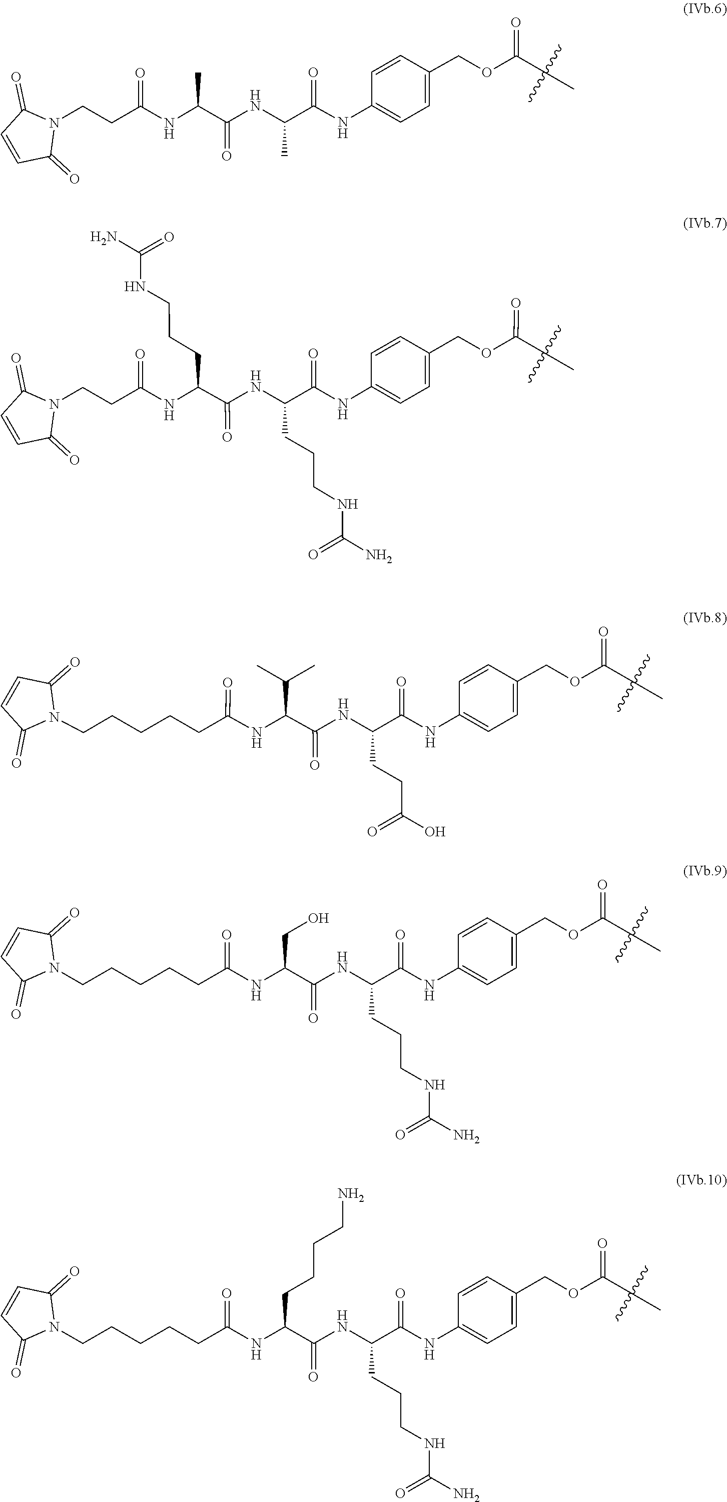 [SMILES: *C(=O)OCc1ccc(NC(=O)[C@H](C)NC(=O)[C@H](C)NC(=O)CCN2C(=O)C=CC2=O)cc1 ; *C(=O)OCc1ccc(NC(=O)[C@H](CCCNC(N)=O)NC(=O)[C@H](CCCNC(N)=O)NC(=O)CCN2C(=O)C=CC2=O)cc1 ; *CC(=O)OCc1ccc(NC(=O)[C@H](CCC(=O)O)NC(=O)[C@@H](NC(=O)CCCCCN2C(=O)C=CC2=O)C(C)C)cc1 ; *C(=O)OCc1ccc(NC(=O)[C@H](CCCNC(N)=O)NC(=O)[C@H](CO)NC(=O)CCCCCN2C(=O)C=CC2=O)cc1 ; *C(=O)OCc1ccc(NC(=O)[C@H](CCCNC(N)=O)NC(=O)[C@H](CCCCN)NC(=O)CCCCCN2C(=O)C=CC2=O)cc1]

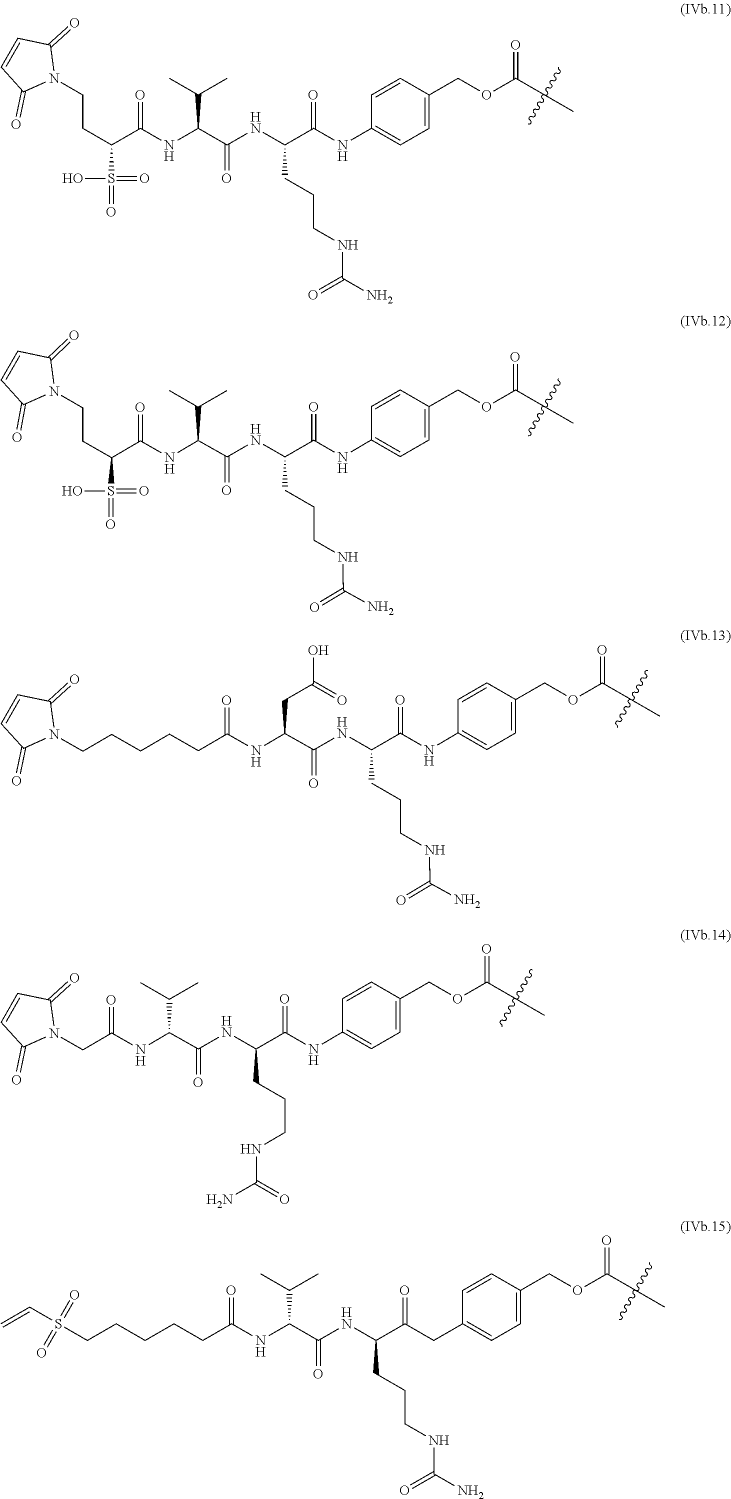

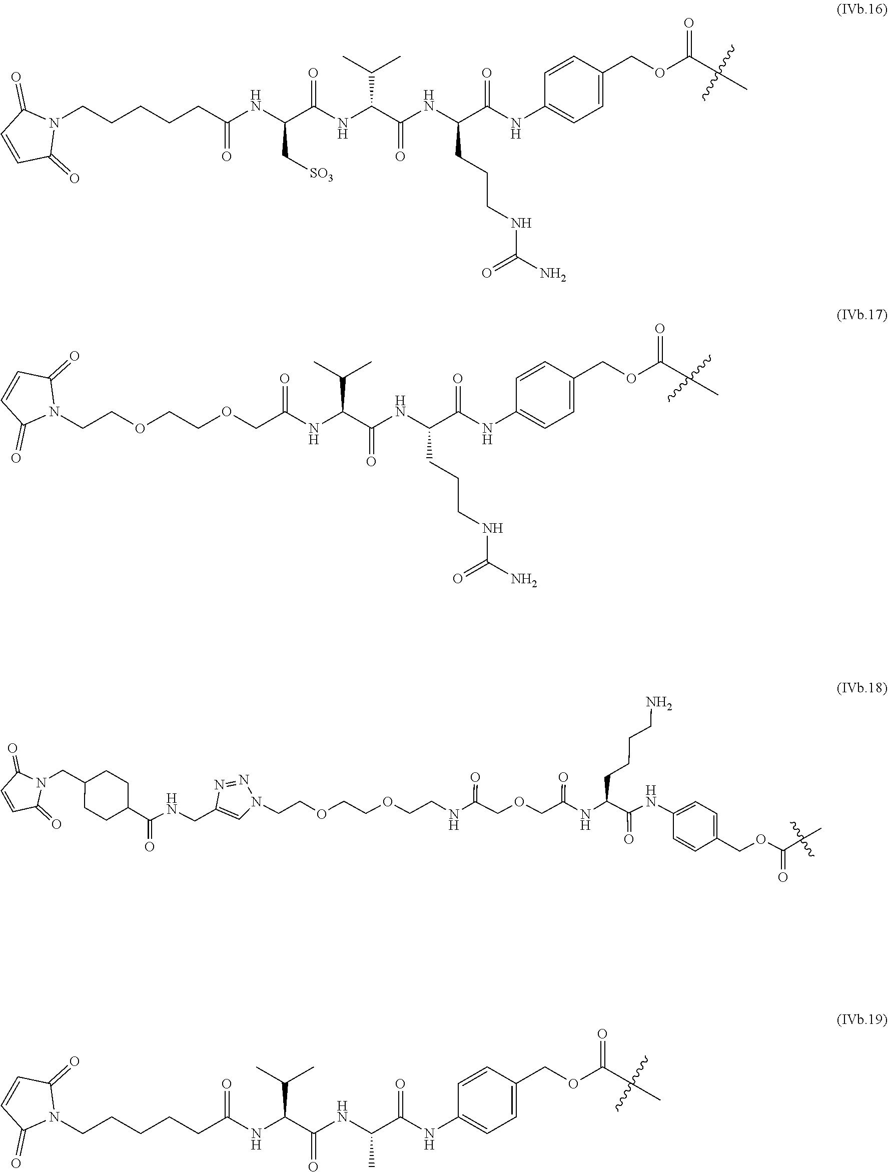

In certain embodiments, the linker comprises an enzymatically cleavable peptide moiety, for example, a linker comprising structural formula (IVc) or (IVd):

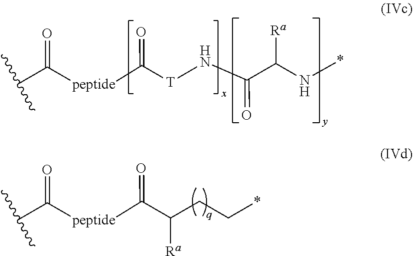

or a salt thereof, wherein: peptide represents a peptide (illustrated C→N and not showing the carboxy and amino “termini”) cleavable by a lysosomal enzyme; T represents a polymer comprising one or more ethylene glycol units or an alkylene chain, or combinations thereof; Ra is selected from hydrogen, alkyl, sulfonate and methyl sulfonate; p is an integer ranging from 0 to 5; q is 0 or 1; x is 0 or 1; y is 0 or 1; x [symbol] represents the point of attachment of the linker to a cytotoxic and/or cytostatic agent; and * represents the point of attachment to the remainder of the linker.

Specific exemplary embodiments of linkers according to structural formula (IVc) that may be included in the anti-glyco-CD44 ADCs of the disclosure include the linkers illustrated below (as illustrated, the linkers include a group suitable for covalently linking the linker to an antibody):

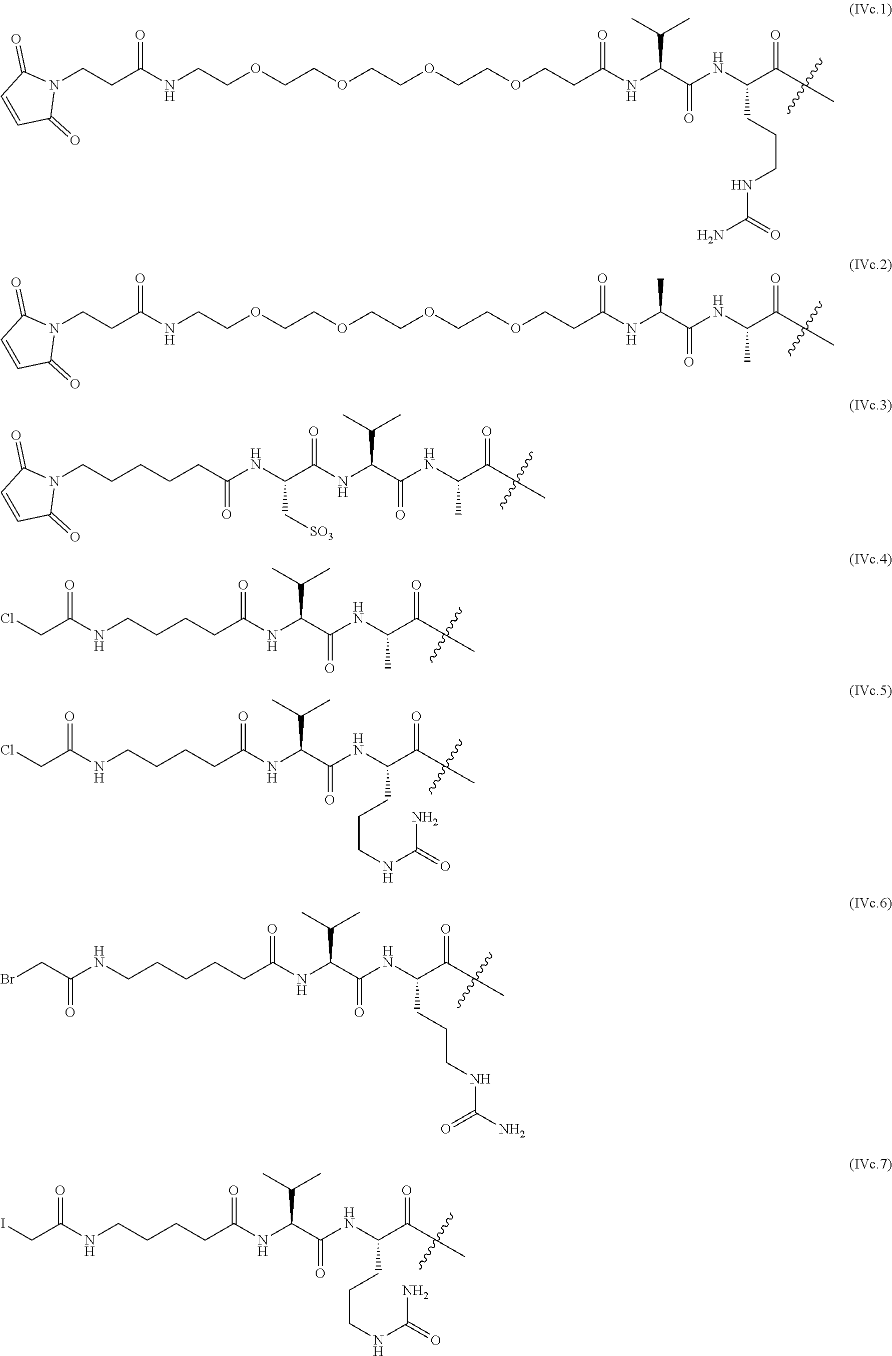

Specific exemplary embodiments of linkers according to structural formula (IVd) that may be included in the anti-glyco-CD44 ADCs of the disclosure include the linkers illustrated below (as illustrated, the linkers include a group suitable for covalently linking the linker to an antibody):

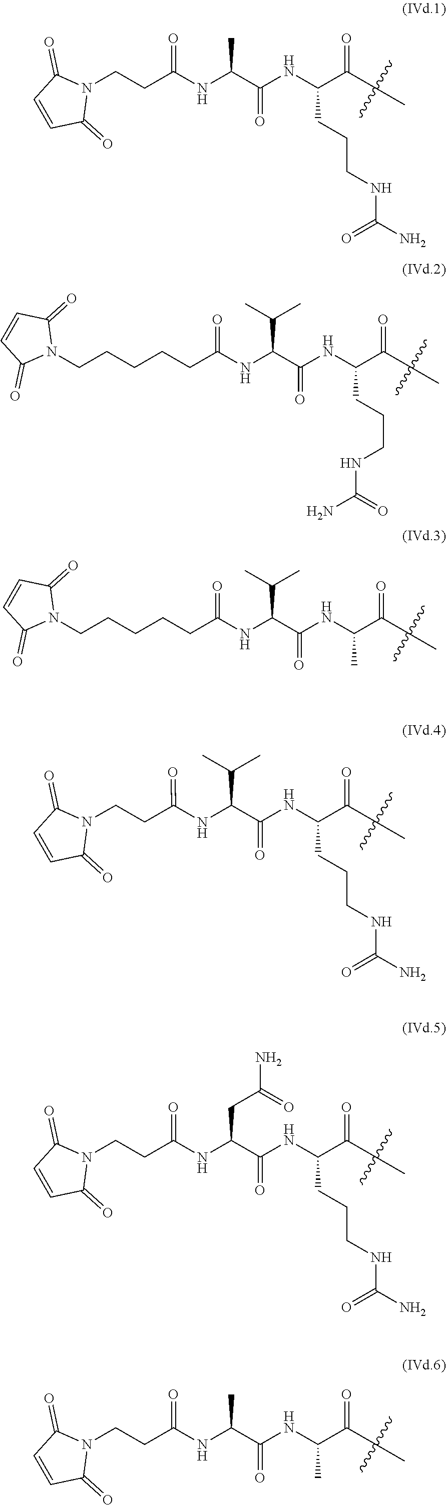 [SMILES: *C(=O)[C@H](CCCNC(N)=O)NC(=O)[C@H](C)NC(=O)CCN1C(=O)C=CC1=O ; *C(=O)[C@H](CCCNC(N)=O)NC(=O)[C@@H](NC(=O)CCCCCN1C(=O)C=CC1=O)C(C)C ; *C(=O)[C@H](C)NC(=O)[C@@H](NC(=O)CCCCCN1C(=O)C=CC1=O)C(C)C ; *C(=O)[C@H](CCCNC(N)=O)NC(=O)[C@@H](NC(=O)CCN1C(=O)C=CC1=O)C(C)C ; *C(=O)[C@H](CCCNC(N)=O)NC(=O)[C@H](CC(N)=O)NC(=O)CCN1C(=O)C=CC1=O ; *C(=O)[C@H](C)NC(=O)[C@H](C)NC(=O)CCN1C(=O)C=CC1=O]

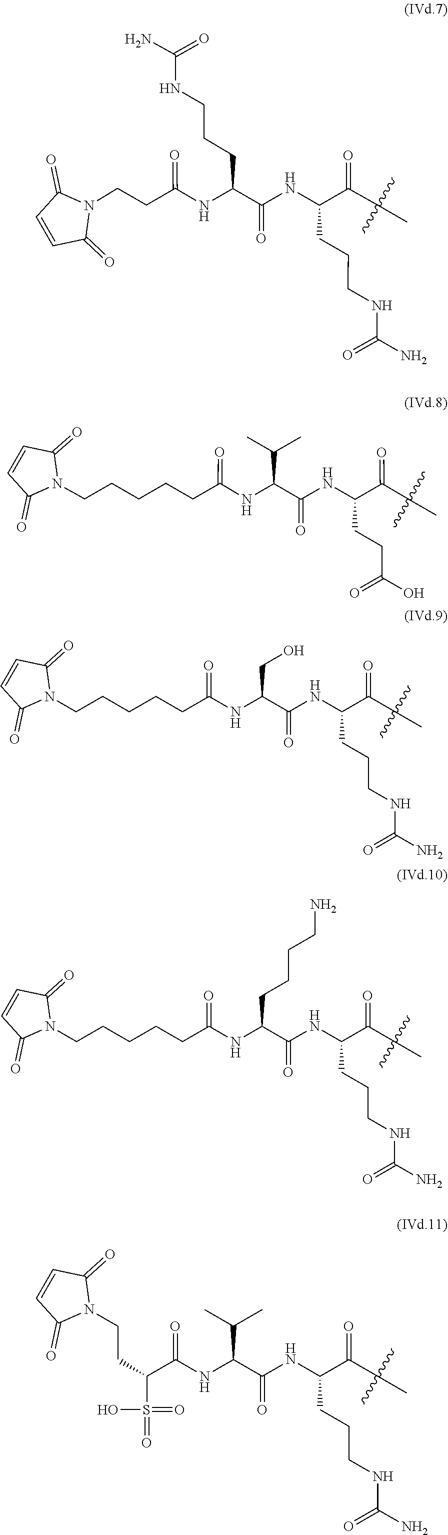 [SMILES: *CC(=O)[C@H](CCCNC(N)=O)NC(=O)[C@H](CCCNC(N)=O)NC(=O)CCN1C(=O)C=CC1=O ; *C(=O)[C@H](CCC(=O)O)NC(=O)[C@@H](NC(=O)CCCCCN1C(=O)C=CC1=O)C(C)C ; *C(=O)[C@H](CCCNC(N)=O)NC(=O)[C@H](CO)NC(=O)CCCCCN1C(=O)C=CC1=O ; *C(=O)[C@H](CCCNC(N)=O)NC(=O)[C@H](CCCCN)NC(=O)CCCCCN1C(=O)C=CC1=O ; *C(=O)[C@H](CCCNC(N)=O)NC(=O)[C@@H](NC(=O)[C@@H](CCN1C(=O)C=CC1=O)S(=O)(=O)O)C(C)C]

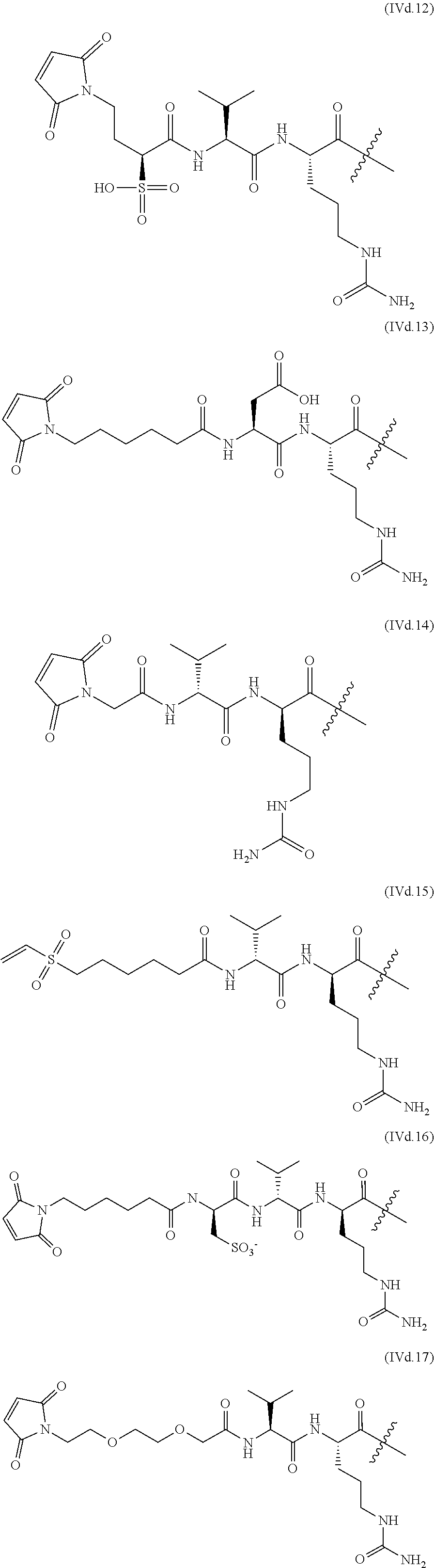

In certain embodiments, the linker comprising structural formula (IVa), (IVb), (IVc), or (IVd) further comprises a carbonate moiety cleavable by exposure to an acidic medium. In particular embodiments, the linker is attached through an oxygen to a cytotoxic and/or cytostatic agent.

6.2.4. Non-Cleavable Linkers

Although cleavable linkers may provide certain advantages, the linkers comprising the anti-glyco-CD44 ADC of the disclosure need not be cleavable. For noncleavable linkers, the release of drug does not depend on the differential properties between the plasma and some cytoplasmic compartments. The release of the drug is postulated to occur after internalization of the ADC via antigen-mediated endocytosis and delivery to lysosomal compartment, where the antibody is degraded to the level of amino acids through intracellular proteolytic degradation. This process releases a drug derivative, which is formed by the drug, the linker, and the amino acid residue to which the linker was covalently attached. The amino acid drug metabolites from conjugates with noncleavable linkers are more hydrophilic and generally less membrane permeable, which leads to less bystander effects and less nonspecific toxicities compared to conjugates with a cleavable linker. In general, ADCs with noncleavable linkers have greater stability in circulation than ADCs with cleavable linkers. Non-cleavable linkers may be alkylene chains, or maybe polymeric in natures, such as, for example, based upon polyalkylene glycol polymers, amide polymers, or may include segments of alkylene chains, polyalkylene glocols and/or amide polymers.

A variety of non-cleavable linkers used to link drugs to antibodies have been described. See, Jeffrey et al., 2006, Bioconjug. Chem. 17; 831-840; Jeffrey et al., 2007, Bioorg. Med. Chem. Lett. 17:2278-2280; and Jiang et al., 2005, J. Am. Chem. Soc. 127:11254-11255, each of which is incorporated herein by reference. All of these linkers may be included in the anti-glyco-CD44 ADCs of the disclosure.

In certain embodiments, the linker is non-cleavable in vivo, for example a linker according to structural formula (VIa), (VIb), (VIc) or (VId) (as illustrated, the linkers include a group suitable for covalently linking the linker to an antibody:

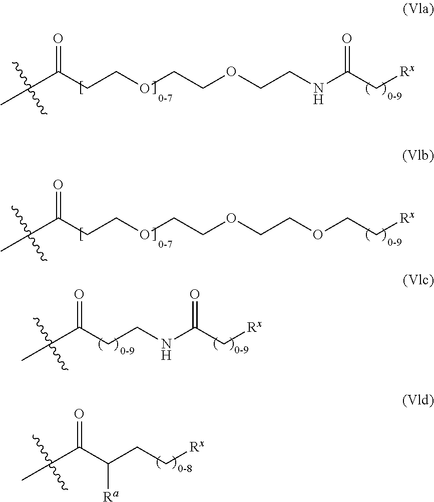

or salts thereof, wherein: R^{a} is selected from hydrogen, alkyl, sulfonate and methyl sulfonate; R^{x} is a moiety including a functional group capable of covalently linking the linker to an antibody; and [symbol] represents the point of attachment of the linker to a cytotoxic and/or cytostatic agent.

Specific exemplary embodiments of linkers according to structural formula (VIa)-(VId) that may be included in the anti-glyco-CD44 ADCs of the disclosure include the linkers illustrated below (as illustrated, the linkers include a group suitable for covalently linking the linker to an antibody, and [symbol] represents the point of attachment to a cytotoxic and/or cytostatic agent):

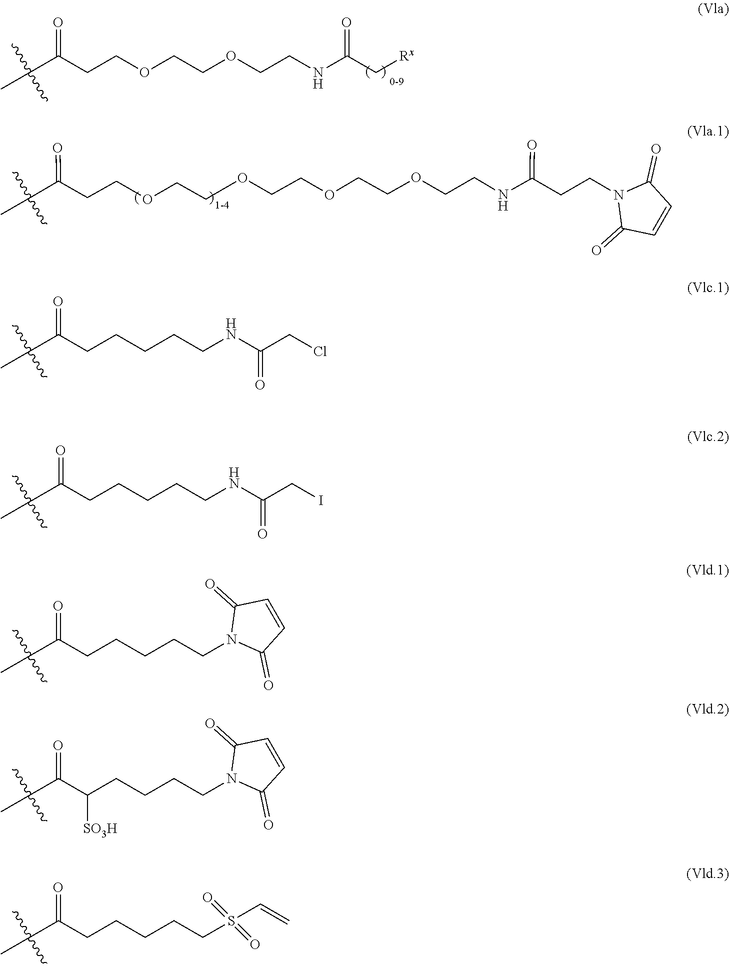

6.2.5. Groups Used to Attach Linkers to Antibodies

A variety of groups may be used to attach linker-drug synthons to antibodies to yield ADCs. Attachment groups can be electrophilic in nature and include: maleimide groups, activated disulfides, active esters such as NHS esters and HOBt esters, haloformates, acid halides, alkyl and benzyl halides such as haloacetamides. As discussed below, there are also emerging technologies related to “self-stabilizing” maleimides and “bridging disulfides” that can be used in accordance with the disclosure. The specific group used will depend, in part, on the site of attachment to the antibody.

One example of a “self-stabilizing” maleimide group that hydrolyzes spontaneously under antibody conjugation conditions to give an ADC species with improved stability is depicted in the schematic below. See US20130309256 A1; also Lyon et al., Nature Biotech published online, doi:10.1038/nbt.2968.

Normal System:

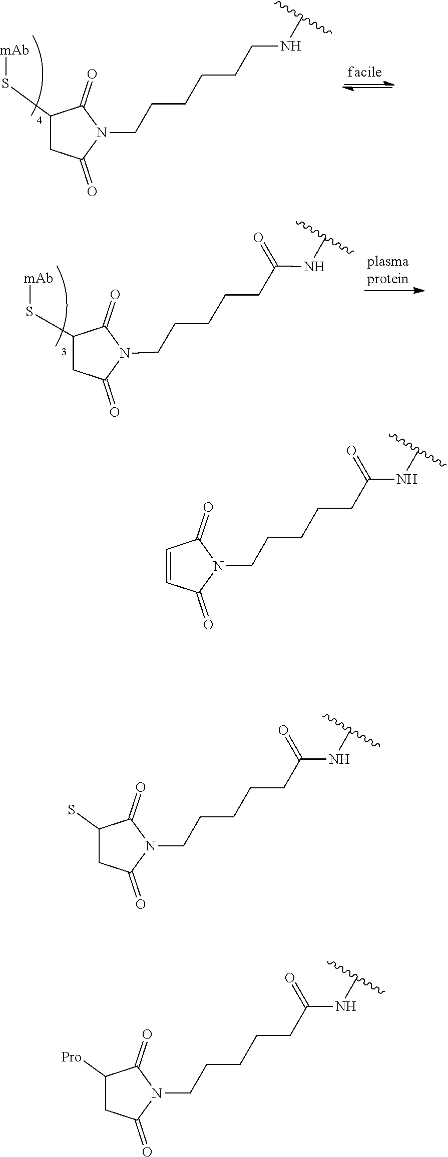

Leads to “DAR loss” over time

SGN MalDPR (maleimido dipropylamino) System:

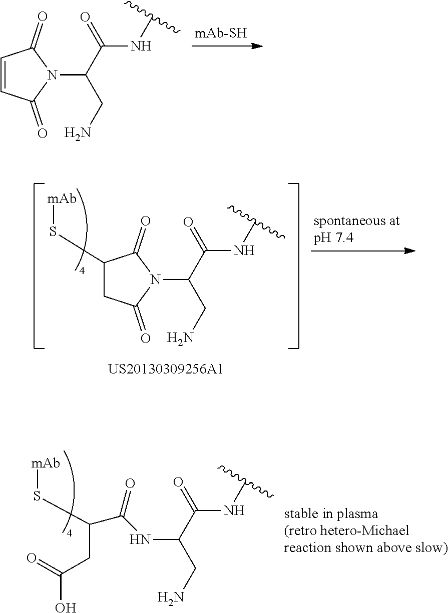

Polytherics has disclosed a method for bridging a pair of sulfhydryl groups derived from reduction of a native hinge disulfide bond. See, Badescu et al., 2014, Bioconjugate Chem. 25:1124-1136. The reaction is depicted in the schematic below. An advantage of this methodology is the ability to synthesize enriched DAR4 ADCs by full reduction of IgGs (to give 4 pairs of sulfhydryls) followed by reaction with 4 equivalents of the alkylating agent. ADCs containing “bridged disulfides” are also claimed to have increased stability.

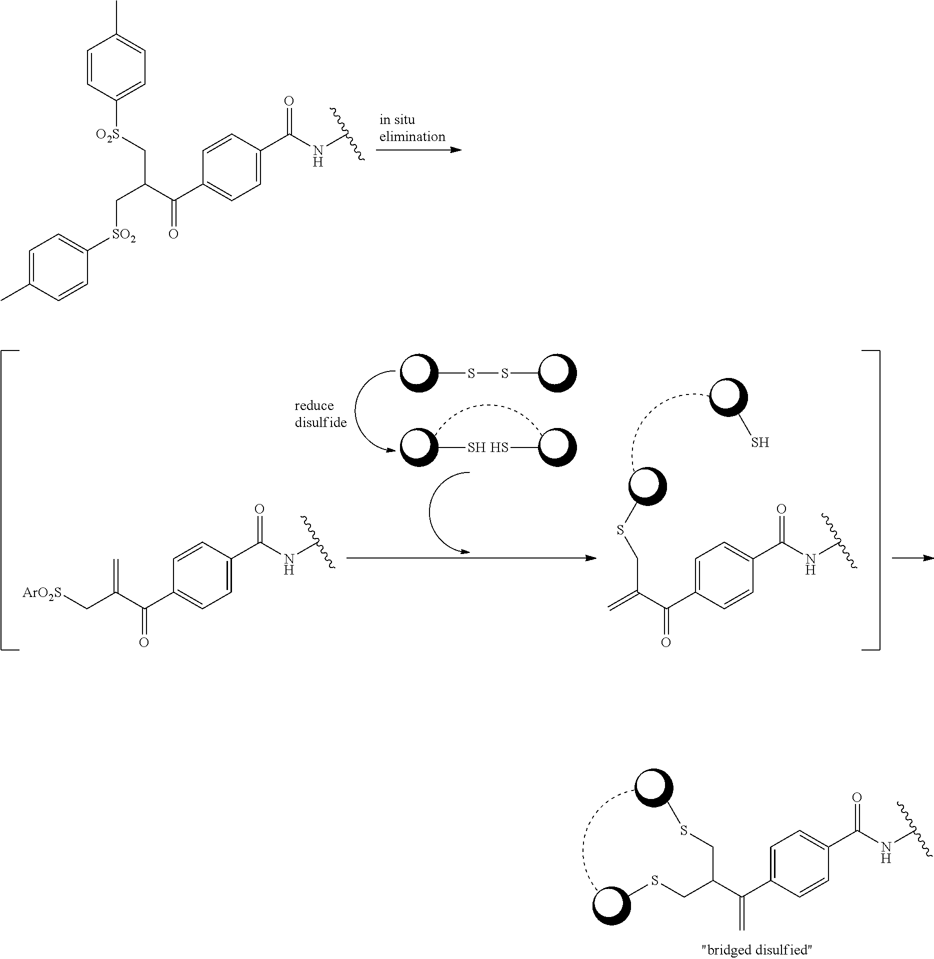

Similarly, as depicted below, a maleimide derivative (1, below) that is capable of bridging a pair of sulfhydryl groups has been developed. See WO2013/085925.

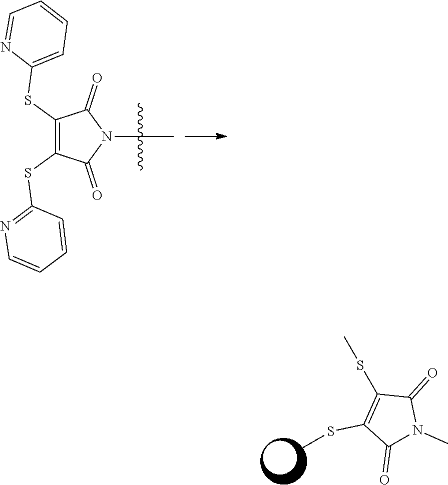

6.2.6. Linker Selection Considerations

As is known by skilled artisans, the linker selected for a particular ADC may be influenced by a variety of factors, including but not limited to, the site of attachment to the antibody (e.g., lys, cys or other amino acid residues), structural constraints of the drug pharmacophore and the lipophilicity of the drug. The specific linker selected for an ADC should seek to balance these different factors for the specific antibody/drug combination. For a review of the factors that are influenced by choice of linkers in ADCs, see Nolting, Chapter 5 “Linker Technology in Antibody-Drug Conjugates,” In: Antibody-Drug Conjugates: Methods in Molecular Biology, vol. 1045, pp. 71-100, Laurent Ducry (Ed.), Springer Science & Business Medica, LLC, 2013.

For example, ADCs have been observed to effect killing of bystander antigen-negative cells present in the vicinity of the antigen-positive tumor cells. The mechanism of bystander cell killing by ADCs has indicated that metabolic products formed during intracellular processing of the ADCs may play a role. Neutral cytotoxic metabolites generated by metabolism of the ADCs in antigen-positive cells appear to play a role in bystander cell killing while charged metabolites may be prevented from diffusing across the membrane into the medium and therefore cannot affect bystander killing. In certain embodiments, the linker is selected to attenuate the bystander killing effect caused by cellular metabolites of the ADC. In certain embodiments, the linker is selected to increase the bystander killing effect.

The properties of the linker may also impact aggregation of the ADC under conditions of use and/or storage. Typically, ADCs reported in the literature contain no more than 3-4 drug molecules per antibody molecule (see, e.g., Chari, 2008, Acc Chem Res 41:98-107). Attempts to obtain higher drug-to-antibody ratios (“DAR”) often failed, particularly if both the drug and the linker were hydrophobic, due to aggregation of the ADC (King et al., 2002, J Med Chem 45:4336-4343; Hollander et al., 2008, Bioconjugate Chem 19:358-361; Burke et al., 2009 Bioconjugate Chem 20:1242-1250). In many instances, DARs higher than 3-4 could be beneficial as a means of increasing potency. In instances where the cytotoxic and/or cytostatic agent is hydrophobic in nature, it may be desirable to select linkers that are relatively hydrophilic as a means of reducing ADC aggregation, especially in instances where DARS greater than 3-4 are desired. Thus, in certain embodiments, the linker incorporates chemical moieties that reduce aggregation of the ADCs during storage and/or use. A linker may incorporate polar or hydrophilic groups such as charged groups or groups that become charged under physiological pH to reduce the aggregation of the ADCs. For example, a linker may incorporate charged groups such as salts or groups that deprotonate, e.g., carboxylates, or protonate, e.g., amines, at physiological pH.

Exemplary polyvalent linkers that have been reported to yield DARs as high as 20 that may be used to link numerous cytotoxic and/or cytostatic agents to an antibody are described in WO 2009/073445; WO 2010/068795; WO 2010/138719; WO 2011/120053; WO 2011/171020; WO 2013/096901; WO 2014/008375; WO 2014/093379; WO 2014/093394; WO 2014/093640, the content of which are incorporated herein by reference in their entireties.

In particular embodiments, the aggregation of the ADCs during storage or use is less than about 10% as determined by size-exclusion chromatography (SEC). In particular embodiments, the aggregation of the ADCs during storage or use is less than 10%, such as less than about 5%, less than about 4%, less than about 3%, less than about 2%, less than about 1%, less than about 0.5%, less than about 0.1%, or even lower, as determined by size-exclusion chromatography (SEC).

6.2.7. Methods of Making Anti-Glyco-CD44 ADCs

The anti-glyco-CD44 ADCs of the disclosure may be synthesized using chemistries that are well-known. The chemistries selected will depend upon, among other things, the identity of the cytotoxic and/or cytostatic agent(s), the linker and the groups used to attach linker to the antibody. Generally, ADCs according to formula (I) may be prepared according to the following scheme:

D-L-R^{x}+Ab-R^{y}→[D-L-XY]ₙ-Ab (I)

where D, L, Ab, XY and n are as previously defined, and R^{x} and R^{y} represent complementary groups capable of forming covalent linkages with one another, as discussed above.

The identities of groups R^{x} and R^{y} will depend upon the chemistry used to link synthon D-L-R^{x} to the antibody. Generally, the chemistry used should not alter the integrity of the antibody, for example its ability to bind its target. Preferably, the binding properties of the conjugated antibody will closely resemble those of the unconjugated antibody. A variety of chemistries and techniques for conjugating molecules to biological molecules such as antibodies are known in the art and in particular to antibodies, are well-known. See, e.g., Amon et al., “Monoclonal Antibodies For Immunotargeting Of Drugs In Cancer Therapy,” in: Monoclonal Antibodies And Cancer Therapy, Reisfeld et al. Eds., Alan R. Liss, Inc., 1985; Hellstrom et al., “Antibodies For Drug Delivery,” in: Controlled Drug Delivery, Robinson et al. Eds., Marcel Dekker, Inc., 2nd Ed. 1987; Thorpe, “Antibody Carriers Of Cytotoxic Agents In Cancer Therapy: A Review,” in: Monoclonal Antibodies '84: Biological And Clinical Applications, Pinchera et al., Eds., 1985; “Analysis, Results, and Future Prospective of the Therapeutic Use of Radiolabeled Antibody In Cancer Therapy,” in: Monoclonal Antibodies For Cancer Detection And Therapy, Baldwin et al., Eds., Academic Press, 1985; Thorpe et al., 1982, Immunol. Rev. 62:119-58; PCT publication WO 89/12624. Any of these chemistries may be used to link the synthons to an antibody.

A number of functional groups R^{x} and chemistries useful for linking synthons to accessible lysine residues are known, and include by way of example and not limitation NHS-esters and isothiocyanates.

A number of functional groups R^{x} and chemistries useful for linking synthons to accessible free sulfhydryl groups of cysteine residues are known, and include by way of example and not limitation haloacetyls and maleimides.

However, conjugation chemistries are not limited to available side chain groups. Side chains such as amines may be converted to other useful groups, such as hydroxyls, by linking an appropriate small molecule to the amine. This strategy can be used to increase the number of available linking sites on the antibody by conjugating multifunctional small molecules to side chains of accessible amino acid residues of the antibody. Functional groups R^{x} suitable for covalently linking the synthons to these “converted” functional groups are then included in the synthons.

The antibody may also be engineered to include amino acid residues for conjugation. An approach for engineering antibodies to include non-genetically encoded amino acid residues useful for conjugating drugs in the context of ADCs is described by Axup et al., 2012, Proc Natl Acad Sci USA. 109(40):16101-16106, as are chemistries and functional group useful for linking synthons to the non-encoded amino acids.

Typically, the synthons are linked to the side chains of amino acid residues of the antibody, including, for example, the primary amino group of accessible lysine residues or the sulfhydryl group of accessible cysteine residues. Free sulfhydryl groups may be obtained by reducing interchain disulfide bonds.

For linkages where R^{y} is a sulfhydryl group (for example, when R^{x} is a maleimide), the antibody is generally first fully or partially reduced to disrupt interchain disulfide bridges between cysteine residues.

Cysteine residues that do not participate in disulfide bridges may engineered into an antibody by mutation of one or more codons. Reducing these unpaired cysteines yields a sulfhydryl group suitable for conjugation. Preferred positions for incorporating engineered cysteines include, by way of example and not limitation, positions S112C, S113C, A114C, S115C, A176C, 5180C, S252C, V286C, V292C, S357C, A359C, S398C, S428C (Kabat numbering) on the human IgG₁ heavy chain and positions V110C, S114C, S121C, S127C, S168C, V205C (Kabat numbering) on the human Ig kappa light chain (see, e.g., U.S. Pat. Nos. 7,521,541, 7,855,275 and 8,455,622).

As will appreciated by skilled artisans, the number of cytotoxic and/or cytostatic agents linked to an antibody molecule may vary, such that a collection of ADCs may be heterogeneous in nature, where some antibodies contain one linked agent, some two, some three, etc. (and some none). The degree of heterogeneity will depend upon, among other things, the chemistries used for linking the cytotoxic and/or cytostatic agents. For example, where the antibodies are reduced to yield sulfhydryl groups for attachment, heterogeneous mixtures of antibodies having zero, 2, 4, 6 or 8 linked agents per molecule are often produced. Furthermore, by limiting the molar ratio of attachment compound, antibodies having zero, 1, 2, 3, 4, 5, 6, 7 or 8 linked agents per molecule are often produced. Thus, it will be understood that depending upon context, stated DARs may be averages for a collection of antibodies. For example, “DAR4” can refer to an ADC preparation that has not been subjected to purification to isolate specific DAR peaks and can comprise a heterogeneous mixture of ADC molecules having different numbers of cytostatic and/or cytotoxic agents attached per antibody (e.g., 0, 2, 4, 6, 8 agents per antibody), but has an average drug-to-antibody ratio of 4. Similarly, in some embodiments, “DAR2” refers to a heterogeneous ADC preparation in which the average drug-to-antibody ratio is 2.

When enriched preparations are desired, antibodies having defined numbers of linked cytotoxic and/or cytostatic agents may be obtained via purification of heterogeneous mixtures, for example, via column chromatography, e.g., hydrophobic interaction chromatography.

Purity may be assessed by a variety of methods, as is known in the art. As a specific example, an ADC preparation may be analyzed via HPLC or other chromatography and the purity assessed by analyzing areas under the curves of the resultant peaks.

6.3 Chimeric Antigen Receptors

The present disclosure provides chimeric antigen receptors (CARs) comprising the anti-glyco-CD44 antibodies or antigen-binding fragments described herein. In some embodiments, the CAR comprises one or more scFvs (e.g., one or two) as described herein. For example, a CAR can comprise two scFvs covalently connected by a linker sequence (e.g., of 4-15 amino acids). Exemplary linkers include GGGGS (SEQ ID NO:183) and (GGGGS)₃ (SEQ ID NO:184).

The CARs of the disclosure typically comprise an extracellular domain operably linked to a transmembrane domain which is in turn operably linked to an intracellular domain for signaling. The CARs can further comprise a signal peptide at the N-terminus of the extracellular domain (e.g., a human CD8 signal peptide). In some embodiments, a CAR of the disclosure comprises a human CD8 signal peptide comprising the amino acid sequence MALPVTALLLPLALLLHAARP (SEQ ID NO:175).

The extracellular domains of the CARs of the disclosure comprise the sequence of an anti-glyco-CD44 antibody or antigen-binding fragment (e.g., as described in Section 6.1 or numbered embodiments 1 to 359).

Exemplary transmembrane domain sequence and intracellular domain sequences are described in Section 6.3.1 and 6.3.2, respectively.

Several fusion proteins described herein (e.g., numbered embodiments 395 to 429) are CARs, and the CAR-related disclosures apply to such fusion proteins.

6.3.1. Transmembrane Domain

With respect to the transmembrane domain, the CAR can be designed to comprise a transmembrane domain that is operably linked (e.g., fused) to the extracellular domain of the CAR.

The transmembrane domain may be derived either from a natural or from a synthetic source. Where the source is natural, the domain may be derived from any membrane-bound or transmembrane protein. Transmembrane regions of particular use in this disclosure may be derived from (i.e., comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. In some instances, a variety of human hinges can be employed as well including the human Ig (immunoglobulin) hinge.

In one embodiment, the transmembrane domain is synthetic (i.e., non-naturally occurring). Examples of synthetic transmembrane domains are peptides comprising predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

In one embodiment, the transmembrane domain in the CAR of the disclosure is the CD8 transmembrane domain. In one embodiment, the CD8 transmembrane domain comprises the amino acid sequence YLHLGALGRDLWGPSPVTGYHPLL (SEQ ID NO:185).

In one embodiment, the transmembrane domain in the CAR of the disclosure is the CD28 transmembrane domain. In one embodiment, the CD28 transmembrane domain comprises the amino acid sequence FVVVLVVVGGVLACYSLLVTVAFIIFWV (SEQ ID NO:186).

In some instances, the transmembrane domain of the CAR of the disclosure is linked to the extracellular domain by a CD8a hinge domain. In one embodiment, the CD8a hinge domain comprises the amino acid sequence TTTPAPRPPTPAPTIASQPLSLRPEACRPAAGGAVHTRGLDFAC (SEQ ID NO:187). In another embodiment, the CD8a hinge domain comprises the amino acid sequence

(SEQ ID NO: 176)
TTTPAPRPPTPAPTIASPLSLRPEACRPAAGGAVHTRGLDFACD.

In some instances, the transmembrane domain of the CAR of the disclosure is linked to the extracellular domain by a human IgG4-short hinge. In one embodiment, the human IgG4-short hinge comprises the amino acid sequence ESKYGPPCPSCP (SEQ ID NO:177).

In some instances, the transmembrane domain of the CAR of the disclosure is linked to the extracellular domain by a human IgG4-long hinge. In one embodiment, the human IgG4-long hinge comprises the amino acid sequence

(SEQ ID NO: 178)
ESKYGPPCPPCPAPPVAGPSVFLFPPKPKDTLMISRTPEVTCVVVDVS
QEDPEVQFNWYVDGVEVHNAKTKPREEQFQSTYRVVSVLTVLHQDWLN
GKEYKCKVSNKGLPSSIEKTISKAKGQPREPQVYTLPPSQEEMTKNQV
SLTCLVKGFYPSDIAVEWESNGQPENNYKTTPPVLDSDGSFFLYSRLT
VDKSRWQEGNVFSCSVMHEALHNHYTQKSLSLSLGKM.

6.3.2. Intracellular Domain

The intracellular signaling domain of the CAR of the disclosure is responsible for activation of at least one of the normal effector functions of the immune cell in which the CAR is expressed. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Preferred examples of intracellular signaling domains for use in the CAR of the disclosure include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

Signals generated through the TCR alone may be insufficient for full activation of the T cell and a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs.

Examples of ITAM containing primary cytoplasmic signaling sequences that are of particular use in the CARs of the disclosure include those derived from TCR zeta, FcR gamma, FcR beta, CD3 gamma, CD3 delta, CD3 epsilon, CD5, CD22, CD79a, CD79b, and CD66d. It is particularly preferred that cytoplasmic signaling molecule in the CAR of the disclosure comprises a cytoplasmic signaling sequence from CD3-zeta.

In a preferred embodiment, the cytoplasmic domain of the CAR is designed to include an ITAM containing primary cytoplasmic signaling sequences domain (e.g., that of CD3-zeta) by itself or combined with any other desired cytoplasmic domain(s) useful in the context of the CAR of the disclosure. For example, the cytoplasmic domain of the CAR can include a CD3 zeta chain portion and a costimulatory signaling region.

The costimulatory signaling region refers to a portion of the CAR comprising the intracellular domain of a costimulatory molecule. A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, DAP10, GITR, and the like.

The cytoplasmic signaling sequences within the cytoplasmic signaling portion of the CAR of the disclosure may be linked to each other in a random or specified order. Optionally, a short oligo- or polypeptide linker, preferably between 2 and 10 amino acids in length may form the linkage. A glycine-serine doublet provides a particularly suitable linker.

In one embodiment, the cytoplasmic domain comprises the signaling domain of CD3-zeta and the signaling domain of CD28. In some embodiments, the signaling domain of CD3-zeta comprises the amino acid sequence RVKFSRSADAPAYQQGQNQLYNELNLGRREEYDVLDKRRGRDPEMGGKPRRKNPQEGLYN ELQKDKMAEAYSEIGMKGERRRGKGHDGLYQGLSTATKDTYDALHMQALPPR (SEQ ID NO: 180). In some embodiments, the signaling domain of CD28 comprises the amino acid acid sequence RSKRSRLLHSDYMNMTPRRPGPTRKHYQPYAPPRDFAAYRS (SEQ ID NO:179).

In another embodiment, the cytoplasmic domain comprises the signaling domain of CD3-zeta and the signaling domain of 4-1BB.

6.4 MicAbodies

The present disclosure provides MicAbodies comprising the anti-glyco-CD44 antibodies and antigen-binding fragments of the disclosure. MicAbodies are fusion proteins comprising an antibody or antigen-binding fragment and an engineered MHC-class I-chain-related (MIC) protein domain. MIC proteins are the natural ligands of human NKG2D receptors expressed on the surface of NK cells, and the α1-α2 domain of MIC proteins provides the binding site for the NKG2D receptor. By fusing an engineered MIC protein domain (e.g. an engineered α1-α2 domain) to a cancer-targeting antibody or antigen-binding fragment, T-cells expressing an engineered NKG2D receptor capable of binding the engineered MIC protein domain can be targeted to cancer cells. Engineered MIC protein domains that can be included in MicAbodies of the disclosure, and NKG2D receptors capable of binding the engineered MIC protein domains, CARs and CAR T cells comprising the NKG2D receptors are described in U.S. publication nos. US 2011/0183893, US2011/0311561, US 2015/0165065, and US 2016/0304578 and PCT publication nos. WO 2016/090278, WO 2017/024131, WO 2017/222556, and WO 2019/191243, the contents of which are incorporated herein by reference in their entireties.

In some embodiments, the MicAbodies of the disclosure comprise α1-α2 domains which are at least 80% identical or homologous to the α1-α2 domain of an NKG2D ligand (e.g., MICA, MICB, ULBP1, ULBP2, ULBP3, ULBP4, ULBP5, ULBP6, or OMCP). Exemplary amino acid sequences of MICA, MICB, ULBP1, ULBP2, ULBP3, ULBP4, ULBP5, ULBP6, and OMCP are set forth as SEQ ID NOs: 1-9 of WO 2019/191243, respectively, the sequences of which are incorporated herein by reference. In other embodiments, the α1-α2 domain is 85% identical to a native or natural α1-α2 domain of an NKG2D ligand. In yet other embodiments, the α1-α2 domain is 90% identical to a native or natural α1-α2 domain of a natural NKG2D ligand protein and binds non-natural NKG2D.

In some embodiments, the MicAbodies of the disclosure comprise α1-α2 domains which are at least 80% identical or homologous to a native or natural α1-α2 domain of a human MICA or MICB protein and bind NKG2D. In some embodiments, the α1-α2 domain is 85% identical to a native or natural α1-α2 domain of a human MICA or MICB protein and binds NKG2D. In other embodiments, the α1-α2 domain is 90%, 95%, 96%, 97%, 98%, or 99% identical to a native or natural α1-α2 platform domain of a human MICA or MICB protein and binds NKG2D.

In some embodiments, specific mutations in α1-α2 domains of NKG2D ligands can be made to create non-natural α1-α2 domains that bind non-natural NKG2D receptors, themselves engineered so as to have reduced affinity for natural NKG2D ligands. This can be done, for example, through genetic engineering. A non-natural NKG2D receptor so modified can be used to create on the surface of NK- or T-cells of the immune system an NKG2D-based CAR that can preferentially bind to and be activated by molecules comprised of the non-natural α1-α2 domains. These pairs of non-natural NKG2D receptors and their cognate non-natural NKG2D ligands can provide important safety, efficacy, and manufacturing advantages for treating cancer and viral infections as compared to traditional CAR-T cells and CAR-NK cells. Activation of CAR-T cells and CAR-NK cells having a NKG2D-based CAR can be controlled by administration of a MicAbody. In the event that an adverse event develops, the dosing regimen of the MicAbody can be modified rather than having to deploy an induced suicide mechanism to destroy the infused CAR cells.

MicAbodies can be generated by attaching an antibody or antigen-binding fragment to an engineered α1-α2 domain via a linker, e.g., APTSSSGGGGS (SEQ ID NO:188) or GGGS (SEQ ID NO:189). For example, an α1-α2 domain can be fused to the C-terminus of an IgG heavy chain or light chain, for example, as described in WO 2019/191243.

In some embodiments, the MicAbodies of the disclosure comprise an engineered α1-α2 domain comprising the amino acid sequence

(MICA25.17)
(SEQ ID NO: 190)
EPHSLRYNLTVLSWDGSVQSGFLTEVHLDGQPFLRCDRQKCRAKPQGQ
WAEDVLGNKTWDRETRDLTGWGTTLLMTLAHIKDQKEGLHSLQEIRVC
EIHEDNSTRSSQHFYYDGELFLSQNLETLEWTMPQSSRAQTLAMNVRN
FLKEDAMETDIGYRLMRADCLSELRRYLKSGVVLRRTV.

In other embodiments, the MicAbodies of the disclosure comprise an engineered α1-α2 domain comprising the amino acid sequence

(MICA25.18)
(SEQ ID NO: 191)
EPHSLRYNLTVLSWDGSVQSGFLTEVHLDGQPFLRCDRQKCRAKPQGQ
WAEDVLGNKTWDRETRDLTGWGTFLRMTLAHIKDQKEGLHSLQEIRVC
EIHEDNSTRSSQHFYYDGELFLSQNLETLEWTMPQSSRAQTLAMNVRN
FLKEDAMETDRSGLLMRADCLSELRRYLKSGVVLRRTV.

In other embodiments, the MicAbodies of the disclosure comprise an engineered α1-α2 domain comprising the amino acid sequence

(ULBP2.S1)
(SEQ ID NO: 192)
AAEPHSLSYDITVIPKFRPGPRWCAVQGQVDEKTFLHYDCGNKTVTPV
SPLGKKLNVTTAWKAQNPVLREVVDILTEQLWDIQLENYTPKEPLTLQ
ARMSCEQKAEGHSSGSWQFSFDGQIFLLFDSEKRMWTTVHPGARKMKE
KWENDKVVATTLYTWSMGDCIGWLEDFLMGMDSTLEPSAGAP.

In other embodiments, the MicAbodies of the disclosure comprise an engineered α1-α2 domain comprising the amino acid sequence

(SEQ ID NO: 193)
AAEPHSLSYDITVIPKFRPGPRWCAVQGQVDEKTFLHYDCGNKTVTPVSP
LGKKLNVTTAWKAQNPVLREVVDILTEQLWDIQLENYTPKEPLTLQARMS
CEQKAEGHSSGSWQFSFDGQIFLLFDSEKRMWTTVHPGARKMKEKWENDK
VVATLMRIWSMGDCIGWLEDFLMGMDSTLEPSAGAP
(ULBP2.S2).

In other embodiments, the MicAbodies of the disclosure comprise an engineered α1-α2 domain comprising the amino acid sequence

(SEQ ID NO: 194)
AAEPHSLSYDITVIPKFRPGPRWCAVQGQVDEKTFLHYDCGNKTVTPVSP
LGKKLNVTTAWKAQNPVLREVVDILTEQLWDIQLENYTPKEPLTLQARMS
CEQKAEGHSSGSWQFSFDGQIFLLFDSEKRMWTTVHPGARKMKEKWENDK
VVATKLYLWSMGDCIGWLEDFLMGMDSTLEPSAGAP
(ULBP2.S3).

In other embodiments, the MicAbodies of the disclosure comprise an engineered α1-α2 domain comprising the amino acid sequence

(SEQ ID NO: 195)
AAEPHSLWYNFTIIHLPRHGQQWCEVQSQVDQKNFLSYDCGSDKVLSMGH
LEEQLYATDAWGKQLEMLREVGQRLRLELADTELEDFTPSGPLTLQVRMS
CESEADGYIRGSWQFSFDGRKFLLFDSNNRKWTVVHAGARRMKEKWEKDS
GLTTDLIRRSMGDCKSWLRDFLMHRKKRLEPTAP
(ULBP3.S1).

In other embodiments, the MicAbodies of the disclosure comprise an engineered α1-α2 domain comprising the amino acid sequence

(SEQ ID NO: 196)
AAEPHSLWYNFTIIHLPRHGQQWCEVQSQVDQKNFLSYDCGSDKVLSMGH
LEEQLYATDAWGKQLEMLREVGQRLRLELADTELEDFTPSGPLTLQVRMS
CESEADGYIRGSWQFSFDGRKFLLFDSNNRKWTVVHAGARRMKEKWEKDS
GLTTYFYLRSMGDCKSWLRDFLMHRKKRLEPTAP
(ULBP3.S2).

In other embodiments, the MicAbodies of the disclosure comprise an engineered α1-α2 domain comprising the amino acid sequence

(SEQ ID NO: 197)
EPHSLSYDITVIPKFRPGPRWCAVQGQVDEKTFLHYDCGNKTVTPVSPLG
KKLNVTTAWKAQNPVLREVVDILTEQLWDIQLENYTPKEPLTLQARMSCE
QKAEGHSSGSWQFSFDGQIFLLFDSEKRMWTTVHPGARKMKEKWENDKVV
ATILWQTSMGDCIGWLEDFLMGMDSTLEPS
(ULBP2.C).

In other embodiments, the MicAbodies of the disclosure comprise an engineered α1-α2 domain comprising the amino acid sequence

(SEQ ID NO: 198)
EPHSLSYDITVIPKFRPGPRWCAVQGQVDEKTFLHYDCGNKTVTPVSPLG
KKLNVTTAWKAQNPVLREVVDILTEQLWDIQLENYTPKEPLTLQARMSCE
QKAEGHSSGSWQFSFDGQIFLLFDSEKRMWTTVHPGARKMKEKWENDKVV
ATLLWGWSMGDCIGWLEDFLMGMDSTLEPS
(ULBP2.R).

In other embodiments, the MicAbodies of the disclosure comprise an engineered α1-α2 domain comprising the amino acid sequence

(SEQ ID NO: 199)
EPHSLSYDITVIPKFRPGPRWCAVQGQVDEKTFLHYDCGNKTVTPVSPLG
KKLNVTTAWKAQNPVLREVVDILTEQLWDIQLENYTPKEPLTLQARMSCE
QKAEGHSSGSWQFSFDGQIFLLFDSEKRMWTTVHPGARKMKEKWENDKVV
ATMFWSWSMGDCIGWLEDFLMGMDSTLEPS
(ULBP2.AA).

In other embodiments, the MicAbodies of the disclosure comprise an engineered α1-α2 domain comprising the amino acid sequence

(SEQ ID NO: 200)
EPHSLSYDITVIPKFRPGPRWCAVQGQVDEKTFLHYDCGNKTVTPVSPLG
KKLNVTTAWKAQNPVLREVVDILTEQLWDIQLENYTPKEPLTLQARMSCE
QKAEGHSSGSWQFSFDGQIFLLFDSEKRMWTTVHPGARKMKEKWENDKVV
ATLMWQWSMGDCIGWLEDFLMGMDSTLEPS
(ULBP2.AB).

An exemplary engineered NKG2D receptor comprises the amino acid sequence NSLFNQEVQIPLTESYCGPCPKNWICYKNNCYQFFDESKNWYESQASCMSQNASLLKVYSKE DQDLLKLVKSYHWMGLVHIPTNGSWQWEDGSILSPNLLTIIEMQKGDCALYASSFKGYIENCST PNTYICMQRTV (SEQ ID NO:201) in which the tyrosine at position 73 has been replaced with another amino acid, for example alanine.

Another exemplary engineered NKG2D receptor comprises the amino acid sequence FLNSLFNQEVQIPLTESYCGPCPKNWICYKNNCYQFFDESKNWYESQASCMSQNASLLKVYS KEDQDLLKLVKSYHWMGLVHIPTNGSWQWEDGSILSPNLLTIIEMQKGDCALYASSFKGYIENC STPNTYICMQRTV (SEQ ID NO:202) in which the tyrosines are positions 75 and 122 have been replaced with another amino acid, for example alanine at position 75 and phenylalanine at position 122.

6.5 Nucleic Acids, Recombinant Vectors and Host Cells

The present disclosure encompasses nucleic acid molecules encoding immunoglobulin light and heavy chain genes for anti-glyco-CD44 antibodies, vectors comprising such nucleic acids, and host cells capable of producing the anti-glyco-CD44 antibodies of the disclosure. In certain aspects, the nucleic acid molecules encode, and the host cells are capable of expressing, the anti-glyco-CD44 antibodies and antibody-binding fragments of the disclosure (e.g., as described in Section 6.1 and numbered embodiments 1 to 384) as well as fusion proteins (e.g., as described in numbered embodiments 385 to 394) and chimeric antigen receptors (e.g., as described in Section 6.3 and numbered embodiments 395 to 429) containing them. Exemplary vectors of the disclosure are described in numbered embodiments 442 to 444 and exemplary host cells are described in numbered embodiments 445 to 448.

An anti-glyco-CD44 antibody of the disclosure can be prepared by recombinant expression of immunoglobulin light and heavy chain genes in a host cell. To express an antibody recombinantly, a host cell is transfected with one or more recombinant expression vectors carrying DNA fragments encoding the immunoglobulin light and heavy chains of the antibody such that the light and heavy chains are expressed in the host cell and, optionally, secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Molecular Cloning; A Laboratory Manual, Second Edition (Sambrook, Fritsch and Maniatis (eds), Cold Spring Harbor, N.Y., 1989), Current Protocols in Molecular Biology (Ausubel, F. M. et al., eds., Greene Publishing Associates, 1989) and in U.S. Pat. No. 4,816,397.

To generate nucleic acids encoding such anti-glyco-CD44 antibodies, DNA fragments encoding the light and heavy chain variable regions are first obtained. These DNAs can be obtained by amplification and modification of germline DNA or cDNA encoding light and heavy chain variable sequences, for example using the polymerase chain reaction (PCR). Germline DNA sequences for human heavy and light chain variable region genes are known in the art (See, e.g., the “VBASE” human germline sequence database; see also Kabat et al., 1991, Sequences of Proteins of Immunological Interest, Fifth Edition, U.S. Department of Health and Human Services, NIH Publication No. 91-3242; Tomlinson et al., 1992, J. Mol. Biol. 22T:116-198; and Cox et al., 1994, Eur. J. Immunol. 24:827-836; the contents of each of which are incorporated herein by reference).

Once DNA fragments encoding anti-glyco-CD44 antibody-related V_{H} and V_{L} segments are obtained, these DNA fragments can be further manipulated by standard recombinant DNA techniques, for example to convert the variable region genes to full-length antibody chain genes, to Fab fragment genes or to a scFv gene. In these manipulations, a V_{H}- or V_{L}-encoding DNA fragment is operatively linked to another DNA fragment encoding another protein, such as an antibody constant region or a flexible linker. The term “operatively linked,” as used in this context, is intended to mean that the two DNA fragments are joined such that the amino acid sequences encoded by the two DNA fragments remain in-frame.

The isolated DNA encoding the V_{H} region can be converted to a full-length heavy chain gene by operatively linking the V_{H}-encoding DNA to another DNA molecule encoding heavy chain constant regions (CH₁, CH₂, CH₃ and, optionally, CH₄). The sequences of human heavy chain constant region genes are known in the art (See, e.g., Kabat et al., 1991, Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG₁, IgG₂, IgG₃, IgG₄, IgA, IgE, IgM or IgD constant region, but in certain embodiments is an IgG₁ or IgG₄ constant region. For a Fab fragment heavy chain gene, the V_{H}-encoding DNA can be operatively linked to another DNA molecule encoding only the heavy chain CH1 constant region.

The isolated DNA encoding the V_{L} region can be converted to a full-length light chain gene (as well as a Fab light chain gene) by operatively linking the V_{L}-encoding DNA to another DNA molecule encoding the light chain constant region, CL. The sequences of human light chain constant region genes are known in the art (See, e.g., Kabat et al., 1991, Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region, but in certain embodiments is a kappa constant region.

To create a scFv gene, the V_{H}- and V_{L}-encoding DNA fragments can be operatively linked to another fragment encoding a flexible linker, e.g., encoding the amino acid sequence (Gly₄˜Ser)₃ (SEQ ID NO:184), such that the V_{H} and V_{L} sequences can be expressed as a contiguous single-chain protein, with the V_{H} and V_{L} regions joined by the flexible linker (See, e.g., Bird et al., 1988, Science 242:423-426; Huston et al., 1988, Proc. Natl. Acad. Sci. USA 85:5879-5883; McCafferty et al., 1990, Nature 348:552-554).

To express the anti-glyco-CD44 antibodies of the disclosure, DNAs encoding partial or full-length light and heavy chains, obtained as described above, are inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vectors or, more typically, both genes are inserted into the same expression vector.

The antibody genes are inserted into the expression vector by standard methods (e.g., ligation of complementary restriction sites on the antibody gene fragment and vector, or blunt end ligation if no restriction sites are present). Prior to insertion of the anti-glyco-CD44 antibody-related light or heavy chain sequences, the expression vector can already carry antibody constant region sequences. For example, one approach to converting the anti-glyco-CD44 monoclonal antibody-related V_{H} and V_{L} sequences to full-length antibody genes is to insert them into expression vectors already encoding heavy chain constant and light chain constant regions, respectively, such that the V_{H} segment is operatively linked to the CH segment(s) within the vector and the V_{L} segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the disclosure carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel, Gene Expression Technology: Methods in Enzymology 185, Academic Press, San Diego, Calif., 1990. It will be appreciated by those skilled in the art that the design of the expression vector, including the selection of regulatory sequences may depend on such factors as the choice of the host cell to be transformed, the level of expression of protein desired, etc. Suitable regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see, e.g., U.S. Pat. No. 5,168,062 by Stinski, U.S. Pat. No. 4,510,245 by Bell et al., and U.S. Pat. No. 4,968,615 by Schaffner et al.

In addition to the antibody chain genes and regulatory sequences, the recombinant expression vectors of the disclosure can carry additional sequences, such as sequences that regulate replication of the vector in host cells (e.g., origins of replication) and selectable marker genes. The selectable marker gene facilitates selection of host cells into which the vector has been introduced (See, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Suitable selectable marker genes include the dihydrofolate reductase (DHFR) gene (for use in DHFR⁻ host cells with methotrexate selection/amplification) and the neo gene (for G418 selection). For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, lipofection, calcium-phosphate precipitation, DEAE—dextran transfection and the like.

It is possible to express the antibodies of the disclosure in either prokaryotic or eukaryotic host cells. In certain embodiments, expression of antibodies is performed in eukaryotic cells, e.g., mammalian host cells, of optimal secretion of a properly folded and immunologically active antibody. Exemplary mammalian host cells for expressing the recombinant antibodies of the disclosure include Chinese Hamster Ovary (CHO cells) (including DHFR⁻ CHO cells, described in Urlaub and Chasin, 1980, Proc. Natl. Acad. Sci. USA 77:4216-4220, used with a DHFR selectable marker, e.g., as described in Kaufman and Sharp, 1982, Mol. Biol. 159:601-621), NSO myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Antibodies can be recovered from the culture medium using standard protein purification methods. Host cells can also be used to produce portions of intact antibodies, such as Fab fragments or scFv molecules. It is understood that variations on the above procedure are within the scope of the present disclosure. For example, it can be desirable to transfect a host cell with DNA encoding either the light chain or the heavy chain (but not both) of an anti-glyco-CD44 antibody of this disclosure.

For expression of a CAR of the disclosure, for example as described in Section 6.3 and in numbered embodiments 395 to 429, it is preferable that the host cell is a T cell, preferably a human T cell. In some embodiments, the host cell exhibits an anti-tumor immunity when the cell is cross-linked with CD44 on a tumor cell. Detailed methods for producing the T cells of the disclosure are described in Section 6.5.1.

Recombinant DNA technology can also be used to remove some or all of the DNA encoding either or both of the light and heavy chains that is not necessary for binding to glyco-CD44. The molecules expressed from such truncated DNA molecules are also encompassed by the antibodies of the disclosure.

For recombinant expression of an anti-glyco-CD44 antibody of the disclosure, the host cell can be co-transfected with two expression vectors of the disclosure, the first vector encoding a heavy chain derived polypeptide and the second vector encoding a light chain derived polypeptide. The two vectors can contain identical selectable markers, or they can each contain a separate selectable marker. Alternatively, a single vector can be used which encodes both heavy and light chain polypeptides.

Once a nucleic acid encoding one or more portions of an anti-glyco-CD44 antibody, further alterations or mutations can be introduced into the coding sequence, for example to generate nucleic acids encoding antibodies with different CDR sequences, antibodies with reduced affinity to the Fc receptor, or antibodies of different subclasses.

The anti-glyco-CD44 antibodies of the disclosure can also be produced by chemical synthesis (e.g., by the methods described in Solid Phase Peptide Synthesis, 2nd ed., 1984 The Pierce Chemical Co., Rockford, Ill.). Variant antibodies can also be generated using a cell-free platform (See, e.g., Chu et al., Biochemia No. 2, 2001 (Roche Molecular Biologicals) and Murray et al., 2013, Current Opinion in Chemical Biology, 17:420-426).

Once an anti-glyco-CD44 antibody of the disclosure has been produced by recombinant expression, it can be purified by any method known in the art for purification of an immunoglobulin molecule, for example, by chromatography (e.g., ion exchange, affinity, and sizing column chromatography), centrifugation, differential solubility, or by any other standard technique for the purification of proteins. Further, the anti-glyco-CD44 antibodies of the present disclosure and/or binding fragments can be fused to heterologous polypeptide sequences described herein or otherwise known in the art to facilitate purification.

Once isolated, the anti-glyco-CD44 antibody can, if desired, be further purified, e.g., by high performance liquid chromatography (see, e.g., Fisher, Laboratory Techniques In Biochemistry And Molecular Biology, Work and Burdon, eds., Elsevier, 1980), or by gel filtration chromatography on a Superdex™ 75 column (Pharmacia Biotech AB, Uppsala, Sweden).

6.5.1. Recombinant Production of CARs in T Cells

In some embodiments, nucleic acids encoding the anti-glyco-CD44 CARs of the disclosure are delivered into cells using a retroviral or lentiviral vector. CAR-expressing retroviral and lentiviral vectors can be delivered into different types of eukaryotic cells as well as into tissues and whole organisms using transduced cells as carriers or cell-free local or systemic delivery of encapsulated, bound or naked vectors. The method used can be for any purpose where stable expression is required or sufficient.

In other embodiments, the CAR sequences are delivered into cells using in vitro transcribed mRNA. In vitro transcribed mRNA CAR can be delivered into different types of eukaryotic cells as well as into tissues and whole organisms using transfected cells as carriers or cell-free local or systemic delivery of encapsulated, bound or naked mRNA. The method used can be for any purpose where transient expression is required or sufficient.

In another embodiment, the desired CAR can be expressed in the cells by way of transponsons.

One advantage of RNA transfection methods of the disclosure is that RNA transfection is essentially transient and a vector-free: an RNA transgene can be delivered to a lymphocyte and expressed therein following a brief in vitro cell activation, as a minimal expressing cassette without the need for any additional viral sequences. Under these conditions, integration of the transgene into the host cell genome is unlikely. Cloning of cells is not necessary because of the efficiency of transfection of the RNA and its ability to uniformly modify the entire lymphocyte population.

Genetic modification of T cells with in vitro-transcribed RNA (IVT-RNA) makes use of two different strategies both of which have been successively tested in various animal models. Cells are transfected with in vitro-transcribed RNA by means of lipofection or electroporation. Preferably, it is desirable to stabilize IVT-RNA using various modifications in order to achieve prolonged expression of transferred IVT-RNA.

Some IVT vectors are known in the literature which are utilized in a standardized manner as template for in vitro transcription and which have been genetically modified in such a way that stabilized RNA transcripts are produced. Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides (SEQ ID NO:204). Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly (A) sequence at the 3′ end. It is not clear, whether this nonphysiological overhang affects the amount of protein produced intracellularly from such a construct.

RNA has several advantages over more traditional plasmid or viral approaches. Gene expression from an RNA source does not require transcription and the protein product is produced rapidly after the transfection. Further, since the RNA has to only gain access to the cytoplasm, rather than the nucleus, and therefore typical transfection methods result in an extremely high rate of transfection. In addition, plasmid based approaches require that the promoter driving the expression of the gene of interest be active in the cells under study.

In another aspect, the RNA construct can be delivered into the cells by electroporation. See, e.g., the formulations and methodology of electroporation of nucleic acid constructs into mammalian cells as taught in US 2004/0014645, US 2005/0052630A1, US 2005/0070841A1, US 2004/0059285A1, US 2004/0092907A1. The various parameters including electric field strength required for electroporation of any known cell type are generally known in the relevant research literature as well as numerous patents and applications in the field. See e.g., U.S. Pat. Nos. 6,678,556, 7,171,264, and 7,173,116. Apparatus for therapeutic application of electroporation are available commercially, e.g., the MedPulser™ DNA Electroporation Therapy System (Inovio/Genetronics, San Diego, Calif.), and are described in patents such as U.S. Pat. Nos. 6,567,694; 6,516,223, 5,993,434, 6,181,964, 6,241,701, and 6,233,482; electroporation may also be used for transfection of cells in vitro as described e.g. in US20070128708A1. Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

6.5.1.1 Sources of T Cells

Prior to expansion and genetic modification, a source of T cells is obtained from a subject. The term “subject” is intended to include living organisms in which an immune response can be elicited (e.g., mammals). Examples of subjects include humans, dogs, cats, mice, rats, and transgenic species thereof. Preferably, subjects are human.

T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present disclosure, any number of T cell lines available in the art, may be used. In certain embodiments of the present disclosure, T cells can be obtained from a unit of blood collected from a subject using any number of techniques known to the skilled artisan, such as Ficoll™ separation. In one preferred embodiment, cells from the circulating blood of an individual are obtained by apheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the disclosure, the cells are washed with phosphate buffered saline (PBS). In an alternative embodiment, the wash solution lacks calcium and may lack magnesium or may lack many if not all divalent cations. Again, surprisingly, initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28′, CD4⁺, CD8⁺, CD45RA⁺ and CD45RO⁺ T cells, can be further isolated by positive or negative selection techniques. For example, in one embodiment, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or, lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this disclosure. In certain embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11 b, CD16, HLA-DR, and CD8. In certain embodiments, it may be desirable to enrich for or positively select for regulatory T cells which typically express CD4⁺, CD25⁺, CD62L^{hi}, GITR⁺, and FoxP3⁺. Alternatively, in certain embodiments, T regulatory cells are depleted by anti-C25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8⁺ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4⁺ T cells express higher levels of CD28 and are more efficiently captured than CD8⁺ T cells in dilute concentrations. In one embodiment, the concentration of cells used is 5×10⁶/ml. In other embodiments, the concentration used can be from about 1×10⁵/ml to 1×10⁶/ml, and any integer value in between.

In other embodiments, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain embodiments, cryopreserved cells are thawed and washed as described herein and allowed to rest for one hour at room temperature prior to activation using the methods of the present disclosure.

Also contemplated in the context of the disclosure is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAM PATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993). In a further embodiment, the cells are isolated for a patient and frozen for later use in conjunction with (e.g., before, simultaneously or following) bone marrow or stem cell transplantation or T cell ablative therapy using either chemotherapy agents such as, fludarabine, external-beam radiation therapy (XRT), cyclophosphamide.

In a further embodiment of the present disclosure, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present disclosure to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

6.5.1.2 Activation and Expansion of T Cells

T cells are activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T cells of the disclosure are expanded by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4⁺ T cells or CD8⁺ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In certain embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present disclosure.

In one embodiment, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the co-stimulatory signal is an anti-CD28 antibody or antigen-binding fragment thereof; and both agents are co-immobilized to the same bead in equivalent molecular amounts. In one embodiment, a 1:1 ratio of each antibody bound to the beads for CD4⁺ T cell expansion and T cell growth is used. In certain aspects of the present disclosure, a ratio of anti CD3:CD28 antibodies bound to the beads is used such that an increase in T cell expansion is observed as compared to the expansion observed using a ratio of 1:1. In one particular embodiment an increase of from about 1 to about 3 fold is observed as compared to the expansion observed using a ratio of 1:1. In one embodiment, the ratio of CD3:CD28 antibody bound to the beads ranges from 100:1 to 1:100 and all integer values there between. In one aspect of the present disclosure, more anti-CD28 antibody is bound to the particles than anti-CD3 antibody, i.e., the ratio of CD3:CD28 is less than one. In certain embodiments of the disclosure, the ratio of anti CD28 antibody to anti CD3 antibody bound to the beads is greater than 2:1. In one particular embodiment, a 1:100 CD3:CD28 ratio of antibody bound to beads is used. In another embodiment, a 1:75 CD3:CD28 ratio of antibody bound to beads is used. In a further embodiment, a 1:50 CD3:CD28 ratio of antibody bound to beads is used. In another embodiment, a 1:30 CD3:CD28 ratio of antibody bound to beads is used. In one preferred embodiment, a 1:10 CD3:CD28 ratio of antibody bound to beads is used. In another embodiment, a 1:3 CD3:CD28 ratio of antibody bound to the beads is used. In yet another embodiment, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T cells. The ratio of anti-CD3- and anti-CD28-coupled particles to T cells that result in T cell stimulation can vary as noted above, however certain preferred values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one preferred ratio being at least 1:1 particles per T cell. In one embodiment, a ratio of particles to cells of 1:1 or less is used. In one particular embodiment, a preferred particle: cell ratio is 1:5. In further embodiments, the ratio of particles to cells can be varied depending on the day of stimulation. For example, in one embodiment, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular embodiment, the ratio of particles to cells is 1:1 on the first day of stimulation and adjusted to 1:5 on the third and fifth days of stimulation. In another embodiment, particles are added on a daily or every other day basis to a final ratio of 1:1 on the first day, and 1:5 on the third and fifth days of stimulation. In another embodiment, the ratio of particles to cells is 2:1 on the first day of stimulation and adjusted to 1:10 on the third and fifth days of stimulation. In another embodiment, particles are added on a daily or every other day basis to a final ratio of 1:1 on the first day, and 1:10 on the third and fifth days of stimulation. One of skill in the art will appreciate that a variety of other ratios may be suitable for use in the present disclosure. In particular, ratios will vary depending on particle size and on cell size and type.

In further embodiments of the present disclosure, the cells, such as T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one embodiment the cells (for example, 10⁴ to 10⁹ T cells) and beads (for example, DYNABEADS® M-450 CD3/CD28 T paramagnetic beads at a ratio of 1:1) are combined in a buffer, preferably PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present disclosure. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/ml is used. In another embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain embodiments. For example, using high concentration of cells allows more efficient selection of CD8+ T cells that normally have weaker CD28 expression.

In one embodiment of the present disclosure, the mixture may be cultured for several hours (about 3 hours) to about 14 days or any hourly integer value in between. In another embodiment, the mixture may be cultured for 21 days. In one embodiment of the disclosure the beads and the T cells are cultured together for about eight days. In another embodiment, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

T cells that have been exposed to varied stimulation times may exhibit different characteristics. For example, typical blood or apheresed peripheral blood mononuclear cell products have a helper T cell population (T_{H}, CD4⁺) that is greater than the cytotoxic or suppressor T cell population (T_{C}, CD8⁺). Ex vivo expansion of T cells by stimulating CD3 and CD28 receptors produces a population of T cells that prior to about days 8-9 consists predominately of T_{H} cells, while after about days 8-9, the population of T cells comprises an increasingly greater population of T_{C} cells. Accordingly, depending on the purpose of treatment, infusing a subject with a T cell population comprising predominately of T_{H} cells may be advantageous. Similarly, if an antigen-specific subset of T_{C} cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

6.6 Compositions

The anti-glyco-CD44 antibodies, fusion proteins, and/or anti-glyco-CD44 ADCs of the disclosure may be in the form of compositions comprising the anti-glyco-CD44 antibody, fusion protein and/or ADC and one or more carriers, excipients and/or diluents. The compositions may be formulated for specific uses, such as for veterinary uses or pharmaceutical uses in humans. The form of the composition (e.g., dry powder, liquid formulation, etc.) and the excipients, diluents and/or carriers used will depend upon the intended uses of the antibody, fusion protein and/or ADC and, for therapeutic uses, the mode of administration.

For therapeutic uses, the compositions may be supplied as part of a sterile, pharmaceutical composition that includes a pharmaceutically acceptable carrier. This composition can be in any suitable form (depending upon the desired method of administering it to a patient). The pharmaceutical composition can be administered to a patient by a variety of routes such as orally, transdermally, subcutaneously, intranasally, intravenously, intramuscularly, intratumorally, intrathecally, topically or locally. The most suitable route for administration in any given case will depend on the particular antibody and/or ADC, the subject, and the nature and severity of the disease and the physical condition of the subject. Typically, the pharmaceutical composition will be administered intravenously or subcutaneously.

Pharmaceutical compositions can be conveniently presented in unit dosage forms containing a predetermined amount of an anti-glyco-CD44 antibody and/or anti-glyco-CD44 ADC of the disclosure per dose. The quantity of antibody and/or ADC included in a unit dose will depend on the disease being treated, as well as other factors as are well known in the art. Such unit dosages may be in the form of a lyophilized dry powder containing an amount of antibody and/or ADC suitable for a single administration, or in the form of a liquid. Dry powder unit dosage forms may be packaged in a kit with a syringe, a suitable quantity of diluent and/or other components useful for administration. Unit dosages in liquid form may be conveniently supplied in the form of a syringe pre-filled with a quantity of antibody and/or ADC suitable for a single administration.

The pharmaceutical compositions may also be supplied in bulk form containing quantities of antibody and/or ADC suitable for multiple administrations.

Pharmaceutical compositions may be prepared for storage as lyophilized formulations or aqueous solutions by mixing an antibody, fusion protein, and/or ADC having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington's Pharmaceutical Sciences, 16th edition (Osol, ed. 1980). Such additives should be nontoxic to the recipients at the dosages and concentrations employed.

Buffering agents help to maintain the pH in the range which approximates physiological conditions. They may be present at a wide variety of concentrations, but will typically be present in concentrations ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives may be added to retard microbial growth, and can be added in amounts ranging from about 0.2%-1% (w/v). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, α-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trehalose; and trisaccacharides such as raffinose; and polysaccharides such as dextran. Stabilizers may be present in amounts ranging from 0.5 to 10 wt % per wt of ADC.

Non-ionic surfactants or detergents (also known as “wetting agents”) may be added to help solubilize the glycoprotein as well as to protect the glycoprotein against agitation-induced aggregation, which also permits the formulation to be exposed to shear surface stressed without causing denaturation of the protein. Suitable non-ionic surfactants include polysorbates (20, 80, etc.), polyoxamers (184, 188 etc.), and pluronic polyols. Non-ionic surfactants may be present in a range of about 0.05 mg/mL to about 1.0 mg/mL, for example about 0.07 mg/mL to about 0.2 mg/mL.

Additional miscellaneous excipients include bulking agents (e.g., starch), chelating agents (e.g., EDTA), antioxidants (e.g., ascorbic acid, methionine, vitamin E), and cosolvents.

6.7 Methods of Use

The anti-glyco-CD44 antibody or binding fragments described herein can be used in various diagnostic assays. For example, the antibodies and binding fragments can be employed in immunoassays, such as competitive binding assays, direct and indirect sandwich assays, and immunoprecipitation assays, including immunohistochemistry, enzyme-linked immunosorbent assay (ELISA), fluorescence-activated cell sorting (FACS), and Western blots.

The anti-glyco-CD44 antibody or binding fragments described herein also are useful for radiographic in vivo imaging, wherein an antibody labeled with a detectable moiety such as a radio-opaque agent or radioisotope is administered to a subject, preferably into the bloodstream, and the presence and location of the labeled antibody in the host is assayed. This imaging technique is useful in the staging and treatment of malignancies.

The anti-glyco-CD44 antibody or binding fragments, fusion proteins, ADCs and CARs described herein are useful for treatment of glyco-CD44 expressing cancers, including breast cancer, lung cancer, pancreatic cancer, colorectal cancer, ovarian cancer, gastric cancer, or head and neck cancer, skin cancer, malignant melanomas, liver cancer, gliomas, thyroid cancer, kidney cancer, prostate cancer and other urogenital cancers, cervical cancer, and endometrial cancer.

Thus, the disclosure provides anti-glyco-CD44 antibodies, binding fragments, fusion proteins, ADCs, and CARs as described herein for use as a medicament, for example for use in the treatment of cancer, e.g., any of the cancers identified in the previous paragraph, for use in a diagnostic assay, and for use in radiographic in vivo imaging. The disclosure further provides for the use of the anti-glyco-CD44 antibodies, binding fragments, fusion proteins, ADCs and CARs as described herein in the manufacture of a medicament, for example for the treatment of cancer, e.g., any of the cancers identified in the previous paragraph.

When using the CARs of the disclosure for therapy, the therapeutic methods of the disclosure comprise administering to a subject with a glyco-CD44-expressing tumor an effective amount of a genetically modified cell engineered to express a CAR of the disclosure, for example a CAR as described in Section 6.3 or in numbered embodiments 395 to 429. Methods of modifying cells, particularly T cells, to express a CAR, are described in Section 6.5.1.

When using the MicAbodies of the disclosure for therapy, the therapeutic methods of the disclosure comprise administering to a subject with a glyco-CD44-expressing tumor therapeutically effective amounts of a MicAbody of the disclosure, for example a MicAbody described in Section 6.4 or numbered embodiments 391 to 394, and a genetically modified T-cell engineered to express a CAR comprising a NKG2D receptor capable of specifically binding the MicAbody.

6.8 CD44v6 Peptides

Also provided herein are isolated CD44v6 glycopeptides comprising the amino acid GYRQTPKEDSHSTTGTAAA (SEQ ID NO:165), or a fragment thereof. In some embodiments, the CD44v6 glycopeptide is glycosylated with GalNAc on the serine and threonine residues shown with bold and underlined text (i.e., threonine at amino acid position 5 of SEQ ID NO: 165 and/or serine at amino acid position 12 of SEQ ID NO: 165), or a fragment thereof. Exemplary isolated CD44v6 glycopeptides are described in numbered embodiments 534-539.

The present disclosure encompasses synthetic synthesis of the isolated CD44v6 glycoproteins and recombinant methods for producing the isolated CD44v6 glycoproteins.

In certain embodiments, the isolated CD44v6 peptides are synthesized using a sold-phase peptide synthesis (SPPS) strategy. SPPS methods are known in the art. SPPS provides for the rapid assembly of a polypeptide through successive reactions of amino acid derivatives on a solid support. Through repeated cycles of alternating N-terminal deprotection and coupling reactions, successive amino acid derivatives are added to the polypeptide. In other embodiments, isolated CD44v6 peptides are synthesized using a solution-phase peptide synthesis strategy. Solution-phase peptide synthesis methods are known in the art.

To ensure propre O-linked glycosylation with GalNAc on the threonine at amino acid position 5 of SEQ ID NO: 165 and serine at amino acid position 12 of SEQ ID NO: 165, pre-synthesized glycosylated amino acids can be used in the elongation reactions, as described in Section 7.1.2.1.

Nucleic acid molecules encoding the isolated CD44v6 glycopeptides, vectors comprising such nucleic acids, and host cells capable of producing the isolated CD44v6 glycopeptides of the disclosure are provided. In certain aspects, the nucleic acid molecules encode, and the host cells are capable of expressing, the isolated CD44v6 glycopeptide as well as fusion proteins that include the isolated CD44v6 glycoproteins.

An isolated CD44v6 glycopeptide of the disclosure can be prepared by recombinant expression in a host cell. To express an isolated CD44v6 glycopeptide recombinantly, a host cell is transfected with a recombinant expression vector carrying DNA encoding the glycopeptide such that the glycopeptide is expressed in the host cell and, optionally, secreted into the medium in which the host cells are cultured, from which medium the glycoproteins can be recovered. Standard recombinant DNA methodologies are used to obtain a CD44v6 glycoprotein gene, incorporate the gene into recombinant expression vectors and introduce the vectors into host cells, such as those described in Molecular Cloning; A Laboratory Manual, Second Edition (Sambrook, Fritsch and Maniatis (eds), Cold Spring Harbor, N.Y., 1989), Current Protocols in Molecular Biology (Ausubel, F. M. et al., eds., Greene Publishing Associates, 1989) and in U.S. Pat. No. 4,816,397.

It is possible to express the antibodies of the disclosure in either prokaryotic or eukaryotic host cells. In certain embodiments, expression of antibodies is performed in eukaryotic cells, e.g., mammalian host cells, of optimal secretion of a properly folded and immunologically active antibody. To produce the isolated CD44v6 glycoproteins of the disclosure, a host cell is selected based on its ability to glycosylate threonine at amino acid position 5 of SEQ ID NO: 165 and serine at amino acid position 12 of SEQ ID NO: 165. An exemplary host cell is the COSMC KO HEK293 cell.

6.8.1. CD44v6 Peptide Compositions

The CD44v6 peptides of the disclosure may be in the form of compositions comprising the CD44v6 peptide and one or more carriers, excipients, diluents and/or adjuvants. The compositions may be formulated for specific uses, such as for veterinary uses or pharmaceutical uses in humans. The form of the composition (e.g., dry powder, liquid formulation, etc.) and the excipients, diluents and/or carriers used will depend upon the intended uses of the antibody, fusion protein and/or ADC and, for therapeutic uses, the mode of administration.

For therapeutic uses, the compositions may be supplied as part of a sterile, pharmaceutical composition that includes a pharmaceutically acceptable carrier and/or a pharmaceutically acceptable adjuvant. This composition can be in any suitable form (depending upon the desired method of administering it to a patient). The pharmaceutical composition can be administered to a patient by a variety of routes such as orally, transdermally, subcutaneously, intranasally, intravenously, intramuscularly, intratumorally, intrathecally, topically or locally. The most suitable route for administration in any given case will depend on the particular CD44v6 peptide, the subject, and the nature and severity of the disease and the physical condition of the subject. Typically, the pharmaceutical composition will be administered intravenously or subcutaneously.

Pharmaceutical compositions can be conveniently presented in unit dosage forms containing a predetermined amount of an CD44v6 peptide of the disclosure per dose. The quantity of CD44v6 peptide included in a unit dose will depend on the disease being treated, as well as other factors as are well known in the art. Such unit dosages may be in the form of a lyophilized dry powder containing an amount of CD44v6 peptide suitable for a single administration, or in the form of a liquid. Dry powder unit dosage forms may be packaged in a kit with a syringe, a suitable quantity of diluent and/or other components useful for administration. Unit dosages in liquid form may be conveniently supplied in the form of a syringe pre-filled with a quantity of CD44v6 peptide suitable for a single administration.

The pharmaceutical compositions may also be supplied in bulk form containing quantities of CD44V6 peptide suitable for multiple administrations.

Pharmaceutical compositions may be prepared for storage as lyophilized formulations or aqueous solutions by mixing an CD44v6 peptide having the desired degree of purity with optional pharmaceutically-acceptable carriers, excipients, adjuvants or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington's Pharmaceutical Sciences, 16th edition (Osol, ed. 1980). Such additives should be nontoxic to the recipients at the dosages and concentrations employed.

In some embodiments, the composition includes one or more pharmaceutically adjuvants in addition to the fusion protein and/or nanoparticle. Adjuvants include, for example, aluminum salts (e.g., amorphous aluminum hydroxyphosphate sulfate (AAHS), aluminum hydroxide, aluminum phosphate,

potassium aluminum sulfate (Alum)), dsRNA analogues, lipid A analogues, flagellin, imidazoquinolines, CpG ODN, saponins (e.g., QS21), C-type lectin ligands (e.g., TDB), CD1d ligans (α-galactosylceramide), MF59, AS01, AS02, AS03, AS04, AS15, AF03, GLA-SE, IC31, CAF01, and virosomes. Other adjuvants known in the art, including chemical adjuvants, genetic adjuvants, protein adjuvants, and lipid adjuvants, can also be included in the compositions.

Buffering agents help to maintain the pH in the range which approximates physiological conditions. They may be present at a wide variety of concentrations, but will typically be present in concentrations ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives may be added to retard microbial growth, and can be added in amounts ranging from about 0.2%-1% (w/v). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, α-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trehalose; and trisaccacharides such as raffinose; and polysaccharides such as dextran. Stabilizers may be present in amounts ranging from 0.5 to 10 wt % per wt of CD44 peptide.

Non-ionic surfactants or detergents (also known as “wetting agents”) may be added to help solubilize the glycoprotein as well as to protect the glycoprotein against agitation-induced aggregation, which also permits the formulation to be exposed to shear surface stressed without causing denaturation of the protein. Suitable non-ionic surfactants include polysorbates (20, 80, etc.), polyoxamers (184, 188 etc.), and pluronic polyols. Non-ionic surfactants may be present in a range of about 0.05 mg/mL to about 1.0 mg/mL, for example about 0.07 mg/mL to about 0.2 mg/mL.

Additional miscellaneous excipients include bulking agents (e.g., starch), chelating agents (e.g., EDTA), antioxidants (e.g., ascorbic acid, methionine, vitamin E), and cosolvents.

Exemplary CD44v6 peptide compositions of the disclosure are described in numbered embodiments 540-541.

6.8.2. Methods of Using CD44v6 Peptides

The CD44v6 peptides described herein can be used in the production of antibodies against a tumor-associated form of CD44v6. The CD44v6 peptide can be administered to an animal. The amount of peptide administered can be effective to cause the animal to produce antibodies against the peptide As used herein, “animal” refers to multicellular eukaryotic organism from the biological kingdom Animalia. In some embodiments, the animal is a mammal. In some embodiments, the animal is a mouse or a rabbit. Resulting antibodies can then be collected from the animal. The CD44v6 peptide can be administered as purified peptide or as part of a composition provided herein.

The CD44v6 peptides described herein can be used to elicit an immune response against a tumor-associated form of CD44v6. The CD44v6 peptide can be administered to an animal in an amount effective to cause the animal to mount an immune response (e.g., produce antibodies) against the peptide.

Exemplary methods for using the CD44v6 peptides of the disclosure are described in numbered embodiments 542-545.

### 7. EXAMPLES

### 7.1 Example 1: Identification and Characterization Of Anti-Glyco-CD44 Antibodies

7.1.1. Overview

Glycans are essential membrane components and neoplastic transformation of human cells is virtually always associated with aberrant glycosylation of proteins and lipids. There are several types of protein glycosylation, including N-glycosylation and many types of O-glycosylation, but one of the most diverse types is the mucin type GalNAc type O-glycosylation (hereafter called O-glycosylation). Cancer associated changes in O-glycans are particularly interesting and the most frequently observed aberrant glycophenotype is expression of the most immature truncated O-glycan structures designated Tn (GalNAcα1-O-Ser/Thr), STn (NeuAcα2-6GalNAcα1-O-Ser/Thr), and T (Galβ1-3GalNAcα1-O-Ser/Thr) antigens. Truncated O-glycans are observed on almost all epithelial cancer cells and strongly correlated with poor prognosis. In addition, it is becoming increasingly clear that glycans also have pivotal roles in cancer development, with truncated O-glycans affecting differentiation, cell-cell and cell-matrix interactions, directly inducing oncogenic features in predisposed cells.

The inventors have identified CD44 glycopeptide epitopes in human cancer cells and used the defined glyco-peptides to develop cancer specific anti-glyco-CD44 monoclonal antibodies.

7.1.2. Materials and Methods

7.1.2.1 Synthesis of CD44v6 Glycopeptide

The CD44v6 glycopeptide, GYRQTPKEDSHSTTGTAAA (SEQ ID NO:165), with 0-linked GalNAc on the serine and threonine residues shown with bold and underlined text, was synthesized using a standard FMOC peptide synthesis strategy. Pre-synthesized glycosylated amino acids were coupled to the elongating peptide at specific locations using solid or solution phase peptide chemistry in a stepwise fashion. After completing the full sequence and removing all protecting groups, the resulting glycopeptide was purified by high-performance liquid chromatography (HPLC) and characterized by mass spectrometry (electrospray ionization in positive mode).

7.1.2.2 Synthesis of recombinant Tn-glycosylated CD44

1×10⁶ COSMC KO HEK293 cells in 30 mL Opti-MEM were transfected using 30 μg of a plasmid encoding his-tagged human CD44 and 60 μL 293Fectin™ Transfection Reagent (Gibco). Following 48 hours of culture, the cells were spun down and the his-tagged recombinant CD44 protein was purified from the supernatant using a 50% Ni-NTA agarose slurry column (Invitrogen), eluting with 250 mM imidazole. To increase purity, this purification step was repeated. The recombinant SC-CD44 protein was concentrated in PBS using Amicon Ultra centrifugal filters.

7.1.2.3 Immunization Protocol

Female Balb/c mice were immunized subcutaneously with the Tn-glycosylated CD44v6 glycopeptide conjugated to KLH (keyhole limpet hemocyanin) through a glutaraldehyde linker or with recombinant Tn-glycosylated CD44. The mice were immunized on days 0, 14, and 35 with 50 μg, 45 μg, and 45 μg of KLH-glycopeptide, respectively. The first immunization used Freund's complete adjuvant. All subsequent immunizations used Freund's incomplete adjuvant. On Day 45, tail bleeds were evaluated for polyclonal response. On day 56 or after, mice to be fused were boosted with 15 ug of KLH-glycopeptide in Freund's incomplete adjuvant 3 to 5 days before hybridoma fusion. Splenocytes from mice were fused with SP2/0-Ag14 (ATCC, cat #CRL-1581) myeloma cells using the Electro Cell Manipulator (ECM2001) from BTX Harvard Apparatus. Hybridomas were seeded in 96-well plates, cultured, scaled, and evaluated and selected for specificity towards CD44-Tn using ELISA, FLOW cytometry, and immunofluorescence to obtain monoclonal antibodies having specificity for CD44-Tn.

New Zealand white rabbits were immunized subcutaneously with the Tn-glycosylated CD44v6 glycopeptide conjugated to KLH (keyhole limpet hemocyanin) through a glutaraldehyde linker or with recombinant Tn-glycosylated CD44. The mice were immunized on days 0, 28, and 47 with 200 μg, 100 μg, and 100 μg of KLH-glycopeptide, respectively. The first immunization used Freund's complete adjuvant. All subsequent immunizations used Freund's incomplete adjuvant. On Day 58, test bleeds were evaluated for polyclonal response. On day 66 or after, mice to be fused were boosted with 50 ug of KLH-glycopeptide in Freund's incomplete adjuvant 3 to 5 days before hybridoma fusion. Splenocytes from rabbits were fused with SP2/0-Ag14 (ATCC, cat #CRL-1581) myeloma cells using the Electro Cell Manipulator (ECM2001) from BTX Harvard Apparatus. Hybridomas were seeded in 96-well plates, cultured, scaled, and evaluated and selected for specificity towards CD44-Tn using ELISA, FLOW cytometry, and immunofluorescence to obtain monoclonal antibodies having specificity for CD44-Tn.

7.1.2.4 ELISA

96-well Corning high bind microplates (Fisher) were coated overnight at 4° C. with various concentrations of protein, peptide, or glycopeptide in 0.2 M bicarbonate-carbonate buffer (pH 9.4). The plates were then blocked for 1 hour at room temperature with Phosphate-buffered saline (PBS) (pH 7.4) containing 2.5% BSA. Contents of the plate were discarded and purified antibody, or hybridoma supernatants, or blood serum for polyclonal responses, were added at various concentrations and incubated for two hours at room temperature. Plates were washed with tris-buffered saline with 0.05% Tween-20 and then incubated for 1 hour at room temperature with a 1:3000 dilution of HRP conjugated goat anti-mouse IgG Fcγ (Sigma). The plates were washed again and developed with TMB chromogen substrate. After proper development (approximately 2-3 min), the reaction was stopped with 0.2 N H₂SO₄ and the absorbance was read at 450 nm. Data was analysed in GraphPad Prism Software.

7.1.2.5 Flow Cytometry

Adherent cells were dissociated with TrypLE select (Gibco) and washed from flask surface with cell culture media (RPMI w/L-glutamine, 1% PenStrep, & 10% FBS). Cells were washed several times by centrifugation at 300*g for 5 min at 4° C. followed by resuspension in PBS with 1% BSA (PBS/1% BSA). Cells were resuspended between 5×10⁵ cells/ml to 2×10⁶ cell/ml and then distributed into a 96 well U-bottom plate. Diluted commercial antibody (0.25-2 ug/ml), or hybridoma supernatants, or blood serum for polyclonal responses, were added to cells and incubated for 1 hr on ice. Following several washes with PBS/1% BSA, cells were incubated for 30 min on ice with a 1:1600 dilution of AlexaFluor647 conjugated F(ab)₂ goat anti-mouse IgG Fcγ (JacksonImmunoResearch). Cells were washed again with PBS/1% BSA and then fixed in 1% formaldehyde in PBS/1% BSA. Cells were analysed on either a 2 or 4 laser Attune NXT flow cytometer. Data was processed in FlowJo Software.

7.1.2.6 Immunofluorescence

Cells were seeded to 50% confluency in glass chamber slides (nunc) and incubated 12-18 hours at 37° C. 5% CO₂. Following overnight growth, media from slides was removed and cells were fixed with 4% formaldehyde in PBS (pH 7.4) for 10 min at room temperature. Slides were washed in PBS. Diluted commercial antibody (1-4 ug/ml), or hybridoma supernatants, or blood serum for polyclonal responses, were added to the slides and the slides were incubated overnight at 4° C. The slides were washed in PBS and stained with a 1:800 dilution of AlexaFluor488 conjugated F(ab)₂ rabbit anti-mouse IgG (H+L) (Invitrogen) for 45 min at room temperature. The slides were washed in PBS and mounted using Prolong Gold Antifade Mountant with DAPI (thermofisher) and examined using an Olympus FV3000 confocal microscope.

7.1.2.7 Immunohistochemistry

Paraffin embedded tissue micro arrays (TMAs) or tissue sections were de-paraffinized with xylene and ethanol, following antigen retrieval with citrate buffer (pH 6.0) and heated in microwave for 18 min. TMAs were stained with Ultra Vison Quanto Detection System HRP DAB. Briefly, TMAs were washed in TBS, incubated with mAb supernatant for 2 hours. After wash in TBS×2, the TMAs was incubated with Primary Antibody Amplifier Quanto for 10 min. After wash in TBS, TMAs were incubated with HRP polymer quanto (10 min) followed by DAB chromogen. Slides were counterstained with hematoxylin, were dehydrated, and mounted.

7.1.3. Results

7.1.3.1 Glycopeptide Specific Antibodies to Tn-CD44

Glycopeptide reactive antibodies were generated using both the Tn-glycosylated CD44v6 glycopeptide and recombinant Tn-glycosylated CD44, but antibodies generated using CD44v6 glycopeptide, including 4C8, 2B2, 18G9, 1D12, and 10H4, proved superior in selectivity. Antibody 4C8 was selected for further characterization.

7.1.3.2 Characterisation of mAb 4C8 Binding Specificity

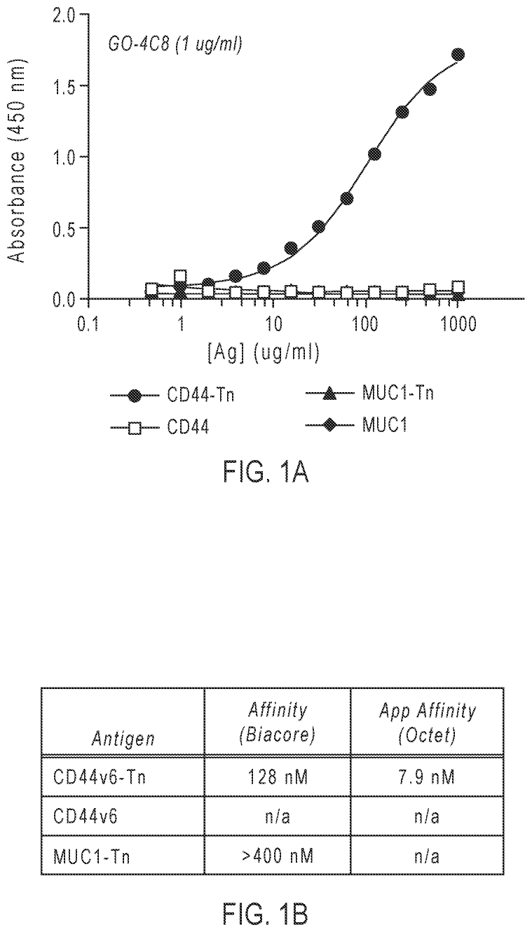

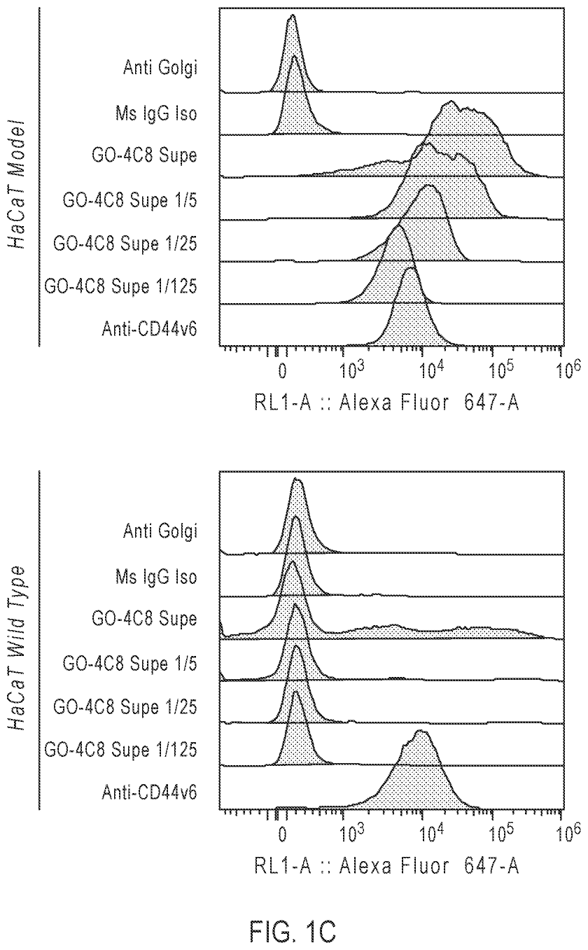

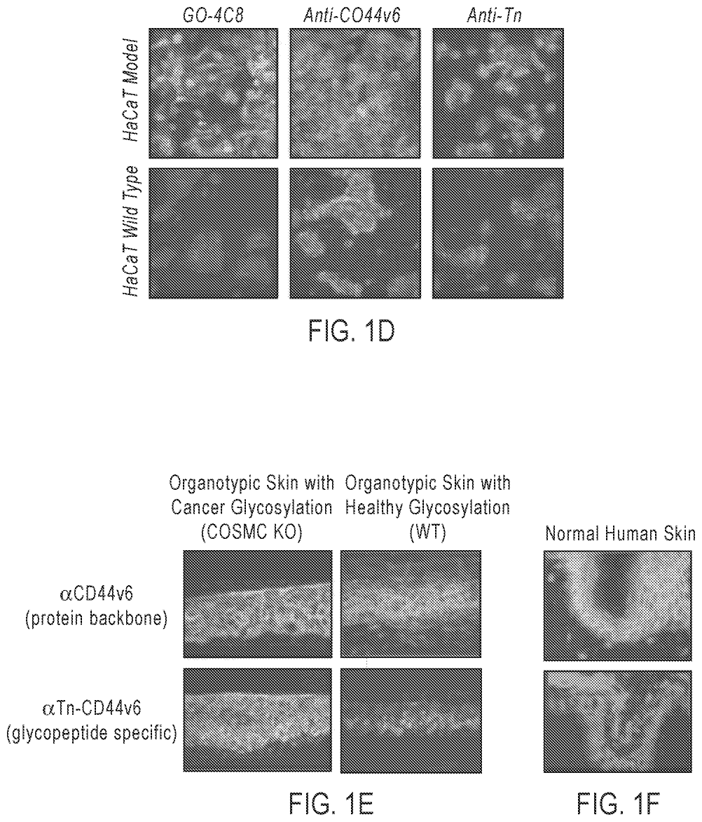

To characterise the binding specificity of 4C8, ELISA against non-glycosylated and Tn-glycosylated CD44 was performed. To assess cross-reactivity with the Tn-antigen, ELISA was also performed against Tn-glycosylated MUC1. It was found that in the context of ELISA, 4C8 only reacted with Tn-glycosylated CD44 and not with its non-glycosylated counterpart nor with MUC1 (FIG. 1A). The affinity of 4C8 against the CD44v6 glycopeptide was determined to be 128 nM when measured on a Biacore. Using an Octet system, the apparent affinity (with avidity factored in) of 4C8 for the CD44v6 glycopeptide was measured to be 7.9 nM (FIG. 1B). Table 4 summarizes dissociation constants (K_{D}) for 4C8 against different glycoforms of CD44v6 peptide, as well as unglycosylated CD44v6 and MUC1-Tn.

TABLE 4
Dissociation Constant (K_{D})

| | CD44v6- | | CD44v6- | CD44v6- | | |
|---|---|---|---|---|---|---|
| | Tn(S + T) | | Tn(S) | Tn(T) | CD44v6 | MUC1-Tn |
| Biacore | 128 | nM | NA | NA | NA | NA |
| Octet | 7.9 | nM | 6.4 nM | ND | ND | ND |

NA indicates affinity was not measured using the given technology, ND (not determined) indicates the affinity was below the detection limits.

To further assess 4C8 specificity in a more natural conformational context, 4C8 was used to stain HaCaT cells for flow cytometry and immunofluorescence. The keratinocyte-derived HaCaT cell line is inherently Tn-negative but can be induced to express the Tn-antigen by KO of the COSMC chaperone. When using 4C8 to stain for flow cytometry, it was found that 4C8 selectively stained COSMC KO HaCaT cells but not their wildtype counterpart, despite both cells staining positive for CD44v6 (FIG. 10). The reactivity of 4C8 could not be ascribed to intracellular staining of Tn-positive intermediary CD44 proteins localised in the secretory pathway, as the cells did not stain positive for a Golgi marker. In agreement with these results, immunofluorescence showed that only CD44v6⁺ Tn⁺ HaCaT COSMC KO cells stained with 4C8, whereas CD44v6⁺ Tn⁻ HaCaT WT cells did not (FIG. 1D). In addition, when the HaCaT cells were used to form an organotypic in vitro skin model, 4C8 stained only the COSMC KO skin, but not the wildtype skin (FIG. 1E). As clinical trials testing other CD44 antibodies have reported severe skin toxicities, 4C8 binding to healthy human skin was also assessed. It was found that although human skin stained positive for CD44v6, 4C8 showed no reactivity with the human skin (FIG. 1F). Taken together, these results demonstrate that 4C8 selectively reacts with Tn-glycosylated CD44 and does not cross-react with healthy human skin.

7.1.3.3 Tissue Expression of the Tn-Glycosylated CD44v6 Epitope Recognised by 4C8

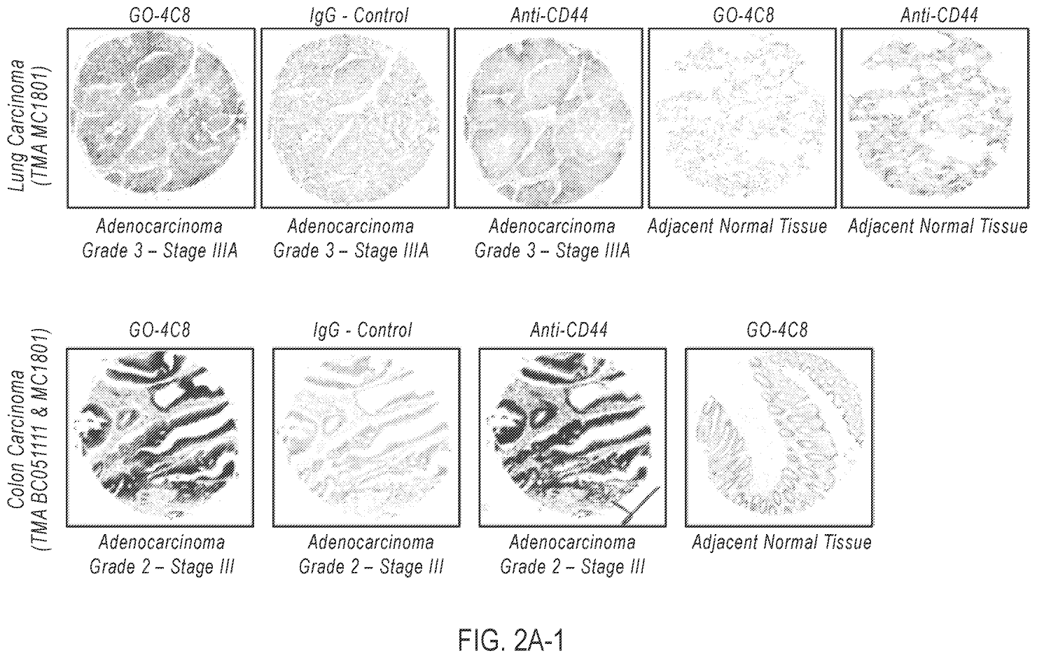

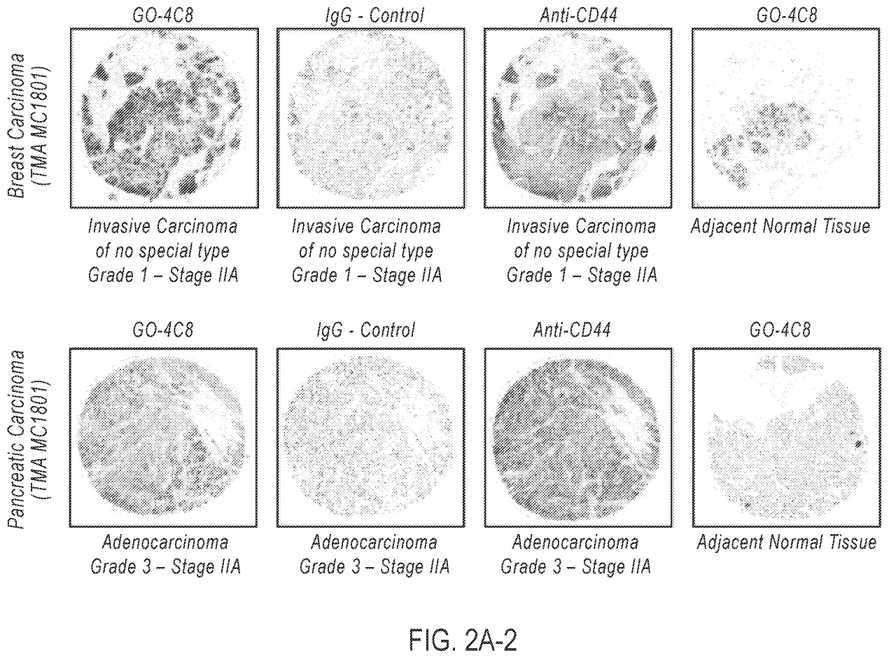

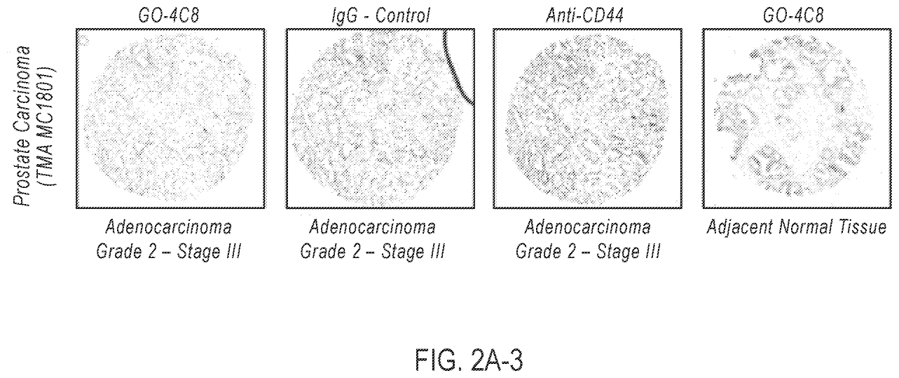

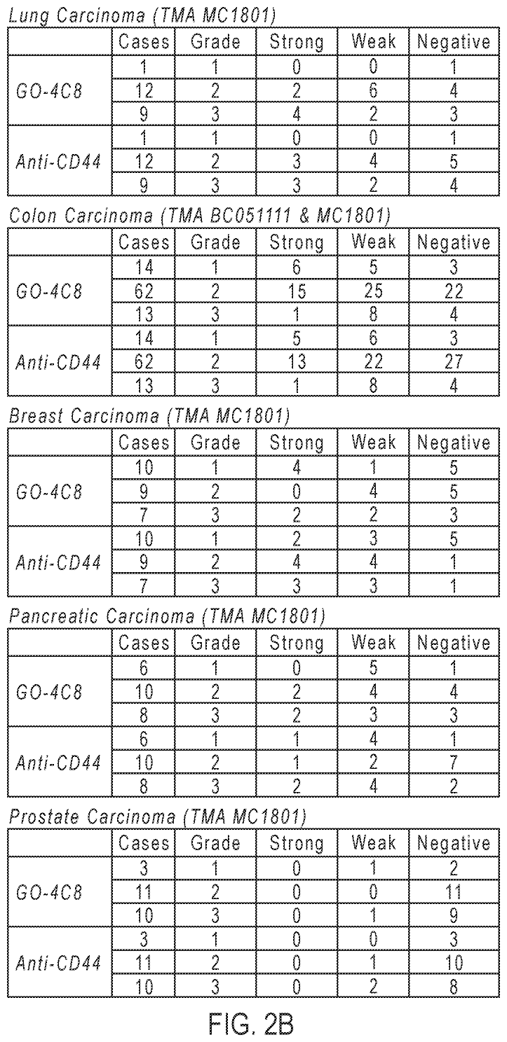

When staining formalin-fixed paraffin embedded tissue sections for immunofluorescence, positive staining was observed with 4C8 in 7/10 lung, 7/24 ovary, 7/14 HNSCC, 3/16 colon, 9/20 gastric, 3/8 kidney, and 3/10 breast cancer sections. Immunohistochemistry of tissue microarrays additionally showed strong staining of 22/89 and weak staining of 38/89 colon carcinomas, strong staining of 4/24 and weak staining of 12/24 pancreatic carcinomas, strong staining of 6/22 and weak staining of 8/22 lung carcinomas, strong staining of 6/26 and weak staining of 7/26 breast carcinomas, and weak staining of 2/24 prostate carcinomas, using 4C8 (FIG. 2A-2B). This staining pattern correlated with staining for normal CD44 expression, showing that CD44 expression in these carcinomas predicted reactivity to 4C8. Importantly, no reactivity when using 4C8 to stain healthy adjacent tissues was observed (FIG. 2A). In conclusion, 4C8 positively was found to react with several cancer tissue sections, but not their healthy counterparts.

### 7.2 Example 2: 4C8-Based CARs

Chimeric antigen receptors (CARs) having VH and VL domains of 4C8 were designed. Selected CARs were then evaluated in target-specific a cytotoxicity assay.

7.2.1. Materials and Methods

7.2.1.1 Vector Design

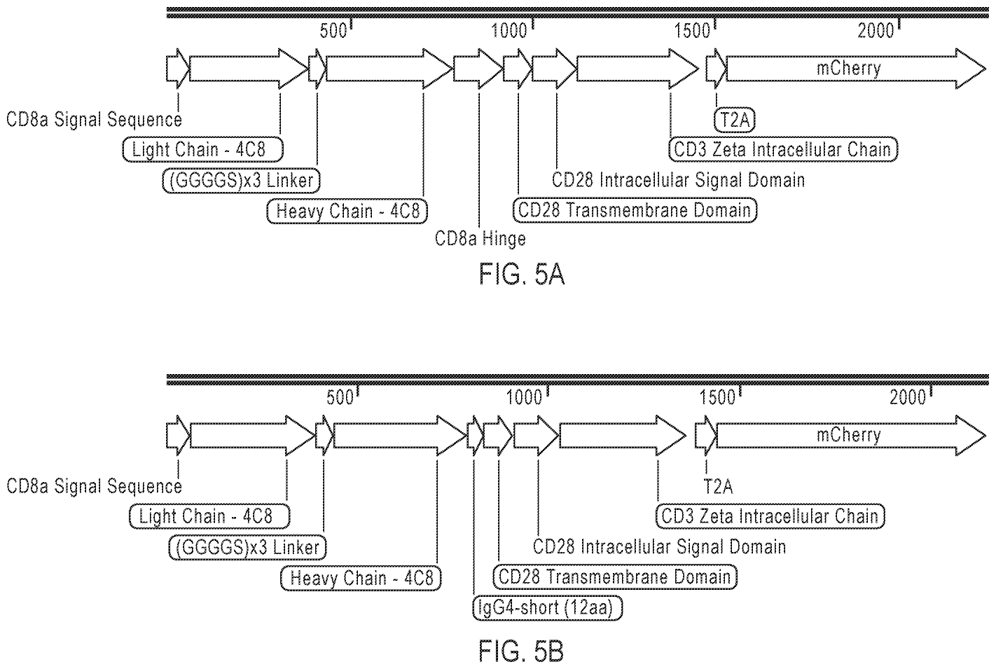

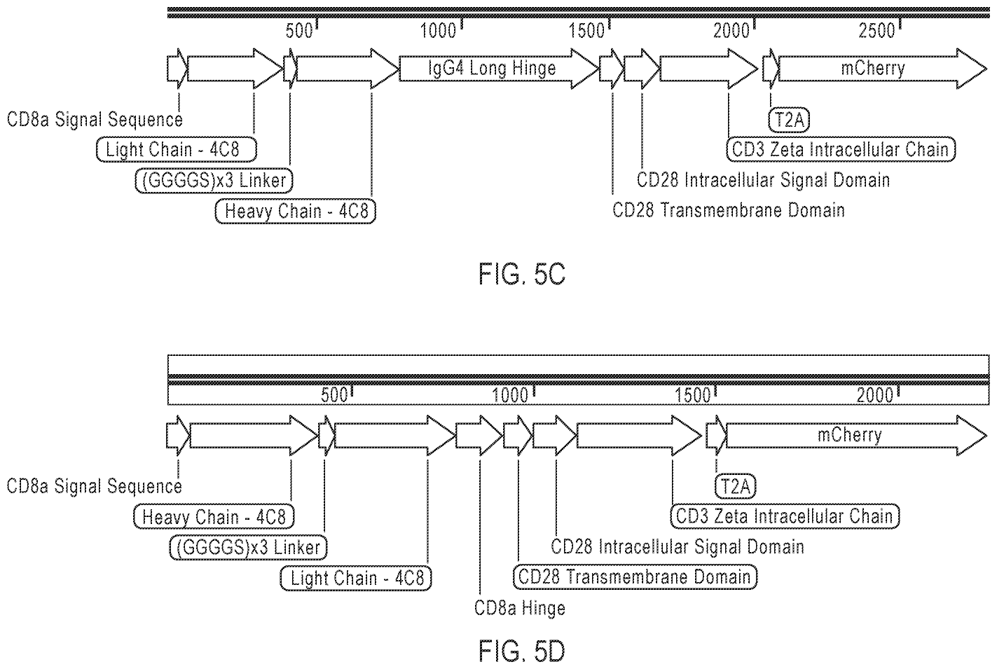

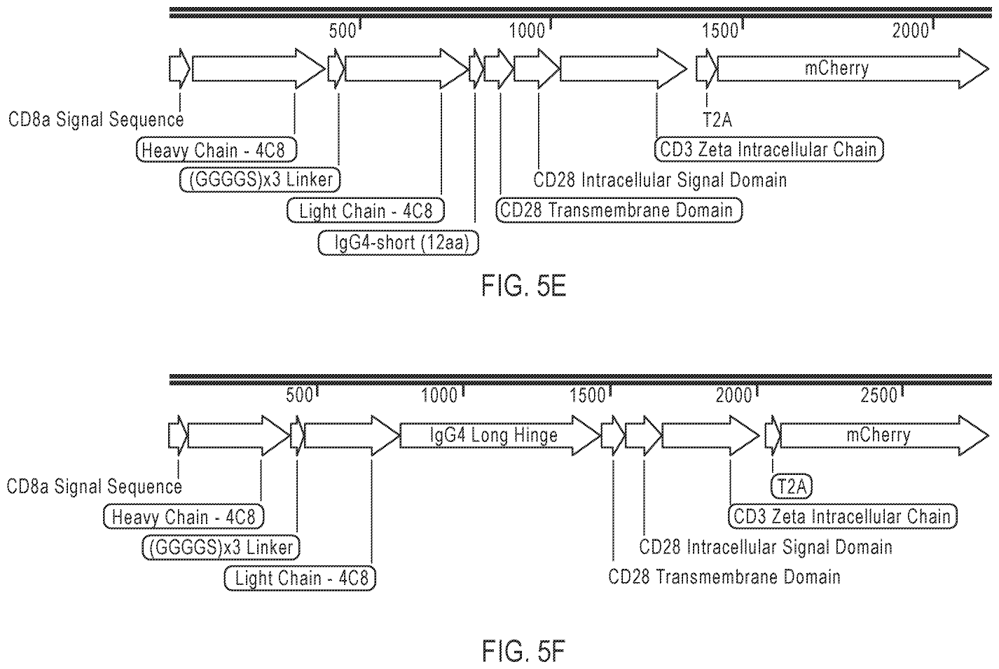

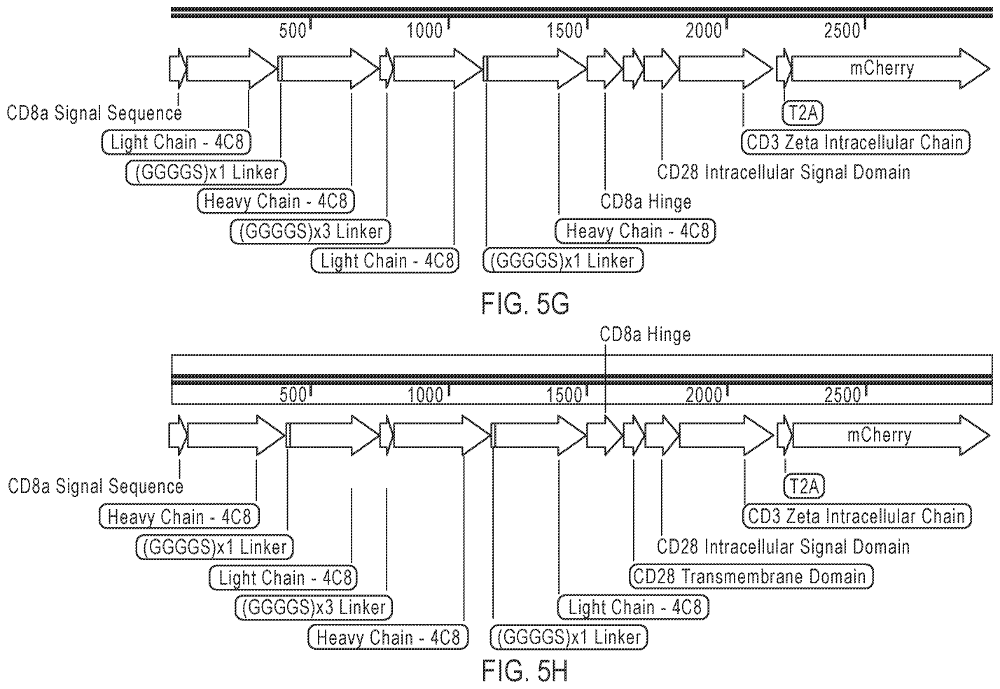

Various CAR constructs having scFvs having VH and VL domains of 4C8 were designed. In some constructs, the VH and VL are attached together with one long linker (GGGGS)₃ (SEQ ID NO:184), while other constructs comprise two scFvs in tandem with one short linker GGGGS (SEQ ID NO:183) between the VH and VL and one long linker (GGGGS)₃ (SEQ ID NO:184) between each scFv (see FIG. 5A-5H). The VH and VL were attached in various orientations to three different hinges (CD8a, IgG4-short, IgG4-long) followed by a second generation CAR-T (CD28 intracellular signal domain, and a CD3-zeta intracellular chain). The N-terminus of the scFvs was attached to a CD8a signal sequence. The 4C8 CAR-Ts were subcloned into the Virapower lentivirus vector pLENTI6.3-V5-DEST (Invitrogen).

Nucleotide sequences encoding the CARs are shown in Table 5A. Amino acid sequences of the CARs are shown in Table 5B.

TABLE 5A
Nucleotide sequences encoding CARs

| | | SEQ |
|---|---|---|
| Con- | | ID |
| struct | Nucleic acid sequence | NO: |
| 1 (LH- | ATGGCCCTGCCCGTGACCGCCCTGCTGCTGCCCCT | 167 |
| 4C8- | GGCCCTGCTGCTGCACGCCGCCAGGCCCCAGGCCG | |
| CD8a- | TGGTGACCCAGGAGAGCGCCCTGACCACCAGCCCC | |
| CART) | GGCGAGACCGTGACCCTGACCTGCAGGACCAGCAC | |
| | CGGCGCCGTGAGCATCAGGAACTACGCCAACTGGG | |
| | TGCAGGAGAAGCCCGACCACCTGTTCACCGGCCTG | |
| | ATCGGCGGCACCAACAACAGGGCCCCCGGCGTGCC | |
| | CGCCAGGTTCAGCGGCAGCCTGATCGGCGACAAGG | |
| | CCGCCCTGACCATCACCGGCGCCCAGCCCGAGGAC | |
| | GAGGCCATCTACTTCTGCGCCCTGCTGTACAGCAA | |
| | CTACTGGGTGTTCGGCGGCGGCACCAAGCTGACCG | |
| | TGCTGGGCGGCGGCGGCAGCCAGGTGCAGCTGCAG | |
| | CAGCCCGGCAGCGAGCTGGTGAGGCCCGGCGCCAG | |
| | CGTGAAGCTGAGCTGCAAGGCCAGCGGCTACACCT | |
| | TCACCAGCTACTGGATGCACTGGGTGAAGCAGAGG | |
| | CCCGGCCAGGGCCTGGAGTGGATCGGCAACATCTA | |
| | CCCCAGGAGCGGCACCACCAACTACGACGGCTACT | |
| | TCAAGAGCAAGGCCACCCTGACCGTGGACACCAGC | |
| | AGCAGCACCGCCTACATGCAGCTGAGCAGCCTGAC | |
| | CAGCGAGGACAGCGCCGTGTACTTCTGCACCAGGA | |
| | GCGGCTACGACTACCCCTTCGTGTACTGGGGCCAG | |
| | GGCACCCTGGTGACCGTGAGCGCCGGCGGCGGCGG | |
| | CAGCGGCGGCGGCGGCAGCGGCGGCGGCGGCAGCC | |
| | AGGCCGTGGTGACCCAGGAGAGCGCCCTGACCACC | |
| | AGCCCCGGCGAGACCGTGACCCTGACCTGCAGGAC | |
| | CAGCACCGGCGCCGTGAGCATCAGGAACTACGCCA | |
| | ACTGGGTGCAGGAGAAGCCCGACCACCTGTTCACC | |
| | GGCCTGATCGGCGGCACCAACAACAGGGCCCCCGG | |
| | CGTGCCCGCCAGGTTCAGCGGCAGCCTGATCGGCG | |
| | ACAAGGCCGCCCTGACCATCACCGGCGCCCAGCCC | |
| | GAGGACGAGGCCATCTACTTCTGCGCCCTGCTGTA | |
| | CAGCAACTACTGGGTGTTCGGCGGCGGCACCAAGC | |
| | TGACCGTGCTGGGCGGCGGCGGCAGCCAGGTGCAG | |
| | CTGCAGCAGCCCGGCAGCGAGCTGGTGAGGCCCGG | |
| | CGCCAGCGTGAAGCTGAGCTGCAAGGCCAGCGGCT | |
| | ACACCTTCACCAGCTACTGGATGCACTGGGTGAAG | |
| | CAGAGGCCCGGCCAGGGCCTGGAGTGGATCGGCAA | |
| | CATCTACCCCAGGAGCGGCACCACCAACTACGACG | |
| | GCTACTTCAAGAGCAAGGCCACCCTGACCGTGGAC | |
| | ACCAGCAGCAGCACCGCCTACATGCAGCTGAGCAG | |
| | CCTGACCAGCGAGGACAGCGCCGTGTACTTCTGCA | |
| | CCAGGAGCGGCTACGACTACCCCTTCGTGTACTGG | |
| | GGCCAGGGCACCCTGGTGACCGTGAGCGCCACCAC | |
| | CACCCCCGCCCCCAGGCCCCCCACCCCCGCCCCCA | |
| | CCATCGCCAGCCCCCTGAGCCTGAGGCCCGAGGCC | |
| | TGCAGGCCCGCCGCCGGCGGCGCCGTGCACACCAG | |
| | GGGCCTGGACTTCGCCTGCGACTTCTGGGTGCTGG | |
| | TGGTGGTGGGCGGCGTGCTGGCCTGCTACAGCCTG | |
| | CTGGTGACCGTGGCCTTCATCATCTTCTGGGTGAG | |
| | GAGCAAGAGGAGCAGGCTGCTGCACAGCGACTACA | |
| | TGAACATGACCCCCAGGAGGCCCGGCCCCACCAGG | |
| | AAGCACTACCAGCCCTACGCCCCCCCCAGGGACTT | |
| | CGCCGCCTACAGGAGCAGGGTGAAGTTCAGCAGGA | |
| | GCGCCGACGCCCCCGCCTACCAGCAGGGCCAGAAC | |
| | CAGCTGTACAACGAGCTGAACCTGGGCAGGAGGGA | |
| | GGAGTACGACGTGCTGGACAAGAGGAGGGGCAGGG | |
| | ACCCCGAGATGGGCGGCAAGCCCAGGAGGAAGAAC | |
| | CCCCAGGAGGGCCTGTACAACGAGCTGCAGAAGGA | |
| | CAAGATGGCCGAGGCCTACAGCGAGATCGGCATGA | |
| | AGGGCGAGAGGAGGAGGGGCAAGGGCCACGACGGC | |
| | CTGTACCAGGGCCTGAGCACCGCCACCAAGGACAC | |
| | CTACGACGCCCTGCACATGCAGGCCCTGCCCCCCA | |
| | GGAGAAAGAGAGGAAGCGGAGAGGGCAGAGGAAGT | |
| | CTGCTAACATGCGGTGACGTCGAGGAGAATCCTGG | |
| | ACCTATGGTGAGCAAGGGCGAGGAGGATAACATGG | |
| | CCATCATCAAGGAGTTCATGCGCTTCAAGGTGCAC | |
| | ATGGAGGGCTCCGTGAACGGCCACGAGTTCGAGAT | |
| | CGAGGGCGAGGGCGAGGGCCGCCCCTACGAGGGCA | |
| | CCCAGACCGCCAAGCTGAAGGTGACCAAGGGTGGC | |
| | CCCCTGCCCTTCGCCTGGGACATCCTGTCCCCTCA | |
| | GTTCATGTACGGCTCCAAGGCCTACGTGAAGCACC | |
| | CCGCCGACATCCCCGACTACTTGAAGCTGTCCTTC | |
| | CCCGAGGGCTTCAAGTGGGAGCGCGTGATGAACTT | |
| | CGAGGACGGCGGCGTGGTGACCGTGACCCAGGACT | |
| | CCTCCCTGCAGGACGGCGAGTTCATCTACAAGGTG | |
| | AAGCTGCGCGGCACCAACTTCCCCTCCGACGGCCC | |
| | CGTAATGCAGAAGAAGACCATGGGCTGGGAGGCCT | |
| | CCTCCGAGCGGATGTACCCCGAGGACGGCGCCCTG | |
| | AAGGGCGAGATCAAGCAGAGGCTGAAGCTGAAGGA | |
| | CGGCGGCCACTACGACGCTGAGGTCAAGACCACCT | |
| | ACAAGGCCAAGAAGCCCGTGCAGCTGCCCGGCGCC | |
| | TACAACGTCAACATCAAGTTGGACATCACCTCCCA | |
| | CAACGAGGACTACACCATCGTGGAACAGTACGAAC | |
| | GCGCCGAGGGCCGCCACTCCACCGGCGGCATGGAC | |
| | GAGCTGTACAAGTAG | |
| 2 (LH- | ATGGCCCTGCCCGTGACCGCCCTGCTGCTGCCCCT | 168 |
| 4C8- | GGCCCTGCTGCTGCACGCCGCCAGGCCCCAGGCCG | |
| IgG4- | TGGTGACCCAGGAGAGCGCCCTGACCACCAGCCCC | |
| CART) | GGCGAGACCGTGACCCTGACCTGCAGGACCAGCAC | |
| | CGGCGCCGTGAGCATCAGGAACTACGCCAACTGGG | |
| | TGCAGGAGAAGCCCGACCACCTGTTCACCGGCCTG | |
| | ATCGGCGGCACCAACAACAGGGCCCCCGGCGTGCC | |
| | CGCCAGGTTCAGCGGCAGCCTGATCGGCGACAAGG | |
| | CCGCCCTGACCATCACCGGCGCCCAGCCCGAGGAC | |
| | GAGGCCATCTACTTCTGCGCCCTGCTGTACAGCAA | |
| | CTACTGGGTGTTCGGCGGCGGCACCAAGCTGACCG | |
| | TGCTGGGCGGCGGCGGCAGCGGCGGCGGCGGCAGC | |
| | GGCGGCGGCGGCAGCCAGGTGCAGCTGCAGCAGCC | |
| | CGGCAGCGAGCTGGTGAGGCCCGGCGCCAGCGTGA | |
| | AGCTGAGCTGCAAGGCCAGCGGCTACACCTTCACC | |
| | AGCTACTGGATGCACTGGGTGAAGCAGAGGCCCGG | |
| | CCAGGGCCTGGAGTGGATCGGCAACATCTACCCCA | |
| | GGAGCGGCACCACCAACTACGACGGCTACTTCAAG | |
| | AGCAAGGCCACCCTGACCGTGGACACCAGCAGCAG | |
| | CACCGCCTACATGCAGCTGAGCAGCCTGACCAGCG | |
| | AGGACAGCGCCGTGTACTTCTGCACCAGGAGCGGC | |
| | TACGACTACCCCTTCGTGTACTGGGGCCAGGGCAC | |
| | CCTGGTGACCGTGAGCGCCGAGAGCAAGTACGGCC | |
| | CCCCCTGCCCCAGCTGCCCCTTCTGGGTGCTGGTG | |
| | GTGGTGGGCGGCGTGCTGGCCTGCTACAGCCTGCT | |
| | GGTGACCGTGGCCTTCATCATCTTCTGGGTGAGGA | |
| | GCAAGAGGAGCAGGCTGCTGCACAGCGACTACATG | |
| | AACATGACCCCCAGGAGGCCCGGCCCCACCAGGAA | |
| | GCACTACCAGCCCTACGCCCCCCCCAGGGACTTCG | |
| | CCGCCTACAGGAGCAGGGTGAAGTTCAGCAGGAGC | |
| | GCCGACGCCCCCGCCTACCAGCAGGGCCAGAACCA | |
| | GCTGTACAACGAGCTGAACCTGGGCAGGAGGGAGG | |
| | AGTACGACGTGCTGGACAAGAGGAGGGGCAGGGAC | |
| | CCCGAGATGGGCGGCAAGCCCAGGAGGAAGAACCC | |
| | CCAGGAGGGCCTGTACAACGAGCTGCAGAAGGACA | |
| | AGATGGCCGAGGCCTACAGCGAGATCGGCATGAAG | |
| | GGCGAGAGGAGGAGGGGCAAGGGCCACGACGGCCT | |
| | GTACCAGGGCCTGAGCACCGCCACCAAGGACACCT | |
| | ACGACGCCCTGCACATGCAGGCCCTGCCCCCCAGG | |
| | AGAAAGAGAGGAAGCGGAGAGGGCAGAGGAAGTCT | |
| | GCTAACATGCGGTGACGTCGAGGAGAATCCTGGAC | |
| | CTATGGTGAGCAAGGGCGAGGAGGATAACATGGCC | |
| | ATCATCAAGGAGTTCATGCGCTTCAAGGTGCACAT | |
| | GGAGGGCTCCGTGAACGGCCACGAGTTCGAGATCG | |
| | AGGGCGAGGGCGAGGGCCGCCCCTACGAGGGCACC | |
| | CAGACCGCCAAGCTGAAGGTGACCAAGGGTGGCCC | |
| | CCTGCCCTTCGCCTGGGACATCCTGTCCCCTCAGT | |
| | TCATGTACGGCTCCAAGGCCTACGTGAAGCACCCC | |
| | GCCGACATCCCCGACTACTTGAAGCTGTCCTTCCC | |
| | CGAGGGCTTCAAGTGGGAGCGCGTGATGAACTTCG | |
| | AGGACGGCGGCGTGGTGACCGTGACCCAGGACTCC | |
| | TCCCTGCAGGACGGCGAGTTCATCTACAAGGTGAA | |
| | GCTGCGCGGCACCAACTTCCCCTCCGACGGCCCCG | |
| | TAATGCAGAAGAAGACCATGGGCTGGGAGGCCTCC | |
| | TCCGAGCGGATGTACCCCGAGGACGGCGCCCTGAA | |
| | GGGCGAGATCAAGCAGAGGCTGAAGCTGAAGGACG | |
| | GCGGCCACTACGACGCTGAGGTCAAGACCACCTAC | |
| | AAGGCCAAGAAGCCCGTGCAGCTGCCCGGCGCCTA | |
| | CAACGTCAACATCAAGTTGGACATCACCTCCCACA | |
| | ACGAGGACTACACCATCGTGGAACAGTACGAACGC | |
| | GCCGAGGGCCGCCACTCCACCGGCGGCATGGACGA | |
| | GCTGTACAAGTAG | |
| 3 (LH- | ATGGCCCTGCCCGTGACCGCCCTGCTGCTGCCCCT | 169 |
| 4C8- | GGCCCTGCTGCTGCACGCCGCCAGGCCCCAGGCCG | |
| IgG4- | TGGTGACCCAGGAGAGCGCCCTGACCACCAGCCCC | |
| Long- | GGCGAGACCGTGACCCTGACCTGCAGGACCAGCAC | |
| CART) | CGGCGCCGTGAGCATCAGGAACTACGCCAACTGGG | |
| | TGCAGGAGAAGCCCGACCACCTGTTCACCGGCCTG | |
| | ATCGGCGGCACCAACAACAGGGCCCCCGGCGTGCC | |
| | CGCCAGGTTCAGCGGCAGCCTGATCGGCGACAAGG | |
| | CCGCCCTGACCATCACCGGCGCCCAGCCCGAGGAC | |
| | GAGGCCATCTACTTCTGCGCCCTGCTGTACAGCAA | |
| | CTACTGGGTGTTCGGCGGCGGCACCAAGCTGACCG | |
| | TGCTGGGCGGCGGCGGCAGCGGCGGCGGCGGCAGC | |
| | GGCGGCGGCGGCAGCCAGGTGCAGCTGCAGCAGCC | |
| | CGGCAGCGAGCTGGTGAGGCCCGGCGCCAGCGTGA | |
| | AGCTGAGCTGCAAGGCCAGCGGCTACACCTTCACC | |
| | AGCTACTGGATGCACTGGGTGAAGCAGAGGCCCGG | |
| | CCAGGGCCTGGAGTGGATCGGCAACATCTACCCCA | |
| | GGAGCGGCACCACCAACTACGACGGCTACTTCAAG | |
| | AGCAAGGCCACCCTGACCGTGGACACCAGCAGCAG | |
| | CACCGCCTACATGCAGCTGAGCAGCCTGACCAGCG | |
| | AGGACAGCGCCGTGTACTTCTGCACCAGGAGCGGC | |
| | TACGACTACCCCTTCGTGTACTGGGGCCAGGGCAC | |
| | CCTGGTGACCGTGAGCGCCGAGAGCAAGTACGGCC | |
| | CCCCCTGCCCCCCCTGCCCCGCCCCCCCCGTGGCC | |
| | GGCCCCAGCGTGTTCCTGTTCCCCCCCAAGCCCAA | |
| | GGACACCCTGATGATCAGCAGGACCCCCGAGGTGA | |
| | CCTGCGTGGTGGTGGACGTGAGCCAGGAGGACCCC | |
| | GAGGTGCAGTTCAACTGGTACGTGGACGGCGTGGA | |
| | GGTGCACAACGCCAAGACCAAGCCCAGGGAGGAGC | |
| | AGTTCCAGAGCACCTACAGGGTGGTGAGCGTGCTG | |
| | ACCGTGCTGCACCAGGACTGGCTGAACGGCAAGGA | |
| | GTACAAGTGCAAGGTGAGCAACAAGGGCCTGCCCA | |
| | GCAGCATCGAGAAGACCATCAGCAAGGCCAAGGGC | |
| | CAGCCCAGGGAGCCCCAGGTGTACACCCTGCCCCC | |
| | CAGCCAGGAGGAGATGACCAAGAACCAGGTGAGCC | |
| | TGACCTGCCTGGTGAAGGGCTTCTACCCCAGCGAC | |
| | ATCGCCGTGGAGTGGGAGAGCAACGGCCAGCCCGA | |
| | GAACAACTACAAGACCACCCCCCCCGTGCTGGACA | |
| | GCGACGGCAGCTTCTTCCTGTACAGCAGGCTGACC | |
| | GTGGACAAGAGCAGGTGGCAGGAGGGCAACGTGTT | |
| | CAGCTGCAGCGTGATGCACGAGGCCCTGCACAACC | |
| | ACTACACCCAGAAGAGCCTGAGCCTGAGCCTGGGC | |
| | AAGATGTTCTGGGTGCTGGTGGTGGTGGGCGGCGT | |
| | GCTGGCCTGCTACAGCCTGCTGGTGACCGTGGCCT | |
| | TCATCATCTTCTGGGTGAGGAGCAAGAGGAGCAGG | |
| | CTGCTGCACAGCGACTACATGAACATGACCCCCAG | |
| | GAGGCCCGGCCCCACCAGGAAGCACTACCAGCCCT | |
| | ACGCCCCCCCCAGGGACTTCGCCGCCTACAGGAGC | |
| | AGGGTGAAGTTCAGCAGGAGCGCCGACGCCCCCGC | |
| | CTACCAGCAGGGCCAGAACCAGCTGTACAACGAGC | |
| | TGAACCTGGGCAGGAGGGAGGAGTACGACGTGCTG | |
| | GACAAGAGGAGGGGCAGGGACCCCGAGATGGGCGG | |
| | CAAGCCCAGGAGGAAGAACCCCCAGGAGGGCCTGT | |
| | ACAACGAGCTGCAGAAGGACAAGATGGCCGAGGCC | |
| | TACAGCGAGATCGGCATGAAGGGCGAGAGGAGGAG | |
| | GGGCAAGGGCCACGACGGCCTGTACCAGGGCCTGA | |
| | GCACCGCCACCAAGGACACCTACGACGCCCTGCAC | |
| | ATGCAGGCCCTGCCCCCCAGGAGAAAGAGAGGAAG | |
| | CGGAGAGGGCAGAGGAAGTCTGCTAACATGCGGTG | |
| | ACGTCGAGGAGAATCCTGGACCTATGGTGAGCAAG | |
| | GGCGAGGAGGATAACATGGCCATCATCAAGGAGTT | |
| | CATGCGCTTCAAGGTGCACATGGAGGGCTCCGTGA | |
| | ACGGCCACGAGTTCGAGATCGAGGGCGAGGGCGAG | |
| | GGCCGCCCCTACGAGGGCACCCAGACCGCCAAGCT | |
| | GAAGGTGACCAAGGGTGGCCCCCTGCCCTTCGCCT | |
| | GGGACATCCTGTCCCCTCAGTTCATGTACGGCTCC | |
| | AAGGCCTACGTGAAGCACCCCGCCGACATCCCCGA | |
| | CTACTTGAAGCTGTCCTTCCCCGAGGGCTTCAAGT | |
| | GGGAGCGCGTGATGAACTTCGAGGACGGCGGCGTG | |
| | GTGACCGTGACCCAGGACTCCTCCCTGCAGGACGG | |
| | CGAGTTCATCTACAAGGTGAAGCTGCGCGGCACCA | |
| | ACTTCCCCTCCGACGGCCCCGTAATGCAGAAGAAG | |
| | ACCATGGGCTGGGAGGCCTCCTCCGAGCGGATGTA | |
| | CCCCGAGGACGGCGCCCTGAAGGGCGAGATCAAGC | |
| | AGAGGCTGAAGCTGAAGGACGGCGGCCACTACGAC | |
| | GCTGAGGTCAAGACCACCTACAAGGCCAAGAAGCC | |
| | CGTGCAGCTGCCCGGCGCCTACAACGTCAACATCA | |
| | AGTTGGACATCACCTCCCACAACGAGGACTACACC | |
| | ATCGTGGAACAGTACGAACGCGCCGAGGGCCGCCA | |
| | CTCCACCGGCGGCATGGACGAGCTGTACAAGTAG | |
| 4 (HL- | ATGGCCCTGCCCGTGACCGCCCTGCTGCTGCCCCT | 170 |
| 4C8- | GGCCCTGCTGCTGCACGCCGCCAGGCCCCAGGTGC | |
| CD8a- | AGCTGCAGCAGCCCGGCAGCGAGCTGGTGAGGCCC | |
| CART) | GGCGCCAGCGTGAAGCTGAGCTGCAAGGCCAGCGG | |
| | CTACACCTTCACCAGCTACTGGATGCACTGGGTGA | |
| | AGCAGAGGCCCGGCCAGGGCCTGGAGTGGATCGGC | |
| | AACATCTACCCCAGGAGCGGCACCACCAACTACGA | |
| | CGGCTACTTCAAGAGCAAGGCCACCCTGACCGTGG | |
| | ACACCAGCAGCAGCACCGCCTACATGCAGCTGAGC | |
| | AGCCTGACCAGCGAGGACAGCGCCGTGTACTTCTG | |
| | CACCAGGAGCGGCTACGACTACCCCTTCGTGTACT | |
| | GGGGCCAGGGCACCCTGGTGACCGTGAGCGCCGGC | |
| | GGCGGCGGCAGCGGCGGCGGCGGCAGCGGCGGCGG | |
| | CGGCAGCCAGGCCGTGGTGACCCAGGAGAGCGCCC | |
| | TGACCACCAGCCCCGGCGAGACCGTGACCCTGACC | |
| | TGCAGGACCAGCACCGGCGCCGTGAGCATCAGGAA | |
| | CTACGCCAACTGGGTGCAGGAGAAGCCCGACCACC | |
| | TGTTCACCGGCCTGATCGGCGGCACCAACAACAGG | |
| | GCCCCCGGCGTGCCCGCCAGGTTCAGCGGCAGCCT | |
| | GATCGGCGACAAGGCCGCCCTGACCATCACCGGCG | |
| | CCCAGCCCGAGGACGAGGCCATCTACTTCTGCGCC | |
| | CTGCTGTACAGCAACTACTGGGTGTTCGGCGGCGG | |
| | CACCAAGCTGACCGTGCTGACCACCACCCCCGCCC | |
| | CCAGGCCCCCCACCCCCGCCCCCACCATCGCCAGC | |
| | CCCCTGAGCCTGAGGCCCGAGGCCTGCAGGCCCGC | |
| | CGCCGGCGGCGCCGTGCACACCAGGGGCCTGGACT | |
| | TCGCCTGCGACTTCTGGGTGCTGGTGGTGGTGGGC | |
| | GGCGTGCTGGCCTGCTACAGCCTGCTGGTGACCGT | |
| | GGCCTTCATCATCTTCTGGGTGAGGAGCAAGAGGA | |
| | GCAGGCTGCTGCACAGCGACTACATGAACATGACC | |
| | CCCAGGAGGCCCGGCCCCACCAGGAAGCACTACCA | |
| | GCCCTACGCCCCCCCCAGGGACTTCGCCGCCTACA | |
| | GGAGCAGGGTGAAGTTCAGCAGGAGCGCCGACGCC | |
| | CCCGCCTACCAGCAGGGCCAGAACCAGCTGTACAA | |
| | CGAGCTGAACCTGGGCAGGAGGGAGGAGTACGACG | |
| | TGCTGGACAAGAGGAGGGGCAGGGACCCCGAGATG | |
| | GGCGGCAAGCCCAGGAGGAAGAACCCCCAGGAGGG | |
| | CCTGTACAACGAGCTGCAGAAGGACAAGATGGCCG | |
| | AGGCCTACAGCGAGATCGGCATGAAGGGCGAGAGG | |
| | AGGAGGGGCAAGGGCCACGACGGCCTGTACCAGGG | |
| | CCTGAGCACCGCCACCAAGGACACCTACGACGCCC | |
| | TGCACATGCAGGCCCTGCCCCCCAGGAGAAAGAGA | |
| | GGAAGCGGAGAGGGCAGAGGAAGTCTGCTAACATG | |
| | CGGTGACGTCGAGGAGAATCCTGGACCTATGGTGA | |
| | GCAAGGGCGAGGAGGATAACATGGCCATCATCAAG | |
| | GAGTTCATGCGCTTCAAGGTGCACATGGAGGGCTC | |
| | CGTGAACGGCCACGAGTTCGAGATCGAGGGCGAGG | |
| | GCGAGGGCCGCCCCTACGAGGGCACCCAGACCGCC | |
| | AAGCTGAAGGTGACCAAGGGTGGCCCCCTGCCCTT | |
| | CGCCTGGGACATCCTGTCCCCTCAGTTCATGTACG | |
| | GCTCCAAGGCCTACGTGAAGCACCCCGCCGACATC | |
| | CCCGACTACTTGAAGCTGTCCTTCCCCGAGGGCTT | |
| | CAAGTGGGAGCGCGTGATGAACTTCGAGGACGGCG | |
| | GCGTGGTGACCGTGACCCAGGACTCCTCCCTGCAG | |
| | GACGGCGAGTTCATCTACAAGGTGAAGCTGCGCGG | |
| | CACCAACTTCCCCTCCGACGGCCCCGTAATGCAGA | |
| | AGAAGACCATGGGCTGGGAGGCCTCCTCCGAGCGG | |
| | ATGTACCCCGAGGACGGCGCCCTGAAGGGCGAGAT | |
| | CAAGCAGAGGCTGAAGCTGAAGGACGGCGGCCACT | |
| | ACGACGCTGAGGTCAAGACCACCTACAAGGCCAAG | |
| | AAGCCCGTGCAGCTGCCCGGCGCCTACAACGTCAA | |
| | CATCAAGTTGGACATCACCTCCCACAACGAGGACT | |
| | ACACCATCGTGGAACAGTACGAACGCGCCGAGGGC | |
| | CGCCACTCCACCGGCGGCATGGACGAGCTGTACAA | |
| | GTAG | |
| 5 (HL- | ATGGCCCTGCCCGTGACCGCCCTGCTGCTGCCCCT | 171 |
| 4C8- | GGCCCTGCTGCTGCACGCCGCCAGGCCCCAGGTGC | |
| IgG4- | AGCTGCAGCAGCCCGGCAGCGAGCTGGTGAGGCCC | |
| CART) | GGCGCCAGCGTGAAGCTGAGCTGCAAGGCCAGCGG | |
| | CTACACCTTCACCAGCTACTGGATGCACTGGGTGA | |
| | AGCAGAGGCCCGGCCAGGGCCTGGAGTGGATCGGC | |
| | AACATCTACCCCAGGAGCGGCACCACCAACTACGA | |
| | CGGCTACTTCAAGAGCAAGGCCACCCTGACCGTGG | |
| | ACACCAGCAGCAGCACCGCCTACATGCAGCTGAGC | |
| | AGCCTGACCAGCGAGGACAGCGCCGTGTACTTCTG | |
| | CACCAGGAGCGGCTACGACTACCCCTTCGTGTACT | |
| | GGGGCCAGGGCACCCTGGTGACCGTGAGCGCCGGC | |
| | GGCGGCGGCAGCGGCGGCGGCGGCAGCGGCGGCGG | |
| | CGGCAGCCAGGCCGTGGTGACCCAGGAGAGCGCCC | |
| | TGACCACCAGCCCCGGCGAGACCGTGACCCTGACC | |
| | TGCAGGACCAGCACCGGCGCCGTGAGCATCAGGAA | |
| | CTACGCCAACTGGGTGCAGGAGAAGCCCGACCACC | |
| | TGTTCACCGGCCTGATCGGCGGCACCAACAACAGG | |
| | GCCCCCGGCGTGCCCGCCAGGTTCAGCGGCAGCCT | |
| | GATCGGCGACAAGGCCGCCCTGACCATCACCGGCG | |
| | CCCAGCCCGAGGACGAGGCCATCTACTTCTGCGCC | |
| | CTGCTGTACAGCAACTACTGGGTGTTCGGCGGCGG | |
| | CACCAAGCTGACCGTGCTGGAGAGCAAGTACGGCC | |
| | CCCCCTGCCCCAGCTGCCCCTTCTGGGTGCTGGTG | |
| | GTGGTGGGCGGCGTGCTGGCCTGCTACAGCCTGCT | |
| | GGTGACCGTGGCCTTCATCATCTTCTGGGTGAGGA | |
| | GCAAGAGGAGCAGGCTGCTGCACAGCGACTACATG | |
| | AACATGACCCCCAGGAGGCCCGGCCCCACCAGGAA | |
| | GCACTACCAGCCCTACGCCCCCCCCAGGGACTTCG | |
| | CCGCCTACAGGAGCAGGGTGAAGTTCAGCAGGAGC | |
| | GCCGACGCCCCCGCCTACCAGCAGGGCCAGAACCA | |
| | GCTGTACAACGAGCTGAACCTGGGCAGGAGGGAGG | |
| | AGTACGACGTGCTGGACAAGAGGAGGGGCAGGGAC | |
| | CCCGAGATGGGCGGCAAGCCCAGGAGGAAGAACCC | |
| | CCAGGAGGGCCTGTACAACGAGCTGCAGAAGGACA | |
| | AGATGGCCGAGGCCTACAGCGAGATCGGCATGAAG | |
| | GGCGAGAGGAGGAGGGGCAAGGGCCACGACGGCCT | |
| | GTACCAGGGCCTGAGCACCGCCACCAAGGACACCT | |
| | ACGACGCCCTGCACATGCAGGCCCTGCCCCCCAGG | |
| | AGAAAGAGAGGAAGCGGAGAGGGCAGAGGAAGTCT | |
| | GCTAACATGCGGTGACGTCGAGGAGAATCCTGGAC | |
| | CTATGGTGAGCAAGGGCGAGGAGGATAACATGGCC | |
| | ATCATCAAGGAGTTCATGCGCTTCAAGGTGCACAT | |
| | GGAGGGCTCCGTGAACGGCCACGAGTTCGAGATCG | |
| | AGGGCGAGGGCGAGGGCCGCCCCTACGAGGGCACC | |
| | CAGACCGCCAAGCTGAAGGTGACCAAGGGTGGCCC | |
| | CCTGCCCTTCGCCTGGGACATCCTGTCCCCTCAGT | |
| | TCATGTACGGCTCCAAGGCCTACGTGAAGCACCCC | |
| | GCCGACATCCCCGACTACTTGAAGCTGTCCTTCCC | |
| | CGAGGGCTTCAAGTGGGAGCGCGTGATGAACTTCG | |
| | AGGACGGCGGCGTGGTGACCGTGACCCAGGACTCC | |
| | TCCCTGCAGGACGGCGAGTTCATCTACAAGGTGAA | |
| | GCTGCGCGGCACCAACTTCCCCTCCGACGGCCCCG | |
| | TAATGCAGAAGAAGACCATGGGCTGGGAGGCCTCC | |
| | TCCGAGCGGATGTACCCCGAGGACGGCGCCCTGAA | |
| | GGGCGAGATCAAGCAGAGGCTGAAGCTGAAGGACG | |
| | GCGGCCACTACGACGCTGAGGTCAAGACCACCTAC | |
| | AAGGCCAAGAAGCCCGTGCAGCTGCCCGGCGCCTA | |
| | CAACGTCAACATCAAGTTGGACATCACCTCCCACA | |
| | ACGAGGACTACACCATCGTGGAACAGTACGAACGC | |
| | GCCGAGGGCCGCCACTCCACCGGCGGCATGGACGA | |
| | GCTGTACAAGTAG | |
| 6 (HL- | ATGGCCCTGCCCGTGACCGCCCTGCTGCTGCCCCT | 172 |
| 4C8- | GGCCCTGCTGCTGCACGCCGCCAGGCCCCAGGTGC | |
| IgG4- | AGCTGCAGCAGCCCGGCAGCGAGCTGGTGAGGCCC | |
| Long- | GGCGCCAGCGTGAAGCTGAGCTGCAAGGCCAGCGG | |
| CART) | CTACACCTTCACCAGCTACTGGATGCACTGGGTGA | |
| | AGCAGAGGCCCGGCCAGGGCCTGGAGTGGATCGGC | |
| | AACATCTACCCCAGGAGCGGCACCACCAACTACGA | |
| | CGGCTACTTCAAGAGCAAGGCCACCCTGACCGTGG | |
| | ACACCAGCAGCAGCACCGCCTACATGCAGCTGAGC | |
| | AGCCTGACCAGCGAGGACAGCGCCGTGTACTTCTG | |
| | CACCAGGAGCGGCTACGACTACCCCTTCGTGTACT | |
| | GGGGCCAGGGCACCCTGGTGACCGTGAGCGCCGGC | |
| | GGCGGCGGCAGCGGCGGCGGCGGCAGCGGCGGCGG | |
| | CGGCAGCCAGGCCGTGGTGACCCAGGAGAGCGCCC | |
| | TGACCACCAGCCCCGGCGAGACCGTGACCCTGACC | |
| | TGCAGGACCAGCACCGGCGCCGTGAGCATCAGGAA | |
| | CTACGCCAACTGGGTGCAGGAGAAGCCCGACCACC | |
| | TGTTCACCGGCCTGATCGGCGGCACCAACAACAGG | |
| | GCCCCCGGCGTGCCCGCCAGGTTCAGCGGCAGCCT | |
| | GATCGGCGACAAGGCCGCCCTGACCATCACCGGCG | |
| | CCCAGCCCGAGGACGAGGCCATCTACTTCTGCGCC | |
| | CTGCTGTACAGCAACTACTGGGTGTTCGGCGGCGG | |
| | CACCAAGCTGACCGTGCTGGAGAGCAAGTACGGCC | |
| | CCCCCTGCCCCCCCTGCCCCGCCCCCCCCGTGGCC | |
| | GGCCCCAGCGTGTTCCTGTTCCCCCCCAAGCCCAA | |
| | GGACACCCTGATGATCAGCAGGACCCCCGAGGTGA | |
| | CCTGCGTGGTGGTGGACGTGAGCCAGGAGGACCCC | |
| | GAGGTGCAGTTCAACTGGTACGTGGACGGCGTGGA | |
| | GGTGCACAACGCCAAGACCAAGCCCAGGGAGGAGC | |
| | AGTTCCAGAGCACCTACAGGGTGGTGAGCGTGCTG | |
| | ACCGTGCTGCACCAGGACTGGCTGAACGGCAAGGA | |
| | GTACAAGTGCAAGGTGAGCAACAAGGGCCTGCCCA | |
| | GCAGCATCGAGAAGACCATCAGCAAGGCCAAGGGC | |
| | CAGCCCAGGGAGCCCCAGGTGTACACCCTGCCCCC | |
| | CAGCCAGGAGGAGATGACCAAGAACCAGGTGAGCC | |
| | TGACCTGCCTGGTGAAGGGCTTCTACCCCAGCGAC | |
| | ATCGCCGTGGAGTGGGAGAGCAACGGCCAGCCCGA | |
| | GAACAACTACAAGACCACCCCCCCCGTGCTGGACA | |
| | GCGACGGCAGCTTCTTCCTGTACAGCAGGCTGACC | |
| | GTGGACAAGAGCAGGTGGCAGGAGGGCAACGTGTT | |
| | CAGCTGCAGCGTGATGCACGAGGCCCTGCACAACC | |
| | ACTACACCCAGAAGAGCCTGAGCCTGAGCCTGGGC | |
| | AAGATGTTCTGGGTGCTGGTGGTGGTGGGCGGCGT | |
| | GCTGGCCTGCTACAGCCTGCTGGTGACCGTGGCCT | |
| | TCATCATCTTCTGGGTGAGGAGCAAGAGGAGCAGG | |
| | CTGCTGCACAGCGACTACATGAACATGACCCCCAG | |
| | GAGGCCCGGCCCCACCAGGAAGCACTACCAGCCCT | |
| | ACGCCCCCCCCAGGGACTTCGCCGCCTACAGGAGC | |
| | AGGGTGAAGTTCAGCAGGAGCGCCGACGCCCCCGC | |
| | CTACCAGCAGGGCCAGAACCAGCTGTACAACGAGC | |
| | TGAACCTGGGCAGGAGGGAGGAGTACGACGTGCTG | |
| | GACAAGAGGAGGGGCAGGGACCCCGAGATGGGCGG | |
| | CAAGCCCAGGAGGAAGAACCCCCAGGAGGGCCTGT | |
| | ACAACGAGCTGCAGAAGGACAAGATGGCCGAGGCC | |
| | TACAGCGAGATCGGCATGAAGGGCGAGAGGAGGAG | |
| | GGGCAAGGGCCACGACGGCCTGTACCAGGGCCTGA | |
| | GCACCGCCACCAAGGACACCTACGACGCCCTGCAC | |
| | ATGCAGGCCCTGCCCCCCAGGAGAAAGAGAGGAAG | |
| | CGGAGAGGGCAGAGGAAGTCTGCTAACATGCGGTG | |
| | ACGTCGAGGAGAATCCTGGACCTATGGTGAGCAAG | |
| | GGCGAGGAGGATAACATGGCCATCATCAAGGAGTT | |
| | CATGCGCTTCAAGGTGCACATGGAGGGCTCCGTGA | |
| | ACGGCCACGAGTTCGAGATCGAGGGCGAGGGCGAG | |
| | GGCCGCCCCTACGAGGGCACCCAGACCGCCAAGCT | |
| | GAAGGTGACCAAGGGTGGCCCCCTGCCCTTCGCCT | |
| | GGGACATCCTGTCCCCTCAGTTCATGTACGGCTCC | |
| | AAGGCCTACGTGAAGCACCCCGCCGACATCCCCGA | |
| | CTACTTGAAGCTGTCCTTCCCCGAGGGCTTCAAGT | |
| | GGGAGCGCGTGATGAACTTCGAGGACGGCGGCGTG | |
| | GTGACCGTGACCCAGGACTCCTCCCTGCAGGACGG | |
| | CGAGTTCATCTACAAGGTGAAGCTGCGCGGCACCA | |
| | ACTTCCCCTCCGACGGCCCCGTAATGCAGAAGAAG | |
| | ACCATGGGCTGGGAGGCCTCCTCCGAGCGGATGTA | |
| | CCCCGAGGACGGCGCCCTGAAGGGCGAGATCAAGC | |
| | AGAGGCTGAAGCTGAAGGACGGCGGCCACTACGAC | |
| | GCTGAGGTCAAGACCACCTACAAGGCCAAGAAGCC | |
| | CGTGCAGCTGCCCGGCGCCTACAACGTCAACATCA | |
| | AGTTGGACATCACCTCCCACAACGAGGACTACACC | |
| | ATCGTGGAACAGTACGAACGCGCCGAGGGCCGCCA | |
| | CTCCACCGGCGGCATGGACGAGCTGTACAAGTAG | |
| 7 | ATGGCCCTGCCCGTGACCGCCCTGCTGCTGCCCCT | 173 |
| (LHx2- | GGCCCTGCTGCTGCACGCCGCCAGGCCCCAGGCCG | |
| 4C8- | TGGTGACCCAGGAGAGCGCCCTGACCACCAGCCCC | |
| CD8- | GGCGAGACCGTGACCCTGACCTGCAGGACCAGCAC | |
| CART) | CGGCGCCGTGAGCATCAGGAACTACGCCAACTGGG | |
| | TGCAGGAGAAGCCCGACCACCTGTTCACCGGCCTG | |
| | ATCGGCGGCACCAACAACAGGGCCCCCGGCGTGCC | |
| | CGCCAGGTTCAGCGGCAGCCTGATCGGCGACAAGG | |
| | CCGCCCTGACCATCACCGGCGCCCAGCCCGAGGAC | |
| | GAGGCCATCTACTTCTGCGCCCTGCTGTACAGCAA | |
| | CTACTGGGTGTTCGGCGGCGGCACCAAGCTGACCG | |
| | TGCTGGGCGGCGGCGGCAGCCAGGTGCAGCTGCAG | |
| | CAGCCCGGCAGCGAGCTGGTGAGGCCCGGCGCCAG | |
| | CGTGAAGCTGAGCTGCAAGGCCAGCGGCTACACCT | |
| | TCACCAGCTACTGGATGCACTGGGTGAAGCAGAGG | |
| | CCCGGCCAGGGCCTGGAGTGGATCGGCAACATCTA | |
| | CCCCAGGAGCGGCACCACCAACTACGACGGCTACT | |
| | TCAAGAGCAAGGCCACCCTGACCGTGGACACCAGC | |
| | AGCAGCACCGCCTACATGCAGCTGAGCAGCCTGAC | |
| | CAGCGAGGACAGCGCCGTGTACTTCTGCACCAGGA | |
| | GCGGCTACGACTACCCCTTCGTGTACTGGGGCCAG | |
| | GGCACCCTGGTGACCGTGAGCGCCGGCGGCGGCGG | |
| | CAGCGGCGGCGGCGGCAGCGGCGGCGGCGGCAGCC | |
| | AGGCCGTGGTGACCCAGGAGAGCGCCCTGACCACC | |
| | AGCCCCGGCGAGACCGTGACCCTGACCTGCAGGAC | |
| | CAGCACCGGCGCCGTGAGCATCAGGAACTACGCCA | |
| | ACTGGGTGCAGGAGAAGCCCGACCACCTGTTCACC | |
| | GGCCTGATCGGCGGCACCAACAACAGGGCCCCCGG | |
| | CGTGCCCGCCAGGTTCAGCGGCAGCCTGATCGGCG | |
| | ACAAGGCCGCCCTGACCATCACCGGCGCCCAGCCC | |
| | GAGGACGAGGCCATCTACTTCTGCGCCCTGCTGTA | |
| | CAGCAACTACTGGGTGTTCGGCGGCGGCACCAAGC | |
| | TGACCGTGCTGGGCGGCGGCGGCAGCCAGGTGCAG | |
| | CTGCAGCAGCCCGGCAGCGAGCTGGTGAGGCCCGG | |
| | CGCCAGCGTGAAGCTGAGCTGCAAGGCCAGCGGCT | |
| | ACACCTTCACCAGCTACTGGATGCACTGGGTGAAG | |
| | CAGAGGCCCGGCCAGGGCCTGGAGTGGATCGGCAA | |
| | CATCTACCCCAGGAGCGGCACCACCAACTACGACG | |
| | GCTACTTCAAGAGCAAGGCCACCCTGACCGTGGAC | |
| | ACCAGCAGCAGCACCGCCTACATGCAGCTGAGCAG | |
| | CCTGACCAGCGAGGACAGCGCCGTGTACTTCTGCA | |
| | CCAGGAGCGGCTACGACTACCCCTTCGTGTACTGG | |
| | GGCCAGGGCACCCTGGTGACCGTGAGCGCCACCAC | |
| | CACCCCCGCCCCCAGGCCCCCCACCCCCGCCCCCA | |
| | CCATCGCCAGCCCCCTGAGCCTGAGGCCCGAGGCC | |
| | TGCAGGCCCGCCGCCGGCGGCGCCGTGCACACCAG | |
| | GGGCCTGGACTTCGCCTGCGACTTCTGGGTGCTGG | |
| | TGGTGGTGGGCGGCGTGCTGGCCTGCTACAGCCTG | |
| | CTGGTGACCGTGGCCTTCATCATCTTCTGGGTGAG | |
| | GAGCAAGAGGAGCAGGCTGCTGCACAGCGACTACA | |
| | TGAACATGACCCCCAGGAGGCCCGGCCCCACCAGG | |
| | AAGCACTACCAGCCCTACGCCCCCCCCAGGGACTT | |
| | CGCCGCCTACAGGAGCAGGGTGAAGTTCAGCAGGA | |
| | GCGCCGACGCCCCCGCCTACCAGCAGGGCCAGAAC | |
| | CAGCTGTACAACGAGCTGAACCTGGGCAGGAGGGA | |
| | GGAGTACGACGTGCTGGACAAGAGGAGGGGCAGGG | |
| | ACCCCGAGATGGGCGGCAAGCCCAGGAGGAAGAAC | |
| | CCCCAGGAGGGCCTGTACAACGAGCTGCAGAAGGA | |
| | CAAGATGGCCGAGGCCTACAGCGAGATCGGCATGA | |
| | AGGGCGAGAGGAGGAGGGGCAAGGGCCACGACGGC | |
| | CTGTACCAGGGCCTGAGCACCGCCACCAAGGACAC | |
| | CTACGACGCCCTGCACATGCAGGCCCTGCCCCCCA | |
| | GGAGAAAGAGAGGAAGCGGAGAGGGCAGAGGAAGT | |
| | CTGCTAACATGCGGTGACGTCGAGGAGAATCCTGG | |
| | ACCTATGGTGAGCAAGGGCGAGGAGGATAACATGG | |
| | CCATCATCAAGGAGTTCATGCGCTTCAAGGTGCAC | |
| | ATGGAGGGCTCCGTGAACGGCCACGAGTTCGAGAT | |
| | CGAGGGCGAGGGCGAGGGCCGCCCCTACGAGGGCA | |
| | CCCAGACCGCCAAGCTGAAGGTGACCAAGGGTGGC | |
| | CCCCTGCCCTTCGCCTGGGACATCCTGTCCCCTCA | |
| | GTTCATGTACGGCTCCAAGGCCTACGTGAAGCACC | |
| | CCGCCGACATCCCCGACTACTTGAAGCTGTCCTTC | |
| | CCCGAGGGCTTCAAGTGGGAGCGCGTGATGAACTT | |
| | CGAGGACGGCGGCGTGGTGACCGTGACCCAGGACT | |
| | CCTCCCTGCAGGACGGCGAGTTCATCTACAAGGTG | |
| | AAGCTGCGCGGCACCAACTTCCCCTCCGACGGCCC | |
| | CGTAATGCAGAAGAAGACCATGGGCTGGGAGGCCT | |
| | CCTCCGAGCGGATGTACCCCGAGGACGGCGCCCTG | |
| | AAGGGCGAGATCAAGCAGAGGCTGAAGCTGAAGGA | |
| | CGGCGGCCACTACGACGCTGAGGTCAAGACCACCT | |
| | ACAAGGCCAAGAAGCCCGTGCAGCTGCCCGGCGCC | |
| | TACAACGTCAACATCAAGTTGGACATCACCTCCCA | |
| | CAACGAGGACTACACCATCGTGGAACAGTACGAAC | |
| | GCGCCGAGGGCCGCCACTCCACCGGCGGCATGGAC | |
| | GAGCTGTACAAGTAG | |
| 8 | ATGGCCCTGCCCGTGACCGCCCTGCTGCTGCCCCT | 174 |
| (HLx2- | GGCCCTGCTGCTGCACGCCGCCAGGCCCCAGGTGC | |
| 4C8- | AGCTGCAGCAGCCCGGCAGCGAGCTGGTGAGGCCC | |
| CD8- | GGCGCCAGCGTGAAGCTGAGCTGCAAGGCCAGCGG | |
| CART) | CTACACCTTCACCAGCTACTGGATGCACTGGGTGA | |
| | AGCAGAGGCCCGGCCAGGGCCTGGAGTGGATCGGC | |
| | AACATCTACCCCAGGAGCGGCACCACCAACTACGA | |
| | CGGCTACTTCAAGAGCAAGGCCACCCTGACCGTGG | |
| | ACACCAGCAGCAGCACCGCCTACATGCAGCTGAGC | |
| | AGCCTGACCAGCGAGGACAGCGCCGTGTACTTCTG | |
| | CACCAGGAGCGGCTACGACTACCCCTTCGTGTACT | |
| | GGGGCCAGGGCACCCTGGTGACCGTGAGCGCCGGC | |
| | GGCGGCGGCAGCCAGGCCGTGGTGACCCAGGAGAG | |
| | CGCCCTGACCACCAGCCCCGGCGAGACCGTGACCC | |
| | TGACCTGCAGGACCAGCACCGGCGCCGTGAGCATC | |
| | AGGAACTACGCCAACTGGGTGCAGGAGAAGCCCGA | |
| | CCACCTGTTCACCGGCCTGATCGGCGGCACCAACA | |
| | ACAGGGCCCCCGGCGTGCCCGCCAGGTTCAGCGGC | |
| | AGCCTGATCGGCGACAAGGCCGCCCTGACCATCAC | |
| | CGGCGCCCAGCCCGAGGACGAGGCCATCTACTTCT | |
| | GCGCCCTGCTGTACAGCAACTACTGGGTGTTCGGC | |
| | GGCGGCACCAAGCTGACCGTGCTGGGCGGCGGCGG | |
| | CAGCGGCGGCGGCGGCAGCGGCGGCGGCGGCAGCC | |
| | AGGTGCAGCTGCAGCAGCCCGGCAGCGAGCTGGTG | |
| | AGGCCCGGCGCCAGCGTGAAGCTGAGCTGCAAGGC | |
| | CAGCGGCTACACCTTCACCAGCTACTGGATGCACT | |
| | GGGTGAAGCAGAGGCCCGGCCAGGGCCTGGAGTGG | |
| | ATCGGCAACATCTACCCCAGGAGCGGCACCACCAA | |
| | CTACGACGGCTACTTCAAGAGCAAGGCCACCCTGA | |
| | CCGTGGACACCAGCAGCAGCACCGCCTACATGCAG | |
| | CTGAGCAGCCTGACCAGCGAGGACAGCGCCGTGTA | |
| | CTTCTGCACCAGGAGCGGCTACGACTACCCCTTCG | |
| | TGTACTGGGGCCAGGGCACCCTGGTGACCGTGAGC | |
| | GCCGGCGGCGGCGGCAGCCAGGCCGTGGTGACCCA | |
| | GGAGAGCGCCCTGACCACCAGCCCCGGCGAGACCG | |
| | TGACCCTGACCTGCAGGACCAGCACCGGCGCCGTG | |
| | AGCATCAGGAACTACGCCAACTGGGTGCAGGAGAA | |
| | GCCCGACCACCTGTTCACCGGCCTGATCGGCGGCA | |
| | CCAACAACAGGGCCCCCGGCGTGCCCGCCAGGTTC | |
| | AGCGGCAGCCTGATCGGCGACAAGGCCGCCCTGAC | |
| | CATCACCGGCGCCCAGCCCGAGGACGAGGCCATCT | |
| | ACTTCTGCGCCCTGCTGTACAGCAACTACTGGGTG | |
| | TTCGGCGGCGGCACCAAGCTGACCGTGCTGACCAC | |
| | CACCCCCGCCCCCAGGCCCCCCACCCCCGCCCCCA | |
| | CCATCGCCAGCCCCCTGAGCCTGAGGCCCGAGGCC | |
| | TGCAGGCCCGCCGCCGGCGGCGCCGTGCACACCAG | |
| | GGGCCTGGACTTCGCCTGCGACTTCTGGGTGCTGG | |
| | TGGTGGTGGGCGGCGTGCTGGCCTGCTACAGCCTG | |
| | CTGGTGACCGTGGCCTTCATCATCTTCTGGGTGAG | |
| | GAGCAAGAGGAGCAGGCTGCTGCACAGCGACTACA | |
| | TGAACATGACCCCCAGGAGGCCCGGCCCCACCAGG | |
| | AAGCACTACCAGCCCTACGCCCCCCCCAGGGACTT | |
| | CGCCGCCTACAGGAGCAGGGTGAAGTTCAGCAGGA | |
| | GCGCCGACGCCCCCGCCTACCAGCAGGGCCAGAAC | |
| | CAGCTGTACAACGAGCTGAACCTGGGCAGGAGGGA | |
| | GGAGTACGACGTGCTGGACAAGAGGAGGGGCAGGG | |
| | ACCCCGAGATGGGCGGCAAGCCCAGGAGGAAGAAC | |
| | CCCCAGGAGGGCCTGTACAACGAGCTGCAGAAGGA | |
| | CAAGATGGCCGAGGCCTACAGCGAGATCGGCATGA | |
| | AGGGCGAGAGGAGGAGGGGCAAGGGCCACGACGGC | |
| | CTGTACCAGGGCCTGAGCACCGCCACCAAGGACAC | |
| | CTACGACGCCCTGCACATGCAGGCCCTGCCCCCCA | |
| | GGAGAAAGAGAGGAAGCGGAGAGGGCAGAGGAAGT | |
| | CTGCTAACATGCGGTGACGTCGAGGAGAATCCTGG | |
| | ACCTATGGTGAGCAAGGGCGAGGAGGATAACATGG | |
| | CCATCATCAAGGAGTTCATGCGCTTCAAGGTGCAC | |
| | ATGGAGGGCTCCGTGAACGGCCACGAGTTCGAGAT | |
| | CGAGGGCGAGGGCGAGGGCCGCCCCTACGAGGGCA | |
| | CCCAGACCGCCAAGCTGAAGGTGACCAAGGGTGGC | |
| | CCCCTGCCCTTCGCCTGGGACATCCTGTCCCCTCA | |
| | GTTCATGTACGGCTCCAAGGCCTACGTGAAGCACC | |
| | CCGCCGACATCCCCGACTACTTGAAGCTGTCCTTC | |
| | CCCGAGGGCTTCAAGTGGGAGCGCGTGATGAACTT | |
| | CGAGGACGGCGGCGTGGTGACCGTGACCCAGGACT | |
| | CCTCCCTGCAGGACGGCGAGTTCATCTACAAGGTG | |
| | AAGCTGCGCGGCACCAACTTCCCCTCCGACGGCCC | |
| | CGTAATGCAGAAGAAGACCATGGGCTGGGAGGCCT | |
| | CCTCCGAGCGGATGTACCCCGAGGACGGCGCCCTG | |
| | AAGGGCGAGATCAAGCAGAGGCTGAAGCTGAAGGA | |
| | CGGCGGCCACTACGACGCTGAGGTCAAGACCACCT | |
| | ACAAGGCCAAGAAGCCCGTGCAGCTGCCCGGCGCC | |
| | TACAACGTCAACATCAAGTTGGACATCACCTCCCA | |
| | CAACGAGGACTACACCATCGTGGAACAGTACGAAC | |
| | GCGCCGAGGGCCGCCACTCCACCGGCGGCATGGAC | |
| | GAGCTGTACAAGTAG | |

TABLE 5B
CAR Amino Acid Sequences

| | | SEQ |
|---|---|---|
| | | ID |
| Construct | Amino acid sequence | NO: |
| 1 (LH-4C8- | MALPVTALLLPLALLLHAARPQAVVTQESALTTSPGETVTLTCRTST | 157 |
| CD8a- | GAVSIRNYANWVQEKPDHLFTGLIGGTNNRAPGVPARFSGSLIGDK | |
| CART) | AALTITGAQPEDEAIYFCALLYSNYWVFGGGTKLTVLGGGGSGGG | |
| | GSGGGGSQVQLQQPGSELVRPGASVKLSCKASGYTFTSYWMHW | |
| | VKQRPGQGLEWIGNIYPRSGTTNYDGYFKSKATLTVDTSSSTAYM | |
| | QLSSLTSEDSAVYFCTRSGYDYPFVYWGQGTLVTVSATTTPAPRP | |
| | PTPAPTIASPLSLRPEACRPAAGGAVHTRGLDFACDFWVLVVVGG | |
| | VLACYSLLVTVAFIIFWVRSKRSRLLHSDYMNMTPRRPGPTRKHYQ | |
| | PYAPPRDFAAYRSRVKFSRSADAPAYQQGQNQLYNELNLGRREE | |
| | YDVLDKRRGRDPEMGGKPRRKNPQEGLYNELQKDKMAEAYSEIG | |
| | MKGERRRGKGHDGLYQGLSTATKDTYDALHMQALPPRRKRGSG | |
| 2 (LH-4C8- | MALPVTALLLPLALLLHAARPQAVVTQESALTTSPGETVTLTCRTST | 158 |
| IgG4- | GAVSIRNYANWVQEKPDHLFTGLIGGTNNRAPGVPARFSGSLIGDK | |
| CART) | AALTITGAQPEDEAIYFCALLYSNYWVFGGGTKLTVLGGGGSGGG | |
| | GSGGGGSQVQLQQPGSELVRPGASVKLSCKASGYTFTSYWMHW | |
| | VKQRPGQGLEWIGNIYPRSGTTNYDGYFKSKATLTVDTSSSTAYM | |
| | QLSSLTSEDSAVYFCTRSGYDYPFVYWGQGTLVTVSAESKYGPPC | |
| | PSCPFWVLVVVGGVLACYSLLVTVAFIIFWVRSKRSRLLHSDYMNM | |
| | TPRRPGPTRKHYQPYAPPRDFAAYRSRVKFSRSADAPAYQQGQN | |
| | QLYNELNLGRREEYDVLDKRRGRDPEMGGKPRRKNPQEGLYNEL | |
| | QKDKMAEAYSEIGMKGERRRGKGHDGLYQGLSTATKDTYDALHM | |
| | QALPPRRKRGSG | |
| 3 (LH-4C8- | MALPVTALLLPLALLLHAARPQAVVTQESALTTSPGETVTLTCRTST | 159 |
| IgG4-Long- | GAVSIRNYANWVQEKPDHLFTGLIGGTNNRAPGVPARFSGSLIGDK | |
| CART) | AALTITGAQPEDEAIYFCALLYSNYWVFGGGTKLTVLGGGGSGGG | |
| | GSGGGGSQVQLQQPGSELVRPGASVKLSCKASGYTFTSYWMHW | |
| | VKQRPGQGLEWIGNIYPRSGTTNYDGYFKSKATLTVDTSSSTAYM | |
| | QLSSLTSEDSAVYFCTRSGYDYPFVYWGQGTLVTVSAESKYGPPC | |
| | PPCPAPPVAGPSVFLFPPKPKDTLMISRTPEVTCVVVDVSQEDPEV | |
| | QFNWYVDGVEVHNAKTKPREEQFQSTYRVVSVLTVLHQDWLNGK | |
| | EYKCKVSNKGLPSSIEKTISKAKGQPREPQVYTLPPSQEEMTKNQV | |
| | SLTCLVKGFYPSDIAVEWESNGQPENNYKTTPPVLDSDGSFFLYSR | |
| | LTVDKSRWQEGNVFSCSVMHEALHNHYTQKSLSLSLGKMFWVLV | |
| | VVGGVLACYSLLVTVAFIIFWVRSKRSRLLHSDYMNMTPRRPGPTR | |
| | KHYQPYAPPRDFAAYRSRVKFSRSADAPAYQQGQNQLYNELNLG | |
| | RREEYDVLDKRRGRDPEMGGKPRRKNPQEGLYNELQKDKMAEAY | |
| | SEIGMKGERRRGKGHDGLYQGLSTATKDTYDALHMQALPPRRKR | |
| | GSG | |
| 4 (HL-4C8- | MALPVTALLLPLALLLHAARPQVQLQQPGSELVRPGASVKLSCKAS | 160 |
| CD8a- | GYTFTSYWMHWVKQRPGQGLEWIGNIYPRSGTTNYDGYFKSKAT | |
| CART) | LTVDTSSSTAYMQLSSLTSEDSAVYFCTRSGYDYPFVYWGQGTLV | |
| | TVSAGGGGSGGGGSGGGGSQAVVTQESALTTSPGETVTLTCRTS | |
| | TGAVSIRNYANWVQEKPDHLFTGLIGGTNNRAPGVPARFSGSLIGD | |
| | KAALTITGAQPEDEAIYFCALLYSNYWVFGGGTKLTVLTTTPAPRPP | |
| | TPAPTIASPLSLRPEACRPAAGGAVHTRGLDFACDFWVLVVVGGVL | |
| | ACYSLLVTVAFIIFWVRSKRSRLLHSDYMNMTPRRPGPTRKHYQPY | |
| | APPRDFAAYRSRVKFSRSADAPAYQQGQNQLYNELNLGRREEYD | |
| | VLDKRRGRDPEMGGKPRRKNPQEGLYNELQKDKMAEAYSEIGMK | |
| | GERRRGKGHDGLYQGLSTATKDTYDALHMQALPPRRKRGSG | |
| 5 (HL-4C8- | MALPVTALLLPLALLLHAARPQVQLQQPGSELVRPGASVKLSCKAS | 161 |
| IgG4- | GYTFTSYWMHWVKQRPGQGLEWIGNIYPRSGTTNYDGYFKSKAT | |
| CART) | LTVDTSSSTAYMQLSSLTSEDSAVYFCTRSGYDYPFVYWGQGTLV | |
| | TVSAGGGGSGGGGSGGGGSQAVVTQESALTTSPGETVTLTCRTS | |
| | TGAVSIRNYANWVQEKPDHLFTGLIGGTNNRAPGVPARFSGSLIGD | |
| | KAALTITGAQPEDEAIYFCALLYSNYWVFGGGTKLTVLESKYGPPC | |
| | PSCPFWVLVVVGGVLACYSLLVTVAFIIFWVRSKRSRLLHSDYMNM | |
| | TPRRPGPTRKHYQPYAPPRDFAAYRSRVKFSRSADAPAYQQGQN | |
| | QLYNELNLGRREEYDVLDKRRGRDPEMGGKPRRKNPQEGLYNEL | |
| | QKDKMAEAYSEIGMKGERRRGKGHDGLYQGLSTATKDTYDALHM | |
| | QALPPRRKRGSG | |
| 6 (HL-4C8- | MALPVTALLLPLALLLHAARPQVQLQQPGSELVRPGASVKLSCKAS | 162 |
| IgG4-Long- | GYTFTSYWMHWVKQRPGQGLEWIGNIYPRSGTTNYDGYFKSKAT | |
| CART) | LTVDTSSSTAYMQLSSLTSEDSAVYFCTRSGYDYPFVYWGQGTLV | |
| | TVSAGGGGSGGGGSGGGGSQAVVTQESALTTSPGETVTLTCRTS | |
| | TGAVSIRNYANWVQEKPDHLFTGLIGGTNNRAPGVPARFSGSLIGD | |
| | KAALTITGAQPEDEAIYFCALLYSNYWVFGGGTKLTVLESKYGPPC | |
| | PPCPAPPVAGPSVFLFPPKPKDTLMISRTPEVTCVVVDVSQEDPEV | |
| | QFNWYVDGVEVHNAKTKPREEQFQSTYRVVSVLTVLHQDWLNGK | |
| | EYKCKVSNKGLPSSIEKTISKAKGQPREPQVYTLPPSQEEMTKNQV | |
| | SLTCLVKGFYPSDIAVEWESNGQPENNYKTTPPVLDSDGSFFLYSR | |
| | LTVDKSRWQEGNVFSCSVMHEALHNHYTQKSLSLSLGKMFWVLV | |
| | VVGGVLACYSLLVTVAFIIFWVRSKRSRLLHSDYMNMTPRRPGPTR | |
| | KHYQPYAPPRDFAAYRSRVKFSRSADAPAYQQGQNQLYNELNLG | |
| | RREEYDVLDKRRGRDPEMGGKPRRKNPQEGLYNELQKDKMAEAY | |
| | SEIGMKGERRRGKGHDGLYQGLSTATKDTYDALHMQALPPRRKR | |
| | GSG | |
| 7 (LHx2- | MALPVTALLLPLALLLHAARPQAVVTQESALTTSPGETVTLTCRTST | 163 |
| 4C8-CD8- | GAVSIRNYANWVQEKPDHLFTGLIGGTNNRAPGVPARFSGSLIGDK | |
| CART) | AALTITGAQPEDEAIYFCALLYSNYWVFGGGTKLTVLGGGGSQVQL | |
| | QQPGSELVRPGASVKLSCKASGYTFTSYWMHWVKQRPGQGLEWI | |
| | GNIYPRSGTTNYDGYFKSKATLTVDTSSSTAYMQLSSLTSEDSAVY | |
| | FCTRSGYDYPFVYWGQGTLVTVSAGGGGSGGGGSGGGGSQAVV | |
| | TQESALTTSPGETVTLTCRTSTGAVSIRNYANWVQEKPDHLFTGLI | |
| | GGTNNRAPGVPARFSGSLIGDKAALTITGAQPEDEAIYFCALLYSNY | |
| | WVFGGGTKLTVLGGGGSQVQLQQPGSELVRPGASVKLSCKASGY | |
| | TFTSYWMHWVKQRPGQGLEWIGNIYPRSGTTNYDGYFKSKATLTV | |
| | DTSSSTAYMQLSSLTSEDSAVYFCTRSGYDYPFVYWGQGTLVTVS | |
| | ATTTPAPRPPTPAPTIASPLSLRPEACRPAAGGAVHTRGLDFACDF | |
| | WVLVVVGGVLACYSLLVTVAFIIFWVRSKRSRLLHSDYMNMTPRRP | |
| | GPTRKHYQPYAPPRDFAAYRSRVKFSRSADAPAYQQGQNQLYNE | |
| | LNLGRREEYDVLDKRRGRDPEMGGKPRRKNPQEGLYNELQKDKM | |
| | AEAYSEIGMKGERRRGKGHDGLYQGLSTATKDTYDALHMQALPPR | |
| | RKRGSG | |
| 8 (HLx2- | MALPVTALLLPLALLLHAARPQVQLQQPGSELVRPGASVKLSCKAS | 164 |
| 4C8-CD8- | GYTFTSYWMHWVKQRPGQGLEWIGNIYPRSGTTNYDGYFKSKAT | |
| CART) | LTVDTSSSTAYMQLSSLTSEDSAVYFCTRSGYDYPFVYWGQGTLV | |
| | TVSAGGGGSQAVVTQESALTTSPGETVTLTCRTSTGAVSIRNYAN | |
| | WVQEKPDHLFTGLIGGTNNRAPGVPARFSGSLIGDKAALTITGAQP | |
| | EDEAIYFCALLYSNYWVFGGGTKLTVLGGGGSGGGGSGGGGSQV | |
| | QLQQPGSELVRPGASVKLSCKASGYTFTSYWMHWVKQRPGQGL | |
| | EWIGNIYPRSGTTNYDGYFKSKATLTVDTSSSTAYMQLSSLTSEDS | |
| | AVYFCTRSGYDYPFVYWGQGTLVTVSAGGGGSQAVVTQESALTT | |
| | SPGETVTLTCRTSTGAVSIRNYANWVQEKPDHLFTGLIGGTNNRAP | |
| | GVPARFSGSLIGDKAALTITGAQPEDEAIYFCALLYSNYWVFGGGT | |
| | KLTVLTTTPAPRPPTPAPTIASPLSLRPEACRPAAGGAVHTRGLDFA | |
| | CDFWVLVVVGGVLACYSLLVTVAFIIFWVRSKRSRLLHSDYMNMTP | |
| | RRPGPTRKHYQPYAPPRDFAAYRSRVKFSRSADAPAYQQGQNQL | |
| | YNELNLGRREEYDVLDKRRGRDPEMGGKPRRKNPQEGLYNELQK | |
| | DKMAEAYSEIGMKGERRRGKGHDGLYQGLSTATKDTYDALHMQA | |
| | LPPRRKRGSG | |

7.2.1.2 Transduction and Expansion

Lentivirus was produced in HEK293T cells transfected with pGO-408, pVSVG, and pPAX2 using PEI overnight. The lentiviral supernatant was harvested after 24 hours. Healthy donor PBMCs were isolated using Lymphoprep density centrifugation followed by plastic adherence to get rid of adherent cells. The non-adherent PBMCs were cultured in RPMI-1640 Dutch modification with 10% FBS, 50 μM 2-mercaptoethanol, and 20 ng/ml rIL-2 and were activated using human T-activator CD3/CD28 Dynabeads. Following activation, the T cells were transduced twice with viral supernatant for 24 hours. Transduced CAR T cells were expanded in culture medium at densities between 0.5×10⁶ cells/mL and 1×10⁶ cells/mL until used for studies.

7.2.1.3 Cytotoxicity Assay

HaCaT WT and COSMC KO cells were seeded at a density of 20,000 cells per well in 96-well plates and allowed to adhere overnight. Two days later, CAR T cells were added at effector-target cell ratios of 5:1 or 3:1 and were incubated for 6 hours. Cytotoxicity of target cells co-cultured with CAR T cells was evaluated by lactate dehydrogenase cytoxicity assay (abcam) following manufacturer's instructions. For 100% cell death controls, 1% tween in PBS was used for complete lysis of all cells. To assess IFN-γ production by the CAR T cells, supernatant was harvested from the co-cultures, and ELISA was performed according to manufacturer's instructions (abcam).

7.2.2. Results

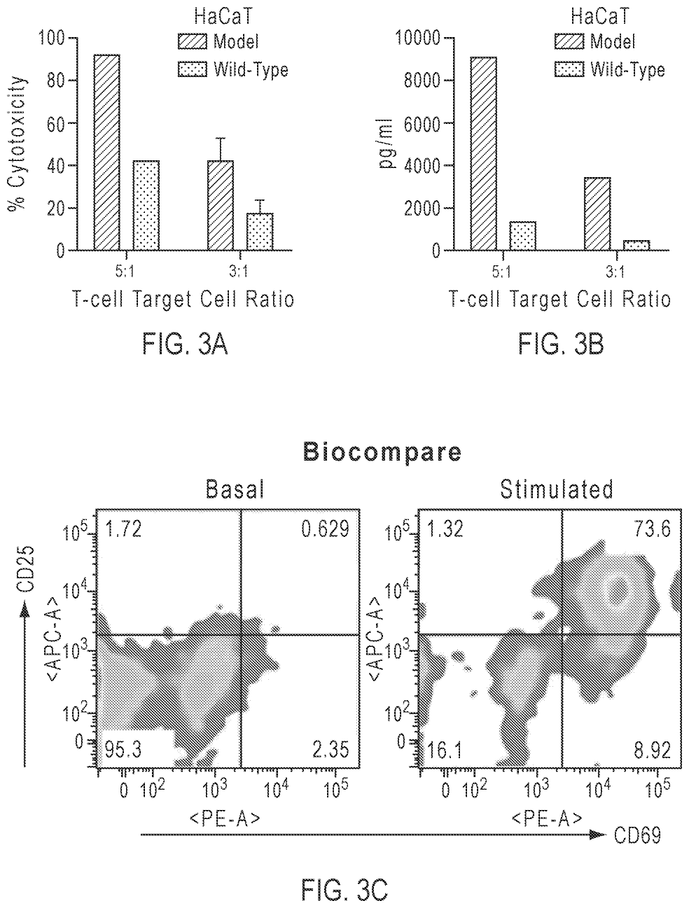

It was found that 4C8 CAR T cells (Construct 1) exerted approximately 2 to 3-fold higher cytotoxicity against COSMC KO HaCaT cells than their wildtype counterpart, although a cytotoxicity of ˜45% was also observed in the HaCaT WT co-culture (FIG. 3A). Consistently, a much higher production of IFN-γ in the co-culture with COSMC KO HaCaT cells than the WT HaCaT cells was found, indicating a much stronger activation of the CAR T cells by the COSMC KO cells (FIG. 3B).

Untransfected T cells did not exhibit significant cytotoxicity to either HaCaT WT cells or COSMC KO HaCaT cells (FIG. 4). Additionally, the CAR-T was observed to be more functional when the VL was placed at the N-terminal side of the VH (FIG. 4).

### 7.3 Example 3: 10H4-Based CARs

Chimeric antigen receptors (CARs) having VH and VL domains of 10H4 were designed.

7.3.1. Materials and Methods

7.3.1.1 Vector Design

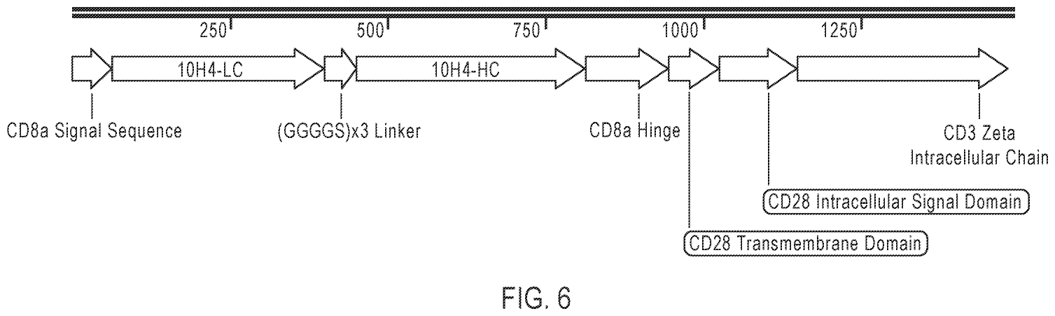

A CAR construct having scFvs having VH and VL domains of 10H4 was designed. The VH and VL were attached together with one long linker (GGGGS)₃ (SEQ ID NO:184) (see FIG. 6). The VH and VL were attached to a CD8a hinge followed by a second generation CAR-T (CD28 intracellular signal domain, and a CD3-zeta intracellular chain). The N-terminus of the scFvs was attached to a CD8a signal sequence. The 10H4 CAR-Ts were subcloned into the Virapower lentivirus vector pLENTI6.3-V5-DEST (Invitrogen).

The nucleotide sequence encoding the 10H4 CAR is shown in Table 6A. The amino acid sequence of the 10H4 CAR is shown in Table 6B.

TABLE 6A
Nucleotide sequences encoding 10H4 CAR

| | | SEQ |
|---|---|---|
| Con- | | ID |
| struct | Nucleic acid sequence | NO: |
| LH- | ATGGCTCTGCCTGTCACCGCGCTGCTCCTTCCCTTA | 260 |
| 10H4- | GCCCTGCTGCTGCACGCTGCTCGTCCAGCGCAGGTC | |
| CD8a- | CTGACTCAGACACCCGCGTCCGTGTCCGCAGCGGTC | |
| CART | GGGGGCACTGTGACCATCAACTGCCAGGCAAGCCAG | |
| | TCCGTGTACAATAACAACCAGCTGAGTTGGTACCAA | |
| | CAGAAGCCTGGGCAGCCCCTAAAGCAGCTGATTTAC | |
| | AAGGCTTCCACCCTTGCTTCCGGCGTCCCCTCTAGG | |
| | TTTAAGGGCTCTGGGAGCGGTTCCCAGTTCACCCTG | |
| | ACCATCAGCGATCTGGAATGCGATGACGCCGCCACC | |
| | TACTTCTGTGCCGGCGGCTACAAGGGGGACATCCAC | |
| | CCCTTCGGTGGCGGTACTGAGGTGGTAGTCAAGGGA | |
| | GACCCGGTGGGCGGTGGCGGCTCCGGAGGCGGTGGT | |
| | TCTGGTGGAGGAGGCAGCCAGTGTCAGTCTCTGGAG | |
| | GAGAGCGGGGGCCGCCTGGTGACCCCCGGTACTCCG | |
| | CTCACTCTGACCTGCACTGCATCTGGCTTCACCATT | |
| | AACACGTACCACATGGGATGGTTCCGCCAGGCCCCT | |
| | GGCAAGGGCCTCCAGTATATTGGCATCGTGAGTCAT | |
| | GACGTGGGCACCTACTACGCTACCTGGGCAAAAGGA | |
| | CGCTTCACGATCTCCAAGACCTCCTCGACCACAGTG | |
| | GACCTGCGCATGCCCAGCCCAACGACCGAGGACACC | |
| | GCCACTTACATCTGCGCCCGTGGGCCGGGCTATTGG | |
| | ACCTTCAACCTGTGGGGCCAGGGCACCCTGGTGACA | |
| | GTTTCCTCGGGACAGCCAAAGACCACAACCCCCGCC | |
| | CCGCGCCCTCCTACCCCGGCCCCTACGATAGCGTCA | |
| | CCCCTGTCTCTCCGCCCCGAGGCTTGTAGGCCTGCC | |
| | GCTGGTGGGGCCGTCCACACCCGTGGCTTGGATTTC | |
| | GCTTGCGACTTCTGGGTCTTGGTGGTGGTGGGGGGA | |
| | GTGCTGGCGTGTTACTCGTTGCTGGTCACCGTGGCG | |
| | TTCATCATCTTCTGGGTACGCAGCAAGCGCAGCCGT | |
| | CTCCTGCACTCCGACTACATGAATATGACACCTAGA | |
| | CGGCCCGGCCCTACCCGGAAGCACTACCAGCCCTAT | |
| | GCTCCCCCGAGGGACTTTGCTGCGTACCGCTCCCGC | |
| | GTGAAATTTTCACGCTCCGCGGACGCTCCGGCCTAT | |
| | CAGCAGGGTCAGAACCAGCTTTACAACGAGCTCAAC | |
| | CTGGGCCGACGTGAAGAGTACGATGTGCTGGACAAG | |
| | CGCCGGGGACGCGATCCAGAGATGGGCGGCAAGCCC | |
| | AGACGCAAGAACCCTCAGGAGGGCTTGTACAACGAG | |
| | CTGCAGAAGGACAAAATGGCCGAGGCCTACTCCGAG | |
| | ATCGGTATGAAGGGGGAGCGGCGCCGCGGCAAAGGC | |
| | CACGACGGCCTCTATCAGGGGCTTTCGACCGCCACT | |
| | AAGGACACCTACGACGCACTTCATATGCAAGCCCTG | |
| | CCCCCGCGCTAG | |

TABLE 6B
CAR Amino Acid Sequences

| | | SEQ |
|---|---|---|
| Con- | | ID |
| struct | Amino acid sequence | NO: |
| LH- | MALPVTALLLPLALLLHAARPAQVLTQTPASVSAAV | 261 |
| 10H4- | GGTVTINCQASQSVYNNNQLSWYQQKPGQPLKQLIY | |
| CD8a- | KASTLASGVPSRFKGSGSGSQFTLTISDLECDDAAT | |
| CART | YFCAGGYKGDIHPFGGGTEVVVKGDPVGGGGSGGGG | |
| | SGGGGSQCQSLEESGGRLVTPGTPLTLTCTASGFTI | |
| | NTYHMGWFRQAPGKGLQYIGIVSHDVGTYYATWAKG | |
| | RFTISKTSSTTVDLRMPSPTTEDTATYICARGPGYW | |
| | TFNLWGQGTLVTVSSGQPKTTTPAPRPPTPAPTIAS | |
| | PLSLRPEACRPAAGGAVHTRGLDFACDFWVLVVVGG | |
| | VLACYSLLVTVAFIIFWVRSKRSRLLHSDYMNMTPR | |
| | RPGPTRKHYQPYAPPRDFAAYRSRVKFSRSADAPAY | |
| | QQGQNQLYNELNLGRREEYDVLDKRRGRDPEMGGKP | |
| | RRKNPQEGLYNELQKDKMAEAYSEIGMKGERRRGKG | |
| | HDGLYQGLSTATKDTYDALHMQALPPR* | |

### 8. SPECIFIC EMBODIMENTS, CITATION OF REFERENCES

While various specific embodiments have been illustrated and described, it will be appreciated that various changes can be made without departing from the spirit and scope of the disclosure(s). The present disclosure is exemplified by the numbered embodiments set forth below.

1. An anti-glyco-CD44 antibody or antigen binding fragment that specifically binds to a CD44v6 peptide GYRQTPKEDSHSTTGTAAA (SEQ ID NO:165) that has been glycosylated with GalNAc on threonine at amino acid position 5 of SEQ ID NO: 165 and serine at amino acid position 12 of SEQ ID NO: 165 (“the CD44v6 glycopeptide”).

2. The anti-glyco-CD44 antibody or antigen binding fragment of embodiment 1, wherein the anti-glyco-CD44 antibody or antigen binding fragment competes with an antibody or antigen binding fragment comprising a heavy chain variable (VH) sequence of SEQ ID NO:1 and a light chain variable (VL) sequence of SEQ ID NO:2 for binding to the CD44v6 glycopeptide.

3. The anti-glyco-CD44 antibody or antigen binding fragment of embodiment 1, wherein the anti-glyco-CD44 antibody or antigen binding fragment competes with an antibody or antigen binding fragment comprising a heavy chain variable (VH) sequence of SEQ ID NO:23 and a light chain variable (VL) sequence of SEQ ID NO:24 for binding to the CD44v6 glycopeptide.

4. The anti-glyco-CD44 antibody or antigen binding fragment of embodiment 1, wherein the anti-glyco-CD44 antibody or antigen binding fragment competes with an antibody or antigen binding fragment comprising a heavy chain variable (VH) sequence of SEQ ID NO:45 and a light chain variable (VL) sequence of SEQ ID NO:46 for binding to the CD44v6 glycopeptide.

5. The anti-glyco-CD44 antibody or antigen binding fragment of embodiment 1, wherein the anti-glyco-CD44 antibody or antigen binding fragment competes with an antibody or antigen binding fragment comprising a heavy chain variable (VH) sequence of SEQ ID NO:67 and a light chain variable (VL) sequence of SEQ ID NO:68 for binding to the CD44v6 glycopeptide.

6. The anti-glyco-CD44 antibody or antigen binding fragment of embodiment 1, wherein the anti-glyco-CD44 antibody or antigen binding fragment competes with an antibody or antigen binding fragment comprising a heavy chain variable (VH) sequence of SEQ ID NO:206 and a light chain variable (VL) sequence of SEQ ID NO:207 for binding to the CD44v6 glycopeptide.

7. The anti-glyco-CD44 antibody or antigen binding fragment of any one of embodiments 1 to 6, which (i) specifically binds to COSMC knock-out HaCaT cells and/or (ii) specifically binds to COSMC knock-out HEK293 cells recombinantly expressing CD44.

8. The anti-glyco-CD44 antibody or antigen binding fragment of embodiment 7, wherein the anti-glyco-CD44 antibody or antigen binding fragment competes with an antibody or antigen binding fragment comprising a heavy chain variable (VH) sequence of SEQ ID NO:1 and a light chain variable (VL) sequence of SEQ ID NO:2 for binding to COSMC knock-out HaCaT cells and/or COSMC knock-out HEK293 cells recombinantly expressing CD44.

9. The anti-glyco-CD44 antibody or antigen binding fragment of embodiment 7, wherein the anti-glyco-CD44 antibody or antigen binding fragment competes with an antibody or antigen binding fragment comprising a heavy chain variable (VH) sequence of SEQ ID NO:23 and a light chain variable (VL) sequence of SEQ ID NO:24 for binding to COSMC knock-out HaCaT cells and/or COSMC knock-out HEK293 cells recombinantly expressing CD44.

10. The anti-glyco-CD44 antibody or antigen binding fragment of embodiment 7, wherein the anti-glyco-CD44 antibody or antigen binding fragment competes with an antibody or antigen binding fragment comprising a heavy chain variable (VH) sequence of SEQ ID NO:45 and a light chain variable (VL) sequence of SEQ ID NO:46 for binding to COSMC knock-out HaCaT cells and/or COSMC knock-out HEK293 cells recombinantly expressing CD44.

11. The anti-glyco-CD44 antibody or antigen binding fragment of embodiment 7, wherein the anti-glyco-CD44 antibody or antigen binding fragment competes with an antibody or antigen binding fragment comprising a heavy chain variable (VH) sequence of SEQ ID NO:67 and a light chain variable (VL) sequence of SEQ ID NO:68 for binding to COSMC knock-out HaCaT cells and/or COSMC knock-out HEK293 cells recombinantly expressing CD44.

12. The anti-glyco-CD44 antibody or antigen binding fragment of embodiment 7, wherein the anti-glyco-CD44 antibody or antigen binding fragment competes with an antibody or antigen binding fragment comprising a heavy chain variable (VH) sequence of SEQ ID NO:206 and a light chain variable (VL) sequence of SEQ ID NO:207 for binding to COSMC knock-out HaCaT cells and/or COSMC knock-out HEK293 cells recombinantly expressing CD44.

13. The anti-glyco-CD44 antibody or antigen-binding fragment of any one of embodiments 1 to 12, comprising:
(a) a complementarity determining region (CDR) H1 comprising the amino acid sequence of SEQ ID NO:89, SEQ ID NO:93, SEQ ID NO:97, SEQ ID NO:125, SEQ ID NO:153, SEQ ID NO:228, SEQ ID NO:232, SEQ ID NO:236, SEQ ID NO:246, or SEQ ID NO: 256;
(b) a CDR-H2 comprising the amino acid sequence of SEQ ID NO:90, SEQ ID NO:94, SEQ ID NO:98, SEQ ID NO:229, SEQ ID NO:233, SEQ ID NO:237;
(c) a CDR-H3 comprising the amino acid sequence of SEQ ID NO:103, SEQ ID NO: 109, SEQ ID NO: 115, SEQ ID NO:121, SEQ ID NO:131, SEQ ID NO:137, SEQ ID NO:143, SEQ ID NO: 149; SEQ ID NO:242, or SEQ ID NO:252;
(d) a CDR-L1 comprising the amino acid sequence of SEQ ID NO:104, SEQ ID NO:110, SEQ ID NO:116, SEQ ID NO:122, SEQ ID NO:132, SEQ ID NO:138, SEQ ID NO:144, SEQ ID NO:150, SEQ ID NO:243, or SEQ ID NO:253;
(e) a CDR-L2 comprising the amino acid sequence of SEQ ID NO:91, SEQ ID NO:95, SEQ ID NO:230, SEQ ID NO:234; and
(f) a CDR-L3 comprising the amino acid sequence of SEQ ID NO:92 or SEQ ID NO:231.

14. The anti-glyco-CD44 antibody or antigen-binding fragment of embodiment 13, wherein the amino acid designated X₁ in SEQ ID NO:89, SEQ ID NO:97, and SEQ ID NO:125 is Y.

15. The anti-glyco-CD44 antibody or antigen-binding fragment of embodiment 13, wherein the amino acid designated X₁ in SEQ ID NO:89, SEQ ID NO:97, and SEQ ID NO:125 is F.

16. The anti-glyco-CD44 antibody or antigen-binding fragment of any one of embodiments 13 to 15, wherein the amino acid designated X₂ in SEQ ID NO:89, SEQ ID NO:97, and SEQ ID NO:125 is T.

17. The anti-glyco-CD44 antibody or antigen-binding fragment of any one of embodiments 13 to 15, wherein the amino acid designated X₂ in SEQ ID NO:89, SEQ ID NO:97, and SEQ ID NO:125 is S.

18. The anti-glyco-CD44 antibody or antigen-binding fragment of any one of embodiments 13 to 17, wherein the amino acid designated X₃ in SEQ ID NO:89, SEQ ID NO:93, SEQ ID NO:97, SEQ ID NO:125, and SEQ ID NO:153 is Y.

19. The anti-glyco-CD44 antibody or antigen-binding fragment of any one of embodiments 13 to 17, wherein the amino acid designated X₃ in SEQ ID NO:89, SEQ ID NO:93, SEQ ID NO:97, SEQ ID NO:125, and SEQ ID NO:153 is F.

20. The anti-glyco-CD44 antibody or antigen-binding fragment of any one of embodiments 13 to 19, wherein the amino acid designated X₄ in SEQ ID NO:89, SEQ ID NO:93, and SEQ ID NO:125 is W.

21. The anti-glyco-CD44 antibody or antigen-binding fragment of any one of embodiments 13 to 19, wherein the amino acid designated X₄ in SEQ ID NO:89, SEQ ID NO:93, and SEQ ID NO:125 is A.

22. The anti-glyco-CD44 antibody or antigen-binding fragment of any one of embodiments 13 to 19, wherein the amino acid designated X₄ in SEQ ID NO:89, SEQ ID NO:93, and SEQ ID NO:125 is G.

23. The anti-glyco-CD44 antibody or antigen-binding fragment of any one of embodiments 13 to 22, wherein the amino acid designated X₅ in SEQ ID NO:93 and SEQ ID NO:125 is M.

24. The anti-glyco-CD44 antibody or antigen-binding fragment of any one of embodiments 13 to 22, wherein the amino acid designated X₅ in SEQ ID NO:93 and SEQ ID NO:125 is I.

25. The anti-glyco-CD44 antibody or antigen-binding fragment of any one of embodiments 13 to 24, wherein the amino acid designated X₆ in SEQ ID NO:93 and SEQ ID NO:125 is H.

26. The anti-glyco-CD44 antibody or antigen-binding fragment of any one of embodiments 13 to 24, wherein the amino acid designated X₆ in SEQ ID NO:93 and SEQ ID NO:125 is S.

27. The anti-glyco-CD44 antibody or antigen-binding fragment of any one of embodiments 13 to 26, wherein the amino acid designated X₇ in SEQ ID NO:94 is N.

28. The anti-glyco-CD44 antibody or antigen-binding fragment of any one of embodiments 13 to 26, wherein the amino acid designated X₇ in SEQ ID NO:94 is E.

29. The anti-glyco-CD44 antibody or antigen-binding fragment of any one of embodiments 13 to 26, wherein the amino acid designated X₇ in SEQ ID NO:94 is Y.

30. The anti-glyco-CD44 antibody or antigen-binding fragment of any one of embodiments 13 to 29, wherein the amino acid designated X₈ in SEQ ID NO:90, SEQ ID NO:94, and SEQ ID NO:98 is Y.

31. The anti-glyco-CD44 antibody or antigen-binding fragment of any one of embodiments 13 to 29, wherein the amino acid designated X₈ in SEQ ID NO:90, SEQ ID NO:94, and SEQ ID NO:98 is S.

32. The anti-glyco-CD44 antibody or antigen-binding fragment of any one of embodiments 13 to 31, wherein the amino acid designated X₉ in SEQ ID NO:90, SEQ ID NO:94, and SEQ ID NO:98 is P.

33. The anti-glyco-CD44 antibody or antigen-binding fragment of any one of embodiments 13 to 31, wherein the amino acid designated X₉ in SEQ ID NO:90, SEQ ID NO:94, and SEQ ID NO:98 is S.

34. The anti-glyco-CD44 antibody or antigen-binding fragment of any one of embodiments 13 to 33, wherein the amino acid designated X₁₀ in SEQ ID NO:90, SEQ ID NO:94, and SEQ ID NO:98 is R.

35. The anti-glyco-CD44 antibody or antigen-binding fragment of any one of embodiments 13 to 33, wherein the amino acid designated X₁₀ in SEQ ID NO:90, SEQ ID NO:94, and SEQ ID NO:98 is G.

36. The anti-glyco-CD44 antibody or antigen-binding fragment of any one of embodiments 13 to 35, wherein the amino acid designated X₁₁ in SEQ ID NO:90, SEQ ID NO:94, and SEQ ID NO:98 is S.

37. The anti-glyco-CD44 antibody or antigen-binding fragment of any one of embodiments 13 to 35, wherein the amino acid designated X₁₁ in SEQ ID NO:90, SEQ ID NO:94, and SEQ ID NO:98 is G.

38. The anti-glyco-CD44 antibody or antigen-binding fragment of any one of embodiments 13 to 37, wherein the amino acid designated X₁₂ in SEQ ID NO:90, SEQ ID NO:94, and SEQ ID NO:98 is G.

39. The anti-glyco-CD44 antibody or antigen-binding fragment of any one of embodiments 13 to 37, wherein the amino acid designated X₁₂ in SEQ ID NO:90, SEQ ID NO:94, and SEQ ID NO:98 is S.

40. The anti-glyco-CD44 antibody or antigen-binding fragment of any one of embodiments 13 to 39, wherein the amino acid designated X₁₃ in SEQ ID NO:90, SEQ ID NO:94, and SEQ ID NO:98 is T.

41. The anti-glyco-CD44 antibody or antigen-binding fragment of any one of embodiments 13 to 39, wherein the amino acid designated X₁₃ in SEQ ID NO:90, SEQ ID NO:94, and SEQ ID NO:98 is Y.

42. The anti-glyco-CD44 antibody or antigen-binding fragment of any one of embodiments 13 to 41, wherein the amino acid designated X₁₄ in SEQ ID NO:90 and SEQ ID NO:94 is T.

43. The anti-glyco-CD44 antibody or antigen-binding fragment of any one of embodiments 13 to 41, wherein the amino acid designated X₁₄ in SEQ ID NO:90 and SEQ ID NO:94 is I.

44. The anti-glyco-CD44 antibody or antigen-binding fragment of any one of embodiments 13 to 43, wherein the amino acid designated X₁₅ in SEQ ID NO:94 is N.

45. The anti-glyco-CD44 antibody or antigen-binding fragment of any one of embodiments 13 to 43, wherein the amino acid designated X₁₅ in SEQ ID NO:94 is Y.

46. The anti-glyco-CD44 antibody or antigen-binding fragment of any one of embodiments 13 to 45, wherein the amino acid designated X₁₆ in SEQ ID NO:94 is D.

47. The anti-glyco-CD44 antibody or antigen-binding fragment of any one of embodiments 13 to 45, wherein the amino acid designated X₁₆ in SEQ ID NO:94 is P.

48. The anti-glyco-CD44 antibody or antigen-binding fragment of any one of embodiments 13 to 45, wherein the amino acid designated X₁₆ in SEQ ID NO:94 is A.

49. The anti-glyco-CD44 antibody or antigen-binding fragment of any one of embodiments 13 to 48, wherein the amino acid designated X₁₇ in SEQ ID NO:94 is G.

50. The anti-glyco-CD44 antibody or antigen-binding fragment of any one of embodiments 13 to 48, wherein the amino acid designated X₁₇ in SEQ ID NO:94 is D.

51. The anti-glyco-CD44 antibody or antigen-binding fragment of any one of embodiments 13 to 50, wherein the amino acid designated X₁₈ in SEQ ID NO:94 is Y.

52. The anti-glyco-CD44 antibody or antigen-binding fragment of any one of embodiments 13 to 50, wherein the amino acid designated X₁₈ in SEQ ID NO:94 is T.

53. The anti-glyco-CD44 antibody or antigen-binding fragment of any one of embodiments 13 to 52, wherein the amino acid designated X₁₉ in SEQ ID NO:94 is F.

54. The anti-glyco-CD44 antibody or antigen-binding fragment of any one of embodiments 13 to 52, wherein the amino acid designated X₁₉ in SEQ ID NO:94 is V.

55. The anti-glyco-CD44 antibody or antigen-binding fragment of any one of embodiments 13 to 54, wherein the amino acid designated X₂₀ in SEQ ID NO:94 is K.

56. The anti-glyco-CD44 antibody or antigen-binding fragment of any one of embodiments 13 to 54, wherein the amino acid designated X₂₀ in SEQ ID NO:94 is T.

57. The anti-glyco-CD44 antibody or antigen-binding fragment of any one of embodiments 13 to 56, wherein the amino acid designated X₂₁ in SEQ ID NO:94 is S.

58. The anti-glyco-CD44 antibody or antigen-binding fragment of any one of embodiments 13 to 56, wherein the amino acid designated X₂₁ in SEQ ID NO:94 is G.

59. The anti-glyco-CD44 antibody or antigen-binding fragment of any one of embodiments 13 to 58, wherein the amino acid designated X₂₂ in SEQ ID NO:91 and SEQ ID NO:95 is G.

60. The anti-glyco-CD44 antibody or antigen-binding fragment of any one of embodiments 13 to 58, wherein the amino acid designated X₂₂ in SEQ ID NO:91 and SEQ ID NO:95 is S.

61. The anti-glyco-CD44 antibody or antigen-binding fragment of any one of embodiments 13 to 58, wherein the amino acid designated X₂₂ in SEQ ID NO:91 and SEQ ID NO:95 is L.

62. The anti-glyco-CD44 antibody or antigen-binding fragment of any one of embodiments 13 to 61, wherein the amino acid designated X₂₃ in SEQ ID NO:91 and SEQ ID NO:95 is T.

63. The anti-glyco-CD44 antibody or antigen-binding fragment of any one of embodiments 13 to 61, wherein the amino acid designated X₂₃ in SEQ ID NO:91 and SEQ ID NO:95 is I.

64. The anti-glyco-CD44 antibody or antigen-binding fragment of any one of embodiments 13 to 63, wherein the amino acid designated X₂₄ in SEQ ID NO:91 and SEQ ID NO:95 is N.

65. The anti-glyco-CD44 antibody or antigen-binding fragment of any one of embodiments 13 to 63, wherein the amino acid designated X₂₄ in SEQ ID NO:91 and SEQ ID NO:95 is S.

66. The anti-glyco-CD44 antibody or antigen-binding fragment of any one of embodiments 13 to 65, wherein the amino acid designated X₂₅ in SEQ ID NO:95 is N.

67. The anti-glyco-CD44 antibody or antigen-binding fragment of any one of embodiments 13 to 65, wherein the amino acid designated X₂₅ in SEQ ID NO:95 is R.

68. The anti-glyco-CD44 antibody or antigen-binding fragment of any one of embodiments 13 to 67, wherein the amino acid designated X₂₆ in SEQ ID NO:95 is R.

69. The anti-glyco-CD44 antibody or antigen-binding fragment of any one of embodiments 13 to 67, wherein the amino acid designated X₂₆ in SEQ ID NO:95 is L.

70. The anti-glyco-CD44 antibody or antigen-binding fragment of any one of embodiments 13 to 69, wherein the amino acid designated X₂₇ in SEQ ID NO:95 is A.

71. The anti-glyco-CD44 antibody or antigen-binding fragment of any one of embodiments 13 to 69, wherein the amino acid designated X₂₇ in SEQ ID NO:95 is H.

72. The anti-glyco-CD44 antibody or antigen-binding fragment of any one of embodiments 13 to 69, wherein the amino acid designated X₂₇ in SEQ ID NO:95 is F.

73. The anti-glyco-CD44 antibody or antigen-binding fragment of any one of embodiments 13 to 72, wherein the amino acid designated X₂₈ in SEQ ID NO:95 is P.

74. The anti-glyco-CD44 antibody or antigen-binding fragment of any one of embodiments 13 to 72, wherein the amino acid designated X₂₈ in SEQ ID NO:95 is S.

75. The anti-glyco-CD44 antibody or antigen-binding fragment of any one of embodiments 13 to 74, wherein the amino acid designated X₂₉ in SEQ ID NO:92 is A.

76. The anti-glyco-CD44 antibody or antigen-binding fragment of any one of embodiments 13 to 74, wherein the amino acid designated X₂₉ in SEQ ID NO:92 is Q.

77. The anti-glyco-CD44 antibody or antigen-binding fragment of any one of embodiments 13 to 74, wherein the amino acid designated X₂₉ in SEQ ID NO:92 is L.

78. The anti-glyco-CD44 antibody or antigen-binding fragment of any one of embodiments 13 to 77, wherein the amino acid designated X₃₀ in SEQ ID NO:92 is L.

79. The anti-glyco-CD44 antibody or antigen-binding fragment of any one of embodiments 13 to 77, wherein the amino acid designated X₃₀ in SEQ ID NO:92 is Q.

80. The anti-glyco-CD44 antibody or antigen-binding fragment of any one of embodiments 13 to 79, wherein the amino acid designated X₃₁ in SEQ ID NO:92 is L.

81. The anti-glyco-CD44 antibody or antigen-binding fragment of any one of embodiments 13 to 79, wherein the amino acid designated X₃₁ in SEQ ID NO:92 is G.

82. The anti-glyco-CD44 antibody or antigen-binding fragment of any one of embodiments 13 to 79, wherein the amino acid designated X₃₁ in SEQ ID NO:92 is W.

83. The anti-glyco-CD44 antibody or antigen-binding fragment of any one of embodiments 13 to 82, wherein the amino acid designated X₃₂ in SEQ ID NO:92 is Y.

84. The anti-glyco-CD44 antibody or antigen-binding fragment of any one of embodiments 13 to 82, wherein the amino acid designated X₃₂ in SEQ ID NO:92 is S.

85. The anti-glyco-CD44 antibody or antigen-binding fragment of any one of embodiments 13 to 82, wherein the amino acid designated X₃₂ in SEQ ID NO:92 is T.

86. The anti-glyco-CD44 antibody or antigen-binding fragment of any one of embodiments 13 to 85, wherein the amino acid designated X₃₃ in SEQ ID NO:92 is S.

87. The anti-glyco-CD44 antibody or antigen-binding fragment of any one of embodiments 13 to 85, wherein the amino acid designated X₃₃ in SEQ ID NO:92 is T.

88. The anti-glyco-CD44 antibody or antigen-binding fragment of any one of embodiments 13 to 85, wherein the amino acid designated X₃₃ in SEQ ID NO:92 is H.

89. The anti-glyco-CD44 antibody or antigen-binding fragment of any one of embodiments 13 to 88, wherein the amino acid designated X₃₄ in SEQ ID NO:92 is N.

90. The anti-glyco-CD44 antibody or antigen-binding fragment of any one of embodiments 13 to 88, wherein the amino acid designated X₃₄ in SEQ ID NO:92 is L.

91. The anti-glyco-CD44 antibody or antigen-binding fragment of any one of embodiments 13 to 88, wherein the amino acid designated X₃₄ in SEQ ID NO:92 is Q.

92. The anti-glyco-CD44 antibody or antigen-binding fragment of any one of embodiments 13 to 91, wherein the amino acid designated X₃₅ in SEQ ID NO:92 is Y.

93. The anti-glyco-CD44 antibody or antigen-binding fragment of any one of embodiments 13 to 91, wherein the amino acid designated X₃₅ in SEQ ID NO:92 is P.

94. The anti-glyco-CD44 antibody or antigen-binding fragment of any one of embodiments 13 to 93, wherein the amino acid designated X₃₆ in SEQ ID NO:92 is W.

95. The anti-glyco-CD44 antibody or antigen-binding fragment of any one of embodiments 13 to 93, wherein the amino acid designated X₃₆ in SEQ ID NO:92 is F.

96. The anti-glyco-CD44 antibody or antigen-binding fragment of any one of embodiments 13 to 95, wherein the amino acid designated X₃₇ in SEQ ID NO:92 is Y.

97. The anti-glyco-CD44 antibody or antigen-binding fragment of any one of embodiments 13 to 95, wherein the amino acid designated X₃₇ in SEQ ID NO:92 is T.

98. The anti-glyco-CD44 antibody or antigen-binding fragment of embodiment 13, wherein the amino acid designated X₄₁ in SEQ ID NO:228, SEQ ID NO:236, and SEQ ID NO:246 is Y.

99. The anti-glyco-CD44 antibody or antigen-binding fragment of embodiment 13, wherein the amino acid designated X₄₁ in SEQ ID NO:228, SEQ ID NO:232, and SEQ ID NO:246 is F.

100. The anti-glyco-CD44 antibody or antigen-binding fragment of any one of embodiments 13, 98, and 99, wherein the amino acid designated X₄₂ in SEQ ID NO:228, SEQ ID NO:236, and SEQ ID NO:246 is F.

101. The anti-glyco-CD44 antibody or antigen-binding fragment of any one of embodiments 13, 98, and 99, wherein the amino acid designated X₄₂ in SEQ ID NO:228, SEQ ID NO:236, and SEQ ID NO:246 is I.

102. The anti-glyco-CD44 antibody or antigen-binding fragment of any one of embodiments 13 and 98 to 101, wherein the amino acid designated X₄₃ in SEQ ID NO:228, SEQ ID NO:236, and SEQ ID NO:246 is T.

103. The anti-glyco-CD44 antibody or antigen-binding fragment of any one of embodiments 13 and 98 to 101, wherein the amino acid designated X₄₃ in SEQ ID NO:228, SEQ ID NO:236, and SEQ ID NO:246 is S.

104. The anti-glyco-CD44 antibody or antigen-binding fragment of any one of embodiments 13 and 98 to 101, wherein the amino acid designated X₄₃ in SEQ ID NO:228, SEQ ID NO:236, and SEQ ID NO:246 is N.

105. The anti-glyco-CD44 antibody or antigen-binding fragment of any one of embodiments 13 and 98 to 104, wherein the amino acid designated X₄₄ in SEQ ID NO:228, SEQ ID NO:232, SEQ ID NO:236, SEQ ID NO:246, and SEQ ID NO:256 is S.

106. The anti-glyco-CD44 antibody or antigen-binding fragment of any one of embodiments 13 and 98 to 104, wherein the amino acid designated X₄₄ in SEQ ID NO:228, SEQ ID NO:232, SEQ ID NO:236, SEQ ID NO:246, and SEQ ID NO:256 is T.

107. The anti-glyco-CD44 antibody or antigen-binding fragment of any one of embodiments 13 and 98 to 106, wherein the amino acid designated X₄₅ in SEQ ID NO:228, SEQ ID NO:232, SEQ ID NO:236, SEQ ID NO:246, and SEQ ID NO:256 is Y.

108. The anti-glyco-CD44 antibody or antigen-binding fragment of any one of embodiments 13 and 98 to 106, wherein the amino acid designated X₄₅ in SEQ ID NO:228, SEQ ID NO:232, SEQ ID NO:236, SEQ ID NO:246, and SEQ ID NO:256 is F.

109. The anti-glyco-CD44 antibody or antigen-binding fragment of any one of embodiments 13 and 98 to 108, wherein the amino acid designated X₄₆ in SEQ ID NO:228, SEQ ID NO:232, and SEQ ID NO:246 is W.

110. The anti-glyco-CD44 antibody or antigen-binding fragment of any one of embodiments 13 and 98 to 108, wherein the amino acid designated X₄₆ in SEQ ID NO:228, SEQ ID NO:232, and SEQ ID NO:246 is A.

111. The anti-glyco-CD44 antibody or antigen-binding fragment of any one of embodiments 13 and 98 to 108, wherein the amino acid designated X₄₆ in SEQ ID NO:228, SEQ ID NO:232, and SEQ ID NO:246 is G.

112. The anti-glyco-CD44 antibody or antigen-binding fragment of any one of embodiments 13 and 98 to 108, wherein the amino acid designated X₄₆ in SEQ ID NO:228, SEQ ID NO:232, and SEQ ID NO:246 is H.

113. The anti-glyco-CD44 antibody or antigen-binding fragment of any one of embodiments 13 and 98 to 112, wherein the amino acid designated X₄₇ in SEQ ID NO:232 and SEQ ID NO:246 is M.

114. The anti-glyco-CD44 antibody or antigen-binding fragment of any one of embodiments 13 and 98 to 112, wherein the amino acid designated X₄₇ in SEQ ID NO:232 and SEQ ID NO:246 is I.

115. The anti-glyco-CD44 antibody or antigen-binding fragment of any one of embodiments 13 and 98 to 114, wherein the amino acid designated X₄₈ in SEQ ID NO:232 and SEQ ID NO:246 is H.

116. The anti-glyco-CD44 antibody or antigen-binding fragment of any one of embodiments 13 and 98 to 114, wherein the amino acid designated X₄₈ in SEQ ID NO:232 and SEQ ID NO:246 is S.

117. The anti-glyco-CD44 antibody or antigen-binding fragment of any one of embodiments 13 and 98 to 114, wherein the amino acid designated X₄₈ in SEQ ID NO:232 and SEQ ID NO:246 is G.

118. The anti-glyco-CD44 antibody or antigen-binding fragment of any one of embodiments 13 and 98 to 117, wherein the amino acid designated X₄₉ in SEQ ID NO:233 is N.

119. The anti-glyco-CD44 antibody or antigen-binding fragment of any one of embodiments 13 and 98 to 117, wherein the amino acid designated X₄₉ in SEQ ID NO:233 is E.

120. The anti-glyco-CD44 antibody or antigen-binding fragment of any one of embodiments 13 and 98 to 117, wherein the amino acid designated X₄₉ in SEQ ID NO:233 is Y.

121. The anti-glyco-CD44 antibody or antigen-binding fragment of any one of embodiments 13 and 98 to 117, wherein the amino acid designated X₄₉ in SEQ ID NO:233 is I.

122. The anti-glyco-CD44 antibody or antigen-binding fragment of any one of embodiments 13 and 98 to 121, wherein the amino acid designated X₅₀ in SEQ ID NO:233 is I.

123. The anti-glyco-CD44 antibody or antigen-binding fragment of any one of embodiments 13 and 98 to 121, wherein the amino acid designated X₅₀ in SEQ ID NO:233 is V.

124. The anti-glyco-CD44 antibody or antigen-binding fragment of any one of embodiments 13 and 98 to 123, wherein the amino acid designated X₅₁ in SEQ ID NO:229, SEQ ID NO:233, and SEQ ID NO:237 is Y.

125. The anti-glyco-CD44 antibody or antigen-binding fragment of any one of embodiments 13 and 98 to 123, wherein the amino acid designated X₅₁ in SEQ ID NO:229, SEQ ID NO:233, and SEQ ID NO:237 is S.

126. The anti-glyco-CD44 antibody or antigen-binding fragment of any one of embodiments 13 and 98 to 125, wherein the amino acid designated X₅₂ in SEQ ID NO:229, SEQ ID NO:233, and SEQ ID NO:237 is P.

127. The anti-glyco-CD44 antibody or antigen-binding fragment of any one of embodiments 13 and 98 to 125, wherein the amino acid designated X₅₂ in SEQ ID NO:229, SEQ ID NO:233, and SEQ ID NO:237 is S.

128. The anti-glyco-CD44 antibody or antigen-binding fragment of any one of embodiments 13 and 98 to 125, wherein the amino acid designated X₅₂ in SEQ ID NO:229, SEQ ID NO:233, and SEQ ID NO:237 is H.

129. The anti-glyco-CD44 antibody or antigen-binding fragment of any one of embodiments 13 and 98 to 128, wherein the amino acid designated X₅₃ in SEQ ID NO:229, SEQ ID NO:233, and SEQ ID NO:237 is R.

130. The anti-glyco-CD44 antibody or antigen-binding fragment of any one of embodiments 13 and 98 to 128, wherein the amino acid designated X₅₃ in SEQ ID NO:229, SEQ ID NO:233, and SEQ ID NO:237 is G.

131. The anti-glyco-CD44 antibody or antigen-binding fragment of any one of embodiments 13 and 98 to 128, wherein the amino acid designated X₅₃ in SEQ ID NO:229, SEQ ID NO:233, and SEQ ID NO:237 is D.

132. The anti-glyco-CD44 antibody or antigen-binding fragment of any one of embodiments 13 and 98 to 131, wherein the amino acid designated X₅₄ in SEQ ID NO:229, SEQ ID NO:233, and SEQ ID NO:237 is S.

133. The anti-glyco-CD44 antibody or antigen-binding fragment of any one of embodiments 13 and 98 to 131, wherein the amino acid designated X₅₄ in SEQ ID NO:90, SEQ ID NO:94, and SEQ ID NO:98 is G.

134. The anti-glyco-CD44 antibody or antigen-binding fragment of any one of embodiments 13 and 98 to 133, wherein the amino acid designated X₅₅ in SEQ ID NO:229, SEQ ID NO:233, and SEQ ID NO:237 is G.

135. The anti-glyco-CD44 antibody or antigen-binding fragment of any one of embodiments 13 and 98 to 133, wherein the amino acid designated X₅₅ in SEQ ID SEQ ID NO:229, SEQ ID NO:233, and SEQ ID NO:237 is S.

136. The anti-glyco-CD44 antibody or antigen-binding fragment of any one of embodiments 13 and 98 to 135, wherein the amino acid designated X₅₆ in SEQ SEQ ID NO:229, SEQ ID NO:233, and SEQ ID NO:237 is T.

137. The anti-glyco-CD44 antibody or antigen-binding fragment of any one of embodiments 13 and 98 to 135, wherein the amino acid designated X₅₆ in SEQ ID NO:229, SEQ ID NO:233, and SEQ ID NO:237 is Y.

138. The anti-glyco-CD44 antibody or antigen-binding fragment of any one of embodiments 13 and 98 to 137, wherein the amino acid designated X₅₇ in SEQ ID NO:229 and SEQ ID NO:233 is T.

139. The anti-glyco-CD44 antibody or antigen-binding fragment of any one of embodiments 13 and 98 to 137, wherein the amino acid designated X₅₇ in SEQ ID NO:229 and SEQ ID NO:233 is I.

140. The anti-glyco-CD44 antibody or antigen-binding fragment of any one of embodiments 13 and 98 to 137, wherein the amino acid designated X₅₇ in SEQ ID NO:229 and SEQ ID NO:233 is absent.

141. The anti-glyco-CD44 antibody or antigen-binding fragment of any one of embodiments 13 and 98 to 140, wherein the amino acid designated X₅₈ in SEQ ID NO:233 is N.

142. The anti-glyco-CD44 antibody or antigen-binding fragment of any one of embodiments 13 and 98 to 140, wherein the amino acid designated X₅₈ in SEQ ID NO:233 is Y.

143. The anti-glyco-CD44 antibody or antigen-binding fragment of any one of embodiments 13 and 98 to 142, wherein the amino acid designated X₅₉ in SEQ ID NO:233 is D.

144. The anti-glyco-CD44 antibody or antigen-binding fragment of any one of embodiments 13 and 98 to 142, wherein the amino acid designated X₅₉ in SEQ ID NO:233 is P.

145. The anti-glyco-CD44 antibody or antigen-binding fragment of any one of embodiments 13 and 98 to 142, wherein the amino acid designated X₅₉ in SEQ ID NO:233 is A.

146. The anti-glyco-CD44 antibody or antigen-binding fragment of any one of embodiments 13 and 98 to 145, wherein the amino acid designated X₆₀ in SEQ ID NO:233 is G.

147. The anti-glyco-CD44 antibody or antigen-binding fragment of any one of embodiments 13 and 98 to 145, wherein the amino acid designated X₆₀ in SEQ ID NO:233 is D.

148. The anti-glyco-CD44 antibody or antigen-binding fragment of any one of embodiments 13 and 98 to 145, wherein the amino acid designated X₆₀ in SEQ ID NO:233 is T.

149. The anti-glyco-CD44 antibody or antigen-binding fragment of any one of embodiments 13 and 98 to 148, wherein the amino acid designated X₆₁ in SEQ ID NO:233 is Y.

150. The anti-glyco-CD44 antibody or antigen-binding fragment of any one of embodiments 13 and 98 to 148, wherein the amino acid designated X₆₁ in SEQ ID NO:233 is T.

151. The anti-glyco-CD44 antibody or antigen-binding fragment of any one of embodiments 13 and 98 to 148, wherein the amino acid designated X₆₁ in SEQ ID NO:233 is W.

152. The anti-glyco-CD44 antibody or antigen-binding fragment of any one of embodiments 13 and 98 to 151, wherein the amino acid designated X₆₂ in SEQ ID NO:233 is F.

153. The anti-glyco-CD44 antibody or antigen-binding fragment of any one of embodiments 13 and 98 to 151, wherein the amino acid designated X₆₂ in SEQ ID NO:233 is V.

154. The anti-glyco-CD44 antibody or antigen-binding fragment of any one of embodiments 13 and 98 to 151, wherein the amino acid designated X₆₂ in SEQ ID NO:233 is A.

155. The anti-glyco-CD44 antibody or antigen-binding fragment of any one of embodiments 13 and 98 to 154, wherein the amino acid designated X₆₃ in SEQ ID NO: 233 is K.

156. The anti-glyco-CD44 antibody or antigen-binding fragment of any one of embodiments 13 and 98 to 154, wherein the amino acid designated X₆₃ in SEQ ID NO: 233 is T.

157. The anti-glyco-CD44 antibody or antigen-binding fragment of any one of embodiments 13 and 98 to 156, wherein the amino acid designated X₆₄ in SEQ ID NO: 233 is S.

158. The anti-glyco-CD44 antibody or antigen-binding fragment of any one of embodiments 13 and 98 to 156, wherein the amino acid designated X₆₄ in SEQ ID NO: 233 is G.

159. The anti-glyco-CD44 antibody or antigen-binding fragment of any one of embodiments 13 and 98 to 158, wherein the amino acid designated X₆₅ in SEQ ID NO:230 and SEQ ID NO:234 is G.

160. The anti-glyco-CD44 antibody or antigen-binding fragment of any one of embodiments 13 and 98 to 158, wherein the amino acid designated X₆₅ in SEQ ID NO:230 and SEQ ID NO:234 is S.

161. The anti-glyco-CD44 antibody or antigen-binding fragment of any one of embodiments 13 and 98 to 158, wherein the amino acid designated X₆₅ in SEQ ID NO:230 and SEQ ID NO:234 is L.

162. The anti-glyco-CD44 antibody or antigen-binding fragment of any one of embodiments 13 and 98 to 158, wherein the amino acid designated X₆₅ in SEQ ID NO:230 and SEQ ID NO:234 is K.

163. The anti-glyco-CD44 antibody or antigen-binding fragment of any one of embodiments 13 and 98 to 162, wherein the amino acid designated X₆₆ in SEQ ID NO:230 and SEQ ID NO:234 is T.

164. The anti-glyco-CD44 antibody or antigen-binding fragment of any one of embodiments 13 and 98 to 162, wherein the amino acid designated X₆₆ in SEQ ID NO:230 and SEQ ID NO:234 is I.

165. The anti-glyco-CD44 antibody or antigen-binding fragment of any one of embodiments 13 and 98 to 162, wherein the amino acid designated X₆₆ in SEQ ID NO:230 and SEQ ID NO:234 is A.

166. The anti-glyco-CD44 antibody or antigen-binding fragment of any one of embodiments 13 and 98 to 165, wherein the amino acid designated X₆₇ in SEQ ID NO:230 and SEQ ID NO:234 is N.

167. The anti-glyco-CD44 antibody or antigen-binding fragment of any one of embodiments 13 and 98 to 165, wherein the amino acid designated X₆₇ in SEQ ID NO:230 and SEQ ID NO:234 is S.

168. The anti-glyco-CD44 antibody or antigen-binding fragment of any one of embodiments 13 and 98 to 167, wherein the amino acid designated X₆₈ in SEQ ID NO:234 is N.

169. The anti-glyco-CD44 antibody or antigen-binding fragment of any one of embodiments 13 and 98 to 167, wherein the amino acid designated X₆₈ in SEQ ID NO: 234 is R.

170. The anti-glyco-CD44 antibody or antigen-binding fragment of any one of embodiments 13 and 98 to 167, wherein the amino acid designated X₆₈ in SEQ ID NO: 234 is T.

171. The anti-glyco-CD44 antibody or antigen-binding fragment of any one of embodiments 13 and 98 to 170, wherein the amino acid designated X₆₉ in SEQ ID NO: 234 is R.

172. The anti-glyco-CD44 antibody or antigen-binding fragment of any one of embodiments 13 and 98 to 170, wherein the amino acid designated X₆₉ in SEQ ID NO: 234 is L.

173. The anti-glyco-CD44 antibody or antigen-binding fragment of any one of embodiments 13 and 98 to 172, wherein the amino acid designated X₇₀ in SEQ ID NO: 234 is A.

174. The anti-glyco-CD44 antibody or antigen-binding fragment of any one of embodiments 13 and 98 to 172, wherein the amino acid designated X₇₀ in SEQ ID NO: 234 is H.

175. The anti-glyco-CD44 antibody or antigen-binding fragment of any one of embodiments 13 and 98 to 172, wherein the amino acid designated X₇₀ in SEQ ID NO: 234 is F.

176. The anti-glyco-CD44 antibody or antigen-binding fragment of any one of embodiments 13 and 98 to 175, wherein the amino acid designated X₇₁ in SEQ ID NO: 234 is P.

177. The anti-glyco-CD44 antibody or antigen-binding fragment of any one of embodiments 13 and 98 to 175, wherein the amino acid designated X₇₁ in SEQ ID NO: 234 is S.

178. The anti-glyco-CD44 antibody or antigen-binding fragment of any one of embodiments 13 and 98 to 177, wherein the amino acid designated X₇₂ in SEQ ID NO:231 is A.

179. The anti-glyco-CD44 antibody or antigen-binding fragment of any one of embodiments 13 and 98 to 177, wherein the amino acid designated X₇₂ in SEQ ID NO: 231 is Q.

180. The anti-glyco-CD44 antibody or antigen-binding fragment of any one of embodiments 13 and 98 to 177, wherein the amino acid designated X₇₂ in SEQ ID NO: 231 is L.

181. The anti-glyco-CD44 antibody or antigen-binding fragment of any one of embodiments 13 and 98 to 180, wherein the amino acid designated X₇₃ in SEQ ID NO: 231 is L.

182. The anti-glyco-CD44 antibody or antigen-binding fragment of any one of embodiments 13 and 98 to 180, wherein the amino acid designated X₇₃ in SEQ ID NO: 231 is Q.

183. The anti-glyco-CD44 antibody or antigen-binding fragment of any one of embodiments 13 and 98 to 180, wherein the amino acid designated X₇₃ in SEQ ID NO: 231 is G.

184. The anti-glyco-CD44 antibody or antigen-binding fragment of any one of embodiments 13 and 98 to 183, wherein the amino acid designated X₇₄ in SEQ ID NO: 231 is L.

185. The anti-glyco-CD44 antibody or antigen-binding fragment of any one of embodiments 13 and 98 to 183, wherein the amino acid designated X₇₄ in SEQ ID NO: 231 is G.

186. The anti-glyco-CD44 antibody or antigen-binding fragment of any one of embodiments 13 and 98 to 183, wherein the amino acid designated X₇₄ in SEQ ID NO: 231 is W.

187. The anti-glyco-CD44 antibody or antigen-binding fragment of any one of embodiments 13 and 98 to 186, wherein the amino acid designated X₇₅ in SEQ ID NO: 231 is Y.

188. The anti-glyco-CD44 antibody or antigen-binding fragment of any one of embodiments 13 and 98 to 186, wherein the amino acid designated X₇₅ in SEQ ID NO: 231 is S.

189. The anti-glyco-CD44 antibody or antigen-binding fragment of any one of embodiments 13 and 98 to 186, wherein the amino acid designated X₇₅ in SEQ ID NO: 231 is T.

190. The anti-glyco-CD44 antibody or antigen-binding fragment of any one of embodiments 13 and 98 to 189, wherein the amino acid designated X₇₆ in SEQ ID NO: 231 is S.

191. The anti-glyco-CD44 antibody or antigen-binding fragment of any one of embodiments 13 and 98 to 189, wherein the amino acid designated X₇₆ in SEQ ID NO: 231 is T.

192. The anti-glyco-CD44 antibody or antigen-binding fragment of any one of embodiments 13 and 98 to 189, wherein the amino acid designated X₇₆ in SEQ ID NO: 231 is H.

193. The anti-glyco-CD44 antibody or antigen-binding fragment of any one of embodiments 13 and 98 to 189, wherein the amino acid designated X₇₆ in SEQ ID NO: 231 is K.

194. The anti-glyco-CD44 antibody or antigen-binding fragment of any one of embodiments 13 and 98 to 193, wherein the amino acid designated X₇₇ in SEQ ID NO: 231 is N.

195. The anti-glyco-CD44 antibody or antigen-binding fragment of any one of embodiments 13 and 98 to 193, wherein the amino acid designated X₇₇ in SEQ ID NO: 231 is L.

196. The anti-glyco-CD44 antibody or antigen-binding fragment of any one of embodiments 13 and 98 to 193, wherein the amino acid designated X₇₇ in SEQ ID NO: 231 is Q.

197. The anti-glyco-CD44 antibody or antigen-binding fragment of any one of embodiments 13 and 98 to 193, wherein the amino acid designated X₇₇ in SEQ ID NO: 231 is G.

198. The anti-glyco-CD44 antibody or antigen-binding fragment of any one of embodiments 13 and 98 to 197, wherein the amino acid designated X₇₈ in SEQ ID NO: 231 is Y.

199. The anti-glyco-CD44 antibody or antigen-binding fragment of any one of embodiments 13 and 98 to 197, wherein the amino acid designated X₇₈ in SEQ ID NO: 231 is P.

200. The anti-glyco-CD44 antibody or antigen-binding fragment of any one of embodiments 13 and 98 to 197, wherein the amino acid designated X₇₈ in SEQ ID NO: 231 is D.

201. The anti-glyco-CD44 antibody or antigen-binding fragment of any one of embodiments 13 and 98 to 200, wherein the amino acid designated X₇₉ in SEQ ID NO: 231 is W.

202. The anti-glyco-CD44 antibody or antigen-binding fragment of any one of embodiments 13 and 98 to 200, wherein the amino acid designated X₇₉ in SEQ ID NO: 231 is F.

203. The anti-glyco-CD44 antibody or antigen-binding fragment of any one of embodiments 13 and 98 to 200, wherein the amino acid designated X₇₉ in SEQ ID NO: 231 is I.

204. The anti-glyco-CD44 antibody or antigen-binding fragment of any one of embodiments 13 and 98 to 203, wherein the amino acid designated X₈₀ in SEQ ID NO: 231 is Y.

205. The anti-glyco-CD44 antibody or antigen-binding fragment of any one of embodiments 13 and 98 to 203, wherein the amino acid designated X₈₀ in SEQ ID NO: 231 is T.

206. The anti-glyco-CD44 antibody or antigen-binding fragment of any one of embodiments 13 and 98 to 203, wherein the amino acid designated X₈₀ in SEQ ID NO: 231 is H.

207. The anti-glyco-CD44 antibody or antigen-binding fragment of any one of embodiments 13 and 98 to 206, wherein the amino acid designated X₈₁ in SEQ ID NO: 231 is P.

208. The anti-glyco-CD44 antibody or antigen-binding fragment of any one of embodiments 13 and 98 to 206, wherein the amino acid designated X₈₁ in SEQ ID NO: 231 is absent.

209. The anti-glyco-CD44 antibody or antigen-binding fragment of any one of embodiments 13 to 208, wherein CDR-H1 comprises the amino acid sequence of SEQ ID NO:89.

210. The anti-glyco-CD44 antibody or antigen-binding fragment of any one of embodiments 13 to 208, wherein CDR-H1 comprises the amino acid sequence of SEQ ID NO:93.

211. The anti-glyco-CD44 antibody or antigen-binding fragment of any one of embodiments 13 to 208, wherein CDR-H1 comprises the amino acid sequence of SEQ ID NO:97.

212. The anti-glyco-CD44 antibody or antigen-binding fragment of any one of embodiments 13 to 208, wherein CDR-H1 comprises the amino acid sequence of SEQ ID NO:125.

213. The anti-glyco-CD44 antibody or antigen-binding fragment of any one of embodiments 13 to 208, wherein CDR-H1 comprises the amino acid sequence of SEQ ID NO:153.

214. The anti-glyco-CD44 antibody or antigen-binding fragment of any one of embodiments 13 to 208, wherein CDR-H1 comprises the amino acid sequence of SEQ ID NO:228.

215. The anti-glyco-CD44 antibody or antigen-binding fragment of any one of embodiments 13 to 208, wherein CDR-H1 comprises the amino acid sequence of SEQ ID NO:232.

216. The anti-glyco-CD44 antibody or antigen-binding fragment of any one of embodiments 13 to 208, wherein CDR-H1 comprises the amino acid sequence of SEQ ID NO:236.

217. The anti-glyco-CD44 antibody or antigen-binding fragment of any one of embodiments 13 to 208, wherein CDR-H1 comprises the amino acid sequence of SEQ ID NO:246.

218. The anti-glyco-CD44 antibody or antigen-binding fragment of any one of embodiments 13 to 208, wherein CDR-H1 comprises the amino acid sequence of SEQ ID NO:256.

219. The anti-glyco-CD44 antibody or antigen-binding fragment of any one of embodiments 13 to 213, wherein CDR-H2 comprises the amino acid sequence of SEQ ID NO:90.

220. The anti-glyco-CD44 antibody or antigen-binding fragment of any one of embodiments 13 to 213, wherein CDR-H2 comprises the amino acid sequence of SEQ ID NO:94.

221. The anti-glyco-CD44 antibody or antigen-binding fragment of any one of embodiments 13 to 213, wherein CDR-H2 comprises the amino acid sequence of SEQ ID NO:98.

222. The anti-glyco-CD44 antibody or antigen-binding fragment of any one of embodiments 13 to 213, wherein CDR-H2 comprises the amino acid sequence of SEQ ID NO:229.

223. The anti-glyco-CD44 antibody or antigen-binding fragment of any one of embodiments 13 to 213, wherein CDR-H2 comprises the amino acid sequence of SEQ ID NO:233.

224. The anti-glyco-CD44 antibody or antigen-binding fragment of any one of embodiments 13 to 213, wherein CDR-H2 comprises the amino acid sequence of SEQ ID NO:237.

225. The anti-glyco-CD44 antibody or antigen-binding fragment of any one of embodiments 13 to 224, wherein CDR-H3 comprises the amino acid sequence of SEQ ID NO:103.

226. The anti-glyco-CD44 antibody or antigen-binding fragment of any one of embodiments 13 to 224, wherein CDR-H3 comprises the amino acid sequence of SEQ ID NO: 109.

227. The anti-glyco-CD44 antibody or antigen-binding fragment of any one of embodiments 13 to 224, wherein CDR-H3 comprises the amino acid sequence of SEQ ID NO: 115.

228. The anti-glyco-CD44 antibody or antigen-binding fragment of any one of embodiments 13 to 224, wherein CDR-H3 comprises the amino acid sequence of SEQ ID NO:121.

229. The anti-glyco-CD44 antibody or antigen-binding fragment of any one of embodiments 13 to 224, wherein CDR-H3 comprises the amino acid sequence of SEQ ID NO:131.

230. The anti-glyco-CD44 antibody or antigen-binding fragment of any one of embodiments 13 to 224, wherein CDR-H3 comprises the amino acid sequence of SEQ ID NO:137.

231. The anti-glyco-CD44 antibody or antigen-binding fragment of any one of embodiments 13 to 224, wherein CDR-H3 comprises the amino acid sequence of SEQ ID NO:143.

232. The anti-glyco-CD44 antibody or antigen-binding fragment of any one of embodiments 13 to 224, wherein CDR-H3 comprises the amino acid sequence of SEQ ID NO:149.

233. The anti-glyco-CD44 antibody or antigen-binding fragment of any one of embodiments 13 to 224, wherein CDR-H3 comprises the amino acid sequence of SEQ ID NO:242.

234. The anti-glyco-CD44 antibody or antigen-binding fragment of any one of embodiments 13 to 224, wherein CDR-H3 comprises the amino acid sequence of SEQ ID NO:252.

235. The anti-glyco-CD44 antibody or antigen-binding fragment of any one of embodiments 13 to 234, wherein CDR-L1 comprises the amino acid sequence of SEQ ID NO:104.

236. The anti-glyco-CD44 antibody or antigen-binding fragment of any one of embodiments 13 to 234, wherein CDR-L1 comprises the amino acid sequence of SEQ ID NO:110.

237. The anti-glyco-CD44 antibody or antigen-binding fragment of any one of embodiments 13 to 234, wherein CDR-L1 comprises the amino acid sequence of SEQ ID NO:116.

238. The anti-glyco-CD44 antibody or antigen-binding fragment of any one of embodiments 13 to 234, wherein CDR-L1 comprises the amino acid sequence of SEQ ID NO:122,

239. The anti-glyco-CD44 antibody or antigen-binding fragment of any one of embodiments 13 to 234, wherein CDR-L1 comprises the amino acid sequence of SEQ ID NO:132.

240. The anti-glyco-CD44 antibody or antigen-binding fragment of any one of embodiments 13 to 234, wherein CDR-L1 comprises the amino acid sequence of SEQ ID NO:138.

241. The anti-glyco-CD44 antibody or antigen-binding fragment of any one of embodiments 13 to 234, wherein CDR-L1 comprises the amino acid sequence of SEQ ID NO:144.

242. The anti-glyco-CD44 antibody or antigen-binding fragment of any one of embodiments 13 to 232, wherein CDR-L1 comprises the amino acid sequence of SEQ ID NO:150.

243. The anti-glyco-CD44 antibody or antigen-binding fragment of any one of embodiments 13 to 232, wherein CDR-L1 comprises the amino acid sequence of SEQ ID NO:243.

244. The anti-glyco-CD44 antibody or antigen-binding fragment of any one of embodiments 13 to 232, wherein CDR-L1 comprises the amino acid sequence of SEQ ID NO:253.

245. The anti-glyco-CD44 antibody or antigen-binding fragment of any one of embodiments 13 to 244, wherein CDR-L2 comprises the amino acid sequence of SEQ ID NO:91.

246. The anti-glyco-CD44 antibody or antigen-binding fragment of any one of embodiments 13 to 244, wherein CDR-L2 comprises the amino acid sequence of SEQ ID NO:95.

247. The anti-glyco-CD44 antibody or antigen-binding fragment of any one of embodiments 13 to 244, wherein CDR-L2 comprises the amino acid sequence of SEQ ID NO:230.

248. The anti-glyco-CD44 antibody or antigen-binding fragment of any one of embodiments 13 to 244, wherein CDR-L2 comprises the amino acid sequence of SEQ ID NO:234.

249. The anti-glyco-CD44 antibody or antigen-binding fragment of any one of embodiments 13 to 248, wherein CDR-L3 comprises the amino acid sequence of SEQ ID NO:92.

250. The anti-glyco-CD44 antibody or antigen-binding fragment of any one of embodiments 13 to 248, wherein CDR-L3 comprises the amino acid sequence of SEQ ID NO:231.

251. The anti-glyco-CD44 antibody or antigen-binding fragment of any one of embodiments 1 to 12, which comprises a VH comprising CDRs of SEQ ID NOs:3-5 and a VL comprising CDRs of SEQ ID NOs:6-8.

252. The anti-glyco-CD44 antibody or antigen-binding fragment of any one of embodiments 1 to 12, which comprises a VH comprising CDRs of SEQ ID NOs:9-11 and a VL comprising CDRs of SEQ ID NOs:12-14.

253. The anti-glyco-CD44 antibody or antigen-binding fragment of any one of embodiments 1 to 12, which comprises a VH comprising CDRs of SEQ ID NOs:15-17 and a VL comprising CDRs of SEQ ID NOs:18-20.

254. The anti-glyco-CD44 antibody or antigen-binding fragment of any one of embodiments 1 to 12, which comprises a VH comprising CDRs of SEQ ID NOs:25-27 and a VL comprising CDRs of SEQ ID NOs:28-30.

255. The anti-glyco-CD44 antibody or antigen-binding fragment of any one of embodiments 1 to 12, which comprises a VH comprising CDRs of SEQ ID NOs:31-33 and a VL comprising CDRs of SEQ ID NOs:34-36.

256. The anti-glyco-CD44 antibody or antigen-binding fragment of any one of embodiments 1 to 12, which comprises a VH comprising CDRs of SEQ ID NOs:37-39 and a VL comprising CDRs of SEQ ID NOs:40-42.

257. The anti-glyco-CD44 antibody or antigen-binding fragment of any one of embodiments 1 to 12, which comprises a VH comprising CDRs of SEQ ID NOs:47-49 and a VL comprising CDRs of SEQ ID NOs:50-52.

258. The anti-glyco-CD44 antibody or antigen-binding fragment of any one of embodiments 1 to 12, which comprises a VH comprising CDRs of SEQ ID NOs:53-55 and a VL comprising CDRs of SEQ ID NOs:56-58.

259. The anti-glyco-CD44 antibody or antigen-binding fragment of any one of embodiments 1 to 12, which comprises a VH comprising CDRs of SEQ ID NOs:59-61 and a VL comprising CDRs of SEQ ID NOs:62-64.

260. The anti-glyco-CD44 antibody or antigen-binding fragment of any one of embodiments 1 to 12, which comprises a VH comprising CDRs of SEQ ID NOs:69-71 and a VL comprising CDRs of SEQ ID NOs:72-74.

261. The anti-glyco-CD44 antibody or antigen-binding fragment of any one of embodiments 1 to 12, which comprises a VH comprising CDRs of SEQ ID NOs:75-77 and a VL comprising CDRs of SEQ ID NOs:78-80.

262. The anti-glyco-CD44 antibody or antigen-binding fragment of any one of embodiments 1 to 12, which comprises a VH comprising CDRs of SEQ ID NOs:81-83 and a VL comprising CDRs of SEQ ID NOs:84-86.

263. The anti-glyco-CD44 antibody or antigen-binding fragment of any one of embodiments 1 to 12, which comprises a VH comprising CDRs of SEQ ID NOs:208-210 and a VL comprising CDRs of SEQ ID NOs:211-213.

264. The anti-glyco-CD44 antibody or antigen-binding fragment of any one of embodiments 1 to 12, which comprises a VH comprising CDRs of SEQ ID NOs:214-216 and a VL comprising CDRs of SEQ ID NOs:217-219.

265. The anti-glyco-CD44 antibody or antigen-binding fragment of any one of embodiments 1 to 12, which comprises a VH comprising CDRs of SEQ ID NOs:220-222 and a VL comprising CDRs of SEQ ID NOs:223-225.

266. The anti-glyco-CD44 antibody or antigen-binding fragment of any one of embodiments 1 to 12, which comprises a VH comprising CDRs of SEQ ID NOs:101-103 and a VL comprising CDRs of SEQ ID NOs:104-106.

267. The anti-glyco-CD44 antibody or antigen-binding fragment of any one of embodiments 1 to 12, which comprises a VH comprising CDRs of SEQ ID NOs:107-109 and a VL comprising CDRs of SEQ ID NOs:110-112.

268. The anti-glyco-CD44 antibody or antigen-binding fragment of any one of embodiments 1 to 12, which comprises a VH comprising CDRs of SEQ ID NOs:113-115 and a VL comprising CDRs of SEQ ID NOs:116-118.

269. The anti-glyco-CD44 antibody or antigen-binding fragment of any one of embodiments 1 to 12, which comprises a VH comprising CDRs of SEQ ID NOs:119-121 and a VL comprising CDRs of SEQ ID NOs:122-124.

270. The anti-glyco-CD44 antibody or antigen-binding fragment of any one of embodiments 1 to 12, which comprises a VH comprising CDRs of SEQ ID NOs:240-242 and a VL comprising CDRs of SEQ ID NOs:243-245.

271. The anti-glyco-CD44 antibody or antigen-binding fragment of any one of embodiments 1 to 12, which comprises a VH comprising CDRs of SEQ ID NOs:129-131 and a VL comprising CDRs of SEQ ID NOs:132-134.

272. The anti-glyco-CD44 antibody or antigen-binding fragment of any one of embodiments 1 to 12, which comprises a VH comprising CDRs of SEQ ID NOs:135-137 and a VL comprising CDRs of SEQ ID NOs:138-140.

273. The anti-glyco-CD44 antibody or antigen-binding fragment of any one of embodiments 1 to 12, which comprises a VH comprising CDRs of SEQ ID NOs:141-143 and a VL comprising CDRs of SEQ ID NOs:144-146.

274. The anti-glyco-CD44 antibody or antigen-binding fragment of any one of embodiments 1 to 12, which comprises a VH comprising CDRs of SEQ ID NOs:147-149 and a VL comprising CDRs of SEQ ID NOs:150-152.

275. The anti-glyco-CD44 antibody or antigen-binding fragment of any one of embodiments 1 to 12, which comprises a VH comprising CDRs of SEQ ID NOs:250-252 and a VL comprising CDRs of SEQ ID NOs:253-255.

276. The anti-glyco-CD44 antibody or antigen-binding fragment of any one of embodiments 1 to 275, which is a chimeric or humanized antibody or antigen-binding fragment of a chimeric or humanized antibody.

277. The anti-glyco-CD44 antibody or antigen-binding fragment of any one of embodiments 1 to 12, which comprises a VH comprising an amino acid sequence having at least 95% sequence identity to SEQ ID NO:1 and a VL comprising an amino acid sequence having at least 95% sequence identity to SEQ ID NO:2.

278. The anti-glyco-CD44 antibody or antigen-binding fragment of any one of embodiments 1 to 12, which comprises a VH comprising an amino acid sequence having at least 97% sequence identity to SEQ ID NO:1 and a VL comprising an amino acid sequence having at least 97% sequence identity to SEQ ID NO:2.

279. The anti-glyco-CD44 antibody or antigen-binding fragment of any one of embodiments 1 to 12, which comprises a VH comprising an amino acid sequence having at least 99% sequence identity to SEQ ID NO:1 and a VL comprising an amino acid sequence having at least 99% sequence identity to SEQ ID NO:2.

280. The anti-glyco-CD44 antibody or antigen-binding fragment of any one of embodiments 1 to 12, which comprises a VH comprising the amino acid sequence of SEQ ID NO:1 and a VL comprising the amino acid sequence of SEQ ID NO:2.

281. The anti-glyco-CD44 antibody or antigen-binding fragment of any one of embodiments 1 to 12, which comprises a VH comprising an amino acid sequence having at least 95% sequence identity to SEQ ID NO:23 and a VL comprising an amino acid sequence having at least 95% sequence identity to SEQ ID NO:24.

282. The anti-glyco-CD44 antibody or antigen-binding fragment of any one of embodiments 1 to 12, which comprises a VH comprising an amino acid sequence having at least 97% sequence identity to SEQ ID NO:23 and a VL comprising an amino acid sequence having at least 97% sequence identity to SEQ ID NO:24.

283. The anti-glyco-CD44 antibody or antigen-binding fragment of any one of embodiments 1 to 12, which comprises a VH comprising an amino acid sequence having at least 99% sequence identity to SEQ ID NO:23 and a VL comprising an amino acid sequence having at least 99% sequence identity to SEQ ID NO:24.

284. The anti-glyco-CD44 antibody or antigen-binding fragment of any one of embodiments 1 to 12, which comprises a VH comprising the amino acid sequence of SEQ ID NO:23 and a VL comprising the amino acid sequence of SEQ ID NO:24.

285. The anti-glyco-CD44 antibody or antigen-binding fragment of any one of embodiments 1 to 12, which comprises a VH comprising an amino acid sequence having at least 95% sequence identity to SEQ ID NO:45 and a VL comprising an amino acid sequence having at least 95% sequence identity to SEQ ID NO:46.

286. The anti-glyco-CD44 antibody or antigen-binding fragment of any one of embodiments 1 to 12, which comprises a VH comprising an amino acid sequence having at least 97% sequence identity to SEQ ID NO:45 and a VL comprising an amino acid sequence having at least 97% sequence identity to SEQ ID NO:46.

287. The anti-glyco-CD44 antibody or antigen-binding fragment of any one of embodiments 1 to 12, which comprises a VH comprising an amino acid sequence having at least 99% sequence identity to SEQ ID NO:45 and a VL comprising an amino acid sequence having at least 99% sequence identity to SEQ ID NO:46.

288. The anti-glyco-CD44 antibody or antigen-binding fragment of any one of embodiments 1 to 12, which comprises a VH comprising the amino acid sequence of SEQ ID NO:45 and a VL comprising the amino acid sequence of SEQ ID NO:46.

289. The anti-glyco-CD44 antibody or antigen-binding fragment of any one of embodiments 1 to 12, which comprises a VH comprising an amino acid sequence having at least 95% sequence identity to SEQ ID NO:67 and a VL comprising an amino acid sequence having at least 95% sequence identity to SEQ ID NO:68.

290. The anti-glyco-CD44 antibody or antigen-binding fragment of any one of embodiments 1 to 12, which comprises a VH comprising an amino acid sequence having at least 97% sequence identity to SEQ ID NO:67 and a VL comprising an amino acid sequence having at least 97% sequence identity to SEQ ID NO:68.

291. The anti-glyco-CD44 antibody or antigen-binding fragment of any one of embodiments 1 to 12, which comprises a VH comprising an amino acid sequence having at least 99% sequence identity to SEQ ID NO:67 and a VL comprising an amino acid sequence having at least 99% sequence identity to SEQ ID NO:68.

292. The anti-glyco-CD44 antibody or antigen-binding fragment of any one of embodiments 1 to 12, which comprises a VH comprising the amino acid sequence of SEQ ID NO:67 and a VL comprising the amino acid sequence of SEQ ID NO:68.

293. The anti-glyco-CD44 antibody or antigen-binding fragment of any one of embodiments 1 to 12, which comprises a VH comprising an amino acid sequence having at least 95% sequence identity to SEQ ID NO:206 and a VL comprising an amino acid sequence having at least 95% sequence identity to SEQ ID NO:207.

294. The anti-glyco-CD44 antibody or antigen-binding fragment of any one of embodiments 1 to 12, which comprises a VH comprising an amino acid sequence having at least 97% sequence identity to SEQ ID NO:206 and a VL comprising an amino acid sequence having at least 97% sequence identity to SEQ ID NO:207.

295. The anti-glyco-CD44 antibody or antigen-binding fragment of any one of embodiments 1 to 12, which comprises a VH comprising an amino acid sequence having at least 99% sequence identity to SEQ ID NO:206 and a VL comprising an amino acid sequence having at least 99% sequence identity to SEQ ID NO:207.

296. The anti-glyco-CD44 antibody or antigen-binding fragment of any one of embodiments 1 to 12, which comprises a VH comprising the amino acid sequence of SEQ ID NO:206 and a VL comprising the amino acid sequence of SEQ ID NO:207.

297. An anti-glyco-CD44 antibody or antigen-binding fragment that competes with a reference antibody or antigen binding fragment comprising (i) a heavy chain variable (VH) sequence of SEQ ID NO:1 and a light chain variable (VL) sequence of SEQ ID NO:2 (ii) a heavy chain variable (VH) sequence of SEQ ID NO:23 and a light chain variable (VL) sequence of SEQ ID NO:24, (iii) a heavy chain variable (VH) sequence of SEQ ID NO:45 and a light chain variable (VL) sequence of SEQ ID NO:46, (iv) a heavy chain variable (VH) sequence of SEQ ID NO:67 and a light chain variable (VL) sequence of SEQ ID NO:68, or (v) a heavy chain variable (VH) sequence of SEQ ID NO:206 and a light chain variable (VL) sequence of SEQ ID NO:207 for binding to a CD44v6 peptide GYRQTPKEDSHSTTGTAAA (SEQ ID NO:165) that has been glycosylated with GalNAc on threonine at amino acid position 5 of SEQ ID NO: 165 and serine at amino acid position 12 of SEQ ID NO: 165 (“the CD44v6 glycopeptide”), the anti-glyco-CD44 antibody or antigen-binding fragment comprising:
(a) a VH sequence with first, second and third CDR means within the VH sequence; and
(b) a VL sequence with fourth, fifth and sixth CDR means within the VL sequence, wherein the first, second, third, fourth, fifth, and sixth CDR means cooperate to effect binding of the anti-glyco-CD44 antibody or antigen-binding fragment to the CD44v6 glycopeptide.

298. The anti-glyco-CD44 antibody or antigen-binding fragment of embodiment 297 wherein the anti-glyco-CD44 antibody or antigen-binding fragment competes with a reference antibody or antigen binding fragment comprising a VH sequence of SEQ ID NO:1 and a VL sequence of SEQ ID NO:2.

299. The anti-glyco-CD44 antibody or antigen-binding fragment of embodiment 297 wherein the anti-glyco-CD44 antibody or antigen-binding fragment competes with a reference antibody or antigen binding fragment comprising a VH sequence of SEQ ID NO:23 and a VL sequence of SEQ ID NO:23.

300. The anti-glyco-CD44 antibody or antigen-binding fragment of embodiment 297 wherein the anti-glyco-CD44 antibody or antigen-binding fragment competes with a reference antibody or antigen binding fragment comprising a VH sequence of SEQ ID NO:45 and a VL sequence of SEQ ID NO:46.

301. The anti-glyco-CD44 antibody or antigen-binding fragment of embodiment 297 wherein the anti-glyco-CD44 antibody or antigen-binding fragment competes with a reference antibody or antigen binding fragment comprising a VH sequence of SEQ ID NO:67 and a VL sequence of SEQ ID NO:68.

302. The anti-glyco-CD44 antibody or antigen-binding fragment of embodiment 297 wherein the anti-glyco-CD44 antibody or antigen-binding fragment competes with a reference antibody or antigen binding fragment comprising a VH sequence of SEQ ID NO:206 and a VL sequence of SEQ ID NO:207.

303. The anti-glyco-CD44 antibody or antigen-binding fragment of any one of embodiments 1 to 302, which preferentially binds to a glyco-CD44 epitope that is overexpressed on cancer cells as compared to normal cells.

304. The anti-glyco-CD44 antibody or antigen-binding fragment of any one of embodiments 1 to 303, which specifically binds to a CD44v6 peptide GYRQTPKEDSHSTTGTAAA (SEQ ID NO:165) that has been glycosylated with STn on threonine at amino acid position 5 of SEQ ID NO: 165 and serine at amino acid position 12 of SEQ ID NO: 165.

305. The anti-glyco-CD44 antibody or antigen-binding fragment of any one of embodiments 1 to 303, which binds to, but does not specifically bind to, a CD44v6 peptide GYRQTPKEDSHSTTGTAAA SEQ (ID NO:165) that has been glycosylated with STn on threonine at amino acid position 5 of SEQ ID NO: 165 and serine at amino acid position 12 of SEQ ID NO: 165.

306. The anti-glyco-CD44 antibody or antigen-binding fragment of any of embodiments 1 to 305, which binds to the CD44v6 glycopeptide with a binding affinity (K_{D}) of 1 nM to 200 nM as measured by surface plasmon resonance or bio-layer interferometry.

307. The anti-glyco-CD44 antibody or antigen-binding fragment of any of embodiments 1 to 305, which binds to the CD44v6 glycopeptide with a binding affinity (K_{D}) of 1 nM to 150 nM as measured by surface plasmon resonance or bio-layer interferometry.

308. The anti-glyco-CD44 antibody or antigen-binding fragment of any of embodiments 1 to 305, which binds to the CD44v6 glycopeptide with a binding affinity (K_{D}) of 1 nM to 100 nM as measured by surface plasmon resonance or bio-layer interferometry.

309. The anti-glyco-CD44 antibody or antigen-binding fragment of any of embodiments 1 to 305, which binds to the CD44v6 glycopeptide with a binding affinity (K_{D}) of 1 nM to 50 nM as measured by surface plasmon resonance or bio-layer interferometry.

310. The anti-glyco-CD44 antibody or antigen-binding fragment of any of embodiments 1 to 305, which binds to the CD44v6 glycopeptide with a binding affinity (K_{D}) of 1 nM to 25 nM as measured by surface plasmon resonance or bio-layer interferometry.

311. The anti-glyco-CD44 antibody or antigen-binding fragment of any of embodiments 1 to 305, which binds to the CD44v6 glycopeptide with a binding affinity (K_{D}) of 1 nM to 15 nM as measured by surface plasmon resonance or bio-layer interferometry

312. The anti-glyco-CD44 antibody or antigen-binding fragment of any of embodiments 1 to 305, which binds to the CD44v6 glycopeptide with a binding affinity (K_{D}) of 1 nM to 10 nM as measured by surface plasmon resonance or bio-layer interferometry.

313. The anti-glyco-CD44 antibody or antigen-binding fragment of any of embodiments 1 to 305, which binds to the CD44v6 glycopeptide with a binding affinity (K_{D}) of 5 nM to 200 nM as measured by surface plasmon resonance or bio-layer interferometry.

314. The anti-glyco-CD44 antibody or antigen-binding fragment of any of embodiments 1 to 305, which binds to the CD44v6 glycopeptide with a binding affinity (K_{D}) of 5 nM to 100 nM as measured by surface plasmon resonance or bio-layer interferometry.

315. The anti-glyco-CD44 antibody or antigen-binding fragment of any of embodiments 1 to 305, which binds to the CD44v6 glycopeptide with a binding affinity (K_{D}) of 5 nM to 50 nM as measured by surface plasmon resonance or bio-layer interferometry.

316. The anti-glyco-CD44 antibody or antigen-binding fragment of any of embodiments 1 to 305, which binds to the CD44v6 glycopeptide with a binding affinity (K_{D}) of 5 nM to 25 nM as measured by surface plasmon resonance or bio-layer interferometry.

317. The anti-glyco-CD44 antibody or antigen-binding fragment of any of embodiments 1 to 305, which binds to the CD44v6 glycopeptide with a binding affinity (K_{D}) of 5 nM to 10 nM as measured by surface plasmon resonance or bio-layer interferometry.

318. The anti-glyco-CD44 antibody or antigen-binding fragment of any of embodiments 1 to 305, which binds to the CD44v6 glycopeptide with a binding affinity (K_{D}) of 10 nM to 200 nM as measured by surface plasmon resonance or bio-layer interferometry.

319. The anti-glyco-CD44 antibody or antigen-binding fragment of any of embodiments 1 to 305, which binds to the CD44v6 glycopeptide with a binding affinity (K_{D}) of 10 nM to 100 nM as measured by surface plasmon resonance or bio-layer interferometry.

320. The anti-glyco-CD44 antibody or antigen-binding fragment of any of embodiments 1 to 305, which binds to the CD44v6 glycopeptide with a binding affinity (K_{D}) of 10 nM to 150 nM as measured by surface plasmon resonance or bio-layer interferometry.

321. The anti-glyco-CD44 antibody or antigen-binding fragment of any of embodiments 1 to 305, which binds to the CD44v6 glycopeptide with a binding affinity (K_{D}) of 10 nM to 100 nM as measured by surface plasmon resonance or bio-layer interferometry.

322. The anti-glyco-CD44 antibody or antigen-binding fragment of any of embodiments 1 to 305, which binds to the CD44v6 glycopeptide with a binding affinity (K_{D}) of 10 nM to 50 nM as measured by surface plasmon resonance or bio-layer interferometry.

323. The anti-glyco-CD44 antibody or antigen-binding fragment of any of embodiments 1 to 305, which binds to the CD44v6 glycopeptide with a binding affinity (K_{D}) of 10 nM to 25 nM as measured by surface plasmon resonance or bio-layer interferometry.

324. The anti-glyco-CD44 antibody or antigen-binding fragment of any of embodiments 1 to 305, which binds to the CD44v6 glycopeptide with a binding affinity (K_{D}) of 50 nM to 200 nM as measured by surface plasmon resonance or bio-layer interferometry.

325. The anti-glyco-CD44 antibody or antigen-binding fragment of any of embodiments 1 to 305, which binds to the CD44v6 glycopeptide with a binding affinity (K_{D}) of 50 nM to 150 nM as measured by surface plasmon resonance or bio-layer interferometry.

326. The anti-glyco-CD44 antibody or antigen-binding fragment of any of embodiments 1 to 305, which binds to the CD44v6 glycopeptide with a binding affinity (K_{D}) of 50 nM to 100 nM as measured by surface plasmon resonance or bio-layer interferometry.

327. The anti-glyco-CD44 antibody or antigen-binding fragment of any of embodiments 1 to 305, which binds to the CD44v6 glycopeptide with a binding affinity (K_{D}) of 100 nM to 200 nM as measured by surface plasmon resonance or bio-layer interferometry.

328. The anti-glyco-CD44 antibody or antigen-binding fragment of any of embodiments 1 to 305, which binds to the CD44v6 glycopeptide with a binding affinity (K_{D}) of 100 nM to 150 nM as measured by surface plasmon resonance or bio-layer interferometry.

329. The anti-glyco-CD44 antibody or antigen-binding fragment of any of embodiments 306 to 328, in which the binding affinity to the CD44v6 glycopeptide is as measured by surface plasmon resonance.

330. The anti-glyco-CD44 antibody or antigen-binding fragment of embodiment 329, in which the measurement by surface plasmon resonance is carried out at a saturating concentration of the CD44v6 glycopeptide as an analyte, wherein the anti-glyco-CD44 antibody or antigen-binding fragment is an immobilized ligand.

331. The anti-glyco-CD44 antibody or antigen-binding fragment of any of embodiments 306 to 328, in which the binding affinity to the CD44v6 glycopeptide is as measured by bio-layer interferometry.

332. The anti-glyco-CD44 antibody or antigen-binding fragment of embodiment 331, in which the measurement by bio-layer interferometry is carried out with the anti-glyco-CD44 antibody or antigen-binding fragment as an analyte and the CD44v6 glycopeptide as an immobilized ligand.

333. The anti-glyco-CD44 antibody or antigen-binding fragment of any of embodiments 1 to 333, which does not specifically bind to an unglycosylated CD44v6 peptide GYRQTPKEDSHSTTGTAAA (SEQ ID NO:165) (the “unglycosylated CD44v6 peptide”).

334. The anti-glyco-CD44 antibody or antigen-binding fragment of any of embodiments 1 to 333, which has a binding affinity to the CD44v6 glycopeptide which is at least 3 times the binding affinity of the anti-glyco-CD44 antibody or antigen-binding fragment to the unglycosylated CD44v6 peptide, optionally wherein the binding affinity is measured by surface plasmon resonance, and further optionally where in the surface plasmon resonance is measured in the presence of saturating amounts of either the anti-glyco-CD44v6 peptide or the unglycosylated CD44v6 peptide (e.g., about 1 μM, about 1.5 μM, or about 2 μM of each peptide).

335. The anti-glyco-CD44 antibody or antigen-binding fragment of any of embodiments 1 to 333, which has a binding affinity to the CD44v6 glycopeptide which is at least 5 times the binding affinity of the anti-glyco-CD44 antibody or antigen-binding fragment to the unglycosylated CD44v6 peptide, optionally wherein the binding affinity is measured by surface plasmon resonance, and further optionally where in the surface plasmon resonance is measured in the presence of saturating amounts of either the anti-glyco-CD44v6 peptide or the unglycosylated CD44v6 peptide (e.g., about 1 μM, about 1.5 μM, or about 2 μM of each peptide).

336. The anti-glyco-CD44 antibody or antigen-binding fragment of any of embodiments 1 to 333, which has a binding affinity to the CD44v6 glycopeptide which is at least 10 times the binding affinity of the anti-glyco-CD44 antibody or antigen-binding fragment to the unglycosylated CD44v6 peptide, optionally wherein the binding affinity is measured by surface plasmon resonance, and further optionally where in the surface plasmon resonance is measured in the presence of saturating amounts of either the anti-glyco-CD44v6 peptide or the unglycosylated CD44v6 peptide (e.g., about 1 μM, about 1.5 μM, or about 2 μM of each peptide).

337. The anti-glyco-CD44 antibody or antigen-binding fragment of any of embodiments 1 to 333, which has a binding affinity to the CD44v6 glycopeptide which is at least 20 times the binding affinity of the anti-glyco-CD44 antibody or antigen-binding fragment to the unglycosylated CD44v6 peptide, optionally wherein the binding affinity is measured by surface plasmon resonance, and further optionally where in the surface plasmon resonance is measured in the presence of saturating amounts of either the anti-glyco-CD44v6 peptide or the unglycosylated CD44v6 peptide (e.g., about 1 μM, about 1.5 μM, or about 2 μM of each peptide).

338. The anti-glyco-CD44 antibody or antigen-binding fragment of any of embodiments 1 to 333, which has a binding affinity to the CD44v6 glycopeptide which is at least 50 times the binding affinity of the anti-glyco-CD44 antibody or antigen-binding fragment to the unglycosylated CD44v6 peptide, optionally wherein the binding affinity is measured by surface plasmon resonance, and further optionally where in the surface plasmon resonance is measured in the presence of saturating amounts of either the anti-glyco-CD44v6 peptide or the unglycosylated CD44v6 peptide (e.g., about 1 μM, about 1.5 μM, or about 2 μM of each peptide).

339. The anti-glyco-CD44 antibody or antigen-binding fragment of any of embodiments 1 to 333, which has a binding affinity to the CD44v6 glycopeptide which is at least 100 times the binding affinity of the anti-glyco-CD44 antibody or antigen-binding fragment to the unglycosylated CD44v6 peptide, optionally wherein the binding affinity is measured by surface plasmon resonance, and further optionally where in the surface plasmon resonance is measured in the presence of saturating amounts of either the anti-glyco-CD44v6 peptide or the unglycosylated CD44v6 peptide (e.g., about 1 μM, about 1.5 μM, or about 2 μM of each peptide).

340. The anti-glyco-CD44 antibody or antigen-binding fragment of any of embodiments 1 to 333, which does not specifically bind to the MUC1 tandem repeat (VTSAPDTRPAPGSTAPPAHG)₃ (SEQ ID NO:205) that has been glycosylated in vitro using purified recombinant human glycosyltransferases GalNAc-T1, GalNAc-T2, and GalNAc-T4 (“the first MUC1 glycopeptide”).

341. The anti-glyco-CD44 antibody or antigen-binding fragment of any of embodiments 1 to 333, which has a binding affinity to the CD44v6 glycopeptide which is at least 3 times the binding affinity of the anti-glyco-CD44 antibody or antigen-binding fragment to the first MUC1 glycopeptide, optionally wherein the binding affinity is measured by surface plasmon resonance, and further optionally where in the surface plasmon resonance is measured in the presence of saturating amounts of either the anti-glyco-CD44v6 peptide or the first MUC1 glycopeptide (e.g., about 1 μM, about 1.5 μM, or about 2 μM of each peptide).

342. The anti-glyco-CD44 antibody or antigen-binding fragment of any of embodiments 1 to 333, which has a binding affinity to the CD44v6 glycopeptide which is at least 5 times the binding affinity of the anti-glyco-CD44 antibody or antigen-binding fragment to the first MUC1 glycopeptide, optionally wherein the binding affinity is measured by surface plasmon resonance, and further optionally where in the surface plasmon resonance is measured in the presence of saturating amounts of either the anti-glyco-CD44v6 peptide or the first MUC1 glycopeptide (e.g., about 1 μM, about 1.5 μM, or about 2 μM of each peptide).

343. The anti-glyco-CD44 antibody or antigen-binding fragment of any of embodiments 1 to 333, which has a binding affinity to the CD44v6 glycopeptide which is at least 10 times the binding affinity of the anti-glyco-CD44 antibody or antigen-binding fragment to the first MUC1 glycopeptide, optionally wherein the binding affinity is measured by surface plasmon resonance, and further optionally where in the surface plasmon resonance is measured in the presence of saturating amounts of either the anti-glyco-CD44v6 peptide or the first MUC1 glycopeptide (e.g., about 1 μM, about 1.5 μM, or about 2 μM of each peptide).

344. The anti-glyco-CD44 antibody or antigen-binding fragment of any of embodiments 1 to 333, which has a binding affinity to the CD44v6 glycopeptide which is at least 20 times the binding affinity of the anti-glyco-CD44 antibody or antigen-binding fragment to the first MUC1 glycopeptide, optionally wherein the binding affinity is measured by surface plasmon resonance, and further optionally where in the surface plasmon resonance is measured in the presence of saturating amounts of either the anti-glyco-CD44v6 peptide or the first MUC1 glycopeptide (e.g., about 1 μM, about 1.5 μM, or about 2 μM of each peptide).

345. The anti-glyco-CD44 antibody or antigen-binding fragment of any of embodiments 1 to 333, which has a binding affinity to the CD44v6 glycopeptide which is at least 50 times the binding affinity of the anti-glyco-CD44 antibody or antigen-binding fragment to the first MUC1 glycopeptide, optionally wherein the binding affinity is measured by surface plasmon resonance, and further optionally where in the surface plasmon resonance is measured in the presence of saturating amounts of either the anti-glyco-CD44v6 peptide or the first MUC1 glycopeptide (e.g., about 1 μM, about 1.5 μM, or about 2 μM of each peptide).

346. The anti-glyco-CD44 antibody or antigen-binding fragment of any of embodiments 1 to 333, which has a binding affinity to the CD44v6 glycopeptide which is at least 100 times the binding affinity of the anti-glyco-CD44 antibody or antigen-binding fragment to the first MUC1 glycopeptide, optionally wherein the binding affinity is measured by surface plasmon resonance, and further optionally where in the surface plasmon resonance is measured in the presence of saturating amounts of either the anti-glyco-CD44v6 peptide or the first MUC1 glycopeptide (e.g., about 1 μM, about 1.5 μM, or about 2 μM of each peptide).

347. The anti-glyco-CD44 antibody or antigen-binding fragment of any of embodiments 1 to 333, which does not specifically bind to the MUC1 TAPPAHGVTSAPDTRPAPGSTAPPAHGVT (SEQ ID NO: 260) that has been glycosylated in vitro with GalNAcon the serine and threonine residues shown with bold and underlined text (“the second MUC1 glycopeptide”).

348. The anti-glyco-CD44 antibody or antigen-binding fragment of any of embodiments 1 to 333, which has a binding affinity to the CD44v6 glycopeptide which is at least 3 times the binding affinity of the anti-glyco-CD44 antibody or antigen-binding fragment to the second MUC1 glycopeptide, optionally wherein the binding affinity is measured by surface plasmon resonance, and further optionally where in the surface plasmon resonance is measured in the presence of saturating amounts of either the anti-glyco-CD44v6 peptide or the second MUC1 glycopeptide (e.g., about 1 μM, about 1.5 μM, or about 2 μM of each peptide).

349. The anti-glyco-CD44 antibody or antigen-binding fragment of any of embodiments 1 to 333, which has a binding affinity to the CD44v6 glycopeptide which is at least 5 times the binding affinity of the anti-glyco-CD44 antibody or antigen-binding fragment to the second MUC1 glycopeptide, optionally wherein the binding affinity is measured by surface plasmon resonance, and further optionally where in the surface plasmon resonance is measured in the presence of saturating amounts of either the anti-glyco-CD44v6 peptide or the second MUC1 glycopeptide (e.g., about 1 μM, about 1.5 μM, or about 2 μM of each peptide).

350. The anti-glyco-CD44 antibody or antigen-binding fragment of any of embodiments 1 to 333, which has a binding affinity to the CD44v6 glycopeptide which is at least 10 times the binding affinity of the anti-glyco-CD44 antibody or antigen-binding fragment to the second MUC1 glycopeptide, optionally wherein the binding affinity is measured by surface plasmon resonance, and further optionally where in the surface plasmon resonance is measured in the presence of saturating amounts of either the anti-glyco-CD44v6 peptide or the second MUC1 glycopeptide (e.g., about 1 μM, about 1.5 μM, or about 2 μM of each peptide).

351. The anti-glyco-CD44 antibody or antigen-binding fragment of any of embodiments 1 to 333, which has a binding affinity to the CD44v6 glycopeptide which is at least 20 times the binding affinity of the anti-glyco-CD44 antibody or antigen-binding fragment to the second MUC1 glycopeptide, optionally wherein the binding affinity is measured by surface plasmon resonance, and further optionally where in the surface plasmon resonance is measured in the presence of saturating amounts of either the anti-glyco-CD44v6 peptide or the second MUC1 glycopeptide (e.g., about 1 μM, about 1.5 μM, or about 2 μM of each peptide).

352. The anti-glyco-CD44 antibody or antigen-binding fragment of any of embodiments 1 to 333, which has a binding affinity to the CD44v6 glycopeptide which is at least 50 times the binding affinity of the anti-glyco-CD44 antibody or antigen-binding fragment to the second MUC1 glycopeptide, optionally wherein the binding affinity is measured by surface plasmon resonance, and further optionally where in the surface plasmon resonance is measured in the presence of saturating amounts of either the anti-glyco-CD44v6 peptide or the second MUC1 glycopeptide (e.g., about 1 μM, about 1.5 μM, or about 2 μM of each peptide).

353. The anti-glyco-CD44 antibody or antigen-binding fragment of any of embodiments 1 to 333, which has a binding affinity to the CD44v6 glycopeptide which is at least 100 times the binding affinity of the anti-glyco-CD44 antibody or antigen-binding fragment to the second MUC1 glycopeptide, optionally wherein the binding affinity is measured by surface plasmon resonance, and further optionally where in the surface plasmon resonance is measured in the presence of saturating amounts of either the anti-glyco-CD44v6 peptide or the second MUC1 glycopeptide (e.g., about 1 μM, about 1.5 μM, or about 2 μM of each peptide).

354. The anti-glyco-CD44 antibody or antigen-binding fragment of any of embodiments 1 to 353, which is multivalent.

355. The anti-glyco-CD44 antibody or antigen-binding fragment of any of embodiments 1 to 353, which is an antigen-binding fragment.

356. The anti-glyco-CD44 antibody or antigen-binding fragment of embodiment 355, wherein the antigen-binding fragment is in the form of a single-chain variable fragment (scFv).

357. The anti-glyco-CD44 antibody or antigen-binding fragment of embodiment 356, wherein the scFv comprises the heavy chain variable fragment N-terminal to the light chain variable fragment.

358. The anti-glyco-CD44 antibody or antigen-binding fragment of embodiment 356, wherein the scFv comprises the heavy chain variable fragment C-terminal to the light chain variable fragment.

359. The anti-glyco-CD44 antibody or antigen-binding fragment of any one of embodiments 356 to 358, wherein the scFv heavy chain variable fragment and light chain variable fragment are covalently bound to a linker sequence, which is optionally 4-15 amino acids.

360. The anti-glyco-CD44 antibody or antigen-binding fragment of any of embodiments 1 to 332, which is in the form of a multispecific antibody.

361. The anti-glyco-CD44 antibody or antigen-binding fragment of embodiment 360 wherein the multispecific antibody is a bispecific antibody that binds to a second epitope that is different from the first epitope.

362. The anti-glyco-CD44 antibody or antigen-binding fragment of embodiment 361, wherein the bispecific antibody is a bottle opener, mAb-Fv, mAb-scFv, central-scFv, one-armed central-scFv, or dual scFv format bispecific antibody.

363. The anti-glyco-CD44 antibody or antigen-binding fragment of embodiment 362, wherein the bispecific antibody is a bottle opener format bispecific antibody.

364. The anti-glyco-CD44 antibody or antigen-binding fragment of embodiment 362, wherein the bispecific antibody is a mAb-Fv format bispecific antibody.

365. The anti-glyco-CD44 antibody or antigen-binding fragment of embodiment 362, wherein the bispecific antibody is a mAb-scFv format bispecific antibody.

366. The anti-glyco-CD44 antibody or antigen-binding fragment of embodiment 362, wherein the bispecific antibody is a central-scFv format bispecific antibody.

367. The anti-glyco-CD44 antibody or antigen-binding fragment of embodiment 362, wherein the bispecific antibody is a one-armed central-scFv format bispecific antibody.

368. The anti-glyco-CD44 antibody or antigen-binding fragment of embodiment 362, wherein the bispecific antibody is a dual scFv format bispecific antibody.

369. The anti-glyco-CD44 antibody or antigen-binding fragment of embodiment 361, wherein the bispecific antibody is a CrossMab, a Fab-arm exchange antibody, a bispecific T-cell engager (BiTE), or a dual-affinity retargeting molecule (DART).

370. The anti-glyco-CD44 antibody or antigen-binding fragment of embodiment 369, wherein the bispecific antibody is a CrossMab.

371. The anti-glyco-CD44 antibody or antigen-binding fragment of embodiment 370, wherein the bispecific antibody is a CrossMabFAB.

372. The anti-glyco-CD44 antibody or antigen-binding fragment of embodiment 370, wherein the bispecific antibody is a CrossMabVH-VL.

373. The anti-glyco-CD44 antibody or antigen-binding fragment of embodiment 370, wherein the bispecific antibody is a CrossMabCH1-CL.

374. The anti-glyco-CD44 antibody or antigen-binding fragment of embodiment 369, wherein the bispecific antibody is a Fab-arm exchange antibody.

375. The anti-glyco-CD44 antibody or antigen-binding fragment of embodiment 369, wherein the bispecific antibody is a dual-affinity retargeting molecule (DART).

376. The anti-glyco-CD44 antibody or antigen-binding fragment of embodiment 369, wherein the bispecific antibody is a bispecific T-cell engager (BiTE).

377. The anti-glyco-CD44 antibody or antigen-binding fragment of any one of embodiments 361 to 376, wherein the second epitope is a CD44 epitope.

378. The anti-glyco-CD44 antibody of antigen-binding fragment of any one of embodiments 361 to 376, wherein the second epitope is a CD44 epitope that is overexpressed on cancer cells as compared to normal cells.

379. The anti-glyco-CD44 antibody or antigen-binding fragment of any one of embodiments 361 to 376, wherein the second epitope is a T-cell epitope.

380. The anti-glyco-CD44 antibody or antigen-binding fragment of embodiment 379, wherein the T-cell epitope comprises a CD3 epitope, a CD8 epitope, a CD16 epitope, a CD25 epitope, a CD28 epitope, or an NKG2D epitope.

381. The anti-glyco-CD44 antibody or antigen-binding fragment of embodiment 380, wherein the T-cell epitope comprises a CD3 epitope, which is optionally an epitope present in human CD3.

382. The anti-glyco-CD44 antibody or antigen-binding fragment of embodiment 381, wherein the CD3 epitope comprises a CD3 gamma epitope, a CD3 delta epitope, a CD3 epsilon epitope, or a CD3 zeta epitope.

383. The anti-glyco-CD44 antibody or antigen-binding fragment of any one of embodiments 1 to 382 which is conjugated to a detectable moiety.

384. The anti-glyco-CD44 antibody or antigen binding fragment of embodiment 383 in which the detectable moiety is an enzyme, a radioisotope, or a fluorescent label.

385. A fusion protein comprising the amino acid sequence of the anti-glyco-CD44 antibody or antigen-binding fragment of any of embodiments 1 to 384 operably linked to at least a second amino acid sequence.

386. The fusion protein of embodiment 385, wherein the second amino acid sequence is that of 4-1BB, CD3-zeta, or a fragment thereof.

387. The fusion protein of embodiment 385, wherein the second amino acid sequence is that of a fusion peptide.

388. The fusion protein of embodiment 387, wherein the fusion peptide is a CD28-CD3-zeta or 4-1BB (CD137)-CD3-zeta fusion peptide.

389. The fusion protein of embodiment 385, wherein the second amino acid sequence is that of a modulator of T cell activation or a fragment thereof.

390. The fusion protein of embodiment 389, wherein the modulator of T cell activation is IL-15 or IL-15Ra.

391. The fusion protein of embodiment 385, wherein the second amino acid sequence is that of a MIC protein domain.

392. The fusion protein of embodiment 391, wherein the MIC protein domain is an α1-α2 domain.

393. The fusion protein of embodiment 392, wherein the α1-α2 domain is a MICA, MICB, ULBP1, ULBP2, ULBP3, ULBP4, ULBP5, ULBP6, or OMCP α1-α2 domain.

394. The fusion protein of any one of embodiments 391 to 393, wherein the MIC protein domain is an engineered MIC protein domain.

395. A chimeric antigen receptor (CAR) comprising one or more antigen-binding fragments according to any one of embodiments 355 to 359.

396. The CAR of embodiment 395, which comprises one or more scFvs according to any one of embodiments 356 to 359.

397. The CAR of embodiment 396, which comprises one scFv according to any one of embodiments 356 to 359.

398. The CAR of embodiment 397, which comprises two scFvs according to any one of embodiments 356 to 359.

399. The CAR of embodiment 398, wherein the two scFvs have the same amino acid sequence.

400. The CAR of embodiment 398 or 399, wherein the two scFvs are covalently bound by a linker sequence, which is optionally 4-15 amino acids.

401. The CAR of any one of embodiments 395 to 400, comprising in amino- to carboxy-terminal order: (i) the one or more antigen-binding fragments, (ii) a transmembrane domain, and (iii) an intracellular signaling domain.

402. The CAR of embodiment 401, wherein the transmembrane domain comprises a CD28 transmembrane domain.

403. The CAR of embodiment 402, wherein the CD28 transmembrane domain comprises the amino acid sequence FVVVLVVVGGVLACYSLLVTVAFIIFVVV (SEQ ID NO:186).

404. The CAR of any one of embodiments 401 to 403, wherein the intracellular signaling domain comprises a co-stimulatory signaling region.

405. The CAR of embodiment 404, wherein the co-stimulatory signaling region comprises the cytoplasmic domain of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, DAP10, GITR, or a combination thereof.

406. The CAR of embodiment 405, wherein the co-stimulatory signaling domain comprises the cytoplasmic domain of CD28.

407. The CAR of embodiment 406, wherein the cytoplasmic domain of CD28 comprises the amino acid sequence

(SEQ ID NO: 179)
RSKRSRLLHSDYMNMTPRRPGPTRKHYQPYAPPRDFAAYRS.

408. The CAR of any one of embodiments 401 to 407, wherein the intracellular signaling domain comprises a T cell signaling domain.

409. The CAR of embodiment 408, wherein the T cell signaling domain is C-terminal to the co-stimulatory signaling region.

410. The CAR of embodiment 408 or 409, wherein the T cell signaling domain comprises a CD3-zeta signaling domain.

411. The CAR of embodiment 410, wherein the CD3-zeta signaling domain comprises the amino acid sequence

(SEQ ID NO: 180)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR.

412. The CAR of any one of embodiments 401 to 411, which further comprises a signal peptide N-terminal to the one or more antibody fragments or one or more scFvs.

413. The CAR of embodiment 411, wherein the signal peptide is a human CD8 signal peptide.

414. The CAR of embodiment 413, wherein the human CD8 signal peptide comprises the amino acid sequence MALPVTALLLPLALLLHAARP (SEQ ID NO:175).

415. The CAR of any one of embodiments 401 to 414, which further comprises a hinge between the one or more antigen-binding fragments and the transmembrane domain.

416. The CAR of embodiment 415, wherein the hinge comprises a human CD8a hinge.

417. The CAR of embodiment 416, wherein the human CD8a hinge comprises the amino acid sequence TTTPAPRPPTPAPTIASPLSLRPEACRPAAGGAVHTRGLDFAC (SEQ ID NO:203).

418. The CAR of embodiment 416, wherein the human CD8a hinge comprises the amino acid sequence TTTPAPRPPTPAPTIASPLSLRPEACRPAAGGAVHTRGLDFACD (SEQ ID NO:176).

419. The CAR of embodiment 415, wherein the hinge comprises a human IgG4-short hinge comprising the amino acid sequence ESKYGPPCPSCP (SEQ ID NO:177).

420. The CAR of embodiment 415, wherein the hinge comprises a human IgG4-long hinge comprising the amino acid sequence

(SEQ ID NO: 178)
ESKYGPPCPPCPAPPVAGPSVFLFPPKPKDTLMISRTPEVTCVVVDVSQE
DPEVQFNWYVDGVEVHNAKTKPREEQFQSTYRVVSVLTVLHQDWLNGKEY
KCKVSNKGLPSSIEKTISKAKGQPREPQVYTLPPSQEEMTKNQVSLTCLV
KGFYPSDIAVEWESNGQPENNYKTTPPVLDSDGSFFLYSRLTVDKSRWQE
GNVFSCSVMHEALHNHYTQKSLSLSLGKM.

421. A chimeric antigen receptor (CAR), whose amino acid sequence comprises the amino acid sequence of SEQ ID NO:157.

422. A chimeric antigen receptor (CAR), whose amino acid sequence comprises the amino acid sequence of SEQ ID NO:158.

423. A chimeric antigen receptor (CAR), whose amino acid sequence comprises the amino acid sequence of SEQ ID NO:159.

424. A chimeric antigen receptor (CAR), whose amino acid sequence comprises the amino acid sequence of SEQ ID NO:160.

425. A chimeric antigen receptor (CAR), whose amino acid sequence comprises the amino acid sequence of SEQ ID NO:161.

426. A chimeric antigen receptor (CAR), whose amino acid sequence comprises the amino acid sequence of SEQ ID NO:162.

427. A chimeric antigen receptor (CAR), whose amino acid sequence comprises the amino acid sequence of SEQ ID NO:163.

428. A chimeric antigen receptor (CAR), whose amino acid sequence comprises the amino acid sequence of SEQ ID NO:164.

429. A chimeric antigen receptor (CAR), whose amino acid sequence comprises the amino acid sequence of SEQ ID NO:261.

430. An antibody-drug conjugate comprising the anti-glyco-CD44 antibody or antigen-binding fragment of any of embodiments 1 to 384 or the fusion protein of any one of embodiments 385 to 394 conjugated to a cytotoxic agent.

431. The antibody-drug conjugate of embodiment 429, wherein the cytotoxic agent is an auristatin, a DNA minor groove binding agent, an alkylating agent, an enediyne, a lexitropsin, a duocarmycin, a taxane, a dolastatin, a maytansinoid, or a vinca alkaloid.

432. The antibody-drug conjugate of embodiment 431, wherein the anti-glyco-CD44 antibody or antigen-binding fragment or bispecific antibody is conjugated to the cytotoxic agent via a linker.

433. The antibody-drug conjugate of embodiment 432, wherein the linker is cleavable under intracellular conditions.

434. The antibody-drug conjugate of embodiment 433, wherein the cleavable linker is cleavable by an intracellular protease.

435. The antibody-drug conjugate of embodiment 434, wherein the linker comprises a dipeptide.

436. The antibody-drug conjugate of embodiment 435, wherein the dipeptide is val-cit or phe-lys.

437. The antibody-drug conjugate of embodiment 433, wherein the cleavable linker is hydrolyzable at a pH of less than 5.5.

438. The antibody-drug conjugate of embodiment 437, wherein the hydrolyzable linker is a hydrazone linker.

439. The antibody-drug conjugate of embodiment 433, wherein the cleavable linker is a disulfide linker.

440. A nucleic acid comprising a coding region for an anti-glyco-CD44 antibody or antigen-binding fragment of any of embodiments 1 to 384, the fusion protein of any one of embodiments 385 to 394, or the CAR of any one of embodiments 395 to 429.

441. The nucleic acid of embodiment 440 in which the coding region is codon-optimized for expression in a human cell.

442. A vector comprising the nucleic acid of embodiment 440 or embodiment 441.

443. The vector of embodiment 442 which is a viral vector.

444. The vector of embodiment 443 wherein the viral vector is a lentiviral vector.

445. A host cell engineered to express the nucleic acid of embodiment 440 or embodiment 441.

446. The host cell of embodiment 445, which is a human T-cell engineered to express the CAR of any one of embodiments 395 to 429.

447. A host cell comprising the vector of any one of embodiments 442 to 444.

448. The host cell of embodiment 447 which is a T-cell and wherein the vector encodes the CAR of any one of embodiments 395 to 429.

449. A pharmaceutical composition comprising (a) the anti-glyco-CD44 antibody or antigen binding fragment of any of embodiments 1 to 384, the fusion protein of any one of embodiments 385 to 394, the CAR of any one of embodiments 395 to 429, the antibody-drug conjugate of any one of embodiments 429 to 439, the nucleic acid of embodiment 440 or embodiment 441, the vector of any one of embodiments 442 to 444, or the host cell of any one of embodiments 445 to 448, and (b) a physiologically suitable buffer, adjuvant, diluent, or combination thereof.

450. A method of treating cancer comprising administering to a subject in need thereof an effective amount of the anti-glyco-CD44 antibody or antigen binding fragment of any of embodiments 1 to 384, the fusion protein of any one of embodiments 385 to 394, the CAR of any one of embodiments 395 to 429, the antibody-drug conjugate of any one of embodiments 429 to 439, the nucleic acid of embodiment 440 or embodiment 441, the vector of any one of embodiments 442 to 444, the host cell of any one of embodiments 445 to 448, or the pharmaceutical composition of embodiment 449.

451. The method of embodiment 450, wherein the subject is suffering from breast cancer, lung cancer, a urogenital cancer, pancreatic cancer, colorectal cancer, ovarian cancer, gastric cancer, or head and neck cancer, skin cancer, malignant melanoma, liver cancer, a glioma, thyroid cancer, cervical cancer, or endometrial cancer.

452. The method of embodiment 451, wherein the subject is suffering from breast cancer.

453. The method of embodiment 451, wherein the subject is suffering from lung cancer.

454. The method of embodiment 453, wherein the lung cancer is non-small cell lung cancer.

455. The method of embodiment 451, wherein the subject is suffering from a urogenital cancer.

456. The method of embodiment 455, wherein the urogenital cancer is prostate cancer.

457. The method of embodiment 455, wherein the urogenital cancer is kidney cancer.

458. The method of embodiment 451, wherein the subject is suffering from pancreatic cancer.

459. The method of embodiment 451, wherein the subject is suffering from colorectal cancer.

460. The method of embodiment 451, wherein the subject is suffering from ovarian cancer.

461. The method of embodiment 451, wherein the subject is suffering from gastric cancer.

462. The method of embodiment 451, wherein the subject is suffering from head and neck cancer.

463. The method of embodiment 462, wherein the head and neck cancer is head and neck squamous cell carcinoma (HNSCC).

464. The method of embodiment 451, wherein the subject is suffering from skin cancer.

465. The method of embodiment 451, wherein the subject is suffering from malignant melanoma.

466. The method of embodiment 451, wherein the subject is suffering from liver cancer.

467. The method of embodiment 451, wherein the subject is suffering from a glioma.

468. The method of embodiment 451, wherein the subject is suffering from thyroid cancer.

469. The method of embodiment 451, wherein the subject is suffering from cervical cancer.

470. The method of embodiment 451, wherein the subject is suffering from endometrial cancer.

471. A method of detecting cancer in a biological sample, comprising contacting a sample with an anti-glyco-CD44 antibody or antigen-binding fragment according to any one of embodiments 1 to 384 and detecting binding of the anti-glyco-CD44 antibody or antigen-binding fragment.

472. The method of embodiment 471, further comprising quantitating the binding of the anti-glyco-CD44 antibody or antigen-binding fragment.

473. The method of embodiment 471 or embodiment 472, wherein the binding is compared to a normal tissue control as a negative/baseline control and/or to a cancerous tissue control as a positive control.

474. The method of any one of embodiments 471 to 473, wherein the cancer is breast cancer, lung cancer, a urogenital cancer, pancreatic cancer, colorectal cancer, ovarian cancer, gastric cancer, head and neck cancer, skin cancer, malignant melanoma, liver cancer, a glioma, thyroid cancer, cervical cancer, or endometrial cancer.

475. The method of embodiment 474, wherein the cancer is breast cancer.

476. The method of embodiment 474, wherein the cancer is lung cancer.

477. The method of embodiment 476, wherein the lung cancer is non-small cell lung cancer.

478. The method of embodiment 474, wherein the cancer is a urogenital cancer.

479. The method of embodiment 478, wherein the cancer is prostate cancer.

480. The method of embodiment 478, wherein the cancer is kidney cancer.

481. The method of embodiment 474, wherein the cancer is pancreatic cancer.

482. The method of embodiment 474, wherein the cancer is colorectal cancer.

483. The method of embodiment 474, wherein the cancer is ovarian cancer.

484. The method of embodiment 474, wherein the cancer is gastric cancer.

485. The method of embodiment 474, wherein the cancer is head and neck cancer.

486. The method of embodiment 485, wherein the head and neck cancer is HNSCC.

487. The method of embodiment 474, wherein the cancer is skin cancer.

488. The method of embodiment 474, wherein the cancer is malignant melanoma.

489. The method of embodiment 474, wherein the cancer is liver cancer.

490. The method of embodiment 474, wherein the cancer is a glioma.

491. The method of embodiment 474, wherein the cancer is thyroid cancer.

492. The method of embodiment 474, wherein the cancer is cervical cancer.

493. The method of embodiment 474, wherein the cancer is endometrial cancer.

494. The method of any one of embodiments 450 to 493, when depending from any one of embodiments 391 to 394, which further comprises administering to the subject genetically modified T-cells engineered to express a CAR comprising a NKG2D receptor capable of specifically binding the MIC protein domain.

495. The anti-glyco-CD44 antibody or antigen binding fragment of any of embodiments 1 to 384, the fusion protein of any one of embodiments 385 to 394, the CAR of any one of embodiments 395 to 429, the antibody-drug conjugate of any one of embodiments 429 to 439, the nucleic acid of embodiment 440 or embodiment 441, the vector of any one of embodiments 442 to 444, the host cell of any one of embodiments 445 to 448, or the pharmaceutical composition of embodiment 449 for use as a medicament.

496. The anti-glyco-CD44 antibody or antigen binding fragment of any of embodiments 1 to 384, the fusion protein of any one of embodiments 385 to 394, the CAR of any one of embodiments 395 to 429, the antibody-drug conjugate of any one of embodiments 429 to 439, the nucleic acid of embodiment 440 or embodiment 441, the vector of any one of embodiments 442 to 444, the host cell of any one of embodiments 445 to 448, or the pharmaceutical composition of embodiment 449 for use in the treatment of cancer, optionally wherein the cancer is breast cancer, lung cancer, a urogenital cancer (e.g., prostate cancer or kidney cancer), pancreatic cancer, colorectal cancer, ovarian cancer, gastric cancer, head and neck cancer, skin cancer, malignant melanoma, liver cancer, a glioma, thyroid cancer or endometrial cancer.

497. The anti-glyco-CD44 antibody or antigen binding fragment of any of embodiments 1 to 384, the fusion protein of any one of embodiments 385 to 394, the CAR of any one of embodiments 395 to 429, the antibody-drug conjugate of any one of embodiments 429 to 439, the nucleic acid of embodiment 440 or embodiment 441, the vector of any one of embodiments 442 to 444, the host cell of any one of embodiments 445 to 448, or the pharmaceutical composition of embodiment 449 for use in the treatment of breast cancer.

498. The anti-glyco-CD44 antibody or antigen binding fragment of any of embodiments 1 to 384, the fusion protein of any one of embodiments 385 to 394, the CAR of any one of embodiments 395 to 429, the antibody-drug conjugate of any one of embodiments 429 to 439, the nucleic acid of embodiment 440 or embodiment 441, the vector of any one of embodiments 442 to 444, the host cell of any one of embodiments 445 to 448, or the pharmaceutical composition of embodiment 449 for use in the treatment of lung cancer, optionally wherein the lung cancer is non-small cell lung cancer.

499. The anti-glyco-CD44 antibody or antigen binding fragment of any of embodiments 1 to 384, the fusion protein of any one of embodiments 385 to 394, the CAR of any one of embodiments 395 to 429, the antibody-drug conjugate of any one of embodiments 429 to 439, the nucleic acid of embodiment 440 or embodiment 441, the vector of any one of embodiments 442 to 444, the host cell of any one of embodiments 445 to 448, or the pharmaceutical composition of embodiment 449 for use in the treatment of a urogenital cancer.

500. The anti-glyco-CD44 antibody or antigen binding fragment of any of embodiments 1 to 384, the fusion protein of any one of embodiments 385 to 394, the CAR of any one of embodiments 395 to 429, the antibody-drug conjugate of any one of embodiments 429 to 439, the nucleic acid of embodiment 440 or embodiment 441, the vector of any one of embodiments 442 to 444, the host cell of any one of embodiments 445 to 448, or the pharmaceutical composition of embodiment 449 for use in the treatment of prostate cancer.

501. The anti-glyco-CD44 antibody or antigen binding fragment of any of embodiments 1 to 384, the fusion protein of any one of embodiments 385 to 394, the CAR of any one of embodiments 395 to 429, the antibody-drug conjugate of any one of embodiments 429 to 439, the nucleic acid of embodiment 440 or embodiment 441, the vector of any one of embodiments 442 to 444, the host cell of any one of embodiments 445 to 448, or the pharmaceutical composition of embodiment 449 for use in the treatment of kidney cancer.

502. The anti-glyco-CD44 antibody or antigen binding fragment of any of embodiments 1 to 384, the fusion protein of any one of embodiments 385 to 394, the CAR of any one of embodiments 395 to 429, the antibody-drug conjugate of any one of embodiments 429 to 439, the nucleic acid of embodiment 440 or embodiment 441, the vector of any one of embodiments 442 to 444, the host cell of any one of embodiments 445 to 448, or the pharmaceutical composition of embodiment 449 for use in the treatment of pancreatic cancer.

503. The anti-glyco-CD44 antibody or antigen binding fragment of any of embodiments 1 to 384, the fusion protein of any one of embodiments 385 to 394, the CAR of any one of embodiments 395 to 429, the antibody-drug conjugate of any one of embodiments 429 to 439, the nucleic acid of embodiment 440 or embodiment 441, the vector of any one of embodiments 442 to 444, the host cell of any one of embodiments 445 to 448, or the pharmaceutical composition of embodiment 449 for use in the treatment of colorectal cancer.

504. The anti-glyco-CD44 antibody or antigen binding fragment of any of embodiments 1 to 384, the fusion protein of any one of embodiments 385 to 394, the CAR of any one of embodiments 395 to 429, the antibody-drug conjugate of any one of embodiments 429 to 439, the nucleic acid of embodiment 440 or embodiment 441, the vector of any one of embodiments 442 to 444, the host cell of any one of embodiments 445 to 448, or the pharmaceutical composition of embodiment 449 for use in the treatment of ovarian cancer.

505. The anti-glyco-CD44 antibody or antigen binding fragment of any of embodiments 1 to 384, the fusion protein of any one of embodiments 385 to 394, the CAR of any one of embodiments 395 to 429, the antibody-drug conjugate of any one of embodiments 429 to 439, the nucleic acid of embodiment 440 or embodiment 441, the vector of any one of embodiments 442 to 444, the host cell of any one of embodiments 445 to 448, or the pharmaceutical composition of embodiment 449 for use in the treatment of gastric cancer.

506. The anti-glyco-CD44 antibody or antigen binding fragment of any of embodiments 1 to 384, the fusion protein of any one of embodiments 385 to 394, the CAR of any one of embodiments 395 to 429, the antibody-drug conjugate of any one of embodiments 429 to 439, the nucleic acid of embodiment 440 or embodiment 441, the vector of any one of embodiments 442 to 444, the host cell of any one of embodiments 445 to 448, or the pharmaceutical composition of embodiment 449 for use in the treatment of head and neck cancer, optionally wherein the cancer is HNSCC.

507. The anti-glyco-CD44 antibody or antigen binding fragment of any of embodiments 1 to 384, the fusion protein of any one of embodiments 385 to 394, the CAR of any one of embodiments 395 to 429, the antibody-drug conjugate of any one of embodiments 429 to 439, the nucleic acid of embodiment 440 or embodiment 441, the vector of any one of embodiments 442 to 444, the host cell of any one of embodiments 445 to 448, or the pharmaceutical composition of embodiment 449 for use in the treatment of skin cancer.

508. The anti-glyco-CD44 antibody or antigen binding fragment of any of embodiments 1 to 384, the fusion protein of any one of embodiments 385 to 394, the CAR of any one of embodiments 395 to 429, the antibody-drug conjugate of any one of embodiments 429 to 439, the nucleic acid of embodiment 440 or embodiment 441, the vector of any one of embodiments 442 to 444, the host cell of any one of embodiments 445 to 448, or the pharmaceutical composition of embodiment 449 for use in the treatment of malignant melanoma.

509. The anti-glyco-CD44 antibody or antigen binding fragment of any of embodiments 1 to 384, the fusion protein of any one of embodiments 385 to 394, the CAR of any one of embodiments 395 to 429, the antibody-drug conjugate of any one of embodiments 429 to 439, the nucleic acid of embodiment 440 or embodiment 441, the vector of any one of embodiments 442 to 444, the host cell of any one of embodiments 445 to 448, or the pharmaceutical composition of embodiment 449 for use in the treatment of liver cancer.

510. The anti-glyco-CD44 antibody or antigen binding fragment of any of embodiments 1 to 384, the fusion protein of any one of embodiments 385 to 394, the CAR of any one of embodiments 395 to 429, the antibody-drug conjugate of any one of embodiments 429 to 439, the nucleic acid of embodiment 440 or embodiment 441, the vector of any one of embodiments 442 to 444, the host cell of any one of embodiments 445 to 448, or the pharmaceutical composition of embodiment 449 for use in the treatment of a glioma.

511. The anti-glyco-CD44 antibody or antigen binding fragment of any of embodiments 1 to 384, the fusion protein of any one of embodiments 385 to 394, the CAR of any one of embodiments 395 to 429, the antibody-drug conjugate of any one of embodiments 429 to 439, the nucleic acid of embodiment 440 or embodiment 441, the vector of any one of embodiments 442 to 444, the host cell of any one of embodiments 445 to 448, or the pharmaceutical composition of embodiment 449 for use in the treatment of thyroid cancer.

512. The anti-glyco-CD44 antibody or antigen binding fragment of any of embodiments 1 to 384, the fusion protein of any one of embodiments 385 to 394, the CAR of any one of embodiments 395 to 429, the antibody-drug conjugate of any one of embodiments 429 to 439, the nucleic acid of embodiment 440 or embodiment 441, the vector of any one of embodiments 442 to 444, the host cell of any one of embodiments 445 to 448, or the pharmaceutical composition of embodiment 449 for use in the treatment of cervical cancer.

513. The anti-glyco-CD44 antibody or antigen binding fragment of any of embodiments 1 to 384, the fusion protein of any one of embodiments 385 to 394, the CAR of any one of embodiments 395 to 429, the antibody-drug conjugate of any one of embodiments 429 to 439, the nucleic acid of embodiment 440 or embodiment 441, the vector of any one of embodiments 442 to 444, the host cell of any one of embodiments 445 to 448, or the pharmaceutical composition of embodiment 449 for use in the treatment of endometrial cancer.

514. Use of the anti-glyco-CD44 antibody or antigen binding fragment of any of embodiments 1 to 384, the fusion protein of any one of embodiments 385 to 394, the CAR of any one of embodiments 395 to 429, the antibody-drug conjugate of any one of embodiments 429 to 439, the nucleic acid of embodiment 440 or embodiment 441, the vector of any one of embodiments 442 to 444, the host cell of any one of embodiments 445 to 448, or the pharmaceutical composition of embodiment 449 for the manufacture of a medicament for the treatment of cancer, optionally wherein the cancer is breast cancer, lung cancer, a urogenital cancer (e.g., prostate cancer or kidney cancer), pancreatic cancer, colorectal cancer, ovarian cancer, gastric cancer, head and neck cancer, skin cancer, malignant melanoma, liver cancer, a glioma, thyroid cancer or endometrial cancer.

515. The use according to embodiment 514, wherein the cancer is breast cancer

516. The use according to embodiment 514, wherein the cancer is lung cancer.

517. The use according to embodiment 516, wherein the cancer is non-small cell lung

518. The use according to embodiment 514, wherein the cancer is a urogenital cancer.

519. The use according to embodiment 518, wherein the cancer is prostate cancer.

520. The use according to embodiment 518, wherein the cancer is kidney cancer.

521. The use according to embodiment 514, wherein the cancer is pancreatic cancer.

522. The use according to embodiment 514, wherein the cancer is colorectal cancer.

523. The use according to embodiment 514, wherein the cancer is ovarian cancer.

524. The use according to embodiment 514, wherein the cancer is gastric cancer.

525. The use according to embodiment 514, wherein the cancer is head and neck cancer.

526. The use according to embodiment 525, wherein the head and neck cancer is HNSCC.

527. The use according to embodiment 514, wherein the cancer is skin cancer.

528. The use according to embodiment 514, wherein the cancer is malignant melanoma.

529. The use according to embodiment 514, wherein the cancer is liver cancer.

530. The use according to embodiment 514, wherein the cancer is a glioma.

531. The use according to embodiment 514, wherein the cancer is thyroid cancer.

532. The use according to embodiment 514, wherein the cancer is cervical cancer.

533. The use according to embodiment 514, wherein the cancer is endometrial cancer.

534. A peptide of 12-30 amino acids in length comprising amino acids 4-13 of SEQ ID NO:165.

535. The peptide of embodiment 534 which is 15-25 amino acids in length

536. The peptide of embodiment 534 which is 18-20 amino acids in length

537. The peptide of embodiment 534 which consists of SEQ ID NO:165.

538. The peptide of any one of embodiments 534 to 537 which is O-glycosylated at the threonine corresponding to position 5 of SEQ ID NO:165 and/or the serine corresponding to position 12 of SEQ ID NO:165.

539. The peptide of embodiment 538, wherein the O-glycosylation comprises or consists of GalNAc.

540. A composition comprising the peptide of embodiment 538 or embodiment 539 and an adjuvant.

541. The composition of embodiment 540, wherein the adjuvant comprises a Freund's adjuvant and/or an aluminum salt (e.g., aluminum hydroxide).

542. A method of generating antibodies against a tumor-associated form of CD44v6, comprising administering to an animal the peptide of embodiment 538 or embodiment 539 or the composition of claim 540 or embodiment 541.

543. The method of embodiment 542, further comprises collecting antibodies from the animal.

544. A method of eliciting an immune response against a tumor-associated form of CD44v6, comprising administering to a subject the peptide of embodiment 538 or embodiment 539 or the composition of embodiment 540 or embodiment 541.

545. The method of any one of embodiments 542 to 544, wherein the animal is a mouse or rabbit.

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

1. An anti-glyco-CD44 antibody or antigen binding fragment that competes with an antibody or antigen binding fragment comprising:
(a) a heavy chain variable (VH) sequence of SEQ ID NO:1 and a light chain variable (VL) sequence of SEQ ID NO:2;
(b) a heavy chain variable (VH) sequence of SEQ ID NO:23 and a light chain variable (VL) sequence of SEQ ID NO:24;
(c) a heavy chain variable (VH) sequence of SEQ ID NO:45 and a light chain variable (VL) sequence of SEQ ID NO:46;
(d) a heavy chain variable (VH) sequence of SEQ ID NO:67 and a light chain variable (VL) sequence of SEQ ID NO:68; or
(e) a heavy chain variable (VH) sequence of SEQ ID NO:206 and a light chain variable (VL) sequence of SEQ ID NO:207
for binding a CD44v6 peptide GYRQTPKEDSHSTTGTAAA (SEQ ID NO:165) that has been glycosylated with GalNAc on threonine at amino acid position 5 of SEQ ID NO: 165 and serine at amino acid position 12 of SEQ ID NO: 165.

2. An anti-glyco-CD44 antibody or antigen binding fragment that competes with an antibody or antigen binding fragment comprising:
(a) a heavy chain variable (VH) sequence of SEQ ID NO:1 and a light chain variable (VL) sequence of SEQ ID NO:2;
(b) a heavy chain variable (VH) sequence of SEQ ID NO:23 and a light chain variable (VL) sequence of SEQ ID NO:24;
(c) a heavy chain variable (VH) sequence of SEQ ID NO:45 and a light chain variable (VL) sequence of SEQ ID NO:46;
(d) a heavy chain variable (VH) sequence of SEQ ID NO:67 and a light chain variable (VL) sequence of SEQ ID NO:68; or
(e) a heavy chain variable (VH) sequence of SEQ ID NO:206 and a light chain variable (VL) sequence of SEQ ID NO:207
for specifically binding to i) COSMC knock-out HaCaT cells and/or (ii) COSMC knock-out HEK293 cells recombinantly expressing CD44.

3. An anti-glyco-CD44 antibody or antigen binding fragment comprising a complementarity determining region (CDR) H1 comprising the amino acid sequence of SEQ ID NO:256; a CDR-H2 comprising the amino acid sequence of SEQ ID NO:257; a CDR-H3 comprising the amino acid sequence of SEQ ID NO:103, SEQ ID NO: 109, SEQ ID NO: 115, SEQ ID NO:121, SEQ ID NO:131, SEQ ID NO:137, SEQ ID NO:143, or SEQ ID NO: 149; SEQ ID NO:242, or SEQ ID NO:252; a CDR-L1 comprising the amino acid sequence of SEQ ID NO:104, SEQ ID NO:110, SEQ ID NO:116, SEQ ID NO:122, SEQ ID NO:132, SEQ ID NO:138, SEQ ID NO:144, or SEQ ID NO:150, SEQ ID NO:243, or SEQ ID NO:253; a CDR-L2 comprising the amino acid sequence of SEQ ID NO:258, and a CDR-L3 comprising the amino acid sequence of SEQ ID NO:259.

4. The anti-glyco-CD44 antibody or antigen-binding fragment of claim 3, wherein CDR-H1 comprises the amino acid sequence of SEQ ID NO:89, SEQ ID NO:93, SEQ ID NO:97 SEQ ID NO:125, SEQ ID NO:153, SEQ ID NO:228, SEQ ID NO:232, SEQ ID NO:236, or SEQ ID NO:246.

5. The anti-glyco-CD44 antibody or antigen-binding fragment of claim 4, wherein:
(a) the amino acid designated X₁ in SEQ ID NO:89, SEQ ID NO:97, and SEQ ID NO:125 is Y or F;
(b) the amino acid designated X₂ in SEQ ID NO:89, SEQ ID NO:97, and SEQ ID NO:125 is T or S;
(c) the amino acid designated X₃ in SEQ ID NO:89, SEQ ID NO:93, SEQ ID NO:97, SEQ ID NO:125, and SEQ ID NO:153 is Y or F;
(d) the amino acid designated X₄ in SEQ ID NO:89, SEQ ID NO:93, and SEQ ID NO:125 is W, A, or G;
(e) the amino acid designated X₅ in SEQ ID NO:93 and SEQ ID NO:125 is M or I; and
(f) the amino acid designated X₆ in SEQ ID NO:93 and SEQ ID NO:125 is H or S.

6. The anti-glyco-CD44 antibody or antigen-binding fragment of claim 4, wherein:
(a) the amino acid designated X₄₁ in SEQ ID NO:228, SEQ ID NO:236, and SEQ ID NO:246 is Y or F;
(b) the amino acid designated X₄₂ in SEQ ID NO:228, SEQ ID NO:236, and SEQ ID NO:246 is F or I;
(c) the amino acid designated X₄₃ in SEQ ID NO:228, SEQ ID NO:236, and SEQ ID NO:246 is T, S, or N;
(d) the amino acid designated X₄₄ in SEQ ID NO:228, SEQ ID NO:232, SEQ ID NO:236, SEQ ID NO:246, and SEQ ID NO:256 is S or T;
(e) the amino acid designated X₄₅ in SEQ ID NO:228, SEQ ID NO:232, SEQ ID NO:236, SEQ ID NO:246, and SEQ ID NO:256 is Y or F;
(f) the amino acid designated X₄₆ in SEQ ID NO:228, SEQ ID NO:232, and SEQ ID NO:246 is W, A, G, or H;
(g) the amino acid designated X₄₇ in SEQ ID NO:232 and SEQ ID NO:246 is M or I; and
(h) the amino acid designated X₄₈ in SEQ ID NO:232 and SEQ ID NO:246 is H, S, or G.

7. The anti-glyco-CD44 antibody or antigen-binding fragment of claim 3, wherein CDR-H2 comprises the amino acid sequence of SEQ ID NO:90, SEQ ID NO:94, SEQ ID NO:98, SEQ ID NO:229, SEQ ID NO:233, or SEQ ID NO:237.

8. The anti-glyco-CD44 antibody or antigen-binding fragment of claim 7, wherein:
(a) the amino acid designated X₇ in SEQ ID NO:94 is N, E, or Y;
(b) the amino acid designated X₈ in SEQ ID NO:90, SEQ ID NO:94, and SEQ ID NO:98 is Y or S;
(c) the amino acid designated X₉ in SEQ ID NO:90, SEQ ID NO:94, and SEQ ID NO:98 is P or S;
(d) wherein the amino acid designated X₁₀ in SEQ ID NO:90, SEQ ID NO:94, and SEQ ID NO:98 is R or G;
(e) the amino acid designated X₁₁ in SEQ ID NO:90, SEQ ID NO:94, and SEQ ID NO:98 is S or G;
(f) the amino acid designated X₁₂ in SEQ ID NO:90, SEQ ID NO:94, and SEQ ID NO:98 is G or S;
(g) the amino acid designated X₁₃ in SEQ ID NO:90, SEQ ID NO:94, and SEQ ID NO:98 is T or Y;
(h) the amino acid designated X₁₄ in SEQ ID NO:90 and SEQ ID NO:94 is T or I;
(i) the amino acid designated X₁₅ in SEQ ID NO:94 is N or Y;
(j) the amino acid designated X₁₆ in SEQ ID NO:94 is D, P, or A;
(k) the amino acid designated X₁₇ in SEQ ID NO:94 is G or D;
(l) the amino acid designated X₁₈ in SEQ ID NO:94 is Y or T;
(m) the amino acid designated X₁₉ in SEQ ID NO:94 is F or V; and
(n) the amino acid designated X₂₀ in SEQ ID NO:94 is K, T, S, or G.

9. The anti-glyco-CD44 antibody or antigen-binding fragment of claim 7, wherein:
(a) the amino acid designated X₄₉ in SEQ ID NO:233 is N, E, Y, or I;
(b) the amino acid designated X₅₀ in SEQ ID NO:233 is I or V;
(c) the amino acid designated X₅₁ in SEQ ID NO:229, SEQ ID NO:233, and SEQ ID NO:237 is Y or S;
(d) the amino acid designated X₅₂ in SEQ ID NO:229, SEQ ID NO:233, and SEQ ID NO:237 is P, S, or H;
(e) the amino acid designated X₅₃ in SEQ ID NO:229, SEQ ID NO:233, and SEQ ID NO:237 is R, G, or D;
(f) the amino acid designated X₅₄ in SEQ ID NO:229, SEQ ID NO:233, and SEQ ID NO:237 is S or G;
(g) the amino acid designated X₅₅ in SEQ ID NO:229, SEQ ID NO:233, and SEQ ID NO:237 is G or S;
(h) the amino acid designated X₅₆ in SEQ SEQ ID NO:229, SEQ ID NO:233, and SEQ ID NO:237 is T or Y;
(i) the amino acid designated X₅₇ in SEQ ID NO:229 and SEQ ID NO:233 is T, I, or absent;
(j) the amino acid designated X₅₈ in SEQ ID NO:233 is N or Y;
(k) the amino acid designated X₆₉ in SEQ ID NO:233 is D, P, or A;
(l) the amino acid designated X₅₀ in SEQ ID NO:233 is G, D, or T;
(m) the amino acid designated X₅₁ in SEQ ID NO:233 is Y, T, or W;
(n) the amino acid designated X₅₂ in SEQ ID NO:233 is F, V, or A;
(o) the amino acid designated X₆₃ in SEQ ID NO: 233 is K or T; and
(p) the amino acid designated X₆₄ in SEQ ID NO: 233 is S or G.

10. The anti-glyco-CD44 antibody or antigen-binding fragment of claim 3, wherein CDR-L2 comprises the amino acid sequence of SEQ ID NO:91, SEQ ID NO:95, SEQ ID NO:230, or SEQ ID NO:234.

11. The anti-glyco-CD44 antibody or antigen-binding fragment of claim 10, wherein:
(a) the amino acid designated X₂₂ in SEQ ID NO:91 and SEQ ID NO:95 is G, S, or L;
(b) the amino acid designated X₂₃ in SEQ ID NO:91 and SEQ ID NO:95 is T or I;
(c) the amino acid designated X₂₄ in SEQ ID NO:91 and SEQ ID NO:95 is N or S;
(d) the amino acid designated X₂₅ in SEQ ID NO:95 is N or R;
(e) the amino acid designated X₂₆ in SEQ ID NO:95 is R or L;
(f) the amino acid designated X₂₇ in SEQ ID NO:95 is A, H, or F; and
(g) the amino acid designated X₂₅ in SEQ ID NO:95 is P or S;

12. The anti-glyco-CD44 antibody or antigen-binding fragment of claim 10, wherein:
(a) the amino acid designated X₅₅ in SEQ ID NO:230 and SEQ ID NO:234 is G, S, L, K;
(b) the amino acid designated X₆₆ in SEQ ID NO:230 and SEQ ID NO:234 is T, I, or A;
(c) the amino acid designated X₆₇ in SEQ ID NO:230 and SEQ ID NO:234 is N or S;
(d) the amino acid designated X₅₈ in SEQ ID NO:234 is N, R, or T;
(e) the amino acid designated X₆₉ in SEQ ID NO: 234 is R or L;
(f) the amino acid designated X₇₀ in SEQ ID NO: 234 is A. H, or F; and
(g) the amino acid designated X₇₁ in SEQ ID NO: 234 is P or S;

13. The anti-glyco-CD44 antibody or antigen-binding fragment of claim 3, wherein CDR-L3 comprises the amino acid sequence of SEQ ID NO:92, or SEQ ID NO:231.

14. The anti-glyco-CD44 antibody or antigen-binding fragment of claim 13, wherein:
(a) the amino acid designated X₂₉ in SEQ ID NO:92 is A, Q, or L;
(b) the amino acid designated X₃₀ in SEQ ID NO:92 is L or Q;
(c) the amino acid designated X₃₁ in SEQ ID NO:92 is L, G, or W;
(d) the amino acid designated X₃₂ in SEQ ID NO:92 is Y, S, or T;
(e) the amino acid designated X₃₃ in SEQ ID NO:92 is S, T, or H;
(f) the amino acid designated X₃₄ in SEQ ID NO:92 is N, L, or Q;
(g) the amino acid designated X₃₅ in SEQ ID NO:92 is Y or P;
(h) wherein the amino acid designated X₃₆ in SEQ ID NO:92 is W or F; and
(i) the amino acid designated X₃₇ in SEQ ID NO:92 is Y or T.

15. The anti-glyco-CD44 antibody or antigen-binding fragment of claim 13, wherein:
(a) the amino acid designated X₇₂ in SEQ ID NO:231 is A, Q, or L
(b) the amino acid designated X₇₃ in SEQ ID NO: 231 is L, Q, or G;
(c) the amino acid designated X₇₄ in SEQ ID NO: 231 is L, G, or W;
(d) the amino acid designated X₇₅ in SEQ ID NO: 231 is Y, S, or T;
(e) the amino acid designated X₇₆ in SEQ ID NO: 231 is S, T, H, or K;
(f) the amino acid designated X₇₇ in SEQ ID NO: 231 is N, L, Q, or G;
(g) the amino acid designated X₇₈ in SEQ ID NO: 231 is Y, P, or D;
(h) the amino acid designated X₇₉ in SEQ ID NO: 231 is W, F, or I;
(i) the amino acid designated X₈₀ in SEQ ID NO: 231 is Y, T, or H; and
(j) the amino acid designated X₈₁ in SEQ ID NO: 231 is P or absent.

16. The anti-glyco-CD44 antibody or antigen-binding fragment of claim 3, which has:
(a) a VH comprising complementarity determining regions (CDRs) of SEQ ID NOS:3-5 and a VL comprising CDRs of SEQ ID NOS:6-8;
(b) a VH comprising complementarity determining regions (CDRs) of SEQ ID NOS:9-11 and a VL comprising CDRs of SEQ ID NOS:12-14;
(c) a VH comprising complementarity determining regions (CDRs) of SEQ ID NOS:15-17 and a VL comprising CDRs of SEQ ID NOS:18-20;
(d) a VH comprising complementarity determining regions (CDRs) of SEQ ID NOS:25-27 and a VL comprising CDRs of SEQ ID NOS:28-30;
(e) a VH comprising complementarity determining regions (CDRs) of SEQ ID NOS:31-33 and a VL comprising CDRs of SEQ ID NOS:34-36;
(f) a VH comprising complementarity determining regions (CDRs) of SEQ ID NOS:37-39 and a VL comprising CDRs of SEQ ID NOS:40-42;
(g) a VH comprising complementarity determining regions (CDRs) of SEQ ID NOS:47-49 and a VL comprising CDRs of SEQ ID NOS:50-52;
(h) a VH comprising complementarity determining regions (CDRs) of SEQ ID NOS:53-55 and a VL comprising CDRs of SEQ ID NOS:56-58;
(i) a VH comprising complementarity determining regions (CDRs) of SEQ ID NOS:59-61 and a VL comprising CDRs of SEQ ID NOS:62-64;
(j) a VH comprising complementarity determining regions (CDRs) of SEQ ID NOS:69-71 and a VL comprising CDRs of SEQ ID NOS:72-74;
(k) a VH comprising complementarity determining regions (CDRs) of SEQ ID NOS:75-77 and a VL comprising CDRs of SEQ ID NOS:78-80;
(l) a VH comprising complementarity determining regions (CDRs) of SEQ ID NOS:81-83 and a VL comprising CDRs of SEQ ID NOS:84-86;
(m) a VH comprising complementarity determining regions (CDRs) of SEQ ID NOS:208-210 and a VL comprising CDRs of SEQ ID NOS:211-213;
(n) a VH comprising complementarity determining regions (CDRs) of SEQ ID NOS:214-216 and a VL comprising CDRs of SEQ ID NOS:217-219;
(o) a VH comprising complementarity determining regions (CDRs) of SEQ ID NOS:220-222 and a VL comprising CDRs of SEQ ID NOS:223-225;
(p) a VH comprising complementarity determining regions (CDRs) of SEQ ID NOS:101-103 and a VL comprising CDRs of SEQ ID NOS:104-106;
(q) a VH comprising complementarity determining regions (CDRs) of SEQ ID NOS:107-109 and a VL comprising CDRs of SEQ ID NOS:110-112;
(r) a VH comprising complementarity determining regions (CDRs) of SEQ ID NOS:113-115 and a VL comprising CDRs of SEQ ID NOS:116-118;
(s) a VH comprising complementarity determining regions (CDRs) of SEQ ID NOS:119-121 and a VL comprising CDRs of SEQ ID NOS:122-124;
(t) a VH comprising complementarity determining regions (CDRs) of SEQ ID NOS:240-242 and a VL comprising CDRs of SEQ ID NOS:243-245;
(u) a VH comprising complementarity determining regions (CDRs) of SEQ ID NOS:129-131 and a VL comprising CDRs of SEQ ID NOS:132-134;
(v) a VH comprising complementarity determining regions (CDRs) of SEQ ID NOS:135-137 and a VL comprising CDRs of SEQ ID NOS:138-140;
(w) a VH comprising complementarity determining regions (CDRs) of SEQ ID NOS:141-143 and a VL comprising CDRs of SEQ ID NOS:144-146;
(x) a VH comprising complementarity determining regions (CDRs) of SEQ ID NOS:147-149 and a VL comprising CDRs of SEQ ID NOS:150-152; or
(y) a VH comprising complementarity determining regions (CDRs) of SEQ ID NOS:250-252 and a VL comprising CDRs of SEQ ID NOS:253-255.

17. The anti-glyco-CD44 antibody or antigen-binding fragment of claim 3, which has:
(a) a VH comprising an amino acid sequence having at least 95% sequence identity to SEQ ID NO:1 and a VL comprising an amino acid sequence having at least 95% sequence identity to SEQ ID NO:2;
(b) a VH comprising an amino acid sequence having at least 97% sequence identity to SEQ ID NO:1 and a VL comprising an amino acid sequence having at least 97% sequence identity to SEQ ID NO:2;
(c) a VH comprising an amino acid sequence having at least 99% sequence identity to SEQ ID NO:1 and a VL comprising an amino acid sequence having at least 99% sequence identity to SEQ ID NO:2.

18. The anti-glyco-CD44 antibody or antigen-binding fragment of claim 3, which has a VH comprising the amino acid sequence of SEQ ID NO:1 and a VL comprising the amino acid sequence of SEQ ID NO:2.

19. The anti-glyco-CD44 antibody or antigen-binding fragment of claim 3, which has:
(a) a VH comprising an amino acid sequence having at least 95% sequence identity to SEQ ID NO:23 and a VL comprising an amino acid sequence having at least 95% sequence identity to SEQ ID NO:24;
(b) a VH comprising an amino acid sequence having at least 97% sequence identity to SEQ ID NO:23 and a VL comprising an amino acid sequence having at least 97% sequence identity to SEQ ID NO:24; or
(c) a VH comprising an amino acid sequence having at least 99% sequence identity to SEQ ID NO:23 and a VL comprising an amino acid sequence having at least 99% sequence identity to SEQ ID NO:24.

20. The anti-glyco-CD44 antibody or antigen-binding fragment of claim 3, which has a VH comprising the amino acid sequence of SEQ ID NO:23 and a VL comprising the amino acid sequence of SEQ ID NO:24.

21. The anti-glyco-CD44 antibody or antigen-binding fragment of claim 3, which has:
(a) a VH comprising an amino acid sequence having at least 95% sequence identity to SEQ ID NO:45 and a VL comprising an amino acid sequence having at least 95% sequence identity to SEQ ID NO:46;
(b) a VH comprising an amino acid sequence having at least 97% sequence identity to SEQ ID NO:45 and a VL comprising an amino acid sequence having at least 97% sequence identity to SEQ ID NO:46; or
(c) a VH comprising an amino acid sequence having at least 99% sequence identity to SEQ ID NO:45 and a VL comprising an amino acid sequence having at least 99% sequence identity to SEQ ID NO:46.

22. The anti-glyco-CD44 antibody or antigen-binding fragment of claim 3, which has a VH comprising the amino acid sequence of SEQ ID NO:45 and a VL comprising the amino acid sequence of SEQ ID NO:46.

23. The anti-glyco-CD44 antibody or antigen-binding fragment of claim 3, which has:
(a) a VH comprising an amino acid sequence having at least 95% sequence identity to SEQ ID NO:67 and a VL comprising an amino acid sequence having at least 95% sequence identity to SEQ ID NO:68;
(b) a VH comprising an amino acid sequence having at least 97% sequence identity to SEQ ID NO:67 and a VL comprising an amino acid sequence having at least 97% sequence identity to SEQ ID NO:68; or
(c) a VH comprising an amino acid sequence having at least 99% sequence identity to SEQ ID NO:67 and a VL comprising an amino acid sequence having at least 99% sequence identity to SEQ ID NO:68.

24. The anti-glyco-CD44 antibody or antigen-binding fragment of claim 3, which has a VH comprising the amino acid sequence of SEQ ID NO:67 and a VL comprising the amino acid sequence of SEQ ID NO:68.

25. The anti-glyco-CD44 antibody or antigen-binding fragment of claim 3, which has:
(a) a VH comprising an amino acid sequence having at least 95% sequence identity to SEQ ID NO:206 and a VL comprising an amino acid sequence having at least 95% sequence identity to SEQ ID NO:207;
(b) a VH comprising an amino acid sequence having at least 97% sequence identity to SEQ ID NO:206 and a VL comprising an amino acid sequence having at least 97% sequence identity to SEQ ID NO:207; or
(c) a VH comprising an amino acid sequence having at least 99% sequence identity to SEQ ID NO:206 and a VL comprising an amino acid sequence having at least 99% sequence identity to SEQ ID NO:207.

26. The anti-glyco-CD44 antibody or antigen-binding fragment of claim 3, which has a VH comprising the amino acid sequence of SEQ ID NO:206 and a VL comprising the amino acid sequence of SEQ ID NO:207.

27. An anti-glyco-CD44 antibody or antigen-binding fragment that competes with a reference antibody or antigen binding fragment comprising (i) a heavy chain variable (VH) sequence of SEQ ID NO:1 and a light chain variable (VL) sequence of SEQ ID NO:2 (ii) a heavy chain variable (VH) sequence of SEQ ID NO:23 and a light chain variable (VL) sequence of SEQ ID NO:24, (iii) a heavy chain variable (VH) sequence of SEQ ID NO:45 and a light chain variable (VL) sequence of SEQ ID NO:46, (iv) a heavy chain variable (VH) sequence of SEQ ID NO:67 and a light chain variable (VL) sequence of SEQ ID NO:68, or (v) a heavy chain variable (VH) sequence of SEQ ID NO:206 and a light chain variable (VL) sequence of SEQ ID NO:207 for binding to a CD44v6 peptide GYRQTPKEDSHSTTGTAAA (SEQ ID NO:165) that has been glycosylated with GalNAc on threonine at amino acid position 5 of SEQ ID NO: 165 and serine at amino acid position 12 of SEQ ID NO: 165, the anti-glyco-CD44 antibody or antigen-binding fragment comprising:
(a) a VH sequence with first, second and third CDR means within the VH sequence; and
(b) a VL sequence with fourth, fifth and sixth CDR means within the VL sequence,
wherein the first, second, third, fourth, fifth, and sixth CDR means cooperate to effect binding of the anti-glyco-CD44 antibody or antigen-binding fragment to the CD44 peptide that competes with binding of the reference antibody or antigen binding fragment.

28. The anti-glyco-CD44 antibody or antigen-binding fragment of claim 1, which preferentially binds to a glyco-CD44 epitope that is overexpressed on cancer cells as compared to normal cells.

29. The anti-glyco-CD44 antibody or antigen-binding fragment of claim 1, which binds to a CD44v6 peptide GYRQTPKEDSHSTTGTAAA (SEQ ID NO:165) that has been glycosylated with STn on threonine at amino acid position 5 of SEQ ID NO: 165 and serine at amino acid position 12 of SEQ ID NO: 165, with a binding affinity (K_{D}) of:
(a) 1 nM to 200 nM as measured by surface plasmon resonance or bio-layer interferometry;
(b) 1 nM to 150 nM as measured by surface plasmon resonance or bio-layer
(c) 1 nM to 100 nM as measured by surface plasmon resonance or bio-layer interferometry;
(d) 1 nM to 50 nM as measured by surface plasmon resonance or bio-layer interferometry;
(e) 5 nM to 200 nM as measured by surface plasmon resonance or bio-layer interferometry;
(f) 5 nM to 100 nM as measured by surface plasmon resonance or bio-layer interferometry;
(g) 5 nM to 50 nM as measured by surface plasmon resonance or bio-layer interferometry;
(h) 5 nM to 25 nM as measured by surface plasmon resonance or bio-layer interferometry;
(i) 5 nM to 10 nM as measured by surface plasmon resonance or bio-layer interferometry;
(j) 10 nM to 200 nM as measured by surface plasmon resonance or bio-layer interferometry;
(k) 10 nM to 150 nM as measured by surface plasmon resonance or bio-layer interferometry;
(l) 10 nM to 100 nM as measured by surface plasmon resonance or bio-layer interferometry;
(m) 10 nM to 50 nM as measured by surface plasmon resonance or bio-layer interferometry;
(n) 10 nM to 25 nM as measured by surface plasmon resonance or bio-layer interferometry;
(o) 50 nM to 200 nM as measured by surface plasmon resonance or bio-layer interferometry;
(p) 50 nM to 150 nM as measured by surface plasmon resonance or bio-layer interferometry;
(q) 50 nM to 100 nM as measured by surface plasmon resonance or bio-layer interferometry;
(r) 100 nM to 200 nM as measured by surface plasmon resonance or bio-layer interferometry; or
(s) 100 nM to 150 nM as measured by surface plasmon resonance or bio-layer interferometry.

30. The anti-glyco-CD44 antibody or antigen-binding fragment of claim 1, which does not specifically bind to a first MUC1 glycopeptide having the sequence (VTSAPDTRPAPGSTAPPAHG)₃ (SEQ ID NO:205) that has been glycosylated in vitro using purified recombinant human glycosyltransferases GalNAc-T1, GalNAc-T2, and GalNAc-T4.

31. The anti-glyco-CD44 antibody or antigen-binding fragment of claim 30, which has a binding affinity to the CD44v6 glycopeptide which is at least 3 times, at least 5 times, at least 10 times, at least 20 times, at least 50 times, or at least 200 times the binding affinity of the anti-glyco-CD44 antibody or antigen-binding fragment to the first MUC1 glycopeptide, optionally wherein the binding affinity is measured by surface plasmon resonance, and further optionally where in the surface plasmon resonance is measured in the presence of saturating amounts of either the anti-glyco-CD44v6 peptide or the second MUC1 glycopeptide.

32. The anti-glyco-CD44 antibody or antigen-binding fragment of claim 1, which does not specifically bind to a second MUC1 glycopeptide having the sequence TAPPAHGVTSAPDTRPAPGSTAPPAHGVT (SEQ ID NO: 260) that has been glycosylated in vitro with GalNAcon the serine and threonine residues shown with bold and underlined text.

33. The anti-glyco-CD44 antibody or antigen-binding fragment of claim 32, which has a binding affinity to the CD44v6 glycopeptide which is at least 3 times, at least 5 times, at least 10 times, at least 20 times, at least 50 times, or at least 200 times the binding affinity of the anti-glyco-CD44 antibody or antigen-binding fragment to the second MUC1 glycopeptide, optionally wherein the binding affinity is measured by surface plasmon resonance, and further optionally where in the surface plasmon resonance is measured in the presence of saturating amounts of either the anti-glyco-CD44v6 peptide or the second MUC1 glycopeptide.

34. The anti-glyco-CD44 antibody or antigen-binding fragment of claim 1, which is multivalent.

35. The anti-glyco-CD44 antibody or antigen-binding fragment of claim 1, which is in the form of a single-chain variable fragment (scFv).

36. The anti-glyco-CD44 antibody or antigen-binding fragment of claim 1, which is in the form of a multispecific antibody.

37. The anti-glyco-CD44 antibody or antigen-binding fragment of claim 36, wherein the multispecific antibody is a bispecific antibody that binds to a second epitope that is different from the first epitope.

38. The anti-glyco-CD44 antibody or antigen-binding fragment of claim 37, wherein the bispecific antibody is a CrossMab, a Fab-arm exchange antibody, a bispecific T-cell engager (BiTE), or a dual-affinity retargeting molecule (DART).

39. The anti-glyco-CD44 antibody or antigen-binding fragment of claim 37, wherein the second epitope is a CD44 epitope.

40. The anti-glyco-CD44 antibody of antigen-binding fragment of claim 39, wherein the second epitope is a CD44 epitope that is overexpressed on cancer cells as compared to normal cells.

41. The anti-glyco-CD44 antibody or antigen-binding fragment of claim 37, wherein the second epitope is a T-cell epitope.

42. The anti-glyco-CD44 antibody or antigen-binding fragment of claim 41, wherein the T-cell epitope comprises a CD3 epitope, a CD8 epitope, a CD16 epitope, a CD25 epitope, a CD28 epitope, or an NKG2D epitope.

43. A fusion protein comprising the amino acid sequence of the anti-glyco-CD44 antibody or antigen-binding fragment of any of claims 1 to 42 operably linked to at least a second amino acid sequence.

44. A chimeric antigen receptor (CAR) comprising the scFv of claim 35.

45. The CAR of claim 44, which comprises the amino acid sequence of SEQ ID NO:157, SEQ ID NO:158, SEQ ID NO:159, SEQ ID NO:160, SEQ ID NO:161, SEQ ID NO:162, SEQ ID NO:163, SEQ ID NO:164, or SEQ ID NO:261.

46. An antibody-drug conjugate comprising the anti-glyco-CD44 antibody or antigen-binding fragment of any of claims 1 to 42 conjugated to a cytotoxic agent.

47. A nucleic acid comprising a coding region for an anti-glyco-CD44 antibody or antigen-binding fragment of any of claims 1 to 42.

48. A vector comprising the nucleic acid of claim 47.

49. A host cell engineered to express the nucleic acid of claim 47.

50. A host cell comprising the vector of claim 48.

51. A pharmaceutical composition comprising (a) the anti-glyco-CD44 antibody or antigen binding fragment of any of claims 1 to 42, and (b) a physiologically suitable buffer, adjuvant or diluent.

52. A method of treating cancer comprising administering to a subject in need thereof an effective amount of the anti-glyco-CD44 antibody or antigen binding fragment of any of claims 1 to 42.

53. The method of claim 52, wherein the subject is suffering from breast cancer, lung cancer, a urogenital cancer, pancreatic cancer, colorectal cancer, ovarian cancer, gastric cancer, head and neck cancer, skin cancer, malignant melanoma, liver cancer, a glioma, thyroid cancer, cervical cancer, or endometrial cancer.

54. A method of detecting cancer in a biological sample, comprising contacting a sample with an anti-glyco-CD44 antibody or antigen-binding fragment according to any one of claims 1 to 42 and detecting binding of the anti-glyco-CD44 antibody or antigen-binding fragment.

55. The method of claim 54, wherein the cancer is breast cancer, lung cancer, a urogenital cancer, pancreatic cancer, colorectal cancer, ovarian cancer, gastric cancer, head and neck cancer, skin cancer, malignant melanoma, liver cancer, a glioma, thyroid cancer, cervical cancer, or endometrial cancer.

56. A peptide of 12-30 amino acids in length comprising amino acids 4-13 of SEQ ID NO:165.

57. The peptide of claim 56 which is 15-25 amino acids in length

58. The peptide of claim 56 which is 18-20 amino acids in length

59. The peptide of claim 56 which consists of SEQ ID NO:165.

60. The peptide of any one of claims 56 to 58 which is O-glycosylated at the threonine corresponding to position 5 of SEQ ID NO:165 and/or the serine corresponding to position 12 of SEQ ID NO:165.

61. The peptide of claim 60, wherein the O-glycosylation comprises or consists of GalNAc.

62. A composition comprising the peptide of claim 60 or claim 61 and an adjuvant.

63. The composition of claim 62, wherein the adjuvant comprises an aluminum salt.

64. A method of generating antibodies against a tumor-associated form of CD44v6, comprising administering to an animal the peptide of claim 60 or claim 61 or the composition of claim 62 or claim 63.

65. The method of claim 64, further comprises collecting antibodies from the animal.

66. A method of eliciting an immune response against a tumor-associated form of CD44v6, comprising administering to a subject the peptide of embodiment claim 60 or claim 61 or the composition of claim 62 or claim 63.

67. The method of any one of claims 64 to 66, wherein the animal is a mouse or rabbit.
